SEQUENCE LISTING (256 sequences)

SEQ ID NO: 1 - DNA, 317 bp, synthetic construct
  source 1..317: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 2 - DNA, 314 bp, synthetic construct
  source 1..314: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccacc

SEQ ID NO: 3 - DNA, 964 bp, synthetic construct
  source 1..964: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacctgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggacctgcctgaaatcactttttttcaggttgggcggccgccacc

SEQ ID NO: 4 - DNA, 409 bp, synthetic construct
  source 1..409: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccacc

SEQ ID NO: 5 - DNA, 508 bp, synthetic construct
  source 1..508: mol_type = other DNA
ggggaggctgctggtgaatattaaccaaggtcaccccagttatcggaggagcaaacagggactaagtccacgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 6 - DNA, 582 bp, synthetic construct
  source 1..582: mol_type = other DNA
gttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 7 - DNA, 537 bp, synthetic construct
  source 1..537: mol_type = other DNA
agccaatgaaatacaaagatgagtctagttaataatctacaattattggttaaagaagtatattagtgctaatttccctccgtttgtcctagcttttctcgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 8 - DNA, 591 bp, synthetic construct
  source 1..591: mol_type = other DNA
gctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 9 - DNA, 573 bp, synthetic construct
  source 1..573: mol_type = other DNA
ctcgaggtcaattcacgcgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgccacc

SEQ ID NO: 10 - DNA, 579 bp, synthetic construct
  source 1..579: mol_type = other DNA
ctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccacc

SEQ ID NO: 11 - DNA, 544 bp, synthetic construct
  source 1..544: mol_type = other DNA
gcgacctgcccagcacaccctggggcagcgccgtgacgtcagcacgccgggcggggaccgggagatccttggggcggtggggggccagcggcagttcccggcggcccgccgcgtaagccacgcccactacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 12 - DNA, 620 bp, synthetic construct
  source 1..620: mol_type = other DNA
gaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataacgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 13 - DNA, 618 bp, synthetic construct
  source 1..618: mol_type = other DNA
gaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataaccgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 14 - DNA, 529 bp, synthetic construct
  source 1..529: mol_type = other DNA
gtcaacgaccgaccttgaggcatacttcaaagactgtttgtttaaagactgggaggagttgggggaggagattaggttaaaggtctttgtacgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 15 - DNA, 437 bp, synthetic construct
  source 1..437: mol_type = other DNA
gccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 16 - DNA, 564 bp, synthetic construct
  source 1..564: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 17 - DNA, 563 bp, synthetic construct
  source 1..563: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaag

SEQ ID NO: 18 - DNA, 581 bp, synthetic construct
  source 1..581: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccacc

SEQ ID NO: 19 - DNA, 964 bp, synthetic construct
  source 1..964: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcctgcctgaaatcactttttttcaggttgggcggccgccacc

SEQ ID NO: 20 - DNA, 586 bp, synthetic construct
  source 1..586: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 21 - DNA, 603 bp, synthetic construct
  source 1..603: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccacc

SEQ ID NO: 22 - DNA, 603 bp, synthetic construct
  source 1..603: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaag

SEQ ID NO: 23 - DNA, 585 bp, synthetic construct
  source 1..585: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaag

SEQ ID NO: 24 - DNA, 581 bp, synthetic construct
  source 1..581: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccacc

SEQ ID NO: 25 - DNA, 567 bp, synthetic construct
  source 1..567: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagggcggccgccacc

SEQ ID NO: 26 - DNA, 659 bp, synthetic construct
  source 1..659: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 27 - DNA, 586 bp, synthetic construct
  source 1..586: mol_type = other DNA
cagttaatgattaacttcgcgcctgtttactttggctcgcgtgcccgtctggactttggactcggcgcgcgagttaatgattaacttcgcgtttattgatttcgcgcgcgcattgcacaatacgcgtgcgcgcgctggactttggactcgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 28 - DNA, 697 bp, synthetic construct
  source 1..697: mol_type = other DNA
ctaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttgggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 29 - DNA, 551 bp, synthetic construct
  source 1..551: mol_type = other DNA
gagttcaagaccagcctgggcaagtctctttaaaaaaaacaaaacaaacaaacaaaaaaattaggcatggtggcacatgcctgtagtcctagctacttaggaggctgacgtaggaggatcgtttggacctgagaggtcaaggctacagtgagccatgattgtgccactgcactccagcctgggtgacagagtgagactggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 30 - DNA, 704 bp, synthetic construct
  source 1..704: mol_type = other DNA
tctttgcccctattttagggataaggattctgaaatgtggagatggtaagtaaaattgcacaactgaagaatgagttacatgacttggctcaaatactggtcattgaactccagagcctgaatattcttaaccacttacatgatgcaagctcaccaaataaatagttcgaatgtattgtgacagagcggcattgatattcatctattcatgtggctttgagtaggaagaagaaaggatatcattctgaccagaggggtgaaaaacaacctgcatctgatcctgaggcataatactattaacacaattcttttatgtttcagttcgatgaatttaaacctcttgtggaagagggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 31 - DNA, 702 bp, synthetic construct
  source 1..702: mol_type = other DNA
caacatggcagagaaaactcgagaacaagatgagcactggagaaatcattgtggatgagtcacactggcactaccaaagtggttttcgtctgctaaaaagacagagccaaatatgaaagcgcttaaacattaaccattttctggtgtacttttatctttgtaaatagaagataaagtatcatgatttgaatggaactctgagacccacattctttaatcatttactttccacttatgtggctctgttatgaaatctttgggagatggtttctgtcttcataacgtttttgttaatggcacaaaacagttcattattgcttttgacagaaacttgttgtttccggggttgggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 32 - DNA, 704 bp, synthetic construct
  source 1..704: mol_type = other DNA
ggaataatgatagaatgagagtactaaaacccccacaactggccctacatgaatggccagctatctcaaaagagggactgtgcttgtcagagggaatcccttcaggggactcttggacaggttaaagtgccatggatatgttgtgtaatgggaagtgtaaacttacagggacttgatttcaaaggtcattagagaagttagtcacaacttctaaagcaactatcagaaaacagcttggactcactcaagtaaacaggggctttattggcttatagacctgggcagtccaggtgtagatctggttccagacaaggctgattcagagactccacgtggtcaaggctctgttgtggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 33 - DNA, 681 bp, synthetic construct
  source 1..681: mol_type = other DNA
ggagtgggctgcctggggttcatacgtcaccacacttgataatttacagaagcctttcacgcgttttttcctgtgtctcaaacggggtaccaggaggctcagggaaacagcgcaggcgcctccccggtggcttctctctggcgccctctggtgggcggggcccgggtgagcggggtcccctcggggcctcagggagccgcagccccagcggaaccaggagggcgccctccgcccagccgcgctgccacttccgggttgtttgctcccggagccataaccgcagctcagcgtgcgggcagagccggctgacaggagacaggacacagcaggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 34 - DNA, 702 bp, synthetic construct
  source 1..702: mol_type = other DNA
atcctcatggaaaacatcttttctaagggtggggctggctcctgaggagcactccagctgaaagaaaggattgctgccactcctagttcccatcctatttaaatctgcaagaggtttggttaatcattggctttgtcctgtgtagacagtcaaccctccctctacgtcctttgttcctcctatgctgttggtatcccagaaattcttgctcaattcccctcagcttgtttgcttccgataagctgttgcaaaggtagcatttgattctccagtcaacaattaggagatctcacattccaggaacagggtggctttgagaatgaaaagaggagcataggagggtatatccggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 35 - DNA, 633 bp, synthetic construct
  source 1..633: mol_type = other DNA
aaacaagatacacatctctctttgaggagtgccctaacttcccatcattttgtccaattaaatgaattgaagaaatttaatgtttctaaactagaccaacaaagaataatagttgtatgacaagtaaataagctttgctgggaagatgttgcttaaatgataaaatggttcagccaacaagtgaaccaaaaattaaatattaactaaggaaaggtaaccatttctgaagtcattcctagcagaggactcagatatatataggattgaagatctctcagttggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 36 - DNA, 673 bp, synthetic construct
  source 1..673: mol_type = other DNA
gcccggagctccgagccttgtagtgtcagcactgtcacctctcaggctccaattgtccctttatgtaagctcctgggatagccaggactgcaaatggctggagctgatcacggggcctccttaccagaagggtgggcaggtctttcccgttcctttttcctcatcctggcccccaacctatactgtcaggttcccagattttgcacacaggttgaagctcctcctttttggcccagctcaaggttttttctctgatccaaaattatctgcaaaggctagtgaagcattatccattgctgtgaggaggagggcagtcctggggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 37 - DNA, 701 bp, synthetic construct
  source 1..701: mol_type = other DNA
tccaaatggaacagaccacacattacataatacatgtttgtatttttcttcagctagctagtgttgttagaactaaagacggtgctaaaattagtgctgtatatttccatctccctttaatttctctttcacttcccaactgaactttgtgcagtccttcatggcttatggaaattcccccatcattacagggctcagtggtcaggagggagttgggcctctgcagggtggggacactaaatatgctgagtggcctcagggccagaggttgtggcttctagccctggcagggccatcactatccacttggcctttagcaagaatcctctggtcctggggcctcactctggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 38 - DNA, 611 bp, synthetic construct
  source 1..611: mol_type = other DNA
ccccgacagacagaggagctgtgcaaacagaaagaaatggctgcgctttgttgctgttgctgtatcttggctggtgtcccccatcctggggtgccaggactgcagacctagacaccctcatccacttctgcttacaggaaccagtgtatccaccaggaggtaccgaggggggaccgggcaggactgggaaacaggacacaaccctcatggctgctgttattatgaaaataggagctcagctgcagcctctccatctgcggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 39 - DNA, 656 bp, synthetic construct
  source 1..656: mol_type = other DNA
actgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtacctggctcagaaaccacagcgtcctgtgtccaaggtggagggggtggcgtggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 40 - DNA, 489 bp, synthetic construct
  source 1..489: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 41 - DNA, 489 bp, synthetic construct
  source 1..489: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtcaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 42 - DNA, 412 bp, synthetic construct
  source 1..412: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagggcggccgccacc

SEQ ID NO: 43 - DNA, 809 bp, synthetic construct
  source 1..809: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcctgcctgaaatcactttttttcaggttgggcggccgccacc

SEQ ID NO: 44 - DNA, 504 bp, synthetic construct
  source 1..504: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggaagaggtaagggtttaagggatggttggttggtggggtattaatgtttaattacctggagcacctgcctgaaatcactttttttcaggttgggcggccgccacc

SEQ ID NO: 45 - DNA, 551 bp, synthetic construct
  source 1..551: mol_type = other DNA
tcccatgtggggctgcaggaaagggtgaggaagcctgcggtatgagttggcaagttgggtttgactgccatggggccctggagcagagccatccaggaggcagctgcctgcgctgcgaggaaaggagagaggtctgcactgcactcagagctctggaggcatcagcagtttctgctaggcctggaagacatgggtagagattgaaaaagccagccagaagggccagagggggaaagcaagaagagccatgttagaactgtgacactcacgactctcggcctggtctccaatcccaggtgggccagtgaggatgctggagctgggccagctcattcccagcaggggcagataggaggttgttggggacagaaggaaacttttcctaaactggcagtacccacagttcccaatctcctgttgcacaactgagtgtcaggaaacagtcaggtggacttcccaggctgggccaactccaggggaaagcccaggccagcctgtttgccctgttcacaacgaatcaaaccctagctaaggtcacaggatccgccacc

SEQ ID NO: 46 - DNA, 886 bp, synthetic construct
  source 1..886: mol_type = other DNA
tggtgggggctggggagggccccagacatgagaccagctcctcccccaggggatgttatcagtgggtccagagggcaaaatagggagcctggtggagggaggggcaaaggcctcgggctctgagcggccttggcccttctccaccaacccctgccctacactaagggggaggcagcggggggcacacagggtgggggcgggtggggggctgctgggtgagcagcactcgcctgcctggattgaaacccagagatggaggtgctgggaggggctgtgagagctcagccctgtaaccaggccttgccggagccactgatgcctggtcttctgtgcctttactccaaacaccccccagcccaagccacccacttgttctcaagtctgaagaagcccctcacccctctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggggatccgccacc

SEQ ID NO: 47 - DNA, 764 bp, synthetic construct
  source 1..764: mol_type = other DNA
tgacagagccattgaaataaatcctgattcagctcagccttacaagtggcgggggaaagcacacagacttctaggccactgggaagaagcagcccatgatcttgcctttgcctgtaaattggattatgatgaagatgctagtgcaatgctgaaagaagttcaacctagggcacagaaaattgcagaacattggagaaagtatgagcgaaaacatgaagagcgagagatcaaagaaagaatagaacgagttaagaaggctcaagaagagcaggagagagcccagagggaggaagaagccagacgacagtcaggagctcactatggcccttttccaggtggctttcctggtggaatgcctggtaattttcccggaggaatgcctggaatgggaggggacatgcctggaatggccggaatgcctggactcaatgaaattcttagtgatccagaggctcttgcagccatgcaggatccagagaggagcagagctggtctcctgccctgacagctgccaggcacatcttgttccctcaggttgcacaactgggataaatgacccgggatgaagaaaccactggcatccaggaacttgtcttagaccgttttgtaggggaaatgacctgcagggactttccccagggaccacatccagcttttcttcgctcccaagaaaccagcagggaaggctcagtataaatagcagccaccgctccctggcaggcagggacccgcagctcagctacagcacagatggatccgccacc

SEQ ID NO: 48 - DNA, 632 bp, synthetic construct
  source 1..632: mol_type = other DNA
cgtctcattacacattaactcaaaaacggacaagatcatttacacttgccctcttacccgaccctcattcccctaacccccatagccctcaaccctgtccctgatttcaattcctttctcctttcttctgctccccaatatctctctgccaagttgcagtaaagtgggataaggttgagagatgagatctacccataatggaataaagacaccatgagctttccatggtatgatgggttgatggtattccatgggttgatatgtcagagctttccagagaaataacttggaatcctgcttcctgttgcactcaagtccaaggacctcagatctcaaaagaatgaacctcaaatatacctgaagtgtacccccttagcctccactaagagctgtaccccctgcctctcaccccatcaccatgagtcttccatgtgcttgtcctctcctcccccatttctccaacttgtttatcctcacataatccctgccccactgggcccatccatagtccctgtcacctgacagggggtgggtaaacagacaggtatatagccccttcctctccagccagggcaggcacagacaccaaggacagagacgctggctaggtaagataaggaggatccgccacc

SEQ ID NO: 49 - DNA, 763 bp, synthetic construct
  source 1..763: mol_type = other DNA
cccgcccctccccagcctactgcctccacccgaagtctacttcctgggtgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggatccgccacc

SEQ ID NO: 50 - DNA, 625 bp, synthetic construct
  source 1..625: mol_type = other DNA
tccttaaaataattatgaaacactgtcttatgggttgtgccctgtcaacaagaatgctattgttaaataattagcatgtttgttgatgctggccaatatttacttttaggtatcatcacttctgatgtggattttttgtcaattttccacataactgcagatataagtttggactcatcaacacagccatttgctttcaagacattaaatgctgtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaggatccgccacc

SEQ ID NO: 51 - DNA, 512 bp, synthetic construct
  source 1..512: mol_type = other DNA
gggaggacagggcgggggaagggggaggggagtgaagtagtctccctggaatgctggtggtgggggaggcagtctccttggtggaggagtcccagcgtccctcccctcccctcctctgccaacacaatggacaatggcaactgcccacacactcccatggaggggaaggggatgagtgcagggaaccccgaccccacccgggagacctgcaagcctgcagacactcccctcccgcccccactgaacccttgacccctgccctgcagcccccgcagcttgctgtttgcccactctatttgcccagccccagggacagagctgatccttgaactcttaagttccacattgccaggaccagtgagcagcaacagggccggggctgggcttatcagcctcccagcccagaccctggctgcagacataaataggccctgcaagagctggctgcttagagactgcgagaaggaggtgcgtcctgctgcctgccccggtcactctggggatccgccacc

SEQ ID NO: 52 - DNA, 712 bp, synthetic construct
  source 1..712: mol_type = other DNA
aggcctgtcagctcctcacccgcccacccacccgcccgcctgcattcttcggtctaaacttaccaggtggaagctacaactctagacaggaagtaactaactgggttggatccggtcgagttccgctgcgcttcttagagccgctgctgggaagaggccgaaacccagtggccgcagcagtagccaccgcctctgttactagggggtctcgcctccatcgctggctgcgaggagtctgagggcacgtgaccggaagtagcaaggcgggccctgggcacgtgactcaagagcgcggccgccatcacaccttccgctagttccctccctcccccacgggttcccgcctgtttcgtttcctctctccggtctcgagcggcgaagggtcctgggatcctggctggcccaaccatttggcctagagctcctccggttccagtcggcgccgtgtctccagcccgcaacggccctgcggtcgcaggccgtagagagcgtaatcgcgaccgcggcctgagggggagggaccacgcggaaggaggggcgtgactcgtcacgtgttgaggaagtgccgttagtagcgttgtttccagtctgcggctctcctggttgacgtcagcaaggaaggggaggaacgcgaggggagactgaagttcacgttccgctcttagtgcgcggtgagttccccggcgctaagccgggagacggatccgccacc

SEQ ID NO: 53 - DNA, 522 bp, synthetic construct
  source 1..522: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcaccataaaagaagccgccctagccacgtcccctcgcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 54 - DNA, 332 bp, synthetic construct
  source 1..332: mol_type = other DNA
tcttttcattctcaaagccaccctgttcctggaatgtgagatctcctaattgttgactggagaatcaaatgctacctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaagatgttttccggatccgccacc

SEQ ID NO: 55 - DNA, 662 bp, synthetic construct
  source 1..662: mol_type = other DNA
tgttctcactcatagatgggaattgaacaatgagaatacttggacacaggaaggggaacatcacactctggggactgttgtggggtggggggaggggggagggaatggcacataaaaaaatgtgtccatggctctgggaggagcatgtttgttttcctcatttcccagtctgtaaataagcaaattgaaaggggttagtgataatgtccatctccagaagctgtcagatttcctttgtcaaactctatgatttgggctgaagtaggtgttggagaggcagctaccacgtgcacccagatggccactcgtttaatatgttaccatttcccattattttcgcaggatagatagccaaagtggagccctgagagatttcttcatttttcctgtcataaagaattggtaattcagtagtcataggagtttgtaataaataactcacattgatttctctgttctgaaataattttgcttcccctcttcccgaagctctgacacctgccccaacaagcaatgttggaaaattatttacatagtggcgcaaactcccttactgctttggatataaatccaggcaggaggaggtagctctaaggcaagagatctaggacttctagcccctgaactttcagccgaatacatcttttccaggatccgccacc

SEQ ID NO: 56 - DNA, 457 bp, synthetic construct
  source 1..457: mol_type = other DNA
agcctggggaccctcaaaggtgcttcatcattaggtttgtggctgggtcctactgaagtaagtcttggcactcagagggataggaattgaatgaagacatgagattcctctgcgggaggcctctctaggaaatctgtggactcacacgtttactaatgttgctgcagccccgcacccaccttggccttgggcagccatactctagggcttttgtaacctctccatgtgaggaactcaaattagacctgggtttggaggcggtgctccgagctggcctttgggggaggttttgtgcgaggcatttcccaagtgctggcaggattgtgtcacagacacagagtaaacttttgctgggctccaagtgaccgcccatagtttattataaaggtgactgcaccctgcagccaccagcactgcctggctccacgtgcctcctggtctcagtggatccgccacc

SEQ ID NO: 57 - DNA, 655 bp, synthetic construct
  source 1..655: mol_type = other DNA
agactagctttccactcctccttgtcttctctctgcagtgccctgggagctgtcattgccctcctgctctggggacagctttttaagaacaactaaccctttttaatgaataaatcattcagttgactttctcatctgcctatttaggctaatcaccagactataaaaccaagttgtgtgatttcttgcaaagagcaatcagagaagacacaataaacacatttactgatttcaggcaaaaggaagtactttctttagagccccacctaagctaggctgcagaaatgtctacaatgggtttgaaaaaactcaaaatgagcctttctgcagtgtgaaaatcctccaagataaagagacagattgatggttcctgccgccgccctgtcctgcccagttgctgatttcaggaaatactttggcaggtttgtgggtcatagagttgccaggtttcttgggatttgtaatagaacatcacaagaaaatcaagtgtgaagcaagagctcaactcttaacaggggtattgtttgtggttttgttactggaaaagatagtgaccttaccagggccaaagtttgtagacacaggaattacgaaattgagaagggggagaagtgagctagtggcagcataaaaagaccagcagggatccgccacc

SEQ ID NO: 58 - DNA, 512 bp, synthetic construct
  source 1..512: mol_type = other DNA
ccctaggctaaaataatagtgttaactgatccctaagctaagaaagttcttttggtaattcaggtgatggcagcaggacccatcttaaggatagactaggtttgcttagttcgaggtcatatctgtttgctctcagccatgtactggaagaagttgcatcacacagcctccaggactgccctcctcctcacagcaatggataatgcttcactagcctttgcagataattttggatcagagaaaaaaccttgagctgggccaaaaaggaggagcttcaacctgtgtgcaaaatctgggaacctgacagtataggttgggggccaggatgaggaaaaaggaacgggaaagacctgcccacccttctggtaaggaggccccgtgatcagctccagccatttgcagtcctggctatcccaggagcttacataaagggacaattggagcctgagaggtgacagtgctgacactacaaggctcggagctccgggcactcagacatcggatccgccacc

SEQ ID NO: 59 - DNA, 602 bp, synthetic construct
  source 1..602: mol_type = other DNA
attttgcccacaggaatcctcgtagcccactacctccagagcatcacgccttgcctggccctgggtggagagctggtctaccaccggcggcctgctgagcaccaggggcagaggcggccagacaacctcagggaggagtgtcctggcgtccccatcctccaaagggcctgggcccgccccgagggggcagcgagaggagcttccccatccccgtcctctccagattacattcatccaggcacaggaaaggacagggtcaggaaaggaggactctgggcggcagcctccacattccccttccacgcttggcccccagattggaggagggtgtctgtattactgggcgaggtgtcctcccttcctggggactgtggggggtggtcaaaagacctctttgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggatccgccacc

SEQ ID NO: 60 - DNA, 553 bp, synthetic construct
  source 1..553: mol_type = other DNA
gttcaaaggaattctctggtatttttctgtgtgtgtacacatgcacccacacacttgtgtgtatatgtgtacagacatatataaacacatgcatataatgtgtatatatgcattacatatatgcacattcatacatctttatgtacaaaatacatatatgtatatataaacaccgatgtacaaacacatacgcacacatctatatacatacacatgtgtgtgcacatatacacatacctgcatatacacacatttcgtggggtgcggagagtcacttaaaggctgcagggccataaggcttcctgcttgactgatattcattaatgtttgctgaattacagcaaacctttgctgtgcccatcctgttctttatcattgaccattgctctcaggagttaatgtttgaacctggccataaaggaatcaacagctgctgacctctggccgctattcgaagggaagggagccccctataaaacagcctacagtggacagtctggtcggcagagccgcaggtcagtcgtgaagagggagctctattggatccgccacc

SEQ ID NO: 61 - DNA, 961 bp, synthetic construct
  source 1..961: mol_type = other DNA
ttttgtgccattaacaaaaacgttatgaagacagaaaccatctcccaaagatttcataacagagccacataagtggaaagtaaatgattaaagaatgtgggtctcagagttccattcaaatcatgatactttatcttctatttacaaagataaaagtacaccagaaaatggttaatgtttaagcgcaaaactaaaaggcagaaatgaaatccacaagcagacagcccgcgccacaccctgggcctggtggttaaagattgacccctgacctaatccgttaggttatctatagattacagacattgtatagaaaagcactgtgaaaatccctattctgttttgttccgatctaattaccggtgcatgcagcccccagtcacgcatcccctgcttgttcaatcgatcacgaccctctcacgtgcacccacttagagttgtgagcccttaaaaggaacagggattgctcactcggggagctcggctcttgagacaggaatcttgcccattccccgaacgaataaaccccttccttaactcagcgtctgaggaattttgtctgcggctcctcctgctacattctgagtggggaaagggactaaggtggtctgaggaccccacagagtcaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggccagtgtgactcatccacaatgatttctccagtgctcatcttgttctcgagttttctctgccggatccgccacc

SEQ ID NO: 62 - DNA, 310 bp, synthetic construct
  source 1..310: mol_type = other DNA
ttttgtgccattaacaaaaacgttatgaagacagaaaccatctcccaaagatttcataacagagccacataagtggaaagtaaatgattaaagaatgtgggtctcagagttccattcaaatcatgatactttatcttctatttacaaagataaaagtacaccagaaaatggttaatgtttaagcgctttcatatttggctctgtctttttagcagacgaaaaccactttggtagtgccagtgtgactcatccacaatgatttctccagtgctcatcttgttctcgagttttctctgccggatccgccacc

SEQ ID NO: 63 - DNA, 282 bp, synthetic construct
  source 1..282: mol_type = other DNA
caggtggccctgaccctgggagagtccagggcagggtgcagctgcattcatgctgctggggaacatgccctcaggttactcaccccatggacatgttggccccagggactgaaaagcttaggaaatggtattgagaaatctggggcagccccaaaaggggagaggccatggggagaagggggggctgagtgggggaaaggcaggagccagataaaaagccagctccagcaggcgctgctcactcctccccatcctctccctctgtccctcggatccgccacc

SEQ ID NO: 64 - DNA, 536 bp, synthetic construct
  source 1..536: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 65 - DNA, 559 bp, synthetic construct
  source 1..559: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccaggggcaggttggtatcaaggttacaagacaggtttaaggagaccaatagaaactgggcatgtggagacagagaagactcttgggtttctgataggcactgactctctctgcctattggtctattttcccacccttaggctgcttcttgccaaccaacc

SEQ ID NO: 66 - DNA, 786 bp, synthetic construct
  source 1..786: mol_type = other DNA
agccaatgaaatacaaagatgagtctagttaataatctacaattattggttaaagaagtatattagtgctaatttccctccgtttgtcctagcttttctcgagttgctggtgcttccccaggctggagattgagttaatattaacaggcccaaggcgatgtgggcttgtgcaatcataggcccggcctcgccaggtcacctgaggagttaatgaatacatatctccttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccaggggcaggttggtatcaaggttacaagacaggtttaaggagaccaatagaaactgggcatgtggagacagagaagactcttgggtttctgataggcactgactctctctgcctattggtctattttcccacccttaggctgcttcttgccaaccaacc

SEQ ID NO: 67 - DNA, 910 bp, synthetic construct
  source 1..910: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccaggggcaggttggtatcaaggttacaagacaggtttaaggagaccaatagaaactgggcatgtggagacaggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtagaagactcttgggtttctgataggcactgactctctctgcctattggtctattttcccacccttaggctgcttcttgccaaccaacc

SEQ ID NO: 68 - DNA, 761 bp, synthetic construct
  source 1..761: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgaggtcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccaccaaccaaccggatccgccacc

SEQ ID NO: 69 - DNA, 996 bp, synthetic construct
  source 1..996: mol_type = other DNA
agccaatgaaatacaaagatgagtctagttaataatctacaattattggttaaagaagtatattagtgctaatttccctccgtttgtcctagcttttctcgaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataacgagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 70 - DNA, 658 bp, synthetic construct
  source 1..658: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcc

SEQ ID NO: 71 - DNA, 663 bp, synthetic construct
  source 1..663: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgatcagtccgcctggagacctcgagccgagtggtcgtggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcc

SEQ ID NO: 72 - DNA, 666 bp, synthetic construct
  source 1..666: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcaggtggattccgagccgagtggtcgtttgggcagggcaggttggtatcaaggttacaagacaggtttaaggagaccaatagaaactgggcatgtggagacagagaagactcttgggtttctgataggcactgactctctctgcctattggtctattttcccacccttaggctgctttccttccaaa

SEQ ID NO: 73 - DNA, 1272 bp, synthetic construct
  source 1..1272: mol_type = other DNA
ctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctcctctagaggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcctcgacggtatcgataagcttgatatcgaattctagtcgtcgaccactttcacaatctgcggcaccggtacc

SEQ ID NO: 74 - DNA, 461 bp, synthetic construct
  source 1..461: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 75 - DNA, 264 bp, synthetic construct
  source 1..264: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcggccgccacc

SEQ ID NO: 76 - DNA, 779 bp, synthetic construct
  source 1..779: mol_type = other DNA
cactgggaggatgttgagtaagatggaaaactactgatgacccttgcagagacagagtattaggacatgtttgaacaggggccgggcgatcagcaggtaggtcttgtgtctgccgggcaatgagcaaggctccttcctgtccaagctccccgcccctccccagcctactgcctccacccgaagtctacttcctgggtgggcaggaactgggcactgtgcccagggcatggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 77 - DNA, 678 bp, synthetic construct
  source 1..678: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccgggcaggttggtatcaaggttacaagacaggtttaaggagaccaatagaaactgggcatgtggagacagagaagactcttgggtttctgataggcactgactctctctgcctattggtctattttcccacccttaggctgctggatccgccacc

SEQ ID NO: 78 - DNA, 666 bp, synthetic construct
  source 1..666: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctcctgggcaggttggtatcaaggttacaagacaggtttaaggagaccaatagaaactgggcatgtggagacagagaagactcttgggtttctgataggcactgactctctctgcctattggtctattttcccacccttaggctgcttcttgccaaccaacc

SEQ ID NO: 79 - DNA, 893 bp, synthetic construct
  source 1..893: mol_type = other DNA
agccaatgaaatacaaagatgagtctagttaataatctacaattattggttaaagaagtatattagtgctaatttccctccgtttgtcctagcttttctcgagttgctggtgcttccccaggctggagattgagttaatattaacaggcccaaggcgatgtgggcttgtgcaatcataggcccggcctcgccaggtcacctgaggagttaatgaatacatatctccttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctcctgggcaggttggtatcaaggttacaagacaggtttaaggagaccaatagaaactgggcatgtggagacagagaagactcttgggtttctgataggcactgactctctctgcctattggtctattttcccacccttaggctgcttcttgccaaccaacc

SEQ ID NO: 80 - DNA, 1017 bp, synthetic construct
  source 1..1017: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctcctgggcaggttggtatcaaggttacaagacaggtttaaggagaccaatagaaactgggcatgtggagacaggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtagaagactcttgggtttctgataggcactgactctctctgcctattggtctattttcccacccttaggctgcttcttgccaaccaacc

SEQ ID NO: 81 - DNA, 868 bp, synthetic construct
  source 1..868: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgaggtcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaaccggatccgccacc

SEQ ID NO: 82 - DNA, 255 bp, synthetic construct
  source 1..255: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgagccaagaggtaagggtttaagggatggttggttggtggggtattaatgtttaattacctggagcacctgcctgaaatcactttttttcaggttggggatccgccacc

SEQ ID NO: 83 - DNA, 283 bp, synthetic construct
  source 1..283: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 84 - DNA, 332 bp, synthetic construct
  source 1..332: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgggtctgtctcttgcttcaacagtgtttggacggaacagatccggggacgagccaagaggtaagggtttaagggatggttggttggtggggtattaatgtttaattacctggagcacctgcctgaaatcactttttttcaggttggggatccgccacc

SEQ ID NO: 85 - DNA, 455 bp, synthetic construct
  source 1..455: mol_type = other DNA
gttaatcattaagtcgttaatttttgtggcccttgcgatgtttgctctggttaataatctcaggacaaacagaggttaataattttccagatctctctgagcaatagtataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaaccgagccaagaggtaagggtttaagggatggttggttggtggggtattaatgtttaattacctggagcacctgcctgaaatcactttttttcaggttggggatccgccacc

SEQ ID NO: 86 - DNA, 460 bp, synthetic construct
  source 1..460: mol_type = other DNA
gttaatcattaagtcgttaatttttgtggcccttgcgatgtttgctctggttaataatctcaggacaaacagaggttaataattttccagatctctctgagcaatagtataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaaccgagccgagccaagaggtaagggtttaagggatggttggttggtggggtattaatgtttaattacctggagcacctgcctgaaatcactttttttcaggttggggatccgccacc

SEQ ID NO: 87 - DNA, 593 bp, synthetic construct
  source 1..593: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatccttgagccaagaggtaagggtttaagggatggttggttggtggggtattaatgtttaattacctggagcacctgcctgaaatcactttttttcaggttggggatccgccacc

SEQ ID NO: 88 - DNA, 1415 bp, synthetic construct
  source 1..1415: mol_type = other DNA
cacacacaccatggacatacacacacaccacacaagcaccgtgcacacaaatatacacaaaccacatacacacgtgacacacatacacacatgcaacatgcacacacaccacaaacacatgcaccatgcacacacatacacaccacaaacatacatacacacgcatgcaccatgcacacacatatacacaccacgcacatacatgcatcatgcacggacacaccccacacatatacacatgcaccatgcacatacacaccacacacacacgcaccatgcacacacatatacagccaccacatacatacatatgtcacacacacatacacaggacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 89 - DNA, 1332 bp, synthetic construct
  source 1..1332: mol_type = other DNA
cacacacactcacacaaagcacatttacacacatgcacacagtcatacatgcacacccttgtacacacaacacacacacatgcacacaggcacacacacatgcacacataacgtgaatatcacgtgcagtcacacacatgcaaacacacacacagttacatacattcaaacacatgtgcacataatacatgcatgcatagagtcacacacacatgcagtcacacacaacacatacatgcacacagtcacacagaagcagtcacacacacatgcaccacatacacagacaaaagcacacaggcacacacactacacacacacacgaacacatatgcacacagtctcacacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 90 - DNA, 1411 bp, synthetic construct
  source 1..1411: mol_type = other DNA
tcgaaaccaaattgggcgacatagtgagacctggtctctacagcaaatacacacacacacacacacacacacttatgcacacatacaccatacatacacatacagttagtcacacatatacacacatacacacaccacacacacatacacacatagatacacaaatacatatacaccacacacatacacacacgccacacaccatacacacatacatacacacatcacacatacatatatacaaatacatatatacacaaatacacacatacacacacatactacacacaccacaaaccatacacacattcatacataccccccacacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 91 - DNA, 1333 bp, synthetic construct
  source 1..1333: mol_type = other DNA
aaaagaacaaaattgttcaaaaggtgatgttttaaaatgtgatatctacagctacatttacgaaatcacaatcgaatgtttaaggaaacagctgtatactgcctaaacacaagtgaaaagcatcatagcttacgtgataccactaaaaccacagacatcagctgagattgtccactacagactgcagtaccatgtcaatattgcacaaattgggacatcacgacatcaagtagtcattcacagccaaccaatcacgccacccaccatgcacagcaaaatatataacacacatttctttcaattattacactgtcataaactcaaaatccttttagaacaattctgttcttacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 92 - DNA, 1377 bp, synthetic construct
  source 1..1377: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 93 - DNA, 1410 bp, synthetic construct
  source 1..1410: mol_type = other DNA
gtggttgctgtaaccaatgaccacaaacctgggggtcatggaaagaacacaaactttacacacacacacacacacacacacacacaggcacatacacacatgcacatatgcatacaaagacaggcacacacgagcatgcacacacacaaacacgtgcacatacacatgcgcacacacatgcacacgcacatgcacacacacgcaggtacacaagcatgcaaacacacatacacatacaaacgcgtgcatgcatacatgcacacatgagcacacaaacacatgcacacacttgcacacatgcacacaaaggcatacacatgcaatgcaacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 94 - DNA, 1317 bp, synthetic construct
  source 1..1317: mol_type = other DNA
acacaccatgcacaaaccacacacacatgcaacactccacagacatacacacagatacacagatacaccacgccacatagacacaccatcacaccacacacacaaaacacatcacatagagacacacaccacacacatacaagatgccacatagatacaccacacacaaaccacacacacacacctcatagacacacaaaccatgcacactatgcacaaaccacacacacgcatacatacccacaacacaccacatacacaccatgcataccacacccacactccacatagacacacaaaccacgcacacatacatacacacaacacaccatatacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 95 - DNA, 1426 bp, synthetic construct
  source 1..1426: mol_type = other DNA
acaaatgcacacacacagatgcagggtacacacgaatgcacacacagcacacacagatgcacagtatacacgaatgcacagacagcacacacatagatgcagggtacacatgaatacacacagcacacacagatgcacagtacacatgaatgcacacacagatgcatggtacacacggatgcacagacagcacacacacagatgcagggtacacatgaatgcacacacagcacacacacagatgcacagtacacatgaatgcacacacagcacacagatgcacagtacacacgaatgcacacacagcacatacacacatgcagggtacacacgaatgcaaagcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 96 - DNA, 1317 bp, synthetic construct
  source 1..1317: mol_type = other DNA
cacacaaatacacacacacacaaatacgcacacacaaatacagaaacacacacacatacacaaatacacacacacacaaatacgcacacacacaaatacagaaacacacacatacacaaatacacaaatacaaaaatacgcacaaatacacaatacacacacaaatacacataaatacacaaatacgcacacacaaatacatataaatacacacaaatacacacaaatacacacagaaatacacacgcacacgcacacacacaaaacacatacacatacacacgaatacacacaaatacacacacaaatacacatacacgcacacacatgcacaacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtttcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 97 - DNA, 621 bp, synthetic construct
  source 1..621: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatcctgatccacaagctcgagccgagtggtcgttgagccaagaggtaagggtttaagggatggttggttggtggggtattaatgtttaattacctggagcacctgcctgaaatcactttttttcaggttggggatccgccacc

SEQ ID NO: 98 - DNA, 536 bp, synthetic construct
  source 1..536: mol_type = other DNA
tccggtaaaacgctagccgccgaaaattgcgcaagcccccaccctaccgcctacgaaaggagccgtttcgggaatccgcggggccgcggggacccccccaagccgccattacgcgaaacgaccacgggaacctcccgctatgtcgggtttcttcccggcggcgggccgagaggaggtcttgtgccgaaatcgtacgtcccggcattcggcgcggcgttggtccacggacatatcgccgaaagcccttcccgaacggccgtctgccgagggccggcgaaagacaaaagccgctttggggcaaaaagccgttacgccatacggaaagctcgggccgaaccgtcgtcggtcggttgtgctctttgaaagtgttttgccggaagagtttcggggactctcttcgggcgtccggccggcttccgttttcggttccgcttgcgactgtcggaacgatcttttcggccgttttctgtttccgggacgggcgggtgccgttttcgccgttagcgccttcttgccaaccaaccggatccgccacc

SEQ ID NO: 99 - DNA, 414 bp, synthetic construct
  source 1..414: mol_type = other DNA
aattgagtccaaagtccagaattattgtgcaataaatttaaggaagaggaatttgttacgtaataattcgatgacctttgacccctggaattctgccccgcccccactaatttgttacgtaataattaatgccacgtcatcaattattacgtaacaaattgttaatgattaactaattcctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 100 - DNA, 506 bp, synthetic construct
  source 1..506: mol_type = other DNA
aattagttaatcattaacaattatcacgtgacaatttttcccagaagaattcttctgggaaaaattgttaatgattaacaatttgacgtcaaattctgattggtccaattggtcacatgacaattaatgccacgtcatcaattattacgtaacaaattattacgtaacaaattatcacgtgataattatgagtcatccaattggatgactcataattgccggcgaattctgacgtcaaattgtgacgtaaaattatgagtcatccaattacttccggtcaatcctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 101 - DNA, 422 bp, synthetic construct
  source 1..422: mol_type = other DNA
atttgagtcatttcaattaatgagtcacaaattgagtccaaagtccagaattatgagtcatccaattattgcacaataattcaaagtaaacaaattctgattggtccaattgaccggaagtaattgatgacgtggcattaattatcacgtgacaattacttccggtcaattgcatccatagaattgttaatcattaaccctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 102 - DNA, 279 bp, synthetic construct
  source 1..279: mol_type = other DNA
cagtttcatttccaattgatgagtcaccaatttgacgtcaaattatgattggtcccctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 103 - DNA, 464 bp, synthetic construct
  source 1..464: mol_type = other DNA
agagtccaaagtccaaatttgacgtcagaattccaggggtcaaaggtcatcgaattaatgagtcacaaattgttaatgattaaggaagaggaattaatgagtcacaaattggaaatgaaactgaacttccggtcaattgatgagtcaccaattgatgagtcaccaattggtcacatgacaattggtgactcatcaattagagtccaaagtccaaattctgattggtccaattatcacgtgcctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 104 - DNA, 546 bp, synthetic construct
  source 1..546: mol_type = other DNA
aattagttaatcattaacaatttgacgtcaaatttttcccagaagaatttgtgactcattaattattgtgcaataatttattgtgcaataatttgacgtcagaattaatgagtcacaaattaatgccacgtcatcaattgtcacgtgataattaatgagtcacaaatttgacgtcagaattgtcatgtgaccaattagttaatcattaacaattccttttccttaaattagagtccaaagtccaaattcctcttccttaaattattgcacaataaattgcatccatagaattgttaatcattaacaattattgcacaataatcctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 105 - DNA, 444 bp, synthetic construct
  source 1..444: mol_type = other DNA
cgtgataattctgacgtcaaattgtcacgtgataattggtgactcatcaattgccggcgaattattgtgcaataaattaatgccacgtcatcaattgtcacgtgataattttacgtcacaattcaaagtaaacaaattatcacgtgataattcgatgacctttgacccctggaattcctcttccttaaattcgccggcaattgttaatgattaactaattcctgacgtcccaatgatggcgcctgctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 106 - DNA, 639 bp, synthetic construct
  source 1..639: mol_type = other DNA
aattgagtccaaagtccagaattattgtgcaataaatttaaggaagaggaatttgttacgtaataattcgatgacctttgacccctggaattctgccccgcccccactaatttgttacgtaataattaatgccacgtcatcaattattacgtaacaaattgttaatgattaactaattccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 107 - DNA, 743 bp, synthetic construct
  source 1..743: mol_type = other DNA
aattagttaatcattaacaattatcacgtgacaatttttcccagaagaattcttctgggaaaaattgttaatgattaacaatttgacgtcaaattctgattggtccaattggtcacatgacaattaatgccacgtcatcaattattacgtaacaaattattacgtaacaaattatcacgtgataattatgagtcatccaattggatgactcataattgccggcgaattctgacgtcaaattgtgacgtaaaattatgagtcatccaattacttccggtcaatccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 108 - DNA, 659 bp, synthetic construct
  source 1..659: mol_type = other DNA
atttgagtcatttcaattaatgagtcacaaattgagtccaaagtccagaattatgagtcatccaattattgcacaataattcaaagtaaacaaattctgattggtccaattgaccggaagtaattgatgacgtggcattaattatcacgtgacaattacttccggtcaattgcatccatagaattgttaatcattaacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 109 - DNA, 516 bp, synthetic construct
  source 1..516: mol_type = other DNA
cagtttcatttccaattgatgagtcaccaatttgacgtcaaattatgattggtccccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 110 - DNA, 701 bp, synthetic construct
  source 1..701: mol_type = other DNA
agagtccaaagtccaaatttgacgtcagaattccaggggtcaaaggtcatcgaattaatgagtcacaaattgttaatgattaaggaagaggaattaatgagtcacaaattggaaatgaaactgaacttccggtcaattgatgagtcaccaattgatgagtcaccaattggtcacatgacaattggtgactcatcaattagagtccaaagtccaaattctgattggtccaattatcacgtgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 111 - DNA, 783 bp, synthetic construct
  source 1..783: mol_type = other DNA
aattagttaatcattaacaatttgacgtcaaatttttcccagaagaatttgtgactcattaattattgtgcaataatttattgtgcaataatttgacgtcagaattaatgagtcacaaattaatgccacgtcatcaattgtcacgtgataattaatgagtcacaaatttgacgtcagaattgtcatgtgaccaattagttaatcattaacaattccttttccttaaattagagtccaaagtccaaattcctcttccttaaattattgcacaataaattgcatccatagaattgttaatcattaacaattattgcacaataatccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 112 - DNA, 681 bp, synthetic construct
  source 1..681: mol_type = other DNA
cgtgataattctgacgtcaaattgtcacgtgataattggtgactcatcaattgccggcgaattattgtgcaataaattaatgccacgtcatcaattgtcacgtgataattttacgtcacaattcaaagtaaacaaattatcacgtgataattcgatgacctttgacccctggaattcctcttccttaaattcgccggcaattgttaatgattaactaattccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgccacc

SEQ ID NO: 113 - DNA, 728 bp, synthetic construct
  source 1..728: mol_type = other DNA
aattgagtccaaagtccagaattattgtgcaataaatttaaggaagaggaatttgttacgtaataattcgatgacctttgacccctggaattctgccccgcccccactaatttgttacgtaataattaatgccacgtcatcaattattacgtaacaaattgttaatgattaactaattgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 114 - DNA, 832 bp, synthetic construct
  source 1..832: mol_type = other DNA
aattagttaatcattaacaattatcacgtgacaatttttcccagaagaattcttctgggaaaaattgttaatgattaacaatttgacgtcaaattctgattggtccaattggtcacatgacaattaatgccacgtcatcaattattacgtaacaaattattacgtaacaaattatcacgtgataattatgagtcatccaattggatgactcataattgccggcgaattctgacgtcaaattgtgacgtaaaattatgagtcatccaattacttccggtcaatgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 115 - DNA, 748 bp, synthetic construct
  source 1..748: mol_type = other DNA
atttgagtcatttcaattaatgagtcacaaattgagtccaaagtccagaattatgagtcatccaattattgcacaataattcaaagtaaacaaattctgattggtccaattgaccggaagtaattgatgacgtggcattaattatcacgtgacaattacttccggtcaattgcatccatagaattgttaatcattaacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 116 - DNA, 605 bp, synthetic construct
  source 1..605: mol_type = other DNA
cagtttcatttccaattgatgagtcaccaatttgacgtcaaattatgattggtccgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 117 - DNA, 790 bp, synthetic construct
  source 1..790: mol_type = other DNA
agagtccaaagtccaaatttgacgtcagaattccaggggtcaaaggtcatcgaattaatgagtcacaaattgttaatgattaaggaagaggaattaatgagtcacaaattggaaatgaaactgaacttccggtcaattgatgagtcaccaattgatgagtcaccaattggtcacatgacaattggtgactcatcaattagagtccaaagtccaaattctgattggtccaattatcacgtggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 118 - DNA, 872 bp, synthetic construct
  source 1..872: mol_type = other DNA
aattagttaatcattaacaatttgacgtcaaatttttcccagaagaatttgtgactcattaattattgtgcaataatttattgtgcaataatttgacgtcagaattaatgagtcacaaattaatgccacgtcatcaattgtcacgtgataattaatgagtcacaaatttgacgtcagaattgtcatgtgaccaattagttaatcattaacaattccttttccttaaattagagtccaaagtccaaattcctcttccttaaattattgcacaataaattgcatccatagaattgttaatcattaacaattattgcacaataatgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 119 - DNA, 770 bp, synthetic construct
  source 1..770: mol_type = other DNA
cgtgataattctgacgtcaaattgtcacgtgataattggtgactcatcaattgccggcgaattattgtgcaataaattaatgccacgtcatcaattgtcacgtgataattttacgtcacaattcaaagtaaacaaattatcacgtgataattcgatgacctttgacccctggaattcctcttccttaaattcgccggcaattgttaatgattaactaattgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagccgggcaggttggtatcaaggttacaagacaggtttaaggagaccaatagaaactgggcatgtggagacagagaagactcttgggtttctgataggcactgactctctctgcctattggtctattttcccacccttaggctgctgccacc

SEQ ID NO: 120 - DNA, 461 bp, synthetic construct
  source 1..461: mol_type = other DNA
aattgagtccaaagtccagaattattgtgcaataaatttaaggaagaggaatttgttacgtaataattcgatgacctttgacccctggaattctgccccgcccccactaatttgttacgtaataattaatgccacgtcatcaattattacgtaacaaattgttaatgattaactaattgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 121 - DNA, 565 bp, synthetic construct
  source 1..565: mol_type = other DNA
aattagttaatcattaacaattatcacgtgacaatttttcccagaagaattcttctgggaaaaattgttaatgattaacaatttgacgtcaaattctgattggtccaattggtcacatgacaattaatgccacgtcatcaattattacgtaacaaattattacgtaacaaattatcacgtgataattatgagtcatccaattggatgactcataattgccggcgaattctgacgtcaaattgtgacgtaaaattatgagtcatccaattacttccggtcaatgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 122 - skipped

SEQ ID NO: 123 - DNA, 481 bp, synthetic construct
  source 1..481: mol_type = other DNA
atttgagtcatttcaattaatgagtcacaaattgagtccaaagtccagaattatgagtcatccaattattgcacaataattcaaagtaaacaaattctgattggtccaattgaccggaagtaattgatgacgtggcattaattatcacgtgacaattacttccggtcaattgcatccatagaattgttaatcattaacgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 124 - DNA, 338 bp, synthetic construct
  source 1..338: mol_type = other DNA
cagtttcatttccaattgatgagtcaccaatttgacgtcaaattatgattggtccgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 125 - DNA, 523 bp, synthetic construct
  source 1..523: mol_type = other DNA
agagtccaaagtccaaatttgacgtcagaattccaggggtcaaaggtcatcgaattaatgagtcacaaattgttaatgattaaggaagaggaattaatgagtcacaaattggaaatgaaactgaacttccggtcaattgatgagtcaccaattgatgagtcaccaattggtcacatgacaattggtgactcatcaattagagtccaaagtccaaattctgattggtccaattatcacgtggttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 126 - DNA, 605 bp, synthetic construct
  source 1..605: mol_type = other DNA
aattagttaatcattaacaatttgacgtcaaatttttcccagaagaatttgtgactcattaattattgtgcaataatttattgtgcaataatttgacgtcagaattaatgagtcacaaattaatgccacgtcatcaattgtcacgtgataattaatgagtcacaaatttgacgtcagaattgtcatgtgaccaattagttaatcattaacaattccttttccttaaattagagtccaaagtccaaattcctcttccttaaattattgcacaataaattgcatccatagaattgttaatcattaacaattattgcacaataatgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 127 - DNA, 503 bp, synthetic construct
  source 1..503: mol_type = other DNA
cgtgataattctgacgtcaaattgtcacgtgataattggtgactcatcaattgccggcgaattattgtgcaataaattaatgccacgtcatcaattgtcacgtgataattttacgtcacaattcaaagtaaacaaattatcacgtgataattcgatgacctttgacccctggaattcctcttccttaaattcgccggcaattgttaatgattaactaattgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 128 - DNA, 796 bp, synthetic construct
  source 1..796: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactagccacc

SEQ ID NO: 129 - DNA, 771 bp, synthetic construct
  source 1..771: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcctcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccgccacc

SEQ ID NO: 130 - DNA, 776 bp, synthetic construct
  source 1..776: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaagccacc

SEQ ID NO: 131 - DNA, 665 bp, synthetic construct
  source 1..665: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtacttgccacc

SEQ ID NO: 132 - DNA, 858 bp, synthetic construct
  source 1..858: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttcagccacc

SEQ ID NO: 133 - DNA, 654 bp, synthetic construct
  source 1..654: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcctctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggggccacc

SEQ ID NO: 134 - DNA, 637 bp, synthetic construct
  source 1..637: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggggccacc

SEQ ID NO: 135 - DNA, 843 bp, synthetic construct
  source 1..843: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagagccacc

SEQ ID NO: 136 - DNA, 662 bp, synthetic construct
  source 1..662: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtgccacc

SEQ ID NO: 137 - DNA, 664 bp, synthetic construct
  source 1..664: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttggccacc

SEQ ID NO: 138 - DNA, 991 bp, synthetic construct
  source 1..991: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctgagccacc

SEQ ID NO: 139 - DNA, 799 bp, synthetic construct
  source 1..799: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctctgccacc

SEQ ID NO: 140 - DNA, 806 bp, synthetic construct
  source 1..806: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggcgccacc

SEQ ID NO: 141 - DNA, 873 bp, synthetic construct
  source 1..873: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggccgccacc

SEQ ID NO: 142 - DNA, 727 bp, synthetic construct
  source 1..727: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccgctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgcgccacc

SEQ ID NO: 143 - DNA, 698 bp, synthetic construct
  source 1..698: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggccccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgacgccacc

SEQ ID NO: 144 - DNA, 813 bp, synthetic construct
  source 1..813: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaaccgccacc

SEQ ID NO: 145 - DNA, 621 bp, synthetic construct
  source 1..621: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctagccacc

SEQ ID NO: 146 - DNA, 1341 bp, synthetic construct
  source 1..1341: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtggccacc

SEQ ID NO: 147 - DNA, 967 bp, synthetic construct
  source 1..967: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtggccacc

SEQ ID NO: 148 - DNA, 748 bp, synthetic construct
  source 1..748: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcctgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctggccacc

SEQ ID NO: 149 - DNA, 1037 bp, synthetic construct
  source 1..1037: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattcgcggcccgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagtttagccacc

SEQ ID NO: 150 - DNA, 305 bp, synthetic construct
  source 1..305: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 151 - DNA, 647 bp, synthetic construct
  source 1..647: mol_type = other DNA
aagaggtaagggtttaagggatggttggttggtggggtattaatgtttaattacctggagcgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacctgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggacctgcctgaaatcactttttttcaggttgg

SEQ ID NO: 152 - DNA, 555 bp, synthetic construct
  source 1..555: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacctgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagg

SEQ ID NO: 153 - DNA, 151 bp, synthetic construct
  source 1..151: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcg

SEQ ID NO: 154 - DNA, 404 bp, synthetic construct
  source 1..404: mol_type = other DNA
cgcgttcgccccgccccggacggtatcgataagcttaggagcttgggctgcaggtcgagggcactgggaggatgttgagtaagatggaaaactactgatgacccttgcagagacagagtattaggacctgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagg

SEQ ID NO: 155 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 156 - DNA, 71 bp, synthetic construct
  source 1..71: mol_type = other DNA
ggggaggctgctggtgaatattaaccaaggtcaccccagttatcggaggagcaaacagggactaagtccac

SEQ ID NO: 157 - DNA, 411 bp, synthetic construct
  source 1..411: mol_type = other DNA
cgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgt

SEQ ID NO: 158 - DNA, 411 bp, synthetic construct
  source 1..411: mol_type = other DNA
cgcgtaagccacgcccactacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgt

SEQ ID NO: 159 - DNA, 16 bp, synthetic construct
  source 1..16: mol_type = other DNA
cgagccgagtggtcgt

SEQ ID NO: 160 - DNA, 18 bp, synthetic construct
  source 1..18: mol_type = other DNA
ctcgagccgagtggtcgt

SEQ ID NO: 161 - DNA, 145 bp, synthetic construct
  source 1..145: mol_type = other DNA
gttaatttttaaaaagcagtcaaaagtccaagtggcccttggcagcatttactctctctgtttgctctggttaataatctcaggagcacaaacattcctggaggcaggagaagaaatcaacatcctggacttatcctctgggcct

SEQ ID NO: 162 - DNA, 100 bp, synthetic construct
  source 1..100: mol_type = other DNA
agccaatgaaatacaaagatgagtctagttaataatctacaattattggttaaagaagtatattagtgctaatttccctccgtttgtcctagcttttctc

SEQ ID NO: 163 - DNA, 154 bp, synthetic construct
  source 1..154: mol_type = other DNA
gctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 164 - DNA, 192 bp, synthetic construct
  source 1..192: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 165 - DNA, 107 bp, synthetic construct
  source 1..107: mol_type = other DNA
gcgacctgcccagcacaccctggggcagcgccgtgacgtcagcacgccgggcggggaccgggagatccttggggcggtggggggccagcggcagttcccggcggccc

SEQ ID NO: 166 - DNA, 183 bp, synthetic construct
  source 1..183: mol_type = other DNA
gaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataac

SEQ ID NO: 167 - DNA, 92 bp, synthetic construct
  source 1..92: mol_type = other DNA
gtcaacgaccgaccttgaggcatacttcaaagactgtttgtttaaagactgggaggagttgggggaggagattaggttaaaggtctttgtac

SEQ ID NO: 168 - DNA, 87 bp, synthetic construct
  source 1..87: mol_type = other DNA
gagttgctggtgcttccccaggctggagattgagttaatattaacaggcccaaggcgatgtgggcttgtgcaatcataggcccggcc

SEQ ID NO: 169 - DNA, 40 bp, synthetic construct
  source 1..40: mol_type = other DNA
tcgccaggtcacctgaggagttaatgaatacatatctcct

SEQ ID NO: 170 - skipped

SEQ ID NO: 171 - DNA, 397 bp, synthetic construct
  source 1..397: mol_type = other DNA
aagaggtaagggtttaagggatggttggttggtggggtattaatgtttaattacctggagcacaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcctgcctgaaatcactttttttcaggttgg

SEQ ID NO: 172 - DNA, 21 bp, synthetic construct
  source 1..21: mol_type = other DNA
aactcctaaaaaaccgccacc

SEQ ID NO: 173 - DNA, 21 bp, synthetic construct
  source 1..21: mol_type = other DNA
aactcctaaaaaaccgccaag

SEQ ID NO: 174 - DNA, 148 bp, synthetic construct
  source 1..148: mol_type = other DNA
agttaatgattaacttcgcgcctgtttactttggctcgcgtgcccgtctggactttggactcggcgcgcgagttaatgattaacttcgcgtttattgatttcgcgcgcgcattgcacaatacgcgtgcgcgcgctggactttggactc

SEQ ID NO: 175 - DNA, 344 bp, synthetic construct
  source 1..344: mol_type = other DNA
ctaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttg

SEQ ID NO: 176 - DNA, 249 bp, synthetic construct
  source 1..249: mol_type = other DNA
ggtacccgggggcgcgccagtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagg

SEQ ID NO: 177 - DNA, 198 bp, synthetic construct
  source 1..198: mol_type = other DNA
gagttcaagaccagcctgggcaagtctctttaaaaaaaacaaaacaaacaaacaaaaaaattaggcatggtggcacatgcctgtagtcctagctacttaggaggctgacgtaggaggatcgtttggacctgagaggtcaaggctacagtgagccatgattgtgccactgcactccagcctgggtgacagagtgagact

SEQ ID NO: 178 - DNA, 351 bp, synthetic construct
  source 1..351: mol_type = other DNA
tctttgcccctattttagggataaggattctgaaatgtggagatggtaagtaaaattgcacaactgaagaatgagttacatgacttggctcaaatactggtcattgaactccagagcctgaatattcttaaccacttacatgatgcaagctcaccaaataaatagttcgaatgtattgtgacagagcggcattgatattcatctattcatgtggctttgagtaggaagaagaaaggatatcattctgaccagaggggtgaaaaacaacctgcatctgatcctgaggcataatactattaacacaattcttttatgtttcagttcgatgaatttaaacctcttgtggaagag

SEQ ID NO: 179 - DNA, 349 bp, synthetic construct
  source 1..349: mol_type = other DNA
caacatggcagagaaaactcgagaacaagatgagcactggagaaatcattgtggatgagtcacactggcactaccaaagtggttttcgtctgctaaaaagacagagccaaatatgaaagcgcttaaacattaaccattttctggtgtacttttatctttgtaaatagaagataaagtatcatgatttgaatggaactctgagacccacattctttaatcatttactttccacttatgtggctctgttatgaaatctttgggagatggtttctgtcttcataacgtttttgttaatggcacaaaacagttcattattgcttttgacagaaacttgttgtttccggggttg

SEQ ID NO: 180 - DNA, 351 bp, synthetic construct
  source 1..351: mol_type = other DNA
ggaataatgatagaatgagagtactaaaacccccacaactggccctacatgaatggccagctatctcaaaagagggactgtgcttgtcagagggaatcccttcaggggactcttggacaggttaaagtgccatggatatgttgtgtaatgggaagtgtaaacttacagggacttgatttcaaaggtcattagagaagttagtcacaacttctaaagcaactatcagaaaacagcttggactcactcaagtaaacaggggctttattggcttatagacctgggcagtccaggtgtagatctggttccagacaaggctgattcagagactccacgtggtcaaggctctgttgt

SEQ ID NO: 181 - DNA, 328 bp, synthetic construct
  source 1..328: mol_type = other DNA
ggagtgggctgcctggggttcatacgtcaccacacttgataatttacagaagcctttcacgcgttttttcctgtgtctcaaacggggtaccaggaggctcagggaaacagcgcaggcgcctccccggtggcttctctctggcgccctctggtgggcggggcccgggtgagcggggtcccctcggggcctcagggagccgcagccccagcggaaccaggagggcgccctccgcccagccgcgctgccacttccgggttgtttgctcccggagccataaccgcagctcagcgtgcgggcagagccggctgacaggagacaggacacagca

SEQ ID NO: 182 - DNA, 349 bp, synthetic construct
  source 1..349: mol_type = other DNA
atcctcatggaaaacatcttttctaagggtggggctggctcctgaggagcactccagctgaaagaaaggattgctgccactcctagttcccatcctatttaaatctgcaagaggtttggttaatcattggctttgtcctgtgtagacagtcaaccctccctctacgtcctttgttcctcctatgctgttggtatcccagaaattcttgctcaattcccctcagcttgtttgcttccgataagctgttgcaaaggtagcatttgattctccagtcaacaattaggagatctcacattccaggaacagggtggctttgagaatgaaaagaggagcataggagggtatatcc

SEQ ID NO: 183 - DNA, 280 bp, synthetic construct
  source 1..280: mol_type = other DNA
aaacaagatacacatctctctttgaggagtgccctaacttcccatcattttgtccaattaaatgaattgaagaaatttaatgtttctaaactagaccaacaaagaataatagttgtatgacaagtaaataagctttgctgggaagatgttgcttaaatgataaaatggttcagccaacaagtgaaccaaaaattaaatattaactaaggaaaggtaaccatttctgaagtcattcctagcagaggactcagatatatataggattgaagatctctcagtt

SEQ ID NO: 184 - DNA, 320 bp, synthetic construct
  source 1..320: mol_type = other DNA
gcccggagctccgagccttgtagtgtcagcactgtcacctctcaggctccaattgtccctttatgtaagctcctgggatagccaggactgcaaatggctggagctgatcacggggcctccttaccagaagggtgggcaggtctttcccgttcctttttcctcatcctggcccccaacctatactgtcaggttcccagattttgcacacaggttgaagctcctcctttttggcccagctcaaggttttttctctgatccaaaattatctgcaaaggctagtgaagcattatccattgctgtgaggaggagggcagtcctgg

SEQ ID NO: 185 - DNA, 348 bp, synthetic construct
  source 1..348: mol_type = other DNA
tccaaatggaacagaccacacattacataatacatgtttgtatttttcttcagctagctagtgttgttagaactaaagacggtgctaaaattagtgctgtatatttccatctccctttaatttctctttcacttcccaactgaactttgtgcagtccttcatggcttatggaaattcccccatcattacagggctcagtggtcaggagggagttgggcctctgcagggtggggacactaaatatgctgagtggcctcagggccagaggttgtggcttctagccctggcagggccatcactatccacttggcctttagcaagaatcctctggtcctggggcctcactct

SEQ ID NO: 186 - DNA, 258 bp, synthetic construct
  source 1..258: mol_type = other DNA
ccccgacagacagaggagctgtgcaaacagaaagaaatggctgcgctttgttgctgttgctgtatcttggctggtgtcccccatcctggggtgccaggactgcagacctagacaccctcatccacttctgcttacaggaaccagtgtatccaccaggaggtaccgaggggggaccgggcaggactgggaaacaggacacaaccctcatggctgctgttattatgaaaataggagctcagctgcagcctctccatctgc

SEQ ID NO: 187 - DNA, 303 bp, synthetic construct
  source 1..303: mol_type = other DNA
actgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtacctggctcagaaaccacagcgtcctgtgtccaaggtggagggggtggcgt

SEQ ID NO: 188 - DNA, 326 bp, synthetic construct
  source 1..326: mol_type = other DNA
cgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 189 - DNA, 531 bp, synthetic construct
  source 1..531: mol_type = other DNA
gtcttgtgtctgccgggcaatgagcaaggctccttcctgtccaagctccccgcccctccccagcctactgcctccacccgaagtctacttcctgggtgggcaggaactgggcactgtgcccagggcatggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagcc

SEQ ID NO: 190 - DNA, 402 bp, synthetic construct
  source 1..402: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccag

SEQ ID NO: 191 - DNA, 401 bp, synthetic construct
  source 1..401: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttcca

SEQ ID NO: 192 - DNA, 64 bp, synthetic construct
  source 1..64: mol_type = other DNA
tataaaagccccttcaccaggagaagccgtcacacagatccacaagctcgagccgagtggtcgt

SEQ ID NO: 193 - DNA, 142 bp, synthetic construct
  source 1..142: mol_type = other DNA
gggcaggttggtatcaaggttacaagacaggtttaaggagaccaatagaaactgggcatgtggagacagagaagactcttgggtttctgataggcactgactctctctgcctattggtctattttcccacccttaggctgct

SEQ ID NO: 194 - DNA, 160 bp, synthetic construct
  source 1..160: mol_type = other DNA
tgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtcta

SEQ ID NO: 195 - DNA, 493 bp, synthetic construct
  source 1..493: mol_type = other DNA
gggcaggttggtatcaaggttacaagacaggtttaaggagaccaatagaaactgggcatgtggagacaggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtagaagactcttgggtttctgataggcactgactctctctgcctattggtctattttcccacccttaggctgct

SEQ ID NO: 196 - DNA, 351 bp, synthetic construct
  source 1..351: mol_type = other DNA
gcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgt

SEQ ID NO: 197 - DNA, 210 bp, synthetic construct
  source 1..210: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgag

SEQ ID NO: 198 - DNA, 35 bp, synthetic construct
  source 1..35: mol_type = other DNA
tcagtccgcctggagacctcgagccgagtggtcgt

SEQ ID NO: 199 - DNA, 513 bp, synthetic construct
  source 1..513: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcaggtggattccgagccgagtggtcgtttgggca

SEQ ID NO: 200 - DNA, 328 bp, synthetic construct
  source 1..328: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgct

SEQ ID NO: 201 - DNA, 524 bp, synthetic construct
  source 1..524: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaacc

SEQ ID NO: 202 - DNA, 509 bp, synthetic construct
  source 1..509: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctcct

SEQ ID NO: 203 - DNA, 508 bp, synthetic construct
  source 1..508: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcct

SEQ ID NO: 204 - DNA, 145 bp, synthetic construct
  source 1..145: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcct

SEQ ID NO: 205 - DNA, 174 bp, synthetic construct
  source 1..174: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgt

SEQ ID NO: 206 - DNA, 173 bp, synthetic construct
  source 1..173: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcctgatccacaagctcgagccgagtggtcgt

SEQ ID NO: 207 - DNA, 178 bp, synthetic construct
  source 1..178: mol_type = other DNA
aattgagtccaaagtccagaattattgtgcaataaatttaaggaagaggaatttgttacgtaataattcgatgacctttgacccctggaattctgccccgcccccactaatttgttacgtaataattaatgccacgtcatcaattattacgtaacaaattgttaatgattaactaatt

SEQ ID NO: 208 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 209 - DNA, 282 bp, synthetic construct
  source 1..282: mol_type = other DNA
aattagttaatcattaacaattatcacgtgacaatttttcccagaagaattcttctgggaaaaattgttaatgattaacaatttgacgtcaaattctgattggtccaattggtcacatgacaattaatgccacgtcatcaattattacgtaacaaattattacgtaacaaattatcacgtgataattatgagtcatccaattggatgactcataattgccggcgaattctgacgtcaaattgtgacgtaaaattatgagtcatccaattacttccggtcaat

SEQ ID NO: 210 - DNA, 198 bp, synthetic construct
  source 1..198: mol_type = other DNA
atttgagtcatttcaattaatgagtcacaaattgagtccaaagtccagaattatgagtcatccaattattgcacaataattcaaagtaaacaaattctgattggtccaattgaccggaagtaattgatgacgtggcattaattatcacgtgacaattacttccggtcaattgcatccatagaattgttaatcattaac

SEQ ID NO: 211 - DNA, 55 bp, synthetic construct
  source 1..55: mol_type = other DNA
cagtttcatttccaattgatgagtcaccaatttgacgtcaaattatgattggtcc

SEQ ID NO: 212 - DNA, 240 bp, synthetic construct
  source 1..240: mol_type = other DNA
agagtccaaagtccaaatttgacgtcagaattccaggggtcaaaggtcatcgaattaatgagtcacaaattgttaatgattaaggaagaggaattaatgagtcacaaattggaaatgaaactgaacttccggtcaattgatgagtcaccaattgatgagtcaccaattggtcacatgacaattggtgactcatcaattagagtccaaagtccaaattctgattggtccaattatcacgtg

SEQ ID NO: 213 - DNA, 322 bp, synthetic construct
  source 1..322: mol_type = other DNA
aattagttaatcattaacaatttgacgtcaaatttttcccagaagaatttgtgactcattaattattgtgcaataatttattgtgcaataatttgacgtcagaattaatgagtcacaaattaatgccacgtcatcaattgtcacgtgataattaatgagtcacaaatttgacgtcagaattgtcatgtgaccaattagttaatcattaacaattccttttccttaaattagagtccaaagtccaaattcctcttccttaaattattgcacaataaattgcatccatagaattgttaatcattaacaattattgcacaataat

SEQ ID NO: 214 - DNA, 220 bp, synthetic construct
  source 1..220: mol_type = other DNA
cgtgataattctgacgtcaaattgtcacgtgataattggtgactcatcaattgccggcgaattattgtgcaataaattaatgccacgtcatcaattgtcacgtgataattttacgtcacaattcaaagtaaacaaattatcacgtgataattcgatgacctttgacccctggaattcctcttccttaaattcgccggcaattgttaatgattaactaatt

SEQ ID NO: 215 - DNA, 402 bp, synthetic construct
  source 1..402: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttatataacccaggggcacaggggctgccctcagtccgcctggagacctcgagccgagtggtcgtctcaggagcc

SEQ ID NO: 216 - DNA, 335 bp, synthetic construct
  source 1..335: mol_type = other DNA
cgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 217 - DNA, 310 bp, synthetic construct
  source 1..310: mol_type = other DNA
tcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacacc

SEQ ID NO: 218 - DNA, 315 bp, synthetic construct
  source 1..315: mol_type = other DNA
gaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaa

SEQ ID NO: 219 - DNA, 204 bp, synthetic construct
  source 1..204: mol_type = other DNA
gcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtactt

SEQ ID NO: 220 - DNA, 397 bp, synthetic construct
  source 1..397: mol_type = other DNA
gcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttca

SEQ ID NO: 221 - DNA, 193 bp, synthetic construct
  source 1..193: mol_type = other DNA
tctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggg

SEQ ID NO: 222 - DNA, 176 bp, synthetic construct
  source 1..176: mol_type = other DNA
cgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggg

SEQ ID NO: 223 - DNA, 382 bp, synthetic construct
  source 1..382: mol_type = other DNA
ggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaaga

SEQ ID NO: 224 - DNA, 201 bp, synthetic construct
  source 1..201: mol_type = other DNA
cgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagt

SEQ ID NO: 225 - DNA, 203 bp, synthetic construct
  source 1..203: mol_type = other DNA
cagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttg

SEQ ID NO: 226 - DNA, 530 bp, synthetic construct
  source 1..530: mol_type = other DNA
cgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctga

SEQ ID NO: 227 - DNA, 338 bp, synthetic construct
  source 1..338: mol_type = other DNA
ctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctct

SEQ ID NO: 228 - DNA, 345 bp, synthetic construct
  source 1..345: mol_type = other DNA
cgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggc

SEQ ID NO: 229 - DNA, 412 bp, synthetic construct
  source 1..412: mol_type = other DNA
caggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggcc

SEQ ID NO: 230 - DNA, 266 bp, synthetic construct
  source 1..266: mol_type = other DNA
gctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgc

SEQ ID NO: 231 - DNA, 237 bp, synthetic construct
  source 1..237: mol_type = other DNA
ccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgac

SEQ ID NO: 232 - DNA, 352 bp, synthetic construct
  source 1..352: mol_type = other DNA
cccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaacc

SEQ ID NO: 233 - DNA, 880 bp, synthetic construct
  source 1..880: mol_type = other DNA
cctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 234 - DNA, 506 bp, synthetic construct
  source 1..506: mol_type = other DNA
cctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 235 - DNA, 287 bp, synthetic construct
  source 1..287: mol_type = other DNA
tgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctg

SEQ ID NO: 236 - DNA, 351 bp, synthetic construct
  source 1..351: mol_type = other DNA
gttaatcattaagtcgttaatttttgtggcccttgcgatgtttgctctggttaataatctcaggacaaacagaggttaataattttccagatctctctgagcaatagtataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaaccgagcc

SEQ ID NO: 237 - DNA, 346 bp, synthetic construct
  source 1..346: mol_type = other DNA
gttaatcattaagtcgttaatttttgtggcccttgcgatgtttgctctggttaataatctcaggacaaacagaggttaataattttccagatctctctgagcaatagtataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaacc

SEQ ID NO: 238 - DNA, 223 bp, synthetic construct
  source 1..223: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgggtctgtctcttgcttcaacagtgtttggacggaacagatccggggac

SEQ ID NO: 239 - DNA, 879 bp, synthetic construct
  source 1..879: mol_type = other DNA
cacacacaccatggacatacacacacaccacacaagcaccgtgcacacaaatatacacaaaccacatacacacgtgacacacatacacacatgcaacatgcacacacaccacaaacacatgcaccatgcacacacatacacaccacaaacatacatacacacgcatgcaccatgcacacacatatacacaccacgcacatacatgcatcatgcacggacacaccccacacatatacacatgcaccatgcacatacacaccacacacacacgcaccatgcacacacatatacagccaccacatacatacatatgtcacacacacatacacaggacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtt

SEQ ID NO: 240 - DNA, 796 bp, synthetic construct
  source 1..796: mol_type = other DNA
cacacacactcacacaaagcacatttacacacatgcacacagtcatacatgcacacccttgtacacacaacacacacacatgcacacaggcacacacacatgcacacataacgtgaatatcacgtgcagtcacacacatgcaaacacacacacagttacatacattcaaacacatgtgcacataatacatgcatgcatagagtcacacacacatgcagtcacacacaacacatacatgcacacagtcacacagaagcagtcacacacacatgcaccacatacacagacaaaagcacacaggcacacacactacacacacacacgaacacatatgcacacagtctcacacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtt

SEQ ID NO: 241 - DNA, 875 bp, synthetic construct
  source 1..875: mol_type = other DNA
tcgaaaccaaattgggcgacatagtgagacctggtctctacagcaaatacacacacacacacacacacacacttatgcacacatacaccatacatacacatacagttagtcacacatatacacacatacacacaccacacacacatacacacatagatacacaaatacatatacaccacacacatacacacacgccacacaccatacacacatacatacacacatcacacatacatatatacaaatacatatatacacaaatacacacatacacacacatactacacacaccacaaaccatacacacattcatacataccccccacacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtt

SEQ ID NO: 242 - DNA, 797 bp, synthetic construct
  source 1..797: mol_type = other DNA
aaaagaacaaaattgttcaaaaggtgatgttttaaaatgtgatatctacagctacatttacgaaatcacaatcgaatgtttaaggaaacagctgtatactgcctaaacacaagtgaaaagcatcatagcttacgtgataccactaaaaccacagacatcagctgagattgtccactacagactgcagtaccatgtcaatattgcacaaattgggacatcacgacatcaagtagtcattcacagccaaccaatcacgccacccaccatgcacagcaaaatatataacacacatttctttcaattattacactgtcataaactcaaaatccttttagaacaattctgttcttacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtt

SEQ ID NO: 243 - DNA, 841 bp, synthetic construct
  source 1..841: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtt

SEQ ID NO: 244 - DNA, 874 bp, synthetic construct
  source 1..874: mol_type = other DNA
gtggttgctgtaaccaatgaccacaaacctgggggtcatggaaagaacacaaactttacacacacacacacacacacacacacacaggcacatacacacatgcacatatgcatacaaagacaggcacacacgagcatgcacacacacaaacacgtgcacatacacatgcgcacacacatgcacacgcacatgcacacacacgcaggtacacaagcatgcaaacacacatacacatacaaacgcgtgcatgcatacatgcacacatgagcacacaaacacatgcacacacttgcacacatgcacacaaaggcatacacatgcaatgcaacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtt

SEQ ID NO: 245 - DNA, 781 bp, synthetic construct
  source 1..781: mol_type = other DNA
acacaccatgcacaaaccacacacacatgcaacactccacagacatacacacagatacacagatacaccacgccacatagacacaccatcacaccacacacacaaaacacatcacatagagacacacaccacacacatacaagatgccacatagatacaccacacacaaaccacacacacacacctcatagacacacaaaccatgcacactatgcacaaaccacacacacgcatacatacccacaacacaccacatacacaccatgcataccacacccacactccacatagacacacaaaccacgcacacatacatacacacaacacaccatatacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtt

SEQ ID NO: 246 - DNA, 890 bp, synthetic construct
  source 1..890: mol_type = other DNA
acaaatgcacacacacagatgcagggtacacacgaatgcacacacagcacacacagatgcacagtatacacgaatgcacagacagcacacacatagatgcagggtacacatgaatacacacagcacacacagatgcacagtacacatgaatgcacacacagatgcatggtacacacggatgcacagacagcacacacacagatgcagggtacacatgaatgcacacacagcacacacacagatgcacagtacacatgaatgcacacacagcacacagatgcacagtacacacgaatgcacacacagcacatacacacatgcagggtacacacgaatgcaaagcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatccaatgatggcgcctgctgaattctgtacgggccagatatacgcgtt

SEQ ID NO: 247 - DNA, 781 bp, synthetic construct
  source 1..781: mol_type = other DNA
cacacaaatacacacacacacaaatacgcacacacaaatacagaaacacacacacatacacaaatacacacacacacaaatacgcacacacacaaatacagaaacacacacatacacaaatacacaaatacaaaaatacgcacaaatacacaatacacacacaaatacacataaatacacaaatacgcacacacaaatacatataaatacacacaaatacacacaaatacacacagaaatacacacgcacacgcacacacacaaaacacatacacatacacacgaatacacacaaatacacacacaaatacacatacacgcacacacatgcacaacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattctgtacgggccagatatacgcgtt

SEQ ID NO: 248 - DNA, 524 bp, synthetic construct; identical to SEQ ID NO: 201
  source 1..524: mol_type = other DNA

SEQ ID NO: 249 - DNA, 576 bp, synthetic construct
  source 1..576: mol_type = other DNA
cgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagttta

SEQ ID NO: 250 - DNA, 326 bp, synthetic construct
  source 1..326: mol_type = other DNA
ggtacccgggggcgcgccagtcaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 251 - DNA, 1761 bp, synthetic construct
  source 1..1761: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgaggtggaaattaatacgacgtggcggatccgccacc

SEQ ID NO: 252 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 253 - DNA, 276 bp, synthetic construct
  source 1..276: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 254 - DNA, 1013 bp, synthetic construct
  source 1..1013: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 255 - DNA, 700 bp, synthetic construct
  source 1..700: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaataatacgactcactatagggaagcttggcattccggtactgttggatccgccacc

SEQ ID NO: 256 - DNA, 324 bp, synthetic construct
  source 1..324: mol_type = other DNA
acattaatttaccagaaacaagatacacatctctctttgaggagtgccctaacttcccatcattttgtccaattaaatgaattgaagaaatttaatgtttctaaactagaccaacaaagaataatagttgtatgacaagtaaataagctttgctgggaagatgttgcttaaatgataaaatggttcagccaacaagtgaaccaaaaattaaatattaactaaggaaaggtaaccatttctgaagtcattcctagcagaggactcagatatatataggattgaagatctctcagttaagtctacatgaaaaggggatccgccacc

(19) United States
(12) Patent Application Publication
(10) Pub. No.: US 2026/0109979 A1
(43) Pub. Date: Apr. 23, 2026

(54) NUCLEIC ACID REGULATORY ELEMENTS FOR GENE EXPRESSION IN THE LIVER AND METHODS OF USE

(71) Applicant: MeiraGTx UK II Limited, London (GB)

(72) Inventors: Alexandria Forbes, New York, NY (US); Josefa Sullivan, New York, NY (US); Chenjin Jin, New York, NY (US); Enrico Mossotto, New York, NY (US); Matthew During, Rowayton, CT (US); Ce Feng Liu, New York, NY (US); Dustin Lee, New York, NY (US); Jessica Schmerler, New York, NY (US)

(21) Appl. No.: 19/117,793

(22) PCT Filed: Oct. 11, 2023
(86) PCT No.: PCT/IB2023/000616

§ 371 Date: Apr. 2, 2025

Related U.S. Application Data
(60) Provisional application No. 63/379,138, filed on Oct. 11, 2022.
(60) Provisional application No. 63/496,554, filed on Apr. 17, 2023.

(51) Int. Cl. C12N 15/113; A61K 31/7088; A61K 48/00; A61P 25/28; C12N 5/071; C12N 15/864

(52) U.S. Cl. CPC C12N 15/113; A61K 31/7088; A61K 48/005; A61P 25/28; C12N 5/067; C12N 15/8645; C12N 2750/14132; C12N 2750/14143; C12N 2830/008; C12N 2830/50

(57) ABSTRACT

The application relates to nucleic acid regulatory elements (NAREs) that are able to enhance expression of genes in the liver. The application further relates to methods employing NAREs and uses of the NAREs. Expression cassettes and vectors containing NAREs are also disclosed. These are particularly useful for applications using gene therapy.

### CROSS-REFERENCE TO RELATED APPLICATIONS

This application claims the benefit under 35 U.S.C. § 119(e) of the earlier filing date of U.S. Provisional Patent Application Ser. No. 63/379,138, filed on Oct. 11, 2022, and U.S. Provisional Patent Application Ser. No. 63/496,554, filed on Apr. 17, 2023, which are hereby incorporated by reference in their entirety.

### REFERENCE TO AN ELECTRONIC SEQUENCE LISTING

The contents of the electronic sequence listing (SeqList-162027-54276.xml; Size: 359,832 bytes; and Date of Creation: Oct. 6, 2023) is herein incorporated by reference in its entirety.

### FIELD

The application relates to nucleic acid regulatory elements that are able to enhance expression of genes in a variety of tissues, or in particular tissues including liver tissue. The application further relates to methods employing these regulatory elements and uses of these elements. Expression cassettes and vectors containing these nucleic acid regulatory elements are also disclosed. These are particularly useful for applications using gene therapy.

### BACKGROUND

A promoter is a DNA region at which transcription of a gene is initiated. Since the promoter region controls when and where a gene of interest is expressed in an organism, promoters are crucial elements for regulating the level and specificity of transgene expression, particularly in the context of gene therapy.

The use of engineered nucleic acid regulatory elements (NAREs) (that may include various components, including, promoters, enhancers, etc.) that are particularly tailored to a given gene therapy provides a variety of benefits. To start, an optimized NARE allows for levels of gene expression that is desired for a specific therapeutic gene. Second, engineered NAREs with increased potency allow administration of smaller amounts of gene therapy vector, thus decreasing immune responses and associated safety risks. Third, for some gene therapies, it is desirable that gene expression is limited to a specific tissue or tissues.

Accordingly, use of nucleic acid regulatory elements with tissue-specific expression can restrict unwanted transgene expression as well as facilitate persistent transgene expression in the tissue(s) or interest. Such tissue-specific NAREs can be used to eliminate the need for tissue-specific viral capsids used for gene delivery (or can be used in combination with tissue-specific viral capsids). Fourth, choosing an appropriate NARE also allows controlling the kinetics of gene expression, which in turn impact durability of gene therapy. Finally, it can be desirable to engineer NAREs with a reduced size (without sacrificing strength or specificity) to allow for efficient packaging of larger transgene cargo into viral vectors.

While efforts have been made to characterize and optimize NARE sequences through conventional low-throughput analyses (rational design) or newer high-throughput methodologies (e.g., MPRA), these approaches still require an expensive and time-consuming in vitro optimization. Accordingly, more efficient methods of engineering NAREs are needed as are nucleic acid regulatory elements with enhanced potency, reduced size, and/or tissue specificity.

### SUMMARY

Provided herein are nucleic acid regulatory elements (NAREs), including NAREs that are particularly suitable for the expression of an operably linked sequence (e.g., a protein or RNA coding sequence) in the liver. Also provided herein are methods for employing NAREs and uses of NAREs. For example, provided herein are methods for the expression of a transgene that is operably linked to one or more of the nucleic acid regulatory elements disclosed herein. Also provided are expression cassettes and vectors containing NAREs and cells comprising such expression cassettes and vectors.

Provided herein is a polynucleotide sequence that comprises a sequence that is at least 80% identical to a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140 (see Table 7). Provided herein is a polynucleotide sequence that comprises a sequence that is at least 90% identical to a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140. Provided herein is provided is a polynucleotide sequence comprising a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140.

Provided herein is a polynucleotide that comprises a sequence that is at least 80% identical to a NARE selected from the group consisting of L10, L13, L15, L17, L45, L56-L61, L71-L79, and L89. Provided herein is a polynucleotide that comprises a sequence that is at least 90% identical to a NARE selected from the group consisting of L10, L13, L15, L17, L45, L56-L61, L71-L79, and L89. Provided herein is a polynucleotide that comprises a NARE selected from the group consisting of L10, L13, L15, L17, L45, L56-L61, L71-L79, and L89.

Provided herein is a polynucleotide that comprises a sequence that is at least 80% identical to a NARE selected from the group consisting of L45, L123, L132, and L133. Provided herein is a polynucleotide that comprises a sequence that is at least 90% identical to a NARE selected from the group consisting of L45, L123, L132, and L133. Provided herein is a polynucleotide that comprises a NARE selected from the group consisting of L45, L123, L132, and L133.

In embodiments, provided is a polynucleotide sequence that comprises a sequence that is at least 80% identical to a NARE sequence provided in Table 7. In embodiments, provided is a polynucleotide sequence that comprises a sequence that is at least 90% identical to a NARE sequence provided in Table 7. In embodiments, provided is a polynucleotide comprising a NARE sequence provided in Table 7.

In one aspect, provided is a NARE comprising: (i) a sequence that is at least 90% identical to SEQ ID NO: 74; and (ii) a sequence that is at least 90% identical to any one of SEQ ID NOs: 193, 194, 207, 209-214, 216-232, 233-235, or 249. In some embodiments, the NARE comprises: (i) a sequence that is at least 95% identical to SEQ ID NO: 74; and (ii) a sequence that is at least 95% identical to any one of SEQ ID NOs: 193, 194, 207, 209-214, 216-232, 233-235, or 249. In some embodiments, the NARE of comprises: (i) SEQ ID NO: 74; and (ii) any one of SEQ ID NOs: 193, 194, 207, 209-214, 216-232, 233-235, or 249.

In one aspect, provided is a NARE comprising: (i) a sequence that is at least 90% identical to SEQ ID NO: 150; and (ii) a sequence that is at least 90% identical to SEQ ID NO: 151 or SEQ ID NO: 155. In some embodiments, the NARE comprises: (i) a sequence that is at least 95% identical to SEQ ID NO: 150; and (ii) a sequence that is at least 95% identical to SEQ ID NO: 151 or SEQ ID NO: 155. In some embodiments, the NARE comprises: (i) SEQ ID NO: 150; and (ii) SEQ ID NO: 151 or SEQ ID NO: 155.

In one aspect, provided is a NARE comprising: (i) a sequence that is at least 90% identical to any one of SEQ ID NOs: 154, 157, or 158; (ii) a sequence that is at least 90% identical to any one of SEQ ID NOs: 153, 156, 161, 162, 163, 166, 167, 168, or 174; and (iii) optionally, a sequence that is at least 90% identical to any one of SEQ ID NOs: 169, 172 or 173. In some embodiments, the NARE comprises: (i) a sequence that is at least 95% identical to any one of SEQ ID NOs: 154, 157, or 158; (ii) a sequence that is at least 95% identical to any one of SEQ ID NOs: 153, 156, 161, 162, 163, 166, 167, 168, or 174; and (iii) optionally, a sequence that is at least 95% identical to any one of SEQ ID NOs: 169, 172 or 173. In some embodiments, the NARE comprises: (i) any one of SEQ ID NOs: 154, 157, or 158 (ii) any one of SEQ ID NOs: 153, 156, 161, 162, 163, 166, 167, 168, or 174; and (iii) optionally any one of SEQ ID NOs: 169, 172 or 173.

In one aspect, provided is a NARE comprising: (i) a sequence that is at least 90% identical to SEQ ID NO: 153; (ii) a sequence that is at least 90% identical to SEQ ID NO: 171; and (iii) a sequence that is at least 90% identical to any one of SEQ ID NOs: 154, 157, 158 or 176. In some embodiments, the NARE comprises: (i) a sequence that is at least 95% identical to SEQ ID NO: 153; (ii) a sequence that is at least 95% identical to SEQ ID NO: 171; and (iii) a sequence that is at least 95% identical to any one of SEQ ID NOs: 154, 157, 158 or 176. In some embodiments, the NARE comprises: (i) SEQ ID NO: 153; (ii) SEQ ID NO: 171; and (iii) any one of SEQ ID NOs: 154, 157, 158 or 176.

In one aspect, provided is a NARE comprising: (i) a sequence that is at least 90% identical to SEQ ID NO: 153; (ii) a sequence that is at least 90% identical to any one of SEQ ID NOs: 154, 157, 158, 176, 188, or 250; and (iii) optionally, SEQ ID NO: 155 or SEQ ID NO: 171. In some embodiments, the NARE comprises: (i) a sequence that is at least 95% identical to SEQ ID NO: 153; (ii) a sequence that is at least 95% identical to any one of SEQ ID NOs: 154, 157, 158, 176, 188, or 250; and (iii) optionally, SEQ ID NO: 155 or SEQ ID NO: 171. In some embodiments, the NARE comprises: (i) SEQ ID NO: 153; (ii) any one of SEQ ID NOs: 154, 157, 158, 176, 188, or 250; and (iii) optionally, SEQ ID NO: 155 or SEQ ID NO: 171.

In one aspect, provided is a NARE comprising: (i) a sequence that is at least 90% identical to SEQ ID NO: 155; (ii) a sequence that is at least 90% identical to SEQ ID NO: 176 and (iii) a sequence that is at least 90% identical to any one of SEQ ID NOs: 175 or 177-187. In some embodiments, the NARE comprises: (i) a sequence that is at least 95% identical to SEQ ID NO: 153; (ii) a sequence that is at least 95% identical to SEQ ID NO: 176 and (iii) a sequence that is at least 95% identical to any one of SEQ ID NOs: 175 or 177-187. In some embodiments, the NARE comprises: (i) SEQ ID NO: 153; (ii) SEQ ID NO: 176 and (iii) any one of SEQ ID NOs: 175 or 177-187.

In one aspect, provided is a NARE comprising: (i) a sequence that is at least 90% identical to SEQ ID NO: 190 or SEQ ID NO: 191; (ii) a sequence that is at least 90% identical to any one of SEQ ID NOs: 193, 194, or 195; and (iii) optionally, a sequence that is at least 90% identical to SEQ ID NO: 162; (iv) optionally, a sequence that is at least 90% identical to SEQ ID NO: 168; and (v) optionally, SEQ ID NO: 169. In some embodiments, the NARE comprises: (i) a sequence that is at least 95% identical to SEQ ID NO: 190 or SEQ ID NO: 191; (ii) a sequence that is at least 95% identical to any one of SEQ ID NOs: 193, 194, or 195; and (iii) optionally, a sequence that is at least 95% identical to SEQ ID NO: 162; (iv) optionally, a sequence that is at least 95% identical to SEQ ID NO: 168; and (v) optionally, SEQ ID NO: 169. In some embodiments, the NARE comprises: (i) SEQ ID NO: 190 or SEQ ID NO: 191; (ii) any one of SEQ ID NOs: 193, 194, or 195; (iii) optionally, SEQ ID NO: 162; (iv) optionally, SEQ ID NO: 168; and (v) optionally, SEQ ID NO: 169.

In one aspect, provided is a NARE comprising: (i) a sequence that is at least 90% identical to SEQ ID NO: 168; (ii) a sequence that is at least 90% identical to any one of SEQ ID NOs: 197, 202 or 203; (iv) optionally, a sequence that is at least 90% identical to any one of SEQ ID NOs: 190, 191, 192, 196, 202, 203, 204, 205 or 206; (v) optionally, a sequence that is at least 90% identical to SEQ ID NO: 155 or SEQ ID NO: 162; and (vi) optionally, a sequence that is at least 90% identical to any one of SEQ ID NOs: 166, 193, or 194. In some embodiments, the NARE comprises: (i) a sequence that is at least 95% identical to SEQ ID NO: 168; (ii) a sequence that is at least 95% identical to any one of SEQ ID NOs: 197, 202 or 203; (iv) optionally, a sequence that is at least 95% identical to any one of SEQ ID NOs: 190, 191, 192, 196, 202, 203, 204, 205 or 206; (v) optionally, a sequence that is at least 95% identical to SEQ ID NO: 155 or SEQ ID NO: 162; and (vi) optionally, a sequence that is at least 95% identical to any one of SEQ ID NOs: 166, 193, or 194. In some embodiments, the NARE comprises: (i) SEQ ID NO: 168; (ii) any one of SEQ ID NOs: 197, 202 or 203; (iv) optionally, any one of SEQ ID NOs: 190, 191, 192, 196, 202, 203, 204, 205 or 206; (v) optionally, SEQ ID NO: 155 or SEQ ID NO: 162; and (vi) optionally any one of SEQ ID NOs: 166, 193, or 194.

In one aspect, provided is a NARE comprising: (i) a sequence that is at least 90% identical to SEQ ID NO: 192; (ii) a sequence that is at least 90% identical to SEQ ID NO: 193 or SEQ ID NO: 194; and (iii) a sequence that is at least 90% identical to any one of SEQ ID NOs: 201-203. In some embodiments, the NARE comprises: (i) a sequence that is at least 95% identical to SEQ ID NO: 192; (ii) a sequence that is at least 95% identical to SEQ ID NO: 193 or SEQ ID NO: 194; and (iii) a sequence that is at least 95% identical to any one of SEQ ID NOs: 201-203. In some embodiments, the NARE comprises: (i) SEQ ID NO: 192; (ii) SEQ ID NO: 193 or SEQ ID NO: 194; and (iii) any one of SEQ ID NOs: 201-203.

In one aspect, provided is a NARE comprising: (i) a sequence that is at least 90% identical to SEQ ID NO: 155; (ii) a sequence that is at least 90% to any one of SEQ ID NOs: 204, 205, 206, or 236-238; (iii) optionally, a sequence that is at 90% identical to SEQ ID NO: 168; (iv) optionally, a sequence that is at least 90% identical to SEQ ID NO: 169; and (v) optionally, a sequence that is at least 90% identical to SEQ ID NO: 197. In some embodiments, the NARE comprises: (i) a sequence that is at least 95% identical to SEQ ID NO: 155; (ii) a sequence that is at least 95% to any one of SEQ ID NOs: 204, 205, 206, or 236-238; (iii) optionally, a sequence that is at 90% identical to SEQ ID NO: 168; (iv) optionally, a sequence that is at least 95% identical to SEQ ID NO: 169; and (v) optionally, a sequence that is at least 95% identical to SEQ ID NO: 197. In some embodiments, the NARE comprises: (i) SEQ ID NO: 155; (ii) a sequence that is at least 95% to any one of SEQ ID NOs: 204, 205, 206, or 236-238; (iii) optionally, a sequence that is at 90% identical to SEQ ID NO: 168; (iv) optionally, a sequence that is at least 95% identical to SEQ ID NO: 169; and (v) optionally, a sequence that is at least 95% identical to SEQ ID NO: 197.

In one aspect, provided is a NARE comprising: (i) a sequence that is at least 90% identical to SEQ ID NO: 248; and (ii) a sequence that is at least 90% identical to any one of SEQ ID NOs: 239-247. In some embodiments, the NARE comprises: (i) a sequence that is at least 95% identical to SEQ ID NO: 248; and (ii) a sequence that is at least 95% identical to any one of SEQ ID NOs: 239-247. In some embodiments, the NARE comprises: (i) SEQ ID NO: 248; and (ii) any one of SEQ ID NOs: 239-247. In one aspect, provided is a NARE comprising: (i) a sequence that is at least 90% identical to SEQ ID NO: 208; and (ii) a sequence that is at least 90% identical to any one of SEQ ID NOs: 209-214.

In some embodiments, the NARE comprises: (i) a sequence that is at least 95% identical to SEQ ID NO: 208; and (ii) a sequence that is at least 95% identical to any one of SEQ ID NOs: 209-214. In some embodiments, the NARE comprises: (i) SEQ ID NO: 208; and (ii) any one of SEQ ID NOs: 209-214. In one aspect, provided is a NARE comprising: (i) a sequence that is at least 90% identical to SEQ ID NO: 193 or SEQ ID NO: 194; (ii) a sequence that is at least 90% identical to SEQ ID NO: 215; and (iii) a sequence that is at least 90% identical to any one of SEQ ID NOs: 207 or 209-215.

In some embodiments, the NARE comprises: (i) a sequence that is at least 95% identical to SEQ ID NO: 193 or SEQ ID NO: 194; (ii) a sequence that is at least 95% identical to SEQ ID NO: 215; and (iii) a sequence that is at least 95% identical to any one of

SEQ ID NOs: 207 or 209-215. In some embodiments, the NARE comprises: (i) SEQ ID NO: 193 or SEQ ID NO: 194; (ii) SEQ ID NO: 215; and (iii) any one of SEQ ID NOs: 207 or 209-215. In one aspect, provided is a NARE comprising: (i) a sequence that is at least 90% identical to SEQ ID NO: 155; (ii) a sequence that is at least 90% identical to SEQ ID NO: 205 or SEQ ID NO: 206; and (iii) a sequence that is at least 90% identical to any one of SEQ ID NOs: 207 or 209-215.

In some embodiments, the NARE comprises: (i) a sequence that is at least 95% identical to SEQ ID NO: 155; (ii) a sequence that is at least 95% identical to SEQ ID NO: 205 or SEQ ID NO: 206; and (iii) a sequence that is at least 95% identical to any one of SEQ ID NOs: 207 or 209-215. In some embodiments, the NARE comprises: (i) SEQ ID NO: 155; (ii) SEQ ID NO: 205 or SEQ ID NO: 206; and (iii) any one of SEQ ID NOs: 207 or 209-215.

In one aspect, provided is a NARE comprising:
a) (i) a sequence that is at least 90% identical to SEQ ID NO: 53;
b) (i) a sequence that is at least 90% identical to SEQ ID NO: 195; and (ii) a sequence that is at least 90% identical to SEQ ID NO: 202;
c) (i) a sequence that is at least 90% identical to a sequence that is at least 90% identical to SEQ ID NO: 168; (ii) a sequence that is at least 90% identical to a sequence that is at least 90% identical to SEQ ID NO: 169; (iii) a sequence that is at least 90% identical to a sequence that is at least 90% identical to SEQ ID NO: 197; and (iv) a sequence that is at least 90% identical to a sequence that is at least 90% identical to SEQ ID NO: 203;
d) (i) a sequence that is at least 90% identical to SEQ ID NO: 74; and (ii) a sequence that is at least 90% identical to a sequence that is at least 90% identical to SEQ ID NO: 220;
e) (i) a sequence that is at least 90% identical to SEQ ID NO: 74; and (ii) a sequence that is at least 90% identical to a sequence that is at least 90% identical to SEQ ID NO: 229; or
f) (i) a sequence that is at least 90% identical to SEQ ID NO: 74; and (ii) a sequence that is at least 90% identical to a sequence that is at least 90% identical to SEQ ID NO: 230.

In some embodiments, the NARE comprises:
a) (i) a sequence that is at least 95% identical to SEQ ID NO: 53;
b) (i) a sequence that is at least 95% identical to SEQ ID NO: 195; and (ii) a sequence that is at least 95% identical to SEQ ID NO: 202;
c) (i) a sequence that is at least 95% identical to a sequence that is at least 95% identical to SEQ ID NO: 168; (ii) a sequence that is at least 95% identical to a sequence that is at least 95% identical to SEQ ID NO: 169; (iii) a sequence that is at least 95% identical to a sequence that is at least 95% identical to SEQ ID NO: 197; and (iv) a sequence that is at least 95% identical to a sequence that is at least 95% identical to SEQ ID NO: 203;
d) (i) a sequence that is at least 95% identical to SEQ ID NO: 74; and (ii) a sequence that is at least 95% identical to a sequence that is at least 95% identical to SEQ ID NO: 220;
e) (i) a sequence that is at least 95% identical to SEQ ID NO: 74; and (ii) a sequence that is at least 95% identical to a sequence that is at least 95% identical to SEQ ID NO: 229; or
f) (i) a sequence that is at least 95% identical to SEQ ID NO: 74; and (ii) a sequence that is at least 95% identical to a sequence that is at least 95% identical to SEQ ID NO: 230.

In some embodiments, the NARE comprises:
a) (i) SEQ ID NO: 53;
b) (i) SEQ ID NO: 195; and (ii) SEQ ID NO: 202;
c) (i) SEQ ID NO: 168; (ii) SEQ ID NO: 169; (iii) SEQ ID NO: 197; and (iv) to SEQ ID NO: 203;
d) (i) SEQ ID NO: 74; and (ii) SEQ ID NO: 220;
e) (i) SEQ ID NO: 74; and (ii) SEQ ID NO: 229; or
f) (i) SEQ ID NO: 74; and (ii) SEQ ID NO: 230.

In one aspect, provided is a NARE comprising: (i) a sequence that is at least 90% identical to SEQ ID NO: 166 and (ii) a sequence that is at least 90% identical to any one of SEQ ID NOs: 154, 157, or 158. In some embodiments, the NARE comprises: (i) a sequence that is at least 95% identical to SEQ ID NO: 166 and (ii) a sequence that is at least 95% identical to any one of SEQ ID NOs: 154, 157, or 158. In some embodiments, the NARE comprises: (i) SEQ ID NO: 166; and (ii) any one of SEQ ID NOs: 154, 157, or 158.

In one aspect, provided is a NARE comprising: (i) a sequence that is at least 90% identical to SEQ ID NO: 165 and (ii) a sequence that at least 90% identical to any one of SEQ ID NOs: 154, 157, or 158. In some embodiments, the NARE comprises: (i) a sequence that is at least 95% identical to SEQ ID NO: 165 and (ii) a sequence that at least 95% identical to any one of SEQ ID NOs: 154, 157, or 158. In some embodiments, the NARE comprises: (i) SEQ ID NO: 165 and (ii) any one of SEQ ID NOs: 154, 157, or 158.

In one aspect, provided is a NARE comprising: (i) a sequence that is at least 90% identical to SEQ ID NO: 199; and (ii) a sequence that is at least 90% identical to SEQ ID NO: 193 or SEQ ID NO: 194. In some embodiments, the NARE comprises: (i) a sequence that is at least 95% identical to SEQ ID NO: 199; and (ii) a sequence that is at least 95% identical to SEQ ID NO: 193 or SEQ ID NO: 194. In some embodiments, the NARE comprises: (i) SEQ ID NO: 199; and (ii) SEQ ID NO: 193 or SEQ ID NO: 194.

In one aspect, provided is a NARE comprising: (i) a sequence that is at least 90% identical to SEQ ID NO: 164; and (ii) a sequence that is least 90% identical to SEQ ID NO: 200. In some embodiments, the NARE comprises: (i) a sequence that is at least 95% identical to SEQ ID NO: 164; and (ii) a sequence that is least 95% identical to SEQ ID NO: 200. In some embodiments, the NARE comprises: (i) SEQ ID NO: 164; and (ii) SEQ ID NO: 200.

In one aspect, provided is a NARE comprising: (i) a sequence that is at least 90% identical to SEQ ID NO: 189 and (ii) a sequence that is at least 90% identical to SEQ ID NO: 193 or SEQ ID NO: 194. In some embodiments, the NARE comprises: (i) a sequence that is at least 95% identical to SEQ ID NO: 189 and (ii) a sequence that is at least 95% identical to SEQ ID NO: 193 or SEQ ID NO: 194. In some embodiments, the NARE comprises: (i) SEQ ID NO: 189 and (ii) SEQ ID NO: 193 or SEQ ID NO: 194.

In one aspect, provided is a NARE comprising: (i) a sequence that is at least 90% identical to SEQ ID NO: 202 or SEQ ID NO: 203; and (ii) a sequence that is at least 90% identical to SEQ ID NO: 195. In some embodiments, the NARE comprises: (i) a sequence that is at least 95% identical to SEQ ID NO: 202 or SEQ ID NO: 203; and (ii) a sequence that is at least 95% identical to SEQ ID NO: 195. In some embodiments, the NARE comprises: (i) SEQ ID NO: 202 or SEQ ID NO: 203; and (ii) SEQ ID NO: 195.

In one aspect, provided is a NARE comprising a sequence that is at least 90% identical to any one of SEQ ID NOs: 3-8, 11-69, 71-73, or 76-149. In some embodiments, the NARE comprises a sequence that is at least 95% identical to any one of SEQ ID NOs: 3-8, 11-69, 71-73, or 76-149. In some embodiments, the NARE comprises any one of SEQ ID NOs: 3-8, 11-69, 71-73, or 76-149. In some embodiments, the NARE comprises a sequence that is at least 90% identical to any one of SEQ ID NOs: 53, 80, 81, 132, 141, or 142. In some embodiments, the NARE comprises a sequence that is at least 95% identical to any one of SEQ ID NOs: 53, 80, 81, 132, 141, or 142. In some embodiments, the NARE comprises any one of SEQ ID NOs: 53, 80, 81, 132, 141, or 142.

In one aspect, provided is an expression construct comprising a NARE disclosed herein and an operatively linked transgene. In some embodiments, the expression construct further comprises a polyadenylation sequence.

In one aspect, provided is a vector comprising an expression construct disclosed herein. In some embodiments, the vector is a non-viral vector. In some embodiments, the vector is a viral vector. In some embodiments, the vector is an adeno-associated virus (AAV) vector. In some embodiments, the vector comprises a nucleic acid sequence comprising (i) the expression construct comprising a NARE disclosed herein and an operatively linked transgene, and (ii) one or more inverted terminal repeats (ITR). In some embodiments, the vector comprises a nucleic acid sequence comprising (i) an expression construct disclosed herein, and (ii) one or more inverted terminal repeats (ITR). In some embodiments, the 5′ ITR and a 3′ ITR are derived from adeno-associated virus (AAV) serotype AAV2.

In one aspect, provided is a cell comprising an expression construct disclosed herein or a vector disclosed herein. In one embodiment, the cell is a liver cell.

In one aspect, provided is a pharmaceutical composition comprising: (i) an expression construct disclosed herein or a vector disclosed herein and (ii) a pharmaceutically acceptable excipient.

In one aspect, provided is a method for expressing a transgene in a cell comprising an expression construct disclosed herein or a vector disclosed herein. In one aspect, provided is a method for regulating transgene expression in a cell comprising an expression construct disclosed herein or a vector disclosed herein. In one embodiment, the cell is a liver cell.

In one aspect, provided is a method of treating Wilson's disease in a subject in need thereof, the method comprising administering to the subject an expression construct disclosed herein, a vector disclosed herein, or a pharmaceutical composition disclosed herein. In one embodiment the subject has a mutation in the ATP7B gene. In one embodiment the subject is a human.

### BRIEF DESCRIPTION OF THE FIGURES

FIG. 1 Diagram of bioinformatics pipeline for the design and optimization of a given promoter sequence utilizing AI. The pipeline is divided in two parallel approaches: (i) Promoter Enhancement, whereby all known/annotated enhancer fragments were collected from the ENCODE database and inserted upstream the promoter sequence; and (ii) Saturation Mutagenesis, in which all possible point mutations (no indels) were generated in silico for each base pair in the original input promoter sequence. Best candidates from both approaches were then tested in vitro in the preferred cell line and activities recorded through fluorescence. Best in vitro performers from both strategies were then combined through rational design in a final in vitro screen.

FIGS. 2A and 2B. Dual reporter design and measurement. FIG. 2A illustrates the design of the dual reporter construct. Candidate NAREs were cloned upstream (5′) of the mClover3 coding sequence. Each construct contains a constant region that includes a tdTomato transgene that is used as a normalization control. FIG. 2B provides an example of flow cytometry data obtained with the dual reporter system, in which mClover3 expression in tdTomato+ cells reflects the level of promoter activity.

FIGS. 3A, 3B, 3C, and 3D. Potency of selected NAREs in liver cells in vitro. NAREs particularly suitable for the expression in the liver were rationally designed by employing established or putative liver-specific core NAREs and enhancers, and incorporating other novel changes based various bioinformatics approaches. Expression was assessed using the dual reporter assay. FIG. 3A: Potency of indicated NAREs as compared to reference promoters CAG, CMV, and AAT in Huh7 cells. FIG. 3B: Tissue specificity for selected NAREs (relative expression in HepG2 liver cells v. relative expression in HEK293T cells). FIG. 3C: Potency of indicated NAREs in a human liver cell line Huh7 cells as compared to the AAT promoter (L1, 305 bp). FIG. 3D: Bright field and mClover3 fluorescence of NARE L45 compared to AAT promoter (L1). Similar cell numbers are present in the bright-field image, but stronger mClover3 fluorescence is observed in L45. NARE L45 drove higher mClover3 expression than AAT after 48 h of transfection in Huh7 cells.

FIGS. 4A, 4B, and 4C. Potency of selected NAREs in liver cells in vitro. FIGS. 4A and 4B: Optimization of NARE L45 led to new NAREs that are up to nine-fold stronger than the parent (L45). NARE modifications included enhancers, cis-regulatory elements, introns or other bioinformatically identified sequences predicted to boost activity. FIG. 4A: Expression of L45 and modified NAREs relative to CAG in Huh7 cells as determined using the dual reporter assay. FIG. 4B: Expression levels of twelve liver-specific NAREs that are up to 4-fold stronger than the CAG reference promoter and stronger than promoters currently used clinically (AAT, LP1, TBG). Dual-reporter plasmids were transiently transfected in human liver cell line (Huh7) and expression of the mClover3 transgene was assayed by flow cytometry. FIG. 4C: Potency of indicated NAREs in Huh7 cells.

### DETAILED DESCRIPTION

Nucleic acid regulatory elements (NAREs) including promoters are essential components of a gene therapy that control the expression level and durability of a therapeutic gene. NAREs can drive cell-specific expression of transgenes independent of, for example, capsid choice. Incorporation of stronger NAREs can increase potency and efficacy at lower viral vector doses, thereby potentially decreasing safety risks, immune responses, and reducing cost of vector production. Moreover, reducing NARE size while maintaining strength and specificity allows efficient packaging of larger transgenes or expression cassettes into AAV. The present application provides libraries of NAREs to improve the safety, efficacy, and durability of therapeutic transgene expression.

While many NAREs and particularly promoter sequences have been characterized/optimized through conventional low-throughput analyses (rational design) or newer high-throughput methodologies (e.g., MPRA), these approaches still require an expensive and time-consuming in vitro optimization. Here, using advanced artificial intelligence models, a convolutional neural network (CNN) was repurposed and optimized to predict promoter potency. A library of NAREs was computationally constructed by cloning all known enhancer elements reported in the ENCODE database upstream of a potent, compact constitutive promoter. Subsequently, the correct spacing between the particularly well-performing enhancer elements and the promoter sequence was optimized. In parallel, in silico saturated mutability was performed, whereby all possible point mutations within the promoter sequence were introduced and those with the best impact on promoter's potency were selected. Particularly well-performing elements were produced and tested in vitro. While most of the NAREs exhibited better or equal performance than the original promoter, the best enhancer elements and point mutation were selected and combined in a second optimization round. After synthesizing and testing this new set in vitro, enhanced NAREs that exhibit increased potency were obtained.

### NAREs

Provided herein are NAREs that may include as part of their sequence a promoter and/or an enhancer. Traditionally, promoters are defined as DNA regions where transcription is initiated. Promoters include specific DNA motifs that transcription factors (TFs) and their complexes can access. On the other hand, enhancers are defined as DNA regions that amplify transcription initiation by directly interplaying with their target promoters. Likewise, the enhancer sequences, distal from their target promoters, contain DNA motifs that act as binding sites for TFs and cofactors. At times, the term promoter may be used as a shorthand to refer to a nucleic acid sequence that comprises multiple regulatory elements. The control promoter CAG, for example, contains a CMV enhancer, β actin promoter region and intron, but may be referred to as a promoter. Specifically, the CAG promoter consists of (1) the cytomegalovirus (CMV) early enhancer element, (2) the promoter, the first exon and the first intron of chicken beta-actin gene, and (3) the splice acceptor of the rabbit beta-globin gene. As used herein, the term “nucleic acid regulatory element” can refer to a promoter defined in the traditional sense as well as a combination of elements that comprises a promoter and/or other nucleic acid regulatory elements that modulate the expression of a gene that is operably linked to the element(s). A nucleic acid regulatory element may be, or include one or more of, e.g., promoters, enhancers, translation initiation signals, introns, and/or splicing enhancers.

As used herein, the term “NARE” can refer to a promoter defined in the traditional sense as well as a combination of nucleic acid regulatory elements that comprises a promoter and/or other nucleic acid regulatory elements that modulate the expression of a gene that is operably linked to the element(s). A nucleic acid regulatory element may be, or include one or more of, e.g., promoters, enhancers, translation initiation signals, introns, and/or splicing enhancers.

As used herein, “transgene” refers to a gene (in particular the coding sequence of the gene) that is transferred into one or more cells of an organism, for example using a vector described herein. A transgene can encode a protein or RNA that is normally expressed in cells of the target organism, or may encode a protein or RNA from a different organism. The transgene may be integrated into the genome of the target cell, or may exist as part of an extrachromosomal expression construct.

As used herein, “operatively linked” refers to a first molecule joined to a second molecule, wherein the molecules are so arranged that the first molecule affects the function of the second molecule. The two molecules may or may not be part of a single contiguous molecule and may or may not be adjacent. For example, a NARE is operatively linked to a transcribable polynucleotide molecule if the NARE modulates transcription of the transcribable polynucleotide molecule of interest in a cell. Additionally, two portions of a transcription regulatory element are operatively linked to one another if they are joined such that the transcription-activating functionality of one portion is not adversely affected by the presence of the other portion. Two transcription regulatory elements may be operatively linked to one another by way of a linker nucleic acid (e.g., an intervening non-coding nucleic acid) or may be operatively linked to one another with no intervening nucleotides present.

Provided herein are NAREs particularly suitable to drive expression in the liver. As used herein, NAREs starting with “L” are particularly useful for the expression of genes in the liver. Liver NAREs can be liver-specific, meaning that they show significantly increased, or preferential, expression of an operably linked transgene in the liver as compared to in other tissues. Table 5 and Table 7 provide NAREs particularly suitable to drive expression in the liver. Table 4 also provides NAREs suitable for driving expression in a variety of tissues, including in the liver.

In some embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE provided in Table 5 or Table 7. In some embodiments, provided is a polynucleotide comprising one or more NAREs provided in Table 5 or Table 7.

Provided herein is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99% identical to NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140. Provided herein is provided is a polynucleotide sequence comprising a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. Methods and computer programs for determining both sequence identity and similarity are publicly available, including, but not limited to, the GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at ncbi.nlm.nih.gov/blast/). In comparing sequences, these methods account for various substitutions, deletions, and other modifications.

Provided herein is a polynucleotide that comprises a sequence that is at least 80%, at least 85%, at least 90%, at least 91%, at least 92%, at least 93%, at least 94%, at least 95%, at least 96%, at least 97%, at least 98%, or at least 99% identical to a NARE selected from the group consisting of L10, L13, L15, L17, L45, L56-L61, L71-L79, and L89. Provided herein is a polynucleotide that comprises a NARE selected from the group consisting of L10, L13, L15, L17, L45, L56-L61, L71-L79, and L89.

Provided herein is a polynucleotide that comprises a sequence that is at least 80%, at least 85%, at least 90%, at least 91%, at least 92%, at least 93%, at least 94%, at least 95%, at least 96%, at least 97%, at least 98%, or at least 99% identical to a NARE selected from the group consisting of L45, L123, L132, and L133. Provided herein is a polynucleotide that comprises a NARE selected from the group consisting of L45, L123, L132, and L133.

Provided herein is a method for expression of a transgene in the liver, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a NARE provided in Table 5 or Table 7. Provided herein is a method for expression of a transgene in the liver, wherein the transgene is operably linked to a NARE provided in Table 5 or Table 7.

Provided herein is a method for expression of a transgene in the liver, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140. Provided herein is a method for expression of a transgene in the liver, wherein the transgene is operably linked to a NARE selected from the group consisting of L2-L7, L9, L10, L10_v2, L11-L13, L13_TISU, L13_v2, L14, L15, L14_L21, L15_L21_TISU, L16-L62, L64, L66, L69-L78, L78b, and L79-L140.

In embodiments, the transgene is ATP7B or an ATB7B minigene.

In embodiments, the NARE comprises an intron or a portion of an intron. The intron or portion of an intron may be selected from: ACTA1 intron, ACTA1e intron, ACTC1 intron, ACTC1.3 intron, ACTC1e intron, ACTC1e-ACTC1p intron, ALDOA intron, APOC intron, APOC1 intron, ATF5 intron, CAMK2A intron, CBA intron, chimeric intron, CMV-rabbit beta globlin intron, CRYAB intron, DES.4 intron, EEF1A1 intron, EEF1B2 intron, EF1a intron, FHL 1 intron, FLOT1 intron, FXYDI intron, GFAP intron, HBB intron, hCPE intron, hEf1a2-intron, HPD intron, IFI27L2 intron, Murine IgG chimeric intron, MVM intron, MVMi-AATp intron, MVMi-SynE-mTTRp intron, Rabit beta globin intron, RBP4 intron, RPL26 intron, RPL27 intron, S100A6 intron, sEEF1A1 intron, SV40 intron, TMSB10 intron, and UCHL1 intron.

In embodiments, the NARE comprises an enhancer or a portion of an enhancer. The enhancer or a portion of an enhancer may be selected from: ACTA1 enhancer, ACTC1 enhancer, CKM enhancer, CMV enhancer, CMV enhancer, MCK enhancer, mDES enhancer, mDES.1 enhancer, minCKM enhancer, minCKM2 enhancer, minDes enhancer, NRGN enhancer 1.1, NRGN enhancer 1.2, NRGN enhancer 2.1, NRGN enhancer 2.2, SV40 enhancer, and SV40 enhancer (SV40e).

In embodiments, the NARE comprises a UTR or a portion of a UTR. The UTR or a portion of a UTR may be selected from: CTNNB1 UTR, hEf1a2-utr1, hEf1a2-utr2, hNSE utr1, hNSE utr2, hSyn1_utr1, hSyn1_utr2, HTLV 5′ UTR, HTLV 5′UTR, L21 UTR, TMSB10_utr1, and TMSB10 utr2.

Also provided herein are NAREs used as references or controls. Some of the NAREs provide for constitutive expression of an operably linked transgene, that is, expression of the transgene is maintained at a constant level. Constitutive NAREs may drive expression of an operably linked transgene in a variety of cell types and tissues. As used herein, promoters starting with “C” are constitutive NAREs. Constitutive NAREs disclosed herein may be useful for the expression of genes in the liver, muscle, and/or central nervous system. Sequences for control NAREs are provided in Table 4.

In some embodiments, the NARE comprises a sequence that is at least 80% identical, at least 85% identical, at least 90% identical, at least 95% identical, at least 96% identical, at least 97% identical, at least 98% identical, or at least 99% identical to a sequence selected from the group consisting of SEQ ID NOs: 3-8, 11-69, 71-73, 76-149, and 256.

In some embodiments, the NARE comprises a sequence selected from the group consisting of SEQ ID NOs: 3-8, 11-69, 71-73, 76-149, and 256.

In some embodiments, the NARE comprises a sequence that is at least 80% identical, at least 85% identical, at least 90% identical, at least 95% identical, at least 96% identical, at least 97% identical, at least 98% identical, or at least 99% identical to a sequence selected from the group consisting of SEQ ID NOs: 15, 45-64, 71, and 98.

In some embodiments, the NARE comprises a sequence that is selected from the group consisting of SEQ ID NOs: 15, 45-64 and 98.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 150 and (ii) a sequence that is at least 80% identical, at least 85% identical, at least 90% identical, at least 95% identical, at least 96% identical, at least 97% identical, at least 98% identical, or at least 99% identical to SEQ ID NO: 151 or SEQ ID NO: 155.

In some embodiments, the NARE comprises: (i) SEQ ID NO: 150 and (ii) SEQ ID NO: 151 or SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 150 and (ii) a sequence that is at least 80% identical, at least 85% identical, at least 90% identical, at least 95% identical, at least 96% identical, at least 97% identical, at least 98% identical, or at least 99% identical to SEQ ID NO: 151.

In some embodiments, the NARE comprises: (i) SEQ ID NO: 150 and (ii) SEQ ID NO: 151.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 150 and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 150 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 153, 156, 161, 162, 163, 166, 167, 168, or 174; and (iii) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 169, 172 or 173.

In some embodiments, the NARE comprises: (i) any one of SEQ ID NOs: 154, 157, or 158 (ii) any one of SEQ ID NOs: 153, 156, 161, 162, 163, 166, 167, 168, or 174; and (iii) optionally any one of SEQ ID NOs: 169, 172 or 173.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 157; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 153, 156, 161, 162, 163, 166, 167, 168, or 174; and (iii) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 169, 172 or 173.

In some embodiments, the NARE comprises: (i) SEQ ID NO: 157 (ii) any one of SEQ ID NOs: 153, 156, 161, 162, 163, 166, 167, 168, or 174; and (iii) optionally any one of SEQ ID NOs: 169, 172 or 173.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 156 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 156 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 156 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 156 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 161 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 161 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 161 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 161 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 162 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 162 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 163 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 163 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 163 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 163 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 166 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 166 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 166 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 166 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 167 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 167 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 167 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 167 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169 and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 168; (ii) SEQ ID NO: 169; and (iii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169 and (iii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 168; (ii) SEQ ID NO: 169; and (iii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises (i) SEQ ID NO: 153 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 157; and (iii) a sequence that is at least 80% identical, at least 85% identical, at least 90% identical, at least 95% identical, at least 96% identical, at least 97% identical, at least 98% identical, or at least 99% identical to SEQ ID NO: 172.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158; and (iii) a sequence that is at least 80% identical, at least 85% identical, at least 90% identical, at least 95% identical, at least 96% identical, at least 97% identical, at least 98% identical, or at least 99% identical to SEQ ID NO: 172 or SEQ ID NO: 173.

In some embodiments, the NARE comprises (i) SEQ ID NO: 153; (ii) any one of SEQ ID NOs: 154, 157, or 158; and (iii) SEQ ID NO: 172 or SEQ ID NO: 173.

In some embodiments, the NARE comprises (i) SEQ ID NO: 153; (ii) SEQ ID NO: 157; and (iii) SEQ ID NO: 172.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158; and (iii) a sequence that is at least 80% identical, at least 85% identical, at least 90% identical, at least 95% identical, at least 96% identical, at least 97% identical, at least 98% identical, or at least 99% identical to SEQ ID NO: 172 or SEQ ID NO: 173.

In some embodiments, the NARE comprises: (i) SEQ ID NO: 153; (ii) any one of SEQ ID NOs: 154, 157, or 158.; and (iii) SEQ ID NO: 172 or SEQ ID NO: 173.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 157; and (iii) a sequence that is at least 80% identical, at least 85% identical, at least 90% identical, at least 95% identical, at least 96% identical, at least 97% identical, at least 98% identical, or at least 99% identical to SEQ ID NO: 173.

In some embodiments, the NARE comprises: (i) SEQ ID NO: 153; (ii) SEQ ID NO: 157; and (iii) SEQ ID NO: 173.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 153 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 153 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 174; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises: (i) SEQ ID NO: 174 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 174; and (ii) a sequence that is at least 80% identical, at least 85% identical, at least 90% identical, at least 95% identical, at least 96% identical, at least 97% identical, at least 98% identical, or at least 99% identical to SEQ ID NO: 157.

In some embodiments, the NARE comprises: (i) SEQ ID NO: 174 and (ii) SEQ ID NO: 157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 171; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, 158 or 176.

In some embodiments, the NARE comprises: (i) SEQ ID NO: 153; (ii) SEQ ID NO: 171; and (iii) any one of SEQ ID NOs: 154, 157, 158 or 176.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 171; and (iii) a sequence that is at least 80% identical, at least 85% identical, at least 90% identical, at least 95% identical, at least 96% identical, at least 97% identical, at least 98% identical, or at least 99% identical to SEQ ID NO: 154 or SEQ ID NO: 176.

In some embodiments, the NARE comprises: (i) SEQ ID NO: 153; (ii) SEQ ID NO: 171; and (iii) SEQ ID NO: 154 or SEQ ID NO: 176.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158; and (iii) a sequence that is at least 80% identical, at least 85% identical, at least 90% identical, at least 95% identical, at least 96% identical, at least 97% identical, at least 98% identical, or at least 99% identical to SEQ ID NO: 171.

In some embodiments, the NARE comprises: (i) SEQ ID NO: 153; (ii) any one of SEQ ID NOs: 154, 157, or 158; and (iii) SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 154; and (iii) a sequence that is at least 80% identical, at least 85% identical, at least 90% identical, at least 95% identical, at least 96% identical, at least 97% identical, at least 98% identical, or at least 99% identical to SEQ ID NO: 171.

In some embodiments, the NARE comprises: (i) SEQ ID NO: 153; (ii) SEQ ID NO: 154; and (iii) SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, 158, 176, 188, or 250; and (iii) optionally, a sequence that is at least 80% identical, at least 85% identical, at least 90% identical, at least 95% identical, at least 96% identical, at least 97% identical, at least 98% identical, or at least 99% identical to SEQ ID NO: 155 or SEQ ID NO: 171.

In some embodiments, the NARE comprises: (i) SEQ ID NO: 153; (ii) any one of SEQ ID NOs: 154, 157, 158, 176, 188, or 250; and (iii) optionally, SEQ ID NO: 155 or SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 176, 188, or 250; and (iii) optionally, a sequence that is at least 80% identical, at least 85% identical, at least 90% identical, at least 95% identical, at least 96% identical, at least 97% identical, at least 98% identical, or at least 99% identical to SEQ ID NO: 155 or SEQ ID NO: 171.

In some embodiments, the NARE comprises: (i) SEQ ID NO: 153; (ii) any one of SEQ ID NOs: 154, 176, 188, or 250; and (iii) optionally, SEQ ID NO: 155 or SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 183 and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 153; (ii) SEQ ID NO: 188; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises (i) SEQ ID NO: 153 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 154.

In some embodiments, the NARE comprises: (i) SEQ ID NO: 153 and (ii) SEQ ID NO: 154.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, and 158; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 153; (ii) any one of SEQ ID NOs: 154, 157, and 158; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 154; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 153; (ii) SEQ ID NO: 154; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to of SEQ ID NO: 153; and (ii) a sequence that is at least 80% identical, at least 85% identical, at least 90% identical, at least 95% identical, at least 96% identical, at least 97% identical, at least 98% identical, or at least 99% identical to SEQ ID NO: 188.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 188.

In some embodiments, the NARE comprises: (i) SEQ ID NO: 153 and (ii) SEQ ID NO: 188.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 250.

In some embodiments, the NARE comprises: (i) SEQ ID NO: 153 and (ii) SEQ ID NO: 250.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153 and (ii) a sequence that is at least 80% identical, at least 85% identical, at least 90% identical, at least 95% identical, at least 96% identical, at least 97% identical, at least 98% identical, or at least 99% identical to SEQ ID NO: 176.

In some embodiments, the NARE comprises: (i) SEQ ID NO: 153 and (ii) SEQ ID NO: 176.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% 80% identical, at least 85% identical, at least 90% identical, at least 95% identical, at least 96% identical, at least 97% identical, at least 98% identical, or at least 99% identical to SEQ ID NO: 171.

In some embodiments, the NARE comprises: (i) SEQ ID NO: 153; (ii) SEQ ID NO: 176; and (iii) SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 153; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 153; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to of SEQ ID NO: 155; (ii) a sequence that is at least 80% identical, at least 85% identical, at least 90% identical, at least 95% identical, at least 96% identical, at least 97% identical, at least 98% identical, or at least 99% identical to SEQ ID NO: 176 and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 175 or 177-187.

In some embodiments, the NARE comprises: (i) SEQ ID NO: 155; (ii) SEQ ID NO: 176; and (iii) any one of SEQ ID NOs: 175 or 177-187.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to of SEQ ID NO: 175; (ii) a sequence that is at least 80% identical, at least 85% identical, at least 90% identical, at least 95% identical, at least 96% identical, at least 97% identical, at least 98% 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 175; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 177; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 177; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 178; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 178; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 179; (ii) a sequence that is at least 80% identical, at least 85% identical, at least 90% identical, at least 95% identical, at least 96% identical, at least 97% identical, at least 98% 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 179; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 180; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 180; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 181; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 181; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 182; (ii) a sequence that is at least 80% identical, at least 85% identical, at least 90% identical, at least 95% identical, at least 96% identical, at least 97% identical, at least 98% 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 182; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 183; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 184; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 184; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 185; (ii) a sequence that is at least 80% identical, at least 85% identical, at least 90% identical, at least 95% identical, at least 96% identical, at least 97% identical, at least 98% 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 185; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 186; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 186; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 187; (ii) a sequence that is at least 80% identical, at least 85% identical, at least 90% identical, at least 95% identical, at least 96% identical, at least 97% identical, at least 98% identical, or at least 99% identical to SEQ ID NO: 176; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 187; (ii) SEQ ID NO: 176; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 193, 194, or 195; (iii) optionally, a sequence that is at least 80% identical, at least 85% identical, at least 90% identical, at least 95% identical, at least 96% identical, at least 97% identical, at least 98% identical, or at least 99% identical to SEQ ID NO: 162; (iv) optionally, a sequence that is at least 80% identical, at least 85% identical, at least 90% identical, at least 95% identical, at least 96% identical, at least 97% identical, at least 98% identical, or at least 99% identical to SEQ ID NO: 168; and (v) optionally, SEQ ID NO: 169.

In some embodiments, the NARE comprises: (i) SEQ ID NO: 190 or SEQ ID NO: 191; (ii) any one of SEQ ID NOs: 193, 194, or 195; (iii) optionally SEQ ID NO: 162; (iv) optionally, SEQ ID NO: 168; and (v) optionally, SEQ ID NO: 169.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 195; and (iii) optionally, a sequence that is at least 80% identical, at least 85% identical, at least 90% identical, at least 95% identical, at least 96% identical, at least 97% identical, at least 98% identical, or at least 99% identical to SEQ ID NO: 162; (iv) optionally, a sequence that is at least 80% identical, at least 85% identical, at least 90% identical, at least 95% identical, at least 96% identical, at least 97% identical, at least 98% identical, or at least 99% identical to SEQ ID NO: 168; and (v) optionally, SEQ ID NO: 169.

In some embodiments, the NARE comprises: (i) SEQ ID NO: 190; (ii) SEQ ID NO: 193 or SEQ ID NO: 195; (iii) optionally SEQ ID NO: 168; (iv) optionally, SEQ ID NO: 168; and (v) optionally, SEQ ID NO: 169.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191; and (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ D NO: 194.

In some embodiments, the NARE comprises: (i) SEQ ID NO: 190 or SEQ ID NO: 191; and (ii) SEQ ID NO: 193 or SEQ D NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; and (ii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 190; and (ii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 168; (iii) a sequence that is at least 80% identical, at least 85% identical, at least 90% identical, at least 95% identical, at least 96% identical, at least 97% identical, at least 98% identical, or at least 99% identical to SEQ ID NO: 169; (iv) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191; and (v) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 162; (ii) SEQ ID NO: 168; (iii) SEQ ID NO: 169; (iv) SEQ ID NO: 190 or SEQ ID NO: 191; and (v) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 168; (iii) a sequence that is at least 80% identical, at least 85% identical, at least 90% identical, at least 95% identical, at least 96% identical, at least 97% identical, at least 98% identical, or at least 99% identical to SEQ ID NO: 169; (iv) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191; and (v) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 162; (ii) SEQ ID NO: 168; (iii) SEQ ID NO: 169; (iv) SEQ ID NO: 190 or SEQ ID NO: 191; and (v) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 168; (iii) a sequence that is at least 80% identical, at least 85% identical, at least 90% identical, at least 95% identical, at least 96% identical, at least 97% identical, at least 98% identical, or at least 99% identical to SEQ ID NO: 169; (iv) a sequence that is at least 80% identical, at least 85% identical, at least 90% identical, at least 95% identical, at least 96% identical, at least 97% identical, at least 98% identical, or at least 99% identical to SEQ ID NO: 190; and (v) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 162; (ii) SEQ ID NO: 168; (iii) SEQ ID NO: 169; (iv) SEQ ID NO: 190; and (v) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191; and (ii) a sequence that is at least 80% identical, at least 85% identical, at least 90% identical, at least 95% identical, at least 96% identical, at least 97% identical, at least 98% identical, or at least 99% identical to SEQ ID NO: 195.

In some embodiments, the NARE comprises (i) SEQ ID NO: 190 or SEQ ID NO: 191 and (ii) SEQ ID NO: 195.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; and (ii) a sequence that is at least 80% identical, at least 85% identical, at least 90% identical, at least 95% identical, at least 96% identical, at least 97% identical, at least 98% identical, or at least 99% identical to SEQ ID NO: 195.

In some embodiments, the NARE comprises: (i) SEQ ID NO: 190 and (ii) SEQ ID NO: 195.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to any one of SEQ ID NOs: 197, 202 or 203; (iv) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 190, 191, 192, 196, 202, 203, 204, 205 or 206; (v) optionally, a sequence that is at least 80% identical, at least 85% identical, at least 90% identical, at least 95% identical, at least 96% identical, at least 97% identical, at least 98% identical, or at least 99% identical to SEQ ID NO: 155 or SEQ ID NO: 162; and (vi) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 166, 193, or 194.

In some embodiments, the NARE comprises: (i) SEQ ID NO: 168; (ii) SEQ ID NO: 169; (iii) any one of SEQ ID NOs: 197, 202 or 203; (iv) optionally, any one of SEQ ID NOs: 190, 191, 192, 196, 203, 204, 205 or 206; (v) optionally, SEQ ID NO: 155 or SEQ ID NO: 162; and (vi) optionally, any one of SEQ ID NOs: 166, 193, or 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197 or SEQ ID NO: 202; (iv) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 191, 192, 196, 203, 204, or 206; (v) optionally, a sequence that is at least 80% identical, at least 85% identical, at least 90% identical, at least 95% identical, at least 96% identical, at least 97% identical, at least 98% identical, or at least 99% identical to SEQ ID NO: 155 or SEQ ID NO: 162; and (vi) optionally, a sequence that is at least 80% identical, at least 85% identical, at least 90% identical, at least 95% identical, at least 96% identical, at least 97% identical, at least 98% identical, or at least 99% identical to SEQ ID NO: 166 or SEQ ID NO: 193.

In some embodiments, the NARE comprises: (i) SEQ ID NO: 168; (ii) SEQ ID NO: 169; (iii) SEQ ID NOs: 197 or SEQ ID NO: 202; (iv) optionally, any one of SEQ ID NOs: 191, 192, 196, 203, 204, or 206; (v) optionally, SEQ ID NO: 155 or SEQ ID NO: 162; and (vi) optionally, SEQ ID NO: 166 or SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; and (iv) a sequence that is at least 80% identical, at least 85% identical, at least 90% identical, at least 95% identical, at least 96% identical, at least 97% identical, at least 98% identical, or at least 99% identical to SEQ ID NO: 190 or SEQ ID NO: 191.

In some embodiments, the NARE comprises: (i) SEQ ID NO: 168; (ii) SEQ ID NO: 169; (iii) SEQ ID NO: 197; and (iv) SEQ ID NO: 190 or SEQ ID NO: 191.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; and (iv) a sequence that is at least 80% identical, at least 85% identical, at least 90% identical, at least 95% identical, at least 96% identical, at least 97% identical, at least 98% identical, or at least 99% identical to SEQ ID NO: 191.

In some embodiments, the NARE comprises: (i) SEQ ID NO: 168; (ii) SEQ ID NO: 169; (iii) SEQ ID NO: 197; and (iv) SEQ ID NO: 191.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 166; (iii) a sequence that is at least 80% identical, at least 85% identical, at least 90% identical, at least 95% identical, at least 96% identical, at least 97% identical, at least 98% identical, or at least 99% identical to SEQ ID NO: 168; (iv) a sequence that is at least 80% identical, at least 85% identical, at least 90% identical, at least 95% identical, at least 96% identical, at least 97% identical, at least 98% identical, or at least 99% identical to SEQ ID NO: 169; (v) a sequence that is at least 80% identical, at least 85% identical, at least 90% identical, at least 95% identical, at least 96% identical, at least 97% identical, at least 98% identical, or at least 99% identical to SEQ ID NO: 197; and (vi) a sequence that is at least 80% identical, at least 85% identical, at least 90% identical, at least 95% identical, at least 96% identical, at least 97% identical, at least 98% identical, or at least 99% identical to SEQ ID NO: 196.

In some embodiments, the NARE comprises: (i) SEQ ID NO: 162; (ii) SEQ ID NO: 166; (iii) SEQ ID NO: 168; (iv) SEQ ID NO: 169; (v) SEQ ID NO: 197; and (vi) SEQ ID NO: 196.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 168; (iii) a sequence that is at least 80% identical, at least 85% identical, at least 90% identical, at least 95% identical, at least 96% identical, at least 97% identical, at least 98% identical, or at least 99% identical to SEQ ID NO: 169; (iv) a sequence that is at least 80% identical, at least 85% identical, at least 90% identical, at least 95% identical, at least 96% identical, at least 97% identical, at least 98% identical, or at least 99% identical to SEQ ID NO: 202 or SEQ ID NO: 203; and (vi) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 162; (ii) SEQ ID NO: 168; (iii) SEQ ID NO: 169; (iv) SEQ ID NO: 202 or SEQ ID NO: 203; and (vi) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 162; (ii) a sequence that is at least 80% identical, at least 85% identical, at least 90% identical, at least 95% identical, at least 96% identical, at least 97% identical, at least 98% identical, or at least 99% identical to SEQ ID NO: 168; (iii) a sequence that is at least 80% identical, at least 85% identical, at least 90% identical, at least 95% identical, at least 96% identical, at least 97% identical, at least 98% identical, or at least 99% identical to SEQ ID NO: 169; (iv) a sequence that is at least 80% identical, at least 85% identical, at least 90% identical, at least 95% identical, at least 96% identical, at least 97% identical, at least 98% identical, or at least 99% identical to SEQ ID NO: 202; and (vi) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 162; (ii) SEQ ID NO: 168; (iii) SEQ ID NO: 169; (iv) SEQ ID NO: 202; and (vi) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; and (iv) a sequence that is at least 80% identical, at least 85% identical, at least 90% identical, at least 95% identical, at least 96% identical, at least 97% identical, at least 98% identical, or at least 99% identical to SEQ ID NO: 202 or SEQ ID NO: 203.

In some embodiments, the NARE comprises: (i) SEQ ID NO: 168; (ii) SEQ ID NO: 169; (iii) SEQ ID NO: 197; and (iv) SEQ ID NO: 202 or SEQ ID NO: 203.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; and (iv) a sequence that is at least 80% identical, at least 85% identical, at least 90% identical, at least 95% identical, at least 96% identical, at least 97% identical, at least 98% identical, or at least 99% identical to SEQ ID NO: 203.

In some embodiments, the NARE comprises: (i) SEQ ID NO: 168; (ii) SEQ ID NO: 169; (iii) SEQ ID NO: 197; and (iv) SEQ ID NO: 203.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; (iv) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (v) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 168; (ii) SEQ ID NO: 169; (iii) SEQ ID NO: 197; (iv) SEQ ID NO: 205 or SEQ ID NO: 206; and (v) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; (iv) a sequence that is at least 80% identical, at least 85% identical, at least 90% identical, at least 95% identical, at least 96% identical, at least 97% identical, at least 98% identical, or at least 99% identical to SEQ ID NO: 206; and (v) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 168; (ii) SEQ ID NO: 169; (iii) SEQ ID NO: 197; (iv) SEQ ID NO: 206; and (v) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 192; (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 201-203.

In some embodiments, the NARE comprises: (i) SEQ ID NO: 192 and (ii) SEQ ID NO: 193 or SEQ ID NO: 194; and (iii) any one of SEQ ID NOs: 201-203.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 192; (ii) a sequence that is at least 80% identical, at least 85% identical, at least 90% identical, at least 95% identical, at least 96% identical, at least 97% identical, at least 98% identical, or at least 99% identical to SEQ ID NO: 193; and (iii) SEQ ID NO: 201 or SEQ ID NO: 202.

In some embodiments, the NARE comprises: (i) SEQ ID NO: 192 (ii) SEQ ID NO: 193; and (iii) SEQ ID NO: 201 or SEQ ID NO: 202.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 201; and (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 201 and (ii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 201; and (ii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 201 and (ii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 202 or SEQ ID NO: 203 and (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 202 or SEQ ID NO: 203 and (ii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 202 or SEQ ID NO: 203; and (ii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 202 and (ii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 155; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 204, 205, 206, or 236-238; (iii) optionally. a sequence that is at least 80% identical, at least 85% identical, at least 90% identical, at least 95% identical, at least 96% identical, at least 97% identical, at least 98% identical, or at least 99% identical to SEQ ID NO: 168; (iv) optionally, a sequence that is at least 80% identical, at least 85% identical, at least 90% identical, at least 95% identical, at least 96% identical, at least 97% identical, at least 98% identical, or at least 99% identical to SEQ ID NO: 169; and (v) optionally, a sequence that is at least 80% identical, at least 85% identical, at least 90% identical, at least 95% identical, at least 96% identical, at least 97% identical, at least 98% identical, or at least 99% identical to SEQ ID NO: 197.

In some embodiments, the NARE comprises: (i) SEQ ID NO: 155; (ii) any one of SEQ ID NOs: 204, 205, 206, or 236-238; (iii) optionally, SEQ ID NO: 168, (iv) optionally, SEQ ID NO: 169; and (v) optionally, SEQ ID NO: 197.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 155; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 204, 205, or 236-238; (iii) optionally, a sequence that is at least 80% identical, at least 85% identical, at least 90% identical, at least 95% identical, at least 96% identical, at least 97% identical, at least 98% identical, or at least 99% identical to SEQ ID NO: 168; (iv) optionally, a sequence that is at least 80% identical, at least 85% identical, at least 90% identical, at least 95% identical, at least 96% identical, at least 97% identical, at least 98% identical, or at least 99% identical to SEQ ID NO: 169; and (v) optionally, a sequence that is at least 80% identical, at least 85% identical, at least 90% identical, at least 95% identical, at least 96% identical, at least 97% identical, at least 98% identical, or at least 99% identical to SEQ ID NO: 197.

In some embodiments, the NARE comprises: (i) SEQ ID NO: 155; (ii) any one of SEQ ID NOs: 204, 205, or 236-238; (iii) optionally, SEQ ID NO: 168, (iv) optionally, SEQ ID NO: 169; and (v) optionally, SEQ ID NO: 197.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 204; and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 204 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 205 or SEQ ID NO: 206 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 205 and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 205 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 238; and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises (i) SEQ ID NO: 238 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 236 or SEQ ID NO: 237 and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 236 or SEQ ID NO: 237 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 237 and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 237 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 236 or SEQ ID NO: 237; and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 236 or SEQ ID NO: 237 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 236 and (ii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 236 and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 168; (ii) a sequence that is at least 80% identical, at least 85% identical, at least 90% identical, at least 95% identical, at least 96% identical, at least 97% identical, at least 98% identical, or at least 99% identical to SEQ ID NO: 169; (iii) a sequence that is at least 80% identical, at least 85% identical, at least 90% identical, at least 95% identical, at least 96% identical, at least 97% identical, at least 98% identical, or at least 99% identical to SEQ ID NO: 197; (iv) a sequence that is at least 80% identical, at least 85% identical, at least 90% identical, at least 95% identical, at least 96% identical, at least 97% identical, at least 98% identical, or at least 99% identical to SEQ ID NO: 204; (v) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 168; (ii) SEQ ID NO: 169; (iii) SEQ ID NO: 197; (iv) SEQ ID NO: 204; and (v) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 248; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 239-247.

In some embodiments, the NARE comprises: (i) SEQ ID NO: 248 and (ii) any one of SEQ ID NOs: 239-247.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 239 and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 239 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 240; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 240 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 241; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 241 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 242; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 242 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 243; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 243 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 244; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 244 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 245; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 245 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 246; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 246 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 247; and (ii) a sequence that is at least 80% identical, at least 85% identical, at least 90% identical, at least 95% identical, at least 96% identical, at least 97% identical, at least 98% identical, or at least 99% identical to SEQ ID NO: 248.

In some embodiments, the NARE comprises: (i) SEQ ID NO: 247 and (ii) SEQ ID NO: 248.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 208; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 209-214.

In some embodiments, the NARE comprises: (i) SEQ ID NO: 208 and (ii) any one of SEQ ID NOs: 209-214.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 207 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 209 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 210 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 211 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 212 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 213 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; and (ii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO: 214 and (ii) SEQ ID NO: 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 74; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 193, 194, 207, 209-214, 216-232, 233-235, or 249.

In some embodiments, the NARE comprises: (i) SEQ ID NO: 74 and (ii) any one of SEQ ID NOs: 193, 194, 207, 209-214, 216-232, 233-235, or 249.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 74; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 194, 207, 209-214, 216-232, 233-235, or 249.

In some embodiments, the NARE comprises: (i) SEQ ID NO: 74 and (ii) any one of SEQ ID NOs: 194, 207, 209-214, 216-232, 233-235, or 249.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 207 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 209 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 210 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 211 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 212 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 213 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 214 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 207 or 209-215.

In some embodiments, the NARE comprises: (i) SEQ ID NO: 193 or SEQ ID NO: 194; (ii) SEQ ID NO: 215; and (iii) any one of SEQ ID NOs: 207 or 209-215.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 207 or 209-215.

In some embodiments, the NARE comprises: (i) SEQ ID NO: 193; (ii) SEQ ID NO: 215; and (iii) any one of SEQ ID NOs: 207 or 209-215.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 207; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 207; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 209; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 209; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 210; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 210; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 211; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 211; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 212; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 212; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 213; (ii) SEQ ID NO: 215; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 213; (ii) SEQ ID NO: 215; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 213; (ii) SEQ ID NO: 214; and (iii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; (ii) a sequence that is at least 80% identical, at least 85% identical, at least 90% identical, at least 95% identical, at least 96% identical, at least 97% identical, at least 98% identical, or at least 99% identical to SEQ ID NO: 215; and (iii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 213; (ii) SEQ ID NO: 214; and (iii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 155; (ii) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 207 or 209-215.

In some embodiments, the NARE comprises: (i) SEQ ID NO: 155; (ii) SEQ ID NO: 205 or SEQ ID NO: 206; and (iii) any one of SEQ ID NOs: 207 or 209-215.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 155; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 207 or 209-215.

In some embodiments, the NARE comprises: (i) SEQ ID NO: 155; (ii) SEQ ID NO: 205; and (iii) any one of SEQ ID NOs: 207 or 209-215.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; (ii) a sequence that is at least 80% identical, at least 85% identical, at least 90% identical, at least 95% identical, at least 96% identical, at least 97% identical, at least 98% sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 207; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 207; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 207; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; (ii) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 209; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 209; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 209; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; (ii) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 210; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 210; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 210; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; (ii) a sequence that is at least 80% identical, at least 85% identical, at least 90% identical, at least 95% identical, at least 96% identical, at least 97% identical, at least 98% sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 211; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 211; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 211; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; (ii) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 212; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 212; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 212; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; (ii) a sequence that is at least 80% identical, at least 85% identical, at least 90% identical, at least 95% identical, at least 96% identical, at least 97% identical, at least 98% identical, or at least 99% identical to SEQ ID NO: 205 or SEQ ID NO: 206; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 213; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 213; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 213; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; (ii) a sequence that is at least 80% identical, at least 85% identical, at least 90% identical, at least 95% identical, at least 96% identical, at least 97% identical, at least 98% sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 214; (ii) SEQ ID NO: 205 or SEQ ID NO: 206; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 214; (ii) a sequence that is at least 80% identical, at least 85% identical, at least 90% identical, at least 95% identical, at least 96% identical, at least 97% identical, at least 98% identical, or at least 99% identical to SEQ ID NO: 205; and (iii) a sequence that is at least 80% identical, at least 85% identical, at least 90% identical, at least 95% identical, at least 96% identical, at least 97% identical, at least 98% identical, or at least 99% identical to SEQ ID NO: 155.

In some embodiments, the NARE comprises: (i) SEQ ID NO: 214; (ii) SEQ ID NO: 205; and (iii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 216; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 216 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 217 and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 217 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 218; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 218 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 219; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 219 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 220; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 220 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 221; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 221 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 222; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 222 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 223; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 223 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 224; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 224 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 225; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 225 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 226; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 226 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 227; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 227 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 228; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 228 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 229; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 229 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 230; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 230 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 231; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 231 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 232; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 232 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 193 or SEQ ID NO: 194 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 194; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 194 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 233; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 233 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 234; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 234 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 235; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 235 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 249; and (ii) a sequence that is at least 80% identical, at least 85% identical, at least 90% identical, at least 95% identical, at least 96% identical, at least 97% identical, at least 98% identical, or at least 99% identical to SEQ ID NO: 74.

In some embodiments, the NARE comprises: (i) SEQ ID NO: 249 and (ii) SEQ ID NO: 74.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 165 and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 154, 157, or 158.

In some embodiments, the NARE comprises (i) SEQ ID NO: 165 and (ii) any one of SEQ ID NOs: 154, 157, or 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 165 and (ii) a sequence that is at least 80% identical, at least 85% identical, at least 90% identical, at least 95% identical, at least 96% identical, at least 97% identical, at least 98% identical, or at least 99% identical to SEQ ID NO: 158.

In some embodiments, the NARE comprises (i) SEQ ID NO: 165 and (ii) SEQ ID NO: 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 199 and (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises (i) SEQ ID NO: 199 and (ii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 199; and (ii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises (i) SEQ ID NO: 199 and (ii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; and (ii) a sequence that is at least 80% identical, at least 85% identical, at least 90% identical, at least 95% identical, at least 96% identical, at least 97% identical, at least 98% identical, or at least 99% identical to SEQ ID NO: 200.

In some embodiments, the NARE comprises: (i) SEQ ID NO: 164 and (ii) SEQ ID NO: 200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 189 and (ii) a sequence that is at least 80% identical, at least 85% identical, at least 90% identical, at least 95% identical, at least 96% identical, at least 97% identical, at least 98% identical, or at least 99% identical to SEQ ID NO: 193 or SEQ ID NO: 194.

In some embodiments, the NARE comprises: (i) SEQ ID NO: 189 and (ii) SEQ ID NO: 193 or SEQ ID NO: 194.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 189 and (ii) a sequence that is at least 80% identical, at least 85% identical, at least 90% identical, at least 95% identical, at least 96% identical, at least 97% identical, at least 98% identical, or at least 99% identical to SEQ ID NO: 193.

In some embodiments, the NARE comprises: (i) SEQ ID NO: 189 and (ii) SEQ ID NO: 193.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 202 or SEQ ID NO: 203; and (ii) a sequence that is at least 80% identical, at least 85% identical, at least 90% identical, at least 95% identical, at least 96% identical, at least 97% identical, at least 98% identical, or at least 99% identical to SEQ ID NO: 195.

In some embodiments, the NARE comprises: (i) SEQ ID NO: 202 or SEQ ID NO: 203 and (ii) SEQ ID NO: 195.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 202; and (ii) a sequence that is at least 80% identical, at least 85% identical, at least 90% identical, at least 95% identical, at least 96% identical, at least 97% identical, at least 98% identical, or at least 99% identical to SEQ ID NO: 195.

In some embodiments, the NARE comprises: (i) SEQ ID NO: 202 and (ii) SEQ ID NO: 195.

Provided herein is a method for expression of a transgene in the hepatocytes (e.g., in the liver), wherein the transgene is operably linked to a NARE disclosed herein particularly suitable to drive expression in in hepatocytes (e.g., in the liver).

### NARE Modifications

A person skilled in the art will appreciate that certain modifications can be made to the NAREs disclosed herein without eliminating the ability of the NARE to drive gene expression in the desired tissue/cell type. Various in vitro and in vivo methods of confirming that a modified NARE is still capable of driving gene expression are known in the art, including, but not limited to, the methods used herein. For example, the strength of a NARE may be assessed by operatively linking the promoter to a transgene encoding a protein and measuring transgene expression, for example by detecting the mRNA encoding the protein, or by measuring presence or activity of the protein, e.g., by ELISA, Western Blot, fluorescence, enzymatic activity of the protein, etc.

Provided herein are NAREs comprising one or more components. Provided herein are NAREs comprising one or more sequence components (or sequence variants thereof) listed in any one of Tables 1-7. Provided herein are NAREs comprising one or more sequence components (or sequence variants thereof) listed in Table 7. Provided herein is a NARE that comprise one or more components (or sequence variants thereof) of a full-length NARE provided in Table 7. Merely to illustrate in a non-limiting example, provided is a NARE that comprises one or more of the components (or sequence variants thereof) of NARE L4 (AATcre-mTTR): (1) AATcre; and (2) mTTRmd promoter. In embodiments, provided is a NARE that comprises one or more components (or sequence variants thereof) of a full-length NARE provided in Table 7, whereby the components (or sequence variants thereof) are arranged from 5′ to 3′ as presented as in Table 7. In embodiments, provided is a NARE that comprises one or more components (or sequence variants thereof) of a full-length NARE provided in Table 7, whereby the components (or sequence variants thereof) are not arranged from 5′ to 3′ as presented as in Table 4, but in a different order.

In embodiments, in NARE of Table 5 or Table 7, one or more of the components have been replaced with a different sequence, including a component (or sequence variants thereof) from a different NARE of Table 5 or Table 7. Further, also contemplated are NAREs that comprise components (or sequence variants thereof) derived from two or more NAREs disclosed Table 5 or Table 7.

Moreover, contemplated are NARE variants that include sequence in addition (e.g., added at either end or inserted within the NARE sequence) to the elements of a NARE disclosed herein. Likewise, provided are NARE variants in which certain sequence has been removed from NAREs disclosed herein (e.g., as a terminal or internal deletion). In embodiments, provided are NAREs comprising one or more components without any additional nucleic acid sequence(s) joining the NARE's components. In embodiments, provided are NAREs comprising one or more components, whereby the individual components are connected by additional nucleic acid sequences. These additional nucleic acid sequences may or may not be relevant for expression of an operatively linked transgene. In embodiments, some of the components are directly linked, while other components are linked to other components via an intervening sequence. As such, contemplated are variants of the NAREs disclosed in Table 5 or Table 7, wherein additional sequence has been added between the different components. Further contemplated are variants of the NAREs disclosed in Table 5 or Table 7, which disclose the same components of a given NARE in Table 5 or Table 7, but differ in the sequence(s) connecting the individual components.

### Nucleic Acid Constructs and Vectors

In one aspect, provided are nucleic acid constructs and vectors and their use for the introduction of a transgene or an expression construct into a cell. Nucleic acid constructs include expression constructs including plasmids. The term “expression construct” refers to a recombinant polynucleotide construct that includes a nucleic acid coding for an RNA capable of being transcribed in a cell. Methods for constructing expression constructs and plasmids through standard recombinant techniques are known in the art.

In some embodiments, the expression construct comprises a sequence encoding ATP7B, wherein the ATP7B-encoding sequence is codon-optimized.

In one aspect, provided is an expression construct comprising:
a) a promoter;
b) a sequence encoding ATP7B, operatively linked to the promoter; and
c) a polyadenylation signal.

In some embodiments, the vectors comprise recombinant DNA constructs that include additional DNA elements, including DNA segments that provide for the replication of the DNA in a host cell and expression of the target gene in target cells at appropriate levels. “Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro or in vivo. In some embodiments, a vector refers to a virion comprising a recombinant viral genome, wherein the viral genome comprises one or more ITRs and a transgene.

In one embodiment, the vector is a viral vector or a combination of multiple viral vectors. In one aspect, provided is a vector comprising any of the nucleic acid constructs disclosed herein.

Provided herein are nucleic acid constructs and vectors comprising a nucleic acid regulatory element disclosed herein operatively linked to a transgene. In embodiments, the nucleic acid constructs or vectors disclosed herein comprise additional regulatory elements, including, but not limited to, promoters, enhancers, translation initiation signals, introns, and/or splicing enhancers. In embodiments, the nucleic acid constructs or vectors disclosed herein comprise a polyadenylation sequence. In embodiments, the nucleic acid constructs or vectors disclosed herein comprise an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. In one embodiment, the IRES is located 3′ of the transgene.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP 1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises a nucleic acid construct disclosed herein.

In some embodiments, the viral vector disclosed herein comprises a nucleic acid comprising an AAV 5′ ITR and 3′ ITR located 5′ and 3′ of the sequence encoding a transgene, respectively. In embodiments, the transgene is UPF1, BDNF, or ATP7B. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to transgene. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, provided is a vector comprising a nucleic acid sequence comprising (i) a nucleic acid construct disclosed herein and (ii) one or more inverted terminal repeats (ITR). In one embodiment, the nucleic acid sequence comprises a 5′ ITR and a 3′ ITR. In one embodiment, the 5′ ITR and a 3′ ITR are derived from adeno-associated virus (AAV) serotype AAV2.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and

AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh. 10 (i.e., an AAV containing AAVrh. 10 ITRs and AAVrh. 10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In some embodiments, the AAV vector contains two or more capsid proteins selected from different serotypes. In some embodiments, the AAV vector contains an rAAV2-retro and an AAVrh.10 capsid protein. In some embodiments, the AAV vector contains rAAV2-retro and AAVrh.10 capsid proteins respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50. In some embodiments, the AAV vector contains AAVrh.10 and rAAV2-retro capsid proteins, respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50.

In some embodiments, a mixture of (1) AAV vectors comprising rAAV2-retro and (2) AAV vectors comprising AAVrh.10 is used. In some embodiments, a ratio of the (1) AAV vectors comprising rAAV2-retro and (2) AAV vectors comprising AAVrh. 10, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used. In some embodiments, a ratio of the (1) AAV vectors comprising AAVrh. 10 and (2) AAV vectors comprising rAAV2-retro, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used.

### Other Viral Vectors

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for example, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells include hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a NARE, an optional spacer interposed between the NARE and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5 (1): 56-63, for the method used in the following examples, employing the plasmid with the transgene, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions can be produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the transgene is isolated from the cell or cell culture in the absence of contaminating helper virus or wildtype AAV.

Expression of a transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

### Cells

In embodiments, the nucleic acid constructs and vectors disclosed herein are used to deliver a NARE operatively linked to a transgene to a cell. Provided herein are cells comprising a NARE, a nucleic acid construct, or a vector disclosed herein. In embodiments, the cell is a liver cell. The cell may be a mammalian cell. The cell may be a human cell. The cell may be isolated.

### Pharmaceutical Compositions

Provided herein are pharmaceutical compositions comprising a nucleic acid construct or vector disclosed herein and a pharmaceutically acceptable excipient.

The nucleic acid construct or vector disclosed herein is preferably assessed for contamination by conventional methods and then formulated into a pharmaceutical composition suitable for storage and/or administration to a patient.

Formulations of the nucleic acid constructs or vectors disclosed herein disclosed herein involve the use of a pharmaceutically and/or physiologically acceptable vehicle or carrier, particularly one suitable for injection, such as buffered saline or other buffers, e.g., HEPES, to maintain pH at appropriate physiological levels. The nucleic acid constructs or vectors disclosed herein can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required.

Pharmaceutical compositions comprising a nucleic acid construct or vector disclosed herein may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

For delayed release, nucleic acid construct or vector disclosed herein may be included in a pharmaceutical composition which is formulated for slow release, such as in microcapsules formed from biocompatible polymers or in liposomal carrier systems according to methods known in the art.

If a vector is to be stored long-term, it may be frozen in the presence of glycerol.

### Methods

Provided herein are methods for inducing expression of a transgene in a cell and/or a tissue. Provided herein is a method of inducing the expression of transgene, the method comprising providing a cell comprising a nucleic acid construct comprising a NARE disclosed herein operatively linked to a transgene and cultivating the cells under conditions allowing for the expression of the transgene. In some embodiments, the cell is a liver cell. Provided herein is a method for inducing expression of a transgene in vivo. Provided herein is a method for inducing expression of a transgene in vitro. Provided herein is a method for inducing expression of a transgene ex vivo.

Provided herein are methods of treating a disease or disorder in a subject in need thereof using nucleic acid constructs, vectors, and pharmaceutical compositions disclosed herein. In some embodiments, the disease is Wilson's Disease (WD). In some embodiments, the subject has a mutation in ATP7B.

Provided herein is a method of treating or preventing dystonia or bradykinesia in a subject suffering from Wilson's Disease in a subject in need thereof, the method comprising administering to the subject a vector or a pharmaceutical composition disclosed herein. Provided herein is a vector or a pharmaceutical composition disclosed herein for use in treating or preventing dystonia or bradykinesia in a subject suffering from Wilson's Disease in a subject in need thereof. Provided herein is the use of a vector in the manufacture of a medicament for treating or preventing dystonia or bradykinesia in a subject suffering from Wilson's Disease in a subject in need thereof.

Provided herein is a method of reducing the incidence of leukopenia or anemia in a subject suffering from Wilson's Disease in a subject in need thereof, the method comprising administering to the subject a vector or a pharmaceutical composition disclosed herein. Provided herein is a vector or a pharmaceutical composition disclosed herein for use in reducing the incidence of leukopenia or anemia in a subject suffering from Wilson's Disease in a subject in need thereof. Provided herein is the use of a vector in the manufacture of a medicament for reducing the incidence of leukopenia or anemia in a subject suffering from Wilson's Disease in a subject in need thereof.

Provided herein is a method of reducing the incidence of cirrhosis in a subject suffering from Wilson's Disease in a subject in need thereof, the method comprising administering to the subject a vector or a pharmaceutical composition disclosed herein. Provided herein is a vector or a pharmaceutical composition disclosed herein for use in reducing the incidence of cirrhosis in a subject suffering from Wilson's Disease in a subject in need thereof. Provided herein is the use of a vector in the manufacture of a medicament for reducing the incidence of cirrhosis in a subject suffering from Wilson's Disease in a subject in need thereof.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with a disease or disorder without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with a disease or disorder without treatment. In some embodiments, treatment results in long term survival of a patient. As used herein, the term “long term survival” refers to a survival time or life expectancy longer than about 40 years, 45 years, 50 years, 55 years, 60 years, or longer.

Methods of administration include, but are not limited to, intracisternal magna, intracerebroventricular, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, the nucleic acid construct or vector described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as silastic membranes, or fibers. In some situations, the nucleic acid construct or vector described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

The compositions described herein can be administered as single administrations or as multiple administrations. Such compositions can be administered at regular intervals, depending on the nature, severity and extent of the subject's condition. In some embodiments, a therapeutically effective amount of the nucleic acid construct or vector is administered intrathecally periodically at regular intervals (e.g., once every year, once every six months, once every five months, once every three months, bimonthly (once every two months), monthly (once every month), biweekly (once every two weeks), or weekly).

The amount of the nucleic acid construct or vector described herein that is effective for treating disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

An effective amount of an rAAV carrying a nucleic acid sequence encoding a transgene (including, but not limited to UPF1, ATP7B, and BDNF) under the control of the NARE may, for example, range between about 1×10⁹ to about 1×10¹⁴ rAAV genome particles (vg)/kg body weight. A “genome particle” is defined herein as an AAV capsid that contains a single stranded DNA molecule that can be quantified with a sequence specific method (such as qPCR or ddPCR). In some embodiments, the rAAV is administered at about 1×10¹² to about 1×10¹³ rAAV vg/kg body weight. In some embodiments, the rAAV is administered at about 5×10¹¹ to about 5×10¹² to vg/mL of cerebrospinal fluid (CSF) volume. In some embodiments, the rAAV is administered at about 7.5×10¹³ to 7.5×10¹⁴ vg total per patient.

In some embodiments, the rAAV is administered to an animal at about 1×10¹¹ to about 1×10¹⁴ rAAV genome particles (vg)/kg body weight.

In some embodiments, the rAAV genome particles are provided in a volume of between about 20 uL to about 50 mL. In some embodiments, the rAAV genome particles are provided in a volume of between about 30 uL to about 30 mL. In some embodiments, the rAAV genome particles are provided in a volume of about 30, about 40, about 50, about 60, about 70, about 80, about 90, about 100, about 200, about 300, about 400, about 500, about 600, about 700, about 800, about 900, about 1000 μL. In some embodiments, the rAAV genome particles are provided in a volume of about 1, about 2, about 3, about 4, about 5, about 6, about 7, about 8, about 9, about 10, about 15, about 20, about 25, about 30, about 35, about 40, about 45, about or 50 mL.

Still other dosages in these ranges may be selected by the attending physician. It is to be understood that for any particular subject, specific dosage regimens can be adjusted over time according to the individual need and the professional judgment of the person administering or supervising the administration of the nucleic acid construct or vector and that dosage ranges set forth herein are exemplary only and are not intended to limit the scope or practice of the claimed invention.

The nucleic acid construct or vectors disclosed herein can also be advantageously provided to a cell ex vivo, followed by administration of the living cell to the subject. Methods for treating disease by implanting a cell that has been modified to express a recombinant protein are also well known. See, for example, U.S. Pat. No. 5,399,346, disclosing methods for introducing a nucleic acid into a primary human cell for introduction into a human. Although use of human cells for ex vivo therapy is preferred in some embodiments, other cells such as bacterial cells may be implanted in a subject's vasculature, continuously releasing a therapeutic agent. See, for example, U.S. Pat. Nos. 4,309,776 and 5,704,910.

This application is related to U.S. Provisional Application No. 63/379,109 entitled “UPF1 EXPRESSION CONSTRUCTS”, U.S. Provisional Application No. 63/379,113, entitled “ATP7B GENE THERAPY” and U.S. Provisional Application No. 63/379,114, entitled “BDNF GENE THERAPY” all filed on Oct. 11, 2022 by the same Applicant, and each of which is incorporated herein by reference in its entirety.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following Examples of specific embodiments are given. The following examples should not be read to limit or define the entire scope of the invention.

### EXAMPLES

### Example 1: NARE Design

Potential NARE candidates were also identified from genes that are highly expressed in humans in the target tissue (liver). Promoter regions were then defined or cis-regulatory elements identified based on chromatin marks, accessibility, conservation, and other genome-wide datasets.

Motif ten element (MTE)/downstream promoter element (DPE) modifications were incorporated to increase transcription, as were various cellular, viral, or synthetic 5′ UTR motifs to increase transcription/translation. Intron choice was changed to include, for example, short cellular introns that naturally have a unique standalone transcriptional start site, which indicates the presence of a promoter within this particular intron. In addition, introns were inserted at different positions of the sequence, and/or introns were truncated while maintaining splicing elements. In addition, individual or multiple point mutations were introduced to increase expression or prevent inactivation. Also, different transcriptional enhancers were introduced upstream of the core promoter. Transcriptional motif insertions, replacements, and/or shuffling was employed in certain instances as was removal of CpG sequences to lower immunogenicity while maintaining promoter activity.

Gene regulation elements were also optimized by the following methods (i) highly expressed transcription factor binding sites (TFBS) were identified, shuffled and cloned upstream of core promoters; (ii) iterative mutation and scoring of promoters using artificial intelligence (AI) algorithms.

### Materials and Methods

### AI-Driven NARE Design

Core prediction model: The prediction of construct potency was based on the predictions from the Xpresso model (xpresso.gs.washington.edu). The original Xpresso model was developed by considering the 10.5 kb region around the transcription start site (TSS, 7.5 kb upstream, 3 kb downstream the TSS). Because of size constraints (fragments longer than ˜1.5 kb were not used), the algorithm was modified to include a “padding” step before the calculation of the predicted activity. This padding step took a sequence and added ambiguous (“N”) nucleotides upstream and downstream of the shorter input fragment according to the expected/annotated TSS. The final model took a sequence, scanned for an upper-case base pair (convention used to label the TSS), padded the sequence to length, converted it into a one-hot-encoded data frame and then passed it through the convolutional neural network (CNN) with weights and structure from Xpresso.

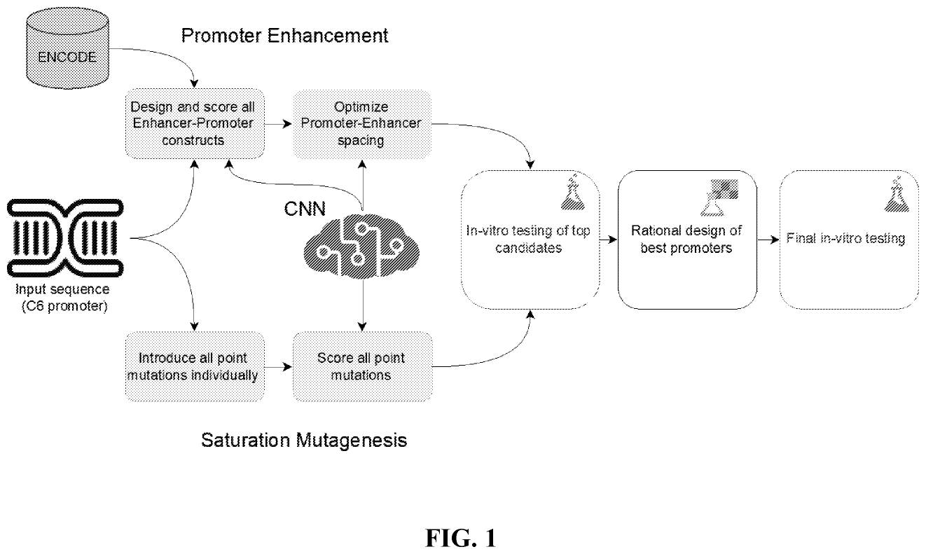

Custom software was written to create and automate the scoring for the following analyses (FIG. 1):

ENCODE+JeT ((6): ENCODE fragment coordinates were downloaded and subsequently used to parse such elements from the reference human genome (GRCh38). All the ENCODE fragments were individually positioned upstream a JeT promoter and then tested through the modified prediction model.

ENCODE. JeT spacing optimization: a custom script was used to insert 300 bp, 400 bp, 500 bp or 600 bp spacing elements between the top ˜2,000 scoring ENCODE elements and the JeT promoter. New “spaced” constructs were again tested with the modified prediction model.

Point-mutation Saturation mutagenesis: a custom script was used to generate all possible point-mutated versions of an input sequence. Resulting sequences were then scored with the modified prediction model. Custom scripts were also developed to iterate this process in through a greedy search (introduce the best result each round).

Through the CNN model, the effect of 708,157 enhancer fragments was predicted in-silico cloned up-stream of the C6 promoter sequence. The majority of enhancers were predicted to have a positive effect on C6 potency with ˜2,000 constructs producing a ˜2× improvement. This subset was further analyzed to identify the optimal spacing between the enhancer element and the promoter sequence. Introducing a spacing sequence improved the potency of the construct with the largest improvement observed with a spacing of 400 bp. Selected constructs were synthesized and tested in vitro (C118 to C127).

### Promoter Syntax and Rational Design of NARES

Potential NARE candidates were also identified from genes that are highly expressed in humans in the target tissues (liver). Promoter regions were then defined or cis-regulatory elements identified based on chromatin marks, accessibility, conservation, and other genome-wide datasets.

Motif ten element (MTE)/downstream promoter element (DPE) modifications were incorporated to increase transcription, as were various cellular, viral, or synthetic 5′ UTR motifs to increase transcription/translation. In CAG-based gene regulation elements (but also in others described herein), intron choice was changed to include, for example, short cellular introns that naturally have a unique standalone transcriptional start site, which indicates the presence of a promoter within this particular intron. In addition, introns were inserted at different positions of the sequence, and/or introns were truncated while maintaining splicing elements. In addition, individual or multiple point mutations were introduced to increase expression or prevent inactivation. Also, different transcriptional enhancers were introduced upstream of the core promoter. Transcriptional motif insertions, replacements, and/or shuffling was employed in certain instances as was removal of CpG sequences to lower immunogenicity while maintaining promoter activity.

Gene regulation elements were also optimized by the following methods (i) highly expressed transcription factor binding sites (TFBS) were identified, shuffled and cloned upstream of core promoters; (ii) iterative mutation and scoring of promoters using artificial intelligence (AI) algorithms.

### Example 2: Dual-Reporter Protein Expression Assay

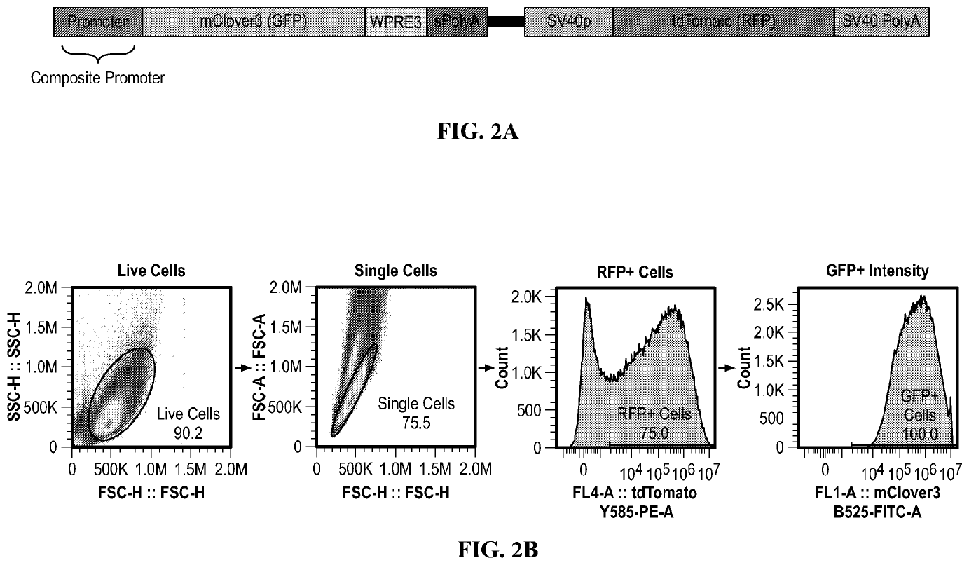

To test the ability of NARE candidates to drive gene expression, a dual reporter assay using flow cytometry was utilized. A diagram of the dual reporter expression construct and assay is provided in FIG. 2A. Different NARE promoter sequences were cloned upstream of a transgene encoding mClover3 (a green/yellow fluorescent protein), which in turn was located upstream of a posttranscriptional regulatory element of woodchuck hepatitis virus (WPRE250) and a synthetic polyadenylation (polyA) signal. mClover3 fluorescence was used as a measure of NARE strength. The plasmid also contained a separate expression cassette (from 5′ to 3′: SV40 promoter-tdTomato (a red fluorescent protein)-SV40 polyA), which was the same in all NARE test constructs (FIG. 2A). tdTomato fluorescence was used as an internal normalization control, allowing to account for variations in transfection efficiency.

Various cell lines were transiently transfected with dual reporter plasmids using TurboFect® transfection reagent. After 48 hours, cells were trypsinized and resuspended for flow cytometry. Raw data was processed to select only live, single cells for analysis. A sample comprising untransfected cells was used to set the gate for tdTomato signal. This gate was then applied to all live single cells to identify the tdTomato-positive (and thus transfected) cells (FIG. 2B). Then, mClover3 fluorescence was determined for the tdTomato-positive population. Because transfection varied slightly between different experiments, the relative NARE potency was calculated by the ratio of mClover3 fluorescence to tdTomato fluorescence. NARE candidates were tested along with known constitutive and tissue-specific reference promoters such as CAG, CMV, or AAT.

### Example 3: Strength of Different NAREs in Liver Cells (In Vitro)

### Materials and Methods

### Cell Culture

Huh-7, AML-12, and HEK 293 immortalized cell lines were maintained 37° C. in 5% CO2. Cells were transiently transfected with plasmids using TurboFect™. After 48 hours (72 hours for AML-12), the cell lysates were collected for protein analysis. After 20 passages, a new aliquot of cells was thawed and passaged twice before using for subsequent experiments.

### Primary Hepatocyte Cultures

Mouse and human hepatocyte cell lines from Gibco were thawed and placed in suspended medium followed by centrifugation at 100×g for 10 minutes at 4° C. Supernatants were aspirated, and cells were resuspended in maintenance medium (+10% FBS) followed by plating at 400,000 cells per well on a 24-well plate. Four hours post plating, the plate was gently tapped to loosen debris, followed by aspiration of the medium, and replacement with non-FBS containing maintenance medium. Cells were maintained at 37° C. in 5% CO2. Every day, full media changes were performed to feed the cultures.

### Transduction

On day 1 and day 3 in vitro for immortalized and primary cell cultures respectively, AAV8 particles were added to the medium at a multiplicity of infection (MOI) of 50,000. Doxorubicin containing medium was added on to the cells to speed up transduction. Media was changed 72 hours later to non-doxorubicin containing medium according to standard protocol.

### Results

A library of NAREs suitable for the expression of genes in the liver was developed. The promoters provided stronger expression in immortalized hepatocyte cells (Huh7, HepG2, AML-12) than commonly used reference promoters. The Huh7 cell line is a hepatocyte-derived cellular carcinoma cell line. HepG2 is a hepatoblastoma cell line. AML-12 cells are an immortal, epithelial cell line derived from mouse liver cells. Reference promoters included: CAG promoter, alpha-1-antitrypsin (AAT) promoter, human thyroxine binding globulin (TBG) promoter, liver-specific promoter 1 (LP1), hybrid liver promoter (HLP), and hepatic combinatorial bundle promoter (HCB). ATT is a compact, liver-specific promoter that is currently used in gene therapy clinical trials. HCB is a potent, NARE that is stronger than AAT. The promoters in the promoter library were of equal or greater strength than CAG while also being significantly smaller.

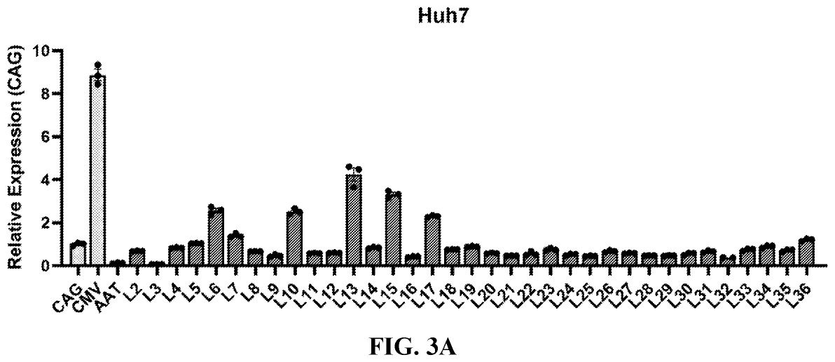

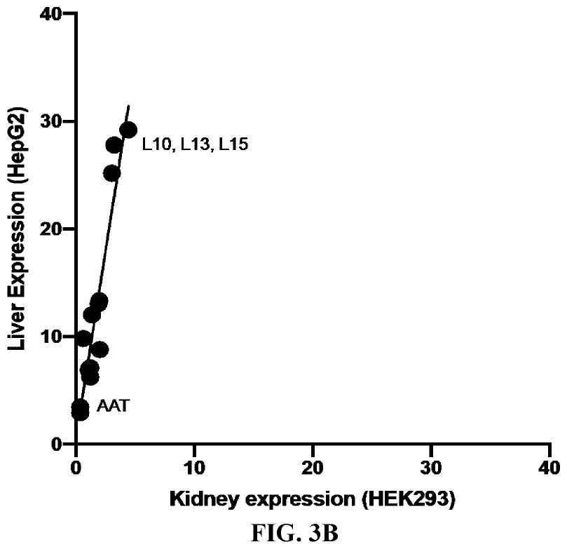

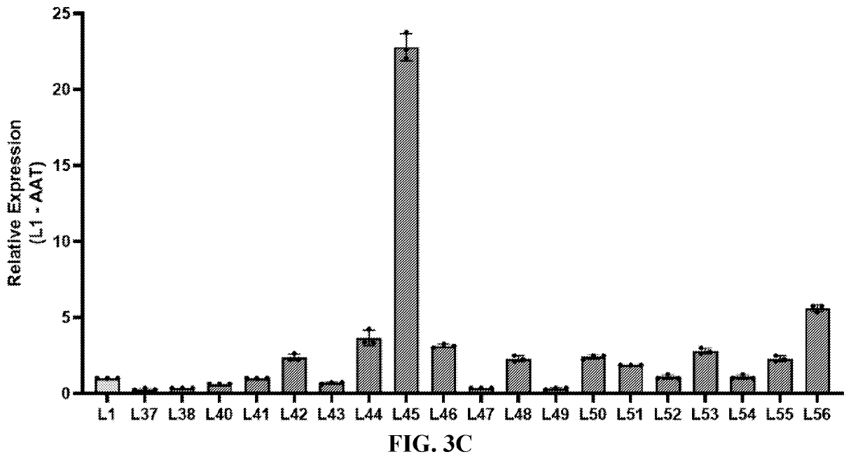

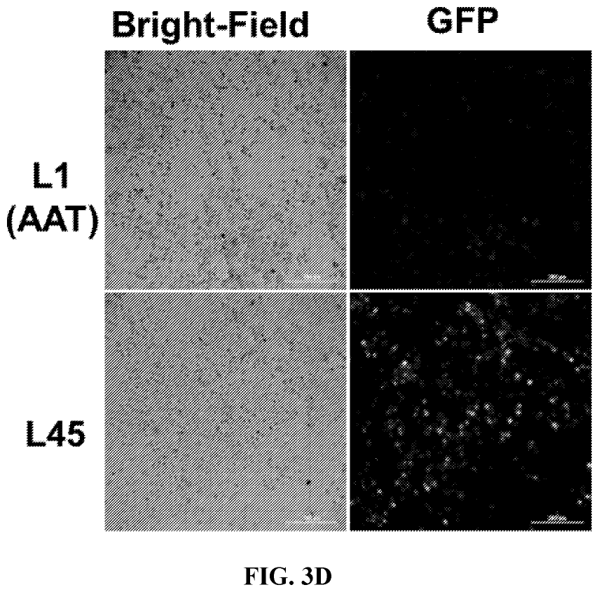

Additional NAREs were bioinformatically identified from top liver-expressed genes in humans and defined using various epigenetic and genomic characteristics (histone acetylation, TF occupancy, sequence conservation, etc.). Potency of selected NAREs in liver cells are shown in FIGS. 3A and 3C. L45 was 22-fold stronger than the AAT promoter (FIGS. 3C and 3D). NAREs L10, L13, and L15 are specific for liver cells (FIG. 3B).

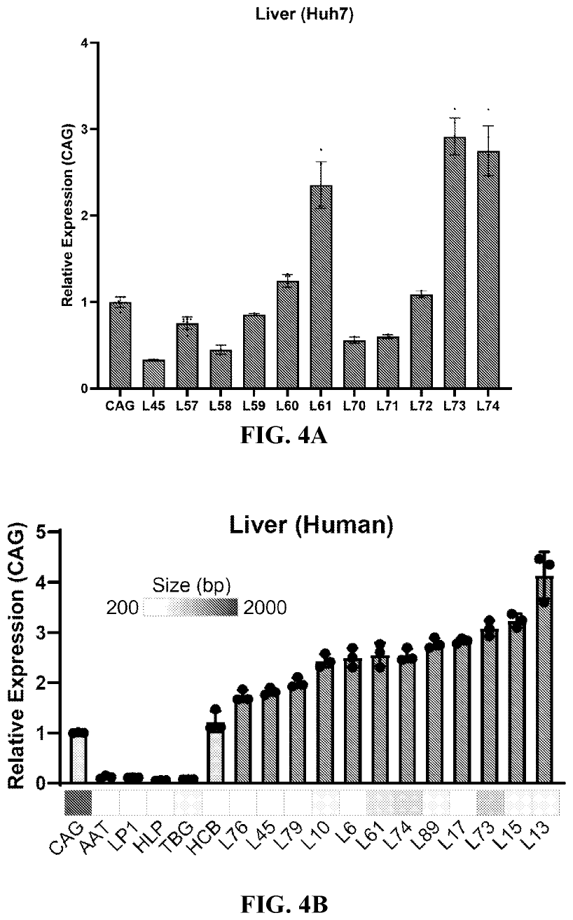

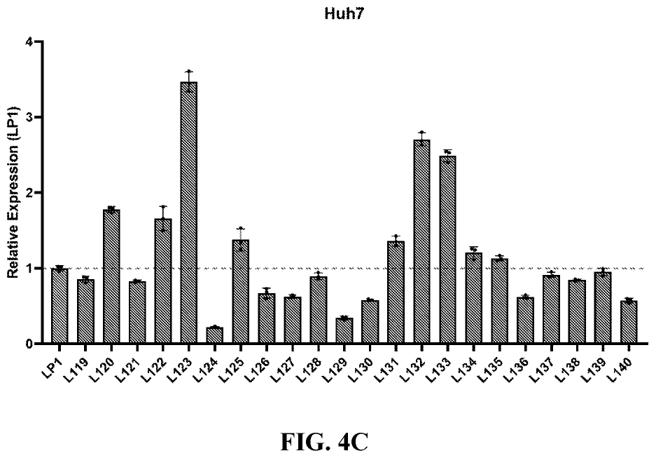

NARE L45 was further optimized, resulting in additionally increased expression (Table 1, FIGS. 4A and 4B). Optimized NAREs were up to ˜3-fold stronger than the 1.6 kb CAG promoter in liver cells. The potency of additional promoters in Huh7, a human liver cell line is shown in FIG. 4C. Potency of derivatives of the AAT promoter (L1, and the HCB promoter in different cell lines is shown in Tables 6 and 7, respectively.

TABLE 1
Potency of L45-derived NAREs. Plasmid transfections.

| | Size | Human |
|---|---|---|
| Promoter | (bp) | Liver (Huh7) |
| C19: CAG | 1761 | 3.0 |
| L1: AAT | 317 | 0.3 |
| L45 | 522 | 1.0 |
| L57 | 536 | 2.3 |
| L58 | 559 | 1.4 |
| L59 | 786 | 2.6 |
| L60 | 910 | 3.6 |
| L61 | 761 | 6.5 |
| L70 | 678 | 1.7 |
| L71 | 666 | 1.8 |
| L72 | 893 | 3.3 |
| L73 | 1017 | 8.3 |
| L74 | 868 | 9.0 |

TABLE 2
Potency of AAT promoter-derived NAREs. Plasmid transfections.

| | Size | Liver | Liver | Liver |
|---|---|---|---|---|
| Name | (bp) | Huh7 | HepG2 | AML12 |
| L1 (AAT) | 317 | 1 | 1 | 1 |
| L2 | 964 | 5.6 | 2.0 | 2.0 |
| L3 | 396 | 0.6 | 1.0 | 1.0 |
| L14 | 964 | 6.9 | 4.1 | 2.7 |
| L16 | 581 | 3.4 | 1.7 | 2.6 |
| L32 | 477 | 4.0 | 2.5 | 3.0 |
| L33 | 489 | 6.1 | 3.5 | 6.6 |
| L35 | 809 | 5.8 | 2.5 | 3.4 |

TABLE 3
Optimization of the HCB promoter. Plasmid transfections.

| | Size | Human |
|---|---|---|
| Promoter | (bp) | Liver (Huh7) |
| C19: CAG | 1761 | 0.8 |
| L1: AAT | 317 | 0.1 |
| L75: HCB1 | 255 | 1.0 |
| L76: HCB2 | 283 | 1.5 |
| L77: HCB3 | 332 | 1.1 |
| L78: HCB4 | 455 | 1.3 |
| L79: HCB5 | 593 | 1.4 |
| L89: HCB6 | 621 | 2.2 |

TABLE 4
Control or reference NAREs.

| | | NARE name | |
|---|---|---|---|
| | | and NARE | |
| SEQ ID NO | Name | components | Sequence |
| 251 | C19 | CAG | ACATTGATTATTGACTAGTTATTAATAGTAATCAATT |
| | | | ACGGGGTCATTAGTTCATAGCCCATATATGGAGTTC |
| | | | CGCGTTACATAACTTACGGTAAATGGCCCGCCTGGC |
| | | | TGACCGCCCAACGACCCCCGCCCATTGACGTCAATA |
| | | | ATGACGTATGTTCCCATAGTAACGCCAATAGGGACT |
| | | | TTCCATTGACGTCAATGGGTGGACTATTTACGGTAA |
| | | | ACTGCCCACTTGGCAGTACATCAAGTGTATCATATG |
| | | | CCAAGTACGCCCCCTATTGACGTCAATGACGGTAAA |
| | | | TGGCCCGCCTGGCATTATGCCCAGTACATGACCTTA |
| | | | TGGGACTTTCCTACTTGGCAGTACATCTACGTATTA |
| | | | GTCATCGCTATTACCATGGGTCGAGGTGAGCCCCAC |
| | | | GTTCTGCTTCACTCTCCCCATCTCCCCCCCCTCCCCA |
| | | | CCCCCAATTTTGTATTTATTTATTTTTTAATTATTTTG |
| | | | TGCAGCGATGGGGGCGGGGGGGGGGGGGGCGCGCG |
| | | | CCAGGCGGGGCGGGGGGGGCGAGGGGCGGGGCG |
| | | | GGGCGAGGCGGAGAGGTGCGGCGGCAGCCAATCAG |
| | | | AGCGGCGCGCTCCGAAAGTTTCCTTTTATGGCGAGG |
| | | | CGGCGGCGGCGGCGGCCCTATAAAAAGCGAAGCGC |
| | | | GCGGCGGGCGGGAGTCGCTGCGTTGCCTTCGCCCCG |
| | | | TGCCCCGCTCCGCGCCGCCTCGCGCCGCCCGCCCCG |
| | | | GCTCTGACTGACCGCGTTACTCCCACAGGTGAGCGG |
| | | | GCGGGACGGCCCTTCTCCTCCGGGCTGTAATTAGCG |
| | | | CTTGGTTTAATGACGGCTCGTTTCTTTTCTGTGGCTG |
| | | | CGTGAAAGCCTTAAAGGGCTCCGGGAGGGCCCTTTG |
| | | | TGCGGGGGGGAGCGGCTCGGGGGGTGCGTGCGTGT |
| | | | GTGTGTGCGTGGGGAGCGCCGCGTGCGGCCCGCGCT |
| | | | GCCCGGCGGCTGTGAGCGCTGCGGGCGCGGCGCGG |
| | | | GGCTTTGTGCGCTCCGCGTGTGCGCGAGGGGAGCGC |
| | | | GGCCGGGGGCGGTGCCCCGCGGTGCGGGGGGGCTG |
| | | | CGAGGGGAACAAAGGCTGCGTGCGGGGTGTGTGCG |
| | | | TGGGGGGGTGAGCAGGGGGTGTGGGCGCGGCGGTC |
| | | | GGGCTGTAACCCCCCCCTGCACCCCCCTCCCCGAGT |
| | | | TGCTGAGCACGGCCCGGCTTCGGGTGCGGGGCTCCG |
| | | | TACGGGGCGTGGCGCGGGGCTCGCCGTGCCGGGCG |
| | | | GGGGGTGGCGGCAGGTGGGGGTGCCGGGCGGGGCG |
| | | | GGGCCGCCTCGGGCCGGGGAGGGCTCGGGGGAGGG |
| | | | GCGCGGCGGCCCCCGGAGCGCCGGCGGCTGTCGAG |
| | | | GCGCGGCGAGCCGCAGCCATTGCCTTTTATGGTAAT |
| | | | CGTGCGAGAGGGCGCAGGGACTTCCTTTGTGCCAAA |
| | | | TCTGTGCGGAGCCGAAATCTGGGAGGCGCCGCCGC |
| | | | ACCCCCTCTAGCGGGCGCGGGGCGAAGCGGTGCGG |
| | | | CGCCGGCAGGAAGGAAATGGGCGGGGAGGGCCTTC |
| | | | GTGCGTCGCCGCGCCGCCGTCCCCTTCTCCCTCTCCA |
| | | | GCCTCGGGGCTGTCCGCGGGGGGACGGCTGCCTTCG |
| | | | GGGGGGACGGGGCAGGGCGGGGTTCGGCTTCTGGC |
| | | | GTGTGACCGGCGGCTCTAGAGCCTCTGCTAACCATG |
| | | | TTCATGCCTTCTTCTTTTTCCTACAGCTCCTGGGCAA |
| | | | CGTGCTGGTTATTGTGCTGTCTCATCATTTTGGCAAA |
| | | | GAATTCGAGGTGGAAATTAATACGACGTGGCGGAT |
| | | | CCGCCACC |
| 252 | | CMV | GACATTGATTATTGACTAGTTATTAATAGTAATCAA |
| | | enhancer | TTACGGGGTCATTAGTTCATAGCCCATATATGGAGT |
| | | | TCCGCGTTACATAACTTACGGTAAATGGCCCGCCTG |
| | | | GCTGACCGCCCAACGACCCCCGCCCATTGACGTCAA |
| | | | TAATGACGTATGTTCCCATAGTAACGCCAATAGGGA |
| | | | CTTTCCATTGACGTCAATGGGTGGACTATTTACGGT |
| | | | AAACTGCCCACTTGGCAGTACATCAAGTGTATCATA |
| | | | TGCCAAGTACGCCCCCTATTGACGTCAATGACGGTA |
| | | | AATGGCCCGCCTGGCATTATGCCCAGTACATGACCT |
| | | | TATGGGACTTTCCTACTTGGCAGTACATCTACGTATT |
| | | | AGTCATCGCTATTACCATG |
| 253 | | Chicken beta | TCGAGGTGAGCCCCACGTTCTGCTTCACTCTCCCCAT |
| | | actin promoter | CTCCCCCCCCTCCCCACCCCCAATTTTGTATTTATTT |
| | | | ATTTTTTAATTATTTTGTGCAGCGATGGGGGCGGGG |
| | | | GGGGGGGGGGGGCGCGCCAGGCGGGGCGGGGCGG |
| | | | GGCGAGGGGCGGGGCGGGGCGAGGCGGAGAGGTG |
| | | | CGGCGGCAGCCAATCAGAGCGGCGCGCTCCGAAAG |
| | | | TTTCCTTTTATGGCGAGGCGGCGGCGGCGGCGGCCC |
| | | | TATAAAAAGCGAAGCGCGCGGCGGGCG |
| 254 | | chimeric | GGAGTCGCTGCGTTGCCTTCGCCCCGTGCCCCGCTC |
| | | intron | CGCGCCGCCTCGCGCCGCCCGCCCCGGCTCTGACTG |
| | | | ACCGCGTTACTCCCACAGGTGAGCGGGCGGGACGG |
| | | | CCCTTCTCCTCCGGGCTGTAATTAGCGCTTGGTTTAA |
| | | | TGACGGCTCGTTTCTTTTCTGTGGCTGCGTGAAAGCC |
| | | | TTAAAGGGCTCCGGGAGGGCCCTTTGTGCGGGGGG |
| | | | GAGCGGCTCGGGGGGTGCGTGCGTGTGTGTGTGCGT |
| | | | GGGGAGCGCCGCGTGCGGCCCGCGCTGCCCGGCGG |
| | | | CTGTGAGCGCTGCGGGCGCGGCGCGGGGCTTTGTGC |
| | | | GCTCCGCGTGTGCGCGAGGGGAGCGCGGCCGGGGG |
| | | | CGGTGCCCCGCGGTGCGGGGGGGCTGCGAGGGGAA |
| | | | CAAAGGCTGCGTGCGGGGTGTGTGCGTGGGGGGGT |
| | | | GAGCAGGGGGTGTGGGCGCGGCGGTCGGGCTGTAA |
| | | | CCCCCCCCTGCACCCCCCTCCCCGAGTTGCTGAGCA |
| | | | CGGCCCGGCTTCGGGTGCGGGGCTCCGTACGGGGCG |
| | | | TGGCGCGGGGCTCGCCGTGCCGGGCGGGGGGTGGC |
| | | | GGCAGGTGGGGGTGCCGGGCGGGGGGGGCCGCCT |
| | | | CGGGCCGGGGAGGGCTCGGGGGAGGGGCGCGGCGG |
| | | | CCCCCGGAGCGCCGGCGGCTGTCGAGGCGCGGCGA |
| | | | GCCGCAGCCATTGCCTTTTATGGTAATCGTGCGAGA |
| | | | GGGCGCAGGGACTTCCTTTGTGCCAAATCTGTGCGG |
| | | | AGCCGAAATCTGGGAGGCGCCGCCGCACCCCCTCTA |
| | | | GCGGGCGCGGGGCGAAGCGGTGCGGCGCCGGCAGG |
| | | | AAGGAAATGGGCGGGGAGGGCCTTCGTGCGTCGCC |
| | | | GCGCCGCCGTCCCCTTCTCCCTCTCCAGCCTCGGGG |
| | | | CTGTCCGCGGGGGGACGGCTGCCTTCGGGGGGGAC |
| | | | GGGGCAGGGCGGGGTTCGGCTTCTGGCGTGTGACCG |
| | | | GCGGCTCTAGAGCCTCTGCTAACCATGTTCATGCCT |
| | | | TCTTCTTTTTCCTACAG |
| 255 | C1 | CMV | GACATTGATTATTGACTAGTTATTAATAGTAATCAA |
| | | | TTACGGGGTCATTAGTTCATAGCCCATATATGGAGT |
| | | | TCCGCGTTACATAACTTACGGTAAATGGCCCGCCTG |
| | | | GCTGACCGCCCAACGACCCCCGCCCATTGACGTCAA |
| | | | TAATGACGTATGTTCCCATAGTAACGCCAATAGGGA |
| | | | CTTTCCATTGACGTCAATGGGTGGACTATTTACGGT |
| | | | AAACTGCCCACTTGGCAGTACATCAAGTGTATCATA |
| | | | TGCCAAGTACGCCCCCTATTGACGTCAATGACGGTA |
| | | | AATGGCCCGCCTGGCATTATGCCCAGTACATGACCT |
| | | | TATGGGACTTTCCTACTTGGCAGTACATCTACGTATT |
| | | | AGTCATCGCTATTACCATGGTGATGCGGTTTTGGCA |
| | | | GTACATCAATGGGCGTGGATAGCGGTTTGACTCACG |
| | | | GGGATTTCCAAGTCTCCACCCCATTGACGTCAATGG |
| | | | GAGTTTGTTTTGGCACCAAAATCAACGGGACTTTCC |
| | | | AAAATGTCGTAACAACTCCGCCCCATTGACGCAAAT |
| | | | GGGCGGTAGGCGTGTACGGTGGGAGGTCTATATAA |
| | | | GCAGAGCTCGTTTAGTGAACCGTCAGATCGCCTGGA |
| | | | GACGCCATCCACGCTGTTTTGACCTCCATAGAATAA |
| | | | TACGACTCACTATAGGGAAGCTTGGCATTCCGGTAC |
| | | | TGTTGGATCCGCCACC |
| 74 | LP1 | | CCCTAAAATGGGCAAACATTGCAAGCAGCAAACAG |
| | | | CAAACACACAGCCCTCCCTGCCTGCTGACCTTGGAG |
| | | | CTGGGGCAGAGGTCAGAGACCTCTCTGGGCCCATGC |
| | | | CACCTCCAACATCCACTCGACCCCTTGGAATTTCGG |
| | | | TGGAGAGGAGCAGAGGTTGTCCTGGCGTGGTTTAGG |
| | | | TAGTGTGAGAGGGGAATGACTCCTTTCGGTAAGTGC |
| | | | AGTGGAAGCTGTACACTGCCCAGGCAAAGCGTCCG |
| | | | GGCAGCGTAGGCGGGCGACTCAGATCCCAGCCAGT |
| | | | GGACTTAGCCCCTGTTTGCTCCTCCGATAACTGGGG |
| | | | TGACCTTGGTTAATATTCACCAGCAGCCTCCCCCGTT |
| | | | GCCCCTCTGGATCCACTGCTTAAATACGGACGAGGA |
| | | | CAGGGCCCTGTCTCCTCAGCTTCAGGCACCACCACT |
| | | | GACCTGGGACAGTGAATTCGCGGCCGCCACC |
| 1 | L1 | AAT | CACCCCCTCCACCTTGGACACAGGACGCTGTGGTTT |
| | | | CTGAGCCAGGTACAATGACTCCTTTCGGTAAGTGCA |
| | | | GTGGAAGCTGTACACTGCCCAGGCAAAGCGTCCGG |
| | | | GCAGCGTAGGCGGGCGACTCAGATCCCAGCCAGTG |
| | | | GACTTAGCCCCTGTTTGCTCCTCCGATAACTGGGGT |
| | | | GACCTTGGTTAATATTCACCAGCAGCCTCCCCCGTT |
| | | | GCCCCTCTGGATCCACTGCTTAAATACGGACGAGGA |
| | | | CAGGGCCCTGTCTCCTCAGCTTCAGGCACCACCACT |
| | | | GACCTGGGACAGTGAATCGGCGGCCGCCACC |
| 75 | L68 | HLP | TGTTTGCTGCTTGCAATGTTTGCCCATTTTAGGGTGG |
| | | | ACACAGGACGCTGTGGTTTCTGAGCCAGGGGGCGA |
| | | | CTCAGATCCCAGCCAGTGGACTTAGCCCCTGTTTGC |
| | | | TCCTCCGATAACTGGGGTGACCTTGGTTAATATTCA |
| | | | CCAGCAGCCTCCCCCGTTGCCCCTCTGGATCCACTG |
| | | | CTTAAATACGGACGAGGACAGGGCCCTGTCTCCTCA |
| | | | GCTTCAGGCACCACCACTGACCTGGGACAGTGAATC |
| | | | GCGGCCGCCACC |
| 70 | L63 | TBG (aka | AGGGCTGGAAGCTACCTTTGACATCATTTCCTCTGC |
| | | TBG.Ci) | GAATGCATGTATAATTTCTACAGAACCTATTAGAAA |
| | | | GGATCACCCAGCCTCTGCTTTTGTACAACTTTCCCTT |
| | | | AAAAAACTGCCAATTCCACTGCTGTTTGGCCCAATA |
| | | | GTGAGAACTTTTTCCTGCTGCCTCTTGGTGCTTTTGC |
| | | | CTATGGCCCCTATTCTGCCTGCTGAAGACACTCTTGC |
| | | | CAGCATGGACTTAAACCCCTCCAGCTCTGACAATCC |
| | | | TCTTTCTCTTTTGTTTTACATGAAGGGTCTGGCAGCC |
| | | | AAAGCAATCACTCAAAGTTCAAACCTTATCATTTTT |
| | | | TGCTTTGTTCCTCTTGGCCTTGGTTTTGTACATCAGC |
| | | | TTTGAAAATACCATCCCAGGGTTAATGCTGGGGTTA |
| | | | ATTTATAACTAAGAGTGCTCTAGTTTTGCAATACAG |
| | | | GACATGCTATAAAAATGGAAAGATGTTGCTTTCTGA |
| | | | GAGACTGCAGAAGTTGGTCGTGAGGCACTGGGCAG |
| | | | GTAAGTATCAAGGTTACAAGACAGGTTTAAGGAGA |
| | | | CCAATAGAAACTGGGCTTGTCGAGACAGAGAAGAC |
| | | | TCTTGCGTTTCTGATAGGCACCTATTGGTCTTACTGA |
| | | | CATCCACTTTGCCTTTCTCTCCACAGGTGTCCAGGCG |
| | | | GCCGCC |
| 204 | | HCB | GTTAATCATTAAGTCGTTAATTTTTGTGGCCCTTGCG |
| | | | ATGTTTGCTCTGGTTAATAATCTCAGGACAAACAGA |
| | | | GGTTAATAATTTTCCAGATCTCTCTGAGCAATAGTA |
| | | | TAAAAGGCCAGCAGCAGCCTGACCACATCTCATCCT |

TABLE 5
NAREs.

| SEQ | | NARE name and | SEQ | | NARE name and |
|---|---|---|---|---|---|
| ID NO | Name | NARE components | ID NO | Name | NARE components |
| 1 | L1 | AAT | 75 | L68 | HLP |
| 2 | L1_v2 | AAT_v2 | 76 | L69 | AAT_v3 |
| 3 | L2 | AAT-MVMi | 77 | L70 | FTL7 |
| | | (SynE-mTTR) | | | |
| 4 | L3 | AAT-MVMi | 78 | L71 | FTL8 |
| 5 | L4 | AATcre-mTTR | 79 | L72 | FTL9 |
| 6 | L5 | ABPcre-mTTR | 80 | L73 | FTL10 |
| 7 | L6 | ALBcre-mTTR | 81 | L74 | FTL11 |
| 8 | L7 | APOEcre-mTTR | 82 | L75 | HCB-MVMi |
| 9 | L8 | AY661265.1_v2 | 83 | L76 | HCB2-MVMi |
| | | (mTTR-based) | | | |
| 10 | L8a | SynE-mTTR | 84 | L77 | HCB3-MVMi |
| 11 | L9 | Chr19F3_R3_v2-mTTR | 85 | L78 | HCB4-MVMi |
| | | (MTE_DPE) | | | |
| 12 | L10 | FGBcre1-mTTR | 86 | L78b | HCB4-MVMi v2 |
| | | (MPE_DPE) | | | |
| 13 | L10_v2 | FGBcre1-mTTR | 87 | L79 | SERPINAe-SynE- |
| | | (MPE_DPE) v2 | | | HCB-MVMi |
| 14 | L11 | HBVcre-mTTR | 88 | L80 | FTL2_EH38E1513438 |
| 15 | L12 | mTTR (MTE_DPE) | 89 | L81 | FTL2_EH38E1514777 |
| 16 | L13 | SERPINA1cre-mTTR | 90 | L82 | FTL2_EH38E1521375 |
| | | (MTE_DPE) | | | |
| 17 | L13_TISU | SERPINA1cre-mTTR | 91 | L83 | FTL2_EH38E1761982 |
| | | (MTE_DPE)-TISU | | | |
| 18 | L13_v2 | SERPINA1cre-mTTR | 92 | L84 | FTL2_EH38E1841027 |
| | | (MTE_DPE) v2 | | | |
| 19 | L14 | SynE-mTTR-MVMi (AAT) | 93 | L85 | FTL2_EH38E2144693 |
| 20 | L15 | SynEnh-mTTR_v2 | 94 | L86 | FTL2_EH38E2147276 |
| | | (MTE_DPE) | | | |
| 21 | L15_L21 | SynEnh-mTTR_v2 | 95 | L87 | FTL2_EH38E2274269 |
| | | (MTE_DPE)-L21 | | | |
| 22 | L15_L21_{—} | SynEnh-mTTR_v2 | 96 | L88 | FTL2_EH38E2724199 |
| | TISU | (MTE_DPE)-L21_TISU | | | |
| 23 | L15_TISU | SynEnh-mTTR_v2 | 97 | L89 | SERPINAe-SynE- |
| | | (MTE_DPE)-TISU | | | HCBmd-MVMi |
| 24 | L16 | synEnh_v2-AAT-MVMi | 98 | L90 | L57_best_mutated |
| 25 | L17 | SynEnh_v2-mTTR | 99 | L91 | LTF7-JeTmd |
| 26 | L18 | SynEnh_v2-mTTR-MVMi | 100 | L92 | LTF11-JeTmd |
| 27 | L19 | SynEnhCFLv0.3 -mTTR | 101 | L93 | LTF22-JeTmd |
| | | (MPE_DPE) | | | |
| 28 | L20 | ALB-EH38E2303193- | 102 | L94 | LTF29-JeTmd |
| | | mTTRp-MVMi | | | |
| 29 | L21 | ALB-EH38E2303194- | 103 | L95 | LTF31-JeTmd |
| | | mTTRp-MVMi | | | |
| 30 | L22 | ALB-EH38E2303202- | 104 | L96 | LTF32-JeTmd |
| | | mTTRp-MVMi | | | |
| 31 | L23 | APOH-EH38E1879540- | 105 | L97 | LTF35-JeTmd |
| | | mTTRp-MVMi | | | |
| 32 | L24 | CRP-EH38E1389831- | 106 | L98 | LTF7-LP1 |
| | | mTTRp-MVMi | | | |
| 33 | L25 | CYP2E1-EH38E1513217- | 107 | L99 | LTF11-LP1 |
| | | mTTRp-MVMi | | | |
| 34 | L26 | FGA-EH38E2337843- | 108 | L100 | LTF22-LP1 |
| | | mTTRp-MVMi | | | |
| 35 | L27 | FGB-EH38E2337831- | 109 | L101 | LTF29-LP1 |
| | | mTTRp-MVMi | | | |
| 36 | L28 | FGG-EH38E2337853- | 110 | L102 | LTF31-LP1 |
| | | mTTRp-MVMi | | | |
| 37 | L29 | SERPINA1-EH38E1738321- | 111 | L103 | LTF32-LP1 |
| | | mTTRp-MVMi | | | |
| 38 | L30 | SERPINA1-EH38E1738327- | 112 | L104 | LTF35-LP1 |
| | | mTTRp-MVMi | | | |
| 39 | L31 | SERPINA1-EH38E1738328- | 113 | L105 | LTF7-AATv3 |
| | | mTTRp-MVMi | | | |
| 40 | L32 | synEnh_v2-AATp | 114 | L106 | LTF11-AATv3 |
| 41 | L33 | synEnh_v2-AAT_v2 | 115 | L107 | LTF22-AATv3 |
| 42 | L34 | SynEnh_v2-mTTRcore | 116 | L108 | LTF29-AATv3 |
| 43 | L35 | SynEnh_v2-mTTRcore | 117 | L109 | LTF31-AATv3 |
| | | MVMi (AAT) | | | |
| 44 | L36 | SynEnh_v2-mTTRcore | 118 | L110 | LTF32-AATv3 |
| | | MVMi | | | |
| 45 | L37 | ORM1 | 119 | L111 | LTF35-AATv3 |
| 46 | L38 | APOC3-MPG | 120 | L112 | LTF7-HCB2 |
| 47 | L39 | Saa1 | 121 | L113 | LTF11-HCB2 |
| 48 | L40 | APOA2 | 123 | L114 | LTF22-HCB2 |
| 49 | L41 | SERPINA1 | 124 | L115 | LTF29-HCB2 |
| 50 | L42 | ALB | 125 | L116 | LTF31-HCB2 |
| 51 | L43 | APOA1 | 126 | L117 | LTF32-HCB2 |
| 52 | L44 | SAA2 | 127 | L118 | LTF35-HCB2 |
| 53 | L45 | FTL | 128 | L119 | LP1-EEF1A1i |
| 54 | L46 | FGA | 129 | L120 | LP1-EEF1B2i |
| 55 | L47 | CRP | 130 | L121 | LP1-TMSB10i |
| 56 | L48 | ORM2 | 131 | L122 | LP1-S100A6i |
| 57 | L49 | HP | 132 | L123 | LP1-RPL27i |
| 256 | L50 | FGB | 133 | L124 | LP1-GFAPi |
| 58 | L51 | FGG | 134 | L125 | LP1-UCHLi |
| 59 | L52 | APOE | 135 | L126 | LP1-ALDOAi |
| 60 | L53 | FABP1 | 136 | L127 | LP1-CAMK2Ai |
| 61 | L54 | APOC1 | 137 | L128 | LP1-FHL1i |
| 62 | L55 | APOH | 138 | L129 | LP1-CRYABi |
| 63 | L56 | C3 | 139 | L130 | LP1-FXYD1i |
| 64 | L57 | FTL2 | 140 | L131 | LP1-RBP4i |
| 65 | L58 | FTL3 | 141 | L132 | LP1-APOCi |
| 66 | L59 | FTL4 | 142 | L133 | LP1-ATF5i |
| 67 | L60 | FTL5 | 143 | L134 | LP1-HPDi |
| 68 | L61 | FTL6 | 144 | L135 | LP1-FLOT1i |
| 69 | L62 | ALBcre-FGBcre- | 145 | L136 | LP1-HBBi1 |
| | | SERPINA1cre- | | | |
| | | SynE-mTTR | | | |
| 70 | L63 | TBG.Ci | 146 | L137 | LP1-HBBi2 |
| 71 | L64 | TBG.MD.Ci | 147 | L138 | LP1-HBBi2xc |
| 72 | L65 | TBG.MD.Ci2 | 148 | L139 | LP1-IFI27L2i |
| 73 | L66 | ApoE-HCR-hAAT | 149 | L140 | LP1-RPL26i |
| 74 | L67 | LP1 | | | |

TABLE 6
NARE components.

| SEQ | NARE name and |
|---|---|
| ID NO | NARE components |
| 150 | AATp |
| 151 | MVMi-SynE- |
| | mTTRp intron |
| 152 | SynE-mTTRp |
| 153 | SynEnh |
| 154 | mTTRp |
| 155 | MVM intron |
| 156 | AATcre |
| 157 | mTTRmd promoter |
| 158 | mTTRmd2 promoter |
| 159 | MTE/DPE |
| 160 | MTE/DPE2 |
| 161 | ABPcre |
| 162 | ALBcre |
| 163 | APOEcre |
| 164 | ApoE-HCR |
| 165 | Chr19-F3/R3 |
| 166 | FGBcre1 |
| 167 | HBVcre |
| 168 | SERPINA1cre1 |
| 169 | SERPINA1cre2 |
| 170 | TISU |
| 171 | MVMi-AATp intron |
| 172 | L21 |
| 173 | L21-tisu |
| 174 | SynEnh_v3 |
| 175 | EH38E2303193 |
| 176 | mTTR3p |
| 177 | EH38E2303194 |
| 178 | EH38E2303202 |
| 179 | EH38E1879540 |
| 180 | EH38E1389831 |
| 181 | EH38E1513217 |
| 182 | EH38E2337843 |
| 183 | EH38E2337831 |
| 184 | EH38E2337853 |
| 185 | EH38E1738321 |
| 186 | EH38E1738327 |
| 187 | EH38E1738328 |
| 188 | AAT_v2_p |
| 189 | AATv3 promoter |
| 190 | FTL-TImd promoter |
| 191 | FTL-Timd |
| | promoter_alt |
| 192 | Timd |
| 193 | HBB intron |
| 194 | HBB intron 1 |
| | inserted at |
| | nucleotide 455 |
| | of LP1 |
| 195 | HBBi-mTTRp intron |
| 196 | mTTRmd promoter5 |
| 197 | SynEnh2 |
| 198 | Imd |
| 199 | TBGmd promoter |
| 200 | hAAT promoter |
| 201 | FTL-TImd2 promoter |
| 202 | FTL-TImd3 promoter |
| 203 | FTL-Timd4 promoter |
| 204 | HCB promoter |
| 205 | HCB2 promoter |
| 206 | HCBmd |
| 207 | LTF7 |
| 208 | JeTmd |
| 209 | LTF11 |
| 210 | LTF22 |
| 211 | LTF29 |
| 212 | LTF31 |
| 213 | LTF32 |
| 214 | LTF35 |
| 215 | AATv4 promoter |
| 216 | EF1-alpha intron A inserted |
| | at nucleotide 455 of LP1 |
| 217 | EEF1B2 intron inserted at |
| | nucleotide 455 of LP1 |
| 218 | TMSB10 intron inserted at |
| | nucleotide 455 of LP1 |
| 219 | S100A6 intron inserted at |
| | nucleotide 455 of LP1 |
| 220 | RPL27 intron inserted at |
| | nucleotide 455 of LP1 |
| 221 | GFAP intron inserted at |
| | nucleotide 455 of LP1 |
| 222 | UCHL intron inserted at |
| | nucleotide 455 of LP1 |
| 223 | ALDOA intron inserted at |
| | nucleotide 455 of LP1 |
| 224 | CAMK2A intron inserted at |
| | nucleotide 455 of LP1 |
| 225 | FHL1 intron inserted at |
| | nucleotide 455 of LP1 |
| 226 | CRYAB intron inserted at |
| | nucleotide 455 of LP1 |
| 227 | FXYD1 intron inserted at |
| | nucleotide 455 of LP1 |
| 228 | RBP4 intron inserted at |
| | nucleotide 455 of LP1 |
| 229 | APOC intron inserted at |
| | nucleotide 455 of LP1 |
| 230 | ATF5 intron inserted at |
| | nucleotide 455 of LP1 |
| 231 | HPD intron inserted at |
| | nucleotide 455 of LP1 |
| 232 | FLOT1 intron inserted at |
| | nucleotide 455 of LP1 |
| 233 | HBB intron 2 inserted at |
| | nucleotide 455 of LP1 |
| 234 | synthetic intron based on |
| | HBB and beta-globin |
| | inserted at nucleotide |
| | 455 of LP1 |
| 235 | IFI27L2 intron inserted at |
| | nucleotide 455 of LP1 |
| 236 | HCB4b promoter |
| 237 | HCB4 promoter |
| 238 | HCB3 promoter |
| 239 | EH38E1513438 |
| 240 | EH38E1514777 |
| 241 | EH38E1521375 |
| 242 | EH38E1761982 |
| 243 | EH38E1841027 |
| 244 | EH38E2144693 |
| 245 | EH38E2147276 |
| 246 | EH38E2274269 |
| 247 | EH38E2724199 |
| 248 | FTLmd |
| 248 | FTLmd |
| 249 | RPL26 intron inserted at |
| | nucleotide 455 of LP1 |
| 250 | AAT_v3_p |

TABLE 7
NAREs and NARE components. SN = SEQ ID NO.

| | | NARE name/ | |
|---|---|---|---|
| SN | Name | components | Sequence |
| 1 | L1 | AAT | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCGGCGGCCGCCACC |
| 2 | L1_v2 | AAT_v2 | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | AGCGGCCGCCACC |
| 3 | L2 | AAT-MVMi | CACCCCCTCCACCTTGGACACAGGACGCTGTGGTTTCT |
| | | (SynE-mTTR) | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCGAAGAGGTAAGGGTTTAAGGGATGGTTGGTTGGT |
| | | | GGGGTATTAATGTTTAATTACCTGGAGCGAGTTAATAA |
| | | | TTACCAGCGCGGGCCAAATAAATAATCCGCGAGGGGC |
| | | | AGCTGGACTTTGGACTCCGCGCGCTGGTAATTATTAAC |
| | | | CTCGCGAATATTGATTCGAGGCCGCGATTGCCGCAATC |
| | | | GCGAGGGGCAGGTGACCTTTGCCCAGCGCGCGTTCGC |
| | | | CCCGCCCCGGACGGTATCGATAAGCTTAGGAGCTTGG |
| | | | GCTGCAGGTCGAGGGCACTGGGAGGATGTTGAGTAAG |
| | | | ATGGAAAACTACTGATGACCCTTGCAGAGACAGAGTA |
| | | | TTAGGACCTGTTTGAACAGGGGCCGGGCGATCAGCAG |
| | | | GTAGCTCTAGAGGATCCCCGTCTGTCTGCACATTTCGT |
| | | | AGAGCGAGTGTTCCGATACTCTAATCTCCCTAGGCAAG |
| | | | GTTCATATTTGTGTAGGTTACTTATTCTCCTTTTGTTGA |
| | | | CTAAGTCAATAATCAGAATCAGCAGGTTTGGAGTCAG |
| | | | CTTGGCAGGGATCAGCAGCCTGGGTTGGAAGGAGGGG |
| | | | GTATAAAAGCCCCTTCACCAGGAGAAGCCGTCACACA |
| | | | GATCCACAAGCTCCTGACAGGACCTGCCTGAAATCACT |
| | | | TTTTTTCAGGTTGGGCGGCCGCCACC |
| 150 | | AATp | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCG |
| 151 | | MVMi-SynE- | AAGAGGTAAGGGTTTAAGGGATGGTTGGTTGGTGGGG |
| | | mTTRp intron | TATTAATGTTTAATTACCTGGAGCGAGTTAATAATTAC |
| | | | CAGCGCGGGCCAAATAAATAATCCGCGAGGGGCAGCT |
| | | | GGACTTTGGACTCCGCGCGCTGGTAATTATTAACCTCG |
| | | | CGAATATTGATTCGAGGCCGCGATTGCCGCAATCGCGA |
| | | | GGGGCAGGTGACCTTTGCCCAGCGCGCGTTCGCCCCGC |
| | | | CCCGGACGGTATCGATAAGCTTAGGAGCTTGGGCTGC |
| | | | AGGTCGAGGGCACTGGGAGGATGTTGAGTAAGATGGA |
| | | | AAACTACTGATGACCCTTGCAGAGACAGAGTATTAGG |
| | | | ACCTGTTTGAACAGGGGCCGGGCGATCAGCAGGTAGC |
| | | | TCTAGAGGATCCCCGTCTGTCTGCACATTTCGTAGAGC |
| | | | GAGTGTTCCGATACTCTAATCTCCCTAGGCAAGGTTCA |
| | | | TATTTGTGTAGGTTACTTATTCTCCTTTTGTTGACTAAG |
| | | | TCAATAATCAGAATCAGCAGGTTTGGAGTCAGCTTGGC |
| | | | AGGGATCAGCAGCCTGGGTTGGAAGGAGGGGGTATAA |
| | | | AAGCCCCTTCACCAGGAGAAGCCGTCACACAGATCCA |
| | | | CAAGCTCCTGACAGGACCTGCCTGAAATCACTTTTTTT |
| | | | CAGGTTGG |
| 152 | | SynE-mTTRp | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACCTGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGG |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 154 | | mTTRp | CGCGTTCGCCCCGCCCCGGACGGTATCGATAAGCTTAG |
| | | | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACCTGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCCTGACAGG |
| 4 | L3 | AAT-MVMi | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCGAAGAGGTAAGGGTTTAAGGGATGGTTGGTTGGT |
| | | | GGGGTATTAATGTTTAATTACCTGGAGCACCTGCCTGA |
| | | | AATCACTTTTTTTCAGGTTGGGCGGCCGCCACC |
| 150 | | AATp | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 5 | L4 | AATcre-mTTR | GGGGAGGCTGCTGGTGAATATTAACCAAGGTCACCCC |
| | | | AGTTATCGGAGGAGCAAACAGGGACTAAGTCCACGCC |
| | | | GCGTTCGCCCCGCCCCCGACGGTATCGATAAGCTTAGG |
| | | | AGCTTGGGCTGCAGGTCGAGGGCACTGGGAGGATGTT |
| | | | GAGTAAGATGGAAAACTACTGATGACCCTTGCAGAGA |
| | | | CAGAGTATTAGGACATGTTTGAACAGGGGCCGGGCGA |
| | | | TCAGCAGGTAGCTCTAGAGGATCCCCGTCTGTCTGCAC |
| | | | ATTTCGTAGAGCGAGTGTTCCGATACTCTAATCTCCCT |
| | | | AGGCAAGGTTCATATTTGTGTAGGTTACTTATTCTCCTT |
| | | | TTGTTGACTAAGTCAATAATCAGAATCAGCAGGTTTGG |
| | | | AGTCAGCTTGGCAGGGATCAGCAGCCTGGGTTGGAAG |
| | | | GAGGGGGTATAAAAGCCCCTTCACCAGGAGAAGCCGT |
| | | | CACACAGATCCACAAGCTCGAGCCGAGTGGTCGTGCC |
| | | | TCCATAGAAGCGGCCGCCACC |
| 156 | | AATcre | GGGGAGGCTGCTGGTGAATATTAACCAAGGTCACCCC |
| | | | AGTTATCGGAGGAGCAAACAGGGACTAAGTCCAC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 6 | L5 | ABPcre-mTTR | GTTAATTTTTAAAAAGCAGTCAAAAGTCCAAGTGGCCC |
| | | | TTGGCAGCATTTACTCTCTCTGTTTGCTCTGGTTAATAA |
| | | | TCTCAGGAGCACAAACATTCCTGGAGGCAGGAGAAGA |
| | | | AATCAACATCCTGGACTTATCCTCTGGGCCTGCCGCGT |
| | | | TCGCCCCGCCCCCGACGGTATCGATAAGCTTAGGAGCT |
| | | | TGGGCTGCAGGTCGAGGGCACTGGGAGGATGTTGAGT |
| | | | AAGATGGAAAACTACTGATGACCCTTGCAGAGACAGA |
| | | | GTATTAGGACATGTTTGAACAGGGGCCGGGCGATCAG |
| | | | CAGGTAGCTCTAGAGGATCCCCGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCGAGCCGAGTGGTCGTGCCTCCAT |
| | | | AGAAGCGGCCGCCACC |
| 161 | | ABPcre | GTTAATTTTTAAAAAGCAGTCAAAAGTCCAAGTGGCCC |
| | | | TTGGCAGCATTTACTCTCTCTGTTTGCTCTGGTTAATAA |
| | | | TCTCAGGAGCACAAACATTCCTGGAGGCAGGAGAAGA |
| | | | AATCAACATCCTGGACTTATCCTCTGGGCCT |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 7 | L6 | ALBcre-mTTR | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTCGCCGCGTTCGCCCC |
| | | | GCCCCCGACGGTATCGATAAGCTTAGGAGCTTGGGCTG |
| | | | CAGGTCGAGGGCACTGGGAGGATGTTGAGTAAGATGG |
| | | | AAAACTACTGATGACCCTTGCAGAGACAGAGTATTAG |
| | | | GACATGTTTGAACAGGGGCCGGGCGATCAGCAGGTAG |
| | | | CTCTAGAGGATCCCCGTCTGTCTGCACATTTCGTAGAG |
| | | | CGAGTGTTCCGATACTCTAATCTCCCTAGGCAAGGTTC |
| | | | ATATTTGTGTAGGTTACTTATTCTCCTTTTGTTGACTAA |
| | | | GTCAATAATCAGAATCAGCAGGTTTGGAGTCAGCTTGG |
| | | | CAGGGATCAGCAGCCTGGGTTGGAAGGAGGGGGTATA |
| | | | AAAGCCCCTTCACCAGGAGAAGCCGTCACACAGATCC |
| | | | ACAAGCTCGAGCCGAGTGGTCGTGCCTCCATAGAAGC |
| | | | GGCCGCCACC |
| 162 | | ALBcre | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 8 | L7 | APOEcre- | GCTGTTTGTGTGCTGCCTCTGAAGTCCACACTGAACAA |
| | | mTTR | ACTTCAGCCTACTCATGTCCCTAAAATGGGCAAACATT |
| | | | GCAAGCAGCAAACAGCAAACACACAGCCCTCCCTGCC |
| | | | TGCTGACCTTGGAGCTGGGGCAGAGGTCAGAGACCTC |
| | | | TCTGGCCGCGTTCGCCCCGCCCCCGACGGTATCGATAA |
| | | | GCTTAGGAGCTTGGGCTGCAGGTCGAGGGCACTGGGA |
| | | | GGATGTTGAGTAAGATGGAAAACTACTGATGACCCTT |
| | | | GCAGAGACAGAGTATTAGGACATGTTTGAACAGGGGC |
| | | | CGGGCGATCAGCAGGTAGCTCTAGAGGATCCCCGTCT |
| | | | GTCTGCACATTTCGTAGAGCGAGTGTTCCGATACTCTA |
| | | | ATCTCCCTAGGCAAGGTTCATATTTGTGTAGGTTACTT |
| | | | ATTCTCCTTTTGTTGACTAAGTCAATAATCAGAATCAG |
| | | | CAGGTTTGGAGTCAGCTTGGCAGGGATCAGCAGCCTG |
| | | | GGTTGGAAGGAGGGGGTATAAAAGCCCCTTCACCAGG |
| | | | AGAAGCCGTCACACAGATCCACAAGCTCGAGCCGAGT |
| | | | GGTCGTGCCTCCATAGAAGCGGCCGCCACC |
| 163 | | APOEcre | GCTGTTTGTGTGCTGCCTCTGAAGTCCACACTGAACAA |
| | | | ACTTCAGCCTACTCATGTCCCTAAAATGGGCAAACATT |
| | | | GCAAGCAGCAAACAGCAAACACACAGCCCTCCCTGCC |
| | | | TGCTGACCTTGGAGCTGGGGCAGAGGTCAGAGACCTC |
| | | | TCTG |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 9 | L8 | AY661265.1 v | CTCGAGGTCAATTCACGCGAGTTAATAATTACCAGCGC |
| | | 2 (mTTR- | GGGCCAAATAAATAATCCGCGAGGGGCAGCTGGACTT |
| | | based) | TGGACTCCGCGCGCTGGTAATTATTAACCTCGCGAATA |
| | | | TTGATTCGAGGCCGCGATTGCCGCAATCGCGAGGGGC |
| | | | AGGTGACCTTTGCCCAGCGCGCGTTCGCCCCGCCCCGG |
| | | | ACGGTATCGATAAGCTTAGGAGCTTGGGCTGCAGGTC |
| | | | GAGGGCACTGGGAGGATGTTGAGTAAGATGGAAAACT |
| | | | ACTGATGACCCTTGCAGAGACAGAGTATTAGGACATG |
| | | | TTTGAACAGGGGCCGGGCGATCAGCAGGTAGCTCTAG |
| | | | AGGATCCCCGTCTGTCTGCACATTTCGTAGAGCGAGTG |
| | | | TTCCGATACTCTAATCTCCCTAGGCAAGGTTCATATTT |
| | | | GTGTAGGTTACTTATTCTCCTTTTGTTGACTAAGTCAAT |
| | | | AATCAGAATCAGCAGGTTTGGAGTCAGCTTGGCAGGG |
| | | | ATCAGCAGCCTGGGTTGGAAGGAGGGGGTATAAAAGC |
| | | | CCCTTCACCAGGAGAAGCCGTCACACAGATCCACAAG |
| | | | CTCCTGCCACC |
| 10 | L8a | SynE-mTTR | CTCGAGGTCAATTCACGCGAGTTAATAATTACCAGCGC |
| | | | GGGCCAAATAAATAATCCGCGAGGGGCAGGTGACGTT |
| | | | TGCCCAGCGCGCGCTGGTAATTATTAACCTCGCGAATA |
| | | | TTGATTCGAGGCCGCGATTGCCGCAATCGCGAGGGGC |
| | | | AGGTGACCTTTGCCCAGCGCGCGTTCGCCCCGCCCCGG |
| | | | ACGGTATCGATAAGCTTAGGAGCTTGGGCTGCAGGTC |
| | | | GAGGGCACTGGGAGGATGTTGAGTAAGATGGAAAACT |
| | | | ACTGATGACCCTTGCAGAGACAGAGTATTAGGACATG |
| | | | TTTGAACAGGGGCCGGGCGATCAGCAGGTAGCTCTAG |
| | | | AGGATCCCCGTCTGTCTGCACATTTCGTAGAGCGAGTG |
| | | | TTCCGATACTCTAATCTCCCTAGGCAAGGTTCATATTT |
| | | | GTGTAGGTTACTTATTCTCCTTTTGTTGACTAAGTCAAT |
| | | | AATCAGAATCAGCAGGTTTGGAGTCAGCTTGGCAGGG |
| | | | ATCAGCAGCCTGGGTTGGAAGGAGGGGGTATAAAAGC |
| | | | CCCTTCACCAGGAGAAGCCGTCACACAGATCCACAAG |
| | | | CTCCTGCGGCCGCCACC |
| 11 | L9 | Chr19F3_R3_v | GCGACCTGCCCAGCACACCCTGGGGCAGCGCCGTGAC |
| | | 2-mTTR | GTCAGCACGCCGGGCGGGGACCGGGAGATCCTTGGGG |
| | | (MTE_DPE) | CGGTGGGGGGCCAGCGGCAGTTCCCGGCGGCCCGCCG |
| | | | CGTAAGCCACGCCCACTACGGTATCGATAAGCTTAGG |
| | | | AGCTTGGGCTGCAGGTCGAGGGCACTGGGAGGATGTT |
| | | | GAGTAAGATGGAAAACTACTGATGACCCTTGCAGAGA |
| | | | CAGAGTATTAGGACATGTTTGAACAGGGGCCGGGCGA |
| | | | TCAGCAGGTAGCTCTAGAGGATCCCCGTCTGTCTGCAC |
| | | | ATTTCGTAGAGCGAGTGTTCCGATACTCTAATCTCCCT |
| | | | AGGCAAGGTTCATATTTGTGTAGGTTACTTATTCTCCTT |
| | | | TTGTTGACTAAGTCAATAATCAGAATCAGCAGGTTTGG |
| | | | AGTCAGCTTGGCAGGGATCAGCAGCCTGGGTTGGAAG |
| | | | GAGGGGGTATAAAAGCCCCTTCACCAGGAGAAGCCGT |
| | | | CACACAGATCCACAAGCTCGAGCCGAGTGGTCGTGCC |
| | | | TCCATAGAAGCGGCCGCCACC |
| 165 | | Chr19-F3/R3 | GCGACCTGCCCAGCACACCCTGGGGCAGCGCCGTGAC |
| | | | GTCAGCACGCCGGGCGGGGACCGGGAGATCCTTGGGG |
| | | | CGGTGGGGGGCCAGCGGCAGTTCCCGGCGGCCC |
| 158 | | mTTRmd2 | CGCGTAAGCCACGCCCACTACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 12 | L10 | FGBcre1- | GAATTTTAATAAATACCTCTCTGTCCTTCTCCTAGCTTC |
| | | mTTR | ACCCTTTGTTTGTTGAAAGGAGATACTTTAGTGCAGTA |
| | | (MPE_DPE) | TTGCAACAGCTTATGTAGACATGCAACCTTGAGGTCAC |
| | | | AGACCTATGAGGGATTGAGACAAAGGCAAATTTAGCA |
| | | | AATATTTTCCAGAGTACTCTGCTGCAATAACGCCGCGT |
| | | | TCGCCCCGCCCCCGACGGTATCGATAAGCTTAGGAGCT |
| | | | TGGGCTGCAGGTCGAGGGCACTGGGAGGATGTTGAGT |
| | | | AAGATGGAAAACTACTGATGACCCTTGCAGAGACAGA |
| | | | GTATTAGGACATGTTTGAACAGGGGCCGGGCGATCAG |
| | | | CAGGTAGCTCTAGAGGATCCCCGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCGAGCCGAGTGGTCGTGCCTCCAT |
| | | | AGAAGCGGCCGCCACC |
| 166 | | FGBcre1 | GAATTTTAATAAATACCTCTCTGTCCTTCTCCTAGCTTC |
| | | | ACCCTTTGTTTGTTGAAAGGAGATACTTTAGTGCAGTA |
| | | | TTGCAACAGCTTATGTAGACATGCAACCTTGAGGTCAC |
| | | | AGACCTATGAGGGATTGAGACAAAGGCAAATTTAGCA |
| | | | AATATTTTCCAGAGTACTCTGCTGCAATAAC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 13 | L10_v | FGBcre1- | GAATTTTAATAAATACCTCTCTGTCCTTCTCCTAGCTTC |
| | 2 | mTTR | ACCCTTTGTTTGTTGAAAGGAGATACTTTAGTGCAGTA |
| | | (MPE_DPE) v2 | TTGCAACAGCTTATGTAGACATGCAACCTTGAGGTCAC |
| | | | AGACCTATGAGGGATTGAGACAAAGGCAAATTTAGCA |
| | | | AATATTTTCCAGAGTACTCTGCTGCAATAACCGCGTTC |
| | | | GCCCCGCCCCGGACGGTATCGATAAGCTTAGGAGCTTG |
| | | | GGCTGCAGGTCGAGGGCACTGGGAGGATGTTGAGTAA |
| | | | GATGGAAAACTACTGATGACCCTTGCAGAGACAGAGT |
| | | | ATTAGGACATGTTTGAACAGGGGCCGGGCGATCAGCA |
| | | | GGTAGCTCTAGAGGATCCCCGTCTGTCTGCACATTTCG |
| | | | TAGAGCGAGTGTTCCGATACTCTAATCTCCCTAGGCAA |
| | | | GGTTCATATTTGTGTAGGTTACTTATTCTCCTTTTGTTG |
| | | | ACTAAGTCAATAATCAGAATCAGCAGGTTTGGAGTCA |
| | | | GCTTGGCAGGGATCAGCAGCCTGGGTTGGAAGGAGGG |
| | | | GGTATAAAAGCCCCTTCACCAGGAGAAGCCGTCACAC |
| | | | AGATCCACAAGCTCGAGCCGAGTGGTCGTGCCTCCATA |
| | | | GAAGCGGCCGCCACC |
| 166 | | FGBcre1 | GAATTTTAATAAATACCTCTCTGTCCTTCTCCTAGCTTC |
| | | | ACCCTTTGTTTGTTGAAAGGAGATACTTTAGTGCAGTA |
| | | | TTGCAACAGCTTATGTAGACATGCAACCTTGAGGTCAC |
| | | | AGACCTATGAGGGATTGAGACAAAGGCAAATTTAGCA |
| | | | AATATTTTCCAGAGTACTCTGCTGCAATAAC |
| 157 | | mTTRmd | CGCGTTCGCCCCGCCCCGGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 14 | L11 | HBVcre-mTTR | GTCAACGACCGACCTTGAGGCATACTTCAAAGACTGTT |
| | | | TGTTTAAAGACTGGGAGGAGTTGGGGGAGGAGATTAG |
| | | | GTTAAAGGTCTTTGTACGCCGCGTTCGCCCCGCCCCCG |
| | | | ACGGTATCGATAAGCTTAGGAGCTTGGGCTGCAGGTC |
| | | | GAGGGCACTGGGAGGATGTTGAGTAAGATGGAAAACT |
| | | | ACTGATGACCCTTGCAGAGACAGAGTATTAGGACATG |
| | | | TTTGAACAGGGGCCGGGCGATCAGCAGGTAGCTCTAG |
| | | | AGGATCCCCGTCTGTCTGCACATTTCGTAGAGCGAGTG |
| | | | TTCCGATACTCTAATCTCCCTAGGCAAGGTTCATATTT |
| | | | GTGTAGGTTACTTATTCTCCTTTTGTTGACTAAGTCAAT |
| | | | AATCAGAATCAGCAGGTTTGGAGTCAGCTTGGCAGGG |
| | | | ATCAGCAGCCTGGGTTGGAAGGAGGGGGTATAAAAGC |
| | | | CCCTTCACCAGGAGAAGCCGTCACACAGATCCACAAG |
| | | | CTCGAGCCGAGTGGTCGTGCCTCCATAGAAGCGGCCG |
| | | | CCACC |
| 167 | | HBVcre | GTCAACGACCGACCTTGAGGCATACTTCAAAGACTGTT |
| | | | TGTTTAAAGACTGGGAGGAGTTGGGGGAGGAGATTAG |
| | | | GTTAAAGGTCTTTGTAC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 15 | L12 | mTTR | GCCGCGTTCGCCCCGCCCCCGACGGTATCGATAAGCTT |
| | | (MTE_DPE) | AGGAGCTTGGGCTGCAGGTCGAGGGCACTGGGAGGAT |
| | | | GTTGAGTAAGATGGAAAACTACTGATGACCCTTGCAG |
| | | | AGACAGAGTATTAGGACATGTTTGAACAGGGGCCGGG |
| | | | CGATCAGCAGGTAGCTCTAGAGGATCCCCGTCTGTCTG |
| | | | CACATTTCGTAGAGCGAGTGTTCCGATACTCTAATCTC |
| | | | CCTAGGCAAGGTTCATATTTGTGTAGGTTACTTATTCTC |
| | | | CTTTTGTTGACTAAGTCAATAATCAGAATCAGCAGGTT |
| | | | TGGAGTCAGCTTGGCAGGGATCAGCAGCCTGGGTTGG |
| | | | AAGGAGGGGGTATAAAAGCCCCTTCACCAGGAGAAGC |
| | | | CGTCACACAGATCCACAAGCTCGAGCCGAGTGGTCGT |
| | | | GCCTCCATAGAAGCGGCCGCCACC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 16 | L13 | SERPINA1cre- | GAGTTGCTGGTGCTTCCCCAGGCTGGAGATTGAGTTAA |
| | | mTTR | TATTAACAGGCCCAAGGCGATGTGGGCTTGTGCAATCA |
| | | (MTE_DPE) | TAGGCCCGGCCTCGCCAGGTCACCTGAGGAGTTAATG |
| | | | AATACATATCTCCTGCCGCGTTCGCCCCGCCCCCGACG |
| | | | GTATCGATAAGCTTAGGAGCTTGGGCTGCAGGTCGAG |
| | | | GGCACTGGGAGGATGTTGAGTAAGATGGAAAACTACT |
| | | | GATGACCCTTGCAGAGACAGAGTATTAGGACATGTTTG |
| | | | AACAGGGGCCGGGCGATCAGCAGGTAGCTCTAGAGGA |
| | | | TCCCCGTCTGTCTGCACATTTCGTAGAGCGAGTGTTCC |
| | | | GATACTCTAATCTCCCTAGGCAAGGTTCATATTTGTGT |
| | | | AGGTTACTTATTCTCCTTTTGTTGACTAAGTCAATAATC |
| | | | AGAATCAGCAGGTTTGGAGTCAGCTTGGCAGGGATCA |
| | | | GCAGCCTGGGTTGGAAGGAGGGGGTATAAAAGCCCCT |
| | | | TCACCAGGAGAAGCCGTCACACAGATCCACAAGCTCG |
| | | | AGCCGAGTGGTCGTGCCTCCATAGAAGCGGCCGCCAC |
| | | | C |
| 168 | | SERPINAlcre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 17 | L13_T | SERPINAlcre- | GAGTTGCTGGTGCTTCCCCAGGCTGGAGATTGAGTTAA |
| | ISU | mTTR | TATTAACAGGCCCAAGGCGATGTGGGCTTGTGCAATCA |
| | | (MTE_DPE)- | TAGGCCCGGCCTCGCCAGGTCACCTGAGGAGTTAATG |
| | | TISU | AATACATATCTCCTGCCGCGTTCGCCCCGCCCCCGACG |
| | | | GTATCGATAAGCTTAGGAGCTTGGGCTGCAGGTCGAG |
| | | | GGCACTGGGAGGATGTTGAGTAAGATGGAAAACTACT |
| | | | GATGACCCTTGCAGAGACAGAGTATTAGGACATGTTTG |
| | | | AACAGGGGCCGGGCGATCAGCAGGTAGCTCTAGAGGA |
| | | | TCCCCGTCTGTCTGCACATTTCGTAGAGCGAGTGTTCC |
| | | | GATACTCTAATCTCCCTAGGCAAGGTTCATATTTGTGT |
| | | | AGGTTACTTATTCTCCTTTTGTTGACTAAGTCAATAATC |
| | | | AGAATCAGCAGGTTTGGAGTCAGCTTGGCAGGGATCA |
| | | | GCAGCCTGGGTTGGAAGGAGGGGGTATAAAAGCCCCT |
| | | | TCACCAGGAGAAGCCGTCACACAGATCCACAAGCTCG |
| | | | AGCCGAGTGGTCGTGCCTCCATAGAAGCGGCCGCAAG |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 170 | | TISU | CAAG |
| 18 | L13_v | SERPINAlcre- | GAGTTGCTGGTGCTTCCCCAGGCTGGAGATTGAGTTAA |
| | 2 | mTTR | TATTAACAGGCCCAAGGCGATGTGGGCTTGTGCAATCA |
| | | (MTE_DPE) | TAGGCCCGGCCTCGCCAGGTCACCTGAGGAGTTAATG |
| | | v2 | AATACATATCTCCTGCCGCGTTCGCCCCGCCCCCGACG |
| | | | GTATCGATAAGCTTAGGAGCTTGGGCTGCAGGTCGAG |
| | | | GGCACTGGGAGGATGTTGAGTAAGATGGAAAACTACT |
| | | | GATGACCCTTGCAGAGACAGAGTATTAGGACATGTTTG |
| | | | AACAGGGGCCGGGCGATCAGCAGGTAGCTCTAGAGGA |
| | | | TCCCCGTCTGTCTGCACATTTCGTAGAGCGAGTGTTCC |
| | | | GATACTCTAATCTCCCTAGGCAAGGTTCATATTTGTGT |
| | | | AGGTTACTTATTCTCCTTTTGTTGACTAAGTCAATAATC |
| | | | AGAATCAGCAGGTTTGGAGTCAGCTTGGCAGGGATCA |
| | | | GCAGCCTGGGTTGGAAGGAGGGGGTATAAAAGCCCCT |
| | | | TCACCAGGAGAAGCCGTCACACAGATCCACAAGCTCG |
| | | | AGCCGAGTGGTCGTGCCTCCATAGAAGCGGCCGCAAC |
| | | | TCCTAAAAAACCGCCACC |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINAlcre | TCGCCAGGTCACCTGAGGAGTTAATGAATACATATCTC |
| | | 2 | CT |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 159 | | MTE/DPE | CGAGCCGAGTGGTCGT |
| 19 | L14 | SynE-mTTR- | GAGTTAATAATTACCAGCGCGGGCCAAATAAATAATC |
| | | MVMi (AAT) | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGGAAGAGGTA |
| | | | AGGGTTTAAGGGATGGTTGGTTGGTGGGGTATTAATGT |
| | | | TTAATTACCTGGAGCACACCCCCTCCACCTTGGACACA |
| | | | GGACGCTGTGGTTTCTGAGCCAGGTACAATGACTCCTT |
| | | | TCGGTAAGTGCAGTGGAAGCTGTACACTGCCCAGGCA |
| | | | AAGCGTCCGGGCAGCGTAGGCGGGCGACTCAGATCCC |
| | | | AGCCAGTGGACTTAGCCCCTGTTTGCTCCTCCGATAAC |
| | | | TGGGGTGACCTTGGTTAATATTCACCAGCAGCCTCCCC |
| | | | CGTTGCCCCTCTGGATCCACTGCTTAAATACGGACGAG |
| | | | GACAGGGCCCTGTCTCCTCAGCTTCAGGCACCACCACT |
| | | | GACCTGGGACAGTGAATCGCCTGCCTGAAATCACTTTT |
| | | | TTTCAGGTTGGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 154 | | mTTRp | CGCGTTCGCCCCGCCCCGGACGGTATCGATAAGCTTAG |
| | | | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCCTGACAGG |
| 171 | | MVMi-AATp | AAGAGGTAAGGGTTTAAGGGATGGTTGGTTGGTGGGG |
| | | intron | TATTAATGTTTAATTACCTGGAGCACACCCCCTCCACC |
| | | | TTGGACACAGGACGCTGTGGTTTCTGAGCCAGGTACAA |
| | | | TGACTCCTTTCGGTAAGTGCAGTGGAAGCTGTACACTG |
| | | | CCCAGGCAAAGCGTCCGGGCAGCGTAGGCGGGCGACT |
| | | | CAGATCCCAGCCAGTGGACTTAGCCCCTGTTTGCTCCT |
| | | | CCGATAACTGGGGTGACCTTGGTTAATATTCACCAGCA |
| | | | GCCTCCCCCGTTGCCCCTCTGGATCCACTGCTTAAATA |
| | | | CGGACGAGGACAGGGCCCTGTCTCCTCAGCTTCAGGC |
| | | | ACCACCACTGACCTGGGACAGTGAATCGCCTGCCTGA |
| | | | AATCACTTTTTTTCAGGTTGG |
| 150 | | AATp | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCG |
| 20 | L15 | SynEnh- | GAGTTAATAATTACCAGCGCGGGCCAAATAAATAATC |
| | | mTTR v2 | CGCGAGGGGCAGCTGGACTTTGGACTCCGCGCGCTGG |
| | | (MTE_DPE) | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCGAGCCGAGTGGTCGTG |
| | | | CCTCCATAGAAGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 157 | | mTTRmd | CGCGTTCGCCCCGCCCCGGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 21 | L15_L | SynEnh- | GAGTTAATAATTACCAGCGCGGGCCAAATAAATAATC |
| | 21 | mTTR v2 | CGCGAGGGGCAGCTGGACTTTGGACTCCGCGCGCTGG |
| | | (MTE_DPE)- | TAATTATTAACCTCGCGAATATTGATTCGAGGCCGCGA |
| | | L21 | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCGAGCCGAGTGGTCGTG |
| | | | CCTCCATAGAAGCGGCCGCAACTCCTAAAAAACCGCC |
| | | | ACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 157 | | mTTRmd | CGCGTTCGCCCCGCCCCGGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 172 | | L21 | AACTCCTAAAAAACCGCCACC |
| 22 | L15_L | SynEnh- | GAGTTAATAATTACCAGCGCGGGCCAAATAAATAATC |
| | 21_TI | mTTR v2 | CGCGAGGGGCAGCTGGACTTTGGACTCCGCGCGCTGG |
| | SU | (MTE_DPE)- | TAATTATTAACCTCGCGAATATTGATTCGAGGCCGCGA |
| | | L21 TISU | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCGAGCCGAGTGGTCGTG |
| | | | CCTCCATAGAAGCGGCCGCAACTCCTAAAAAACCGCC |
| | | | AAG |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 157 | | mTTRmd | CGCGTTCGCCCCGCCCCGGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 173 | | L21-tisu | AACTCCTAAAAAACCGCCAAG |
| 23 | L15_T | SynEnh- | GAGTTAATAATTACCAGCGCGGGCCAAATAAATAATC |
| | ISU | mTTR_v2 | CGCGAGGGGCAGCTGGACTTTGGACTCCGCGCGCTGG |
| | | (MTE_DPE)- | TAATTATTAACCTCGCGAATATTGATTCGAGGCCGCGA |
| | | TISU | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCGAGCCGAGTGGTCGTG |
| | | | CCTCCATAGAAGCGGCCGCAAG |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 157 | | mTTRmd | CGCGTTCGCCCCGCCCCGGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 170 | | TISU | CAAG |
| 24 | L16 | synEnh_v2- | GAGTTAATAATTACCAGCGCGGGCCAAATAAATAATC |
| | | AAT-MVMi | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACCACCCCCTCCACCTTG |
| | | | GACACAGGACGCTGTGGTTTCTGAGCCAGGTACAATG |
| | | | ACTCCTTTCGGTAAGTGCAGTGGAAGCTGTACACTGCC |
| | | | CAGGCAAAGCGTCCGGGCAGCGTAGGCGGGCGACTCA |
| | | | GATCCCAGCCAGTGGACTTAGCCCCTGTTTGCTCCTCC |
| | | | GATAACTGGGGTGACCTTGGTTAATATTCACCAGCAGC |
| | | | CTCCCCCGTTGCCCCTCTGGATCCACTGCTTAAATACG |
| | | | GACGAGGACAGGGCCCTGTCTCCTCAGCTTCAGGCACC |
| | | | ACCACTGACCTGGGACAGTGAATCGAAGAGGTAAGGG |
| | | | TTTAAGGGATGGTTGGTTGGTGGGGTATTAATGTTTAA |
| | | | TTACCTGGAGCACCTGCCTGAAATCACTTTTTTTCAGG |
| | | | TTGGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 188 | | AAT_v2_p | CGCGTTCGCCCCGCCCCGGACCACCCCCTCCACCTTGG |
| | | | ACACAGGACGCTGTGGTTTCTGAGCCAGGTACAATGA |
| | | | CTCCTTTCGGTAAGTGCAGTGGAAGCTGTACACTGCCC |
| | | | AGGCAAAGCGTCCGGGCAGCGTAGGCGGGCGACTCAG |
| | | | ATCCCAGCCAGTGGACTTAGCCCCTGTTTGCTCCTCCG |
| | | | ATAACTGGGGTGACCTTGGTTAATATTCACCAGCAGCC |
| | | | TCCCCCGTTGCCCCTCTGGATCCACTGCTTAAATACGG |
| | | | ACGAGGACAGGGCCCTGTCTCCTCAGCTTCAGGCACCA |
| | | | CCACTGACCTGGGACAGTGAATCG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 25 | L17 | SynEnh_v2- | GAGTTAATAATTACCAGCGCGGGCCAAATAAATAATC |
| | | mTTR | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGGGCGGCCGC |
| | | | CACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 154 | | mTTRp | CGCGTTCGCCCCGCCCCGGACGGTATCGATAAGCTTAG |
| | | | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCCTGACAGG |
| 26 | L18 | SynEnh_v2- | GAGTTAATAATTACCAGCGCGGGCCAAATAAATAATC |
| | | mTTR-MVMi | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAGGGCACTGGGAGGATG |
| | | | TTGAGTAAGATGGAAAACTACTGATGACCCTTGCAGA |
| | | | GACAGAGTATTAGGACATGTTTGAACAGGGGCCGGGC |
| | | | GATCAGCAGGTAGCTCTAGAGGATCCCCGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGGAAGAGGTA |
| | | | AGGGTTTAAGGGATGGTTGGTTGGTGGGGTATTAATGT |
| | | | TTAATTACCTGGAGCACCTGCCTGAAATCACTTTTTTTC |
| | | | AGGTTGGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 154 | | mTTRp | CGCGTTCGCCCCGCCCCGGACGGTATCGATAAGCTTAG |
| | | | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 27 | L19 | SynEnhCFLv0. | CAGTTAATGATTAACTTCGCGCCTGTTTACTTTGGCTCG |
| | | 3 -mTTR | CGTGCCCGTCTGGACTTTGGACTCGGCGCGCGAGTTAA |
| | | | TGATTAACTTCGCGTTTATTGATTTCGCGCGCGCATTGC |
| | | | ACAATACGCGTGCGCGCGCTGGACTTTGGACTCGCCGC |
| | | | GTTCGCCCCGCCCCCGACGGTATCGATAAGCTTAGGAG |
| | | | CTTGGGCTGCAGGTCGAGGGCACTGGGAGGATGTTGA |
| | | | GTAAGATGGAAAACTACTGATGACCCTTGCAGAGACA |
| | | | GAGTATTAGGACATGTTTGAACAGGGGCCGGGCGATC |
| | | | AGCAGGTAGCTCTAGAGGATCCCCGTCTGTCTGCACAT |
| | | | TTCGTAGAGCGAGTGTTCCGATACTCTAATCTCCCTAG |
| | | | GCAAGGTTCATATTTGTGTAGGTTACTTATTCTCCTTTT |
| | | | GTTGACTAAGTCAATAATCAGAATCAGCAGGTTTGGA |
| | | | GTCAGCTTGGCAGGGATCAGCAGCCTGGGTTGGAAGG |
| | | | AGGGGGTATAAAAGCCCCTTCACCAGGAGAAGCCGTC |
| | | | ACACAGATCCACAAGCTCGAGCCGAGTGGTCGTGCCT |
| | | | CCATAGAAGCGGCCGCCACC |
| 174 | | SynEnh_v3 | AGTTAATGATTAACTTCGCGCCTGTTTACTTTGGCTCGC |
| | | | GTGCCCGTCTGGACTTTGGACTCGGCGCGCGAGTTAAT |
| | | | GATTAACTTCGCGTTTATTGATTTCGCGCGCGCATTGC |
| | | | ACAATACGCGTGCGCGCGCTGGACTTTGGACTC |
| 157 | | mTTRmd | CGCGTTCGCCCCGCCCCCGACGGTATCGATAAGCTTAG |
| | | promoter | GAGCTTGGGCTGCAGGTCGAGGGCACTGGGAGGATGT |
| | | | TGAGTAAGATGGAAAACTACTGATGACCCTTGCAGAG |
| | | | ACAGAGTATTAGGACATGTTTGAACAGGGGCCGGGCG |
| | | | ATCAGCAGGTAGCTCTAGAGGATCCCCGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCGAGCCGAGTGGTCGT |
| 28 | L20 | ALB- | CTAAAATGTTCAAGTGGGAAATTACAGTTAAATACCAT |
| | | EH38E230319 | GGTAATGAATAAAAGGTACAAATCGTTTAAACTCTTAT |
| | | 3-mTTRp- | GTAAAATTTGATAAGATGTTTTACACAACTTTAATACA |
| | | MVMi | TTGACAAGGTCTTGTGGAGAAAACAGTTCCAGATGGT |
| | | | AAATATACACAAGGGATTTAGTCAAACAATTTTTTGGC |
| | | | AAGAATATTATGAATTTTGTAATCGGTTGGCAGCCAAT |
| | | | GAAATACAAAGATGAGTCTAGTTAATAATCTACAATTA |
| | | | TTGGTTAAAGAAGTATATTAGTGCTAATTTCCCTCCGT |
| | | | TTGTCCTAGCTTTTCTCTTCTGTCAACCCCACACGCCTT |
| | | | TGGGTACCCGGGGGCGCGCCAGTGTCTGTCTGCACATT |
| | | | TCGTAGAGCGAGTGTTCCGATACTCTAATCTCCCTAGG |
| | | | CAAGGTTCATATTTGTGTAGGTTACTTATTCTCCTTTTG |
| | | | TTGACTAAGTCAATAATCAGAATCAGCAGGTTTGGAGT |
| | | | CAGCTTGGCAGGGATCAGCAGCCTGGGTTGGAAGGAG |
| | | | GGGGTATAAAAGCCCCTTCACCAGGAGAAGCCGTCAC |
| | | | ACAGATCCACAAGCTCCTGACAGGAAGAGGTAAGGGT |
| | | | TTAAGGGATGGTTGGTTGGTGGGGTATTAATGTTTAAT |
| | | | TACCTGGAGCACCTGCCTGAAATCACTTTTTTTCAGGT |
| | | | TGGGCGGCCGCCACC |
| 175 | | EH38E2303193 | CTAAAATGTTCAAGTGGGAAATTACAGTTAAATACCAT |
| | | | GGTAATGAATAAAAGGTACAAATCGTTTAAACTCTTAT |
| | | | GTAAAATTTGATAAGATGTTTTACACAACTTTAATACA |
| | | | TTGACAAGGTCTTGTGGAGAAAACAGTTCCAGATGGT |
| | | | AAATATACACAAGGGATTTAGTCAAACAATTTTTTGGC |
| | | | AAGAATATTATGAATTTTGTAATCGGTTGGCAGCCAAT |
| | | | GAAATACAAAGATGAGTCTAGTTAATAATCTACAATTA |
| | | | TTGGTTAAAGAAGTATATTAGTGCTAATTTCCCTCCGT |
| | | | TTGTCCTAGCTTTTCTCTTCTGTCAACCCCACACGCCTT |
| | | | TG |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 29 | L21 | ALB- | GAGTTCAAGACCAGCCTGGGCAAGTCTCTTTAAAAAA |
| | | EH38E230319 | AACAAAACAAACAAACAAAAAAATTAGGCATGGTGGC |
| | | 4-mTTRp- | ACATGCCTGTAGTCCTAGCTACTTAGGAGGCTGACGTA |
| | | MVMi | GGAGGATCGTTTGGACCTGAGAGGTCAAGGCTACAGT |
| | | | GAGCCATGATTGTGCCACTGCACTCCAGCCTGGGTGAC |
| | | | AGAGTGAGACTGGTACCCGGGGGCGCGCCAGTGTCTG |
| | | | TCTGCACATTTCGTAGAGCGAGTGTTCCGATACTCTAA |
| | | | TCTCCCTAGGCAAGGTTCATATTTGTGTAGGTTACTTAT |
| | | | TCTCCTTTTGTTGACTAAGTCAATAATCAGAATCAGCA |
| | | | GGTTTGGAGTCAGCTTGGCAGGGATCAGCAGCCTGGG |
| | | | TTGGAAGGAGGGGGTATAAAAGCCCCTTCACCAGGAG |
| | | | AAGCCGTCACACAGATCCACAAGCTCCTGACAGGAAG |
| | | | AGGTAAGGGTTTAAGGGATGGTTGGTTGGTGGGGTATT |
| | | | AATGTTTAATTACCTGGAGCACCTGCCTGAAATCACTT |
| | | | TTTTTCAGGTTGGGCGGCCGCCACC |
| 177 | | EH38E2303194 | GAGTTCAAGACCAGCCTGGGCAAGTCTCTTTAAAAAA |
| | | | AACAAAACAAACAAACAAAAAAATTAGGCATGGTGGC |
| | | | ACATGCCTGTAGTCCTAGCTACTTAGGAGGCTGACGTA |
| | | | GGAGGATCGTTTGGACCTGAGAGGTCAAGGCTACAGT |
| | | | GAGCCATGATTGTGCCACTGCACTCCAGCCTGGGTGAC |
| | | | AGAGTGAGACT |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 30 | L22 | ALB- | TCTTTGCCCCTATTTTAGGGATAAGGATTCTGAAATGT |
| | | EH38E230320 | GGAGATGGTAAGTAAAATTGCACAACTGAAGAATGAG |
| | | 2-mTTRp- | TTACATGACTTGGCTCAAATACTGGTCATTGAACTCCA |
| | | MVMi | GAGCCTGAATATTCTTAACCACTTACATGATGCAAGCT |
| | | | CACCAAATAAATAGTTCGAATGTATTGTGACAGAGCG |
| | | | GCATTGATATTCATCTATTCATGTGGCTTTGAGTAGGA |
| | | | AGAAGAAAGGATATCATTCTGACCAGAGGGGTGAAAA |
| | | | ACAACCTGCATCTGATCCTGAGGCATAATACTATTAAC |
| | | | ACAATTCTTTTATGTTTCAGTTCGATGAATTTAAACCTC |
| | | | TTGTGGAAGAGGGTACCCGGGGGCGCGCCAGTGTCTG |
| | | | TCTGCACATTTCGTAGAGCGAGTGTTCCGATACTCTAA |
| | | | TCTCCCTAGGCAAGGTTCATATTTGTGTAGGTTACTTAT |
| | | | TCTCCTTTTGTTGACTAAGTCAATAATCAGAATCAGCA |
| | | | GGTTTGGAGTCAGCTTGGCAGGGATCAGCAGCCTGGG |
| | | | TTGGAAGGAGGGGGTATAAAAGCCCCTTCACCAGGAG |
| | | | AAGCCGTCACACAGATCCACAAGCTCCTGACAGGAAG |
| | | | AGGTAAGGGTTTAAGGGATGGTTGGTTGGTGGGGTATT |
| | | | AATGTTTAATTACCTGGAGCACCTGCCTGAAATCACTT |
| | | | TTTTTCAGGTTGGGCGGCCGCCACC |
| 178 | | EH38E2303202 | TCTTTGCCCCTATTTTAGGGATAAGGATTCTGAAATGT |
| | | | GGAGATGGTAAGTAAAATTGCACAACTGAAGAATGAG |
| | | | TTACATGACTTGGCTCAAATACTGGTCATTGAACTCCA |
| | | | GAGCCTGAATATTCTTAACCACTTACATGATGCAAGCT |
| | | | CACCAAATAAATAGTTCGAATGTATTGTGACAGAGCG |
| | | | GCATTGATATTCATCTATTCATGTGGCTTTGAGTAGGA |
| | | | AGAAGAAAGGATATCATTCTGACCAGAGGGGTGAAAA |
| | | | ACAACCTGCATCTGATCCTGAGGCATAATACTATTAAC |
| | | | ACAATTCTTTTATGTTTCAGTTCGATGAATTTAAACCTC |
| | | | TTGTGGAAGAG |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 31 | L23 | APOH- | CAACATGGCAGAGAAAACTCGAGAACAAGATGAGCAC |
| | | EH38E187954 | TGGAGAAATCATTGTGGATGAGTCACACTGGCACTACC |
| | | 0-mTTRp- | AAAGTGGTTTTCGTCTGCTAAAAAGACAGAGCCAAAT |
| | | MVMi | ATGAAAGCGCTTAAACATTAACCATTTTCTGGTGTACT |
| | | | TTTATCTTTGTAAATAGAAGATAAAGTATCATGATTTG |
| | | | AATGGAACTCTGAGACCCACATTCTTTAATCATTTACT |
| | | | TTCCACTTATGTGGCTCTGTTATGAAATCTTTGGGAGA |
| | | | TGGTTTCTGTCTTCATAACGTTTTTGTTAATGGCACAAA |
| | | | ACAGTTCATTATTGCTTTTGACAGAAACTTGTTGTTTCC |
| | | | GGGGTTGGGTACCCGGGGGCGCGCCAGTGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGGAAGAGGTA |
| | | | AGGGTTTAAGGGATGGTTGGTTGGTGGGGTATTAATGT |
| | | | TTAATTACCTGGAGCACCTGCCTGAAATCACTTTTTTTC |
| | | | AGGTTGGGCGGCCGCCACC |
| 179 | | EH38E1879540 | CAACATGGCAGAGAAAACTCGAGAACAAGATGAGCAC |
| | | | TGGAGAAATCATTGTGGATGAGTCACACTGGCACTACC |
| | | | AAAGTGGTTTTCGTCTGCTAAAAAGACAGAGCCAAAT |
| | | | ATGAAAGCGCTTAAACATTAACCATTTTCTGGTGTACT |
| | | | TTTATCTTTGTAAATAGAAGATAAAGTATCATGATTTG |
| | | | AATGGAACTCTGAGACCCACATTCTTTAATCATTTACT |
| | | | TTCCACTTATGTGGCTCTGTTATGAAATCTTTGGGAGA |
| | | | TGGTTTCTGTCTTCATAACGTTTTTGTTAATGGCACAAA |
| | | | ACAGTTCATTATTGCTTTTGACAGAAACTTGTTGTTTCC |
| | | | GGGGTTG |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 32 | L24 | CRP- | GGAATAATGATAGAATGAGAGTACTAAAACCCCCACA |
| | | EH38E138983 | ACTGGCCCTACATGAATGGCCAGCTATCTCAAAAGAG |
| | | 1-mTTRp- | GGACTGTGCTTGTCAGAGGGAATCCCTTCAGGGGACTC |
| | | MVMi | TTGGACAGGTTAAAGTGCCATGGATATGTTGTGTAATG |
| | | | GGAAGTGTAAACTTACAGGGACTTGATTTCAAAGGTC |
| | | | ATTAGAGAAGTTAGTCACAACTTCTAAAGCAACTATCA |
| | | | GAAAACAGCTTGGACTCACTCAAGTAAACAGGGGCTT |
| | | | TATTGGCTTATAGACCTGGGCAGTCCAGGTGTAGATCT |
| | | | GGTTCCAGACAAGGCTGATTCAGAGACTCCACGTGGTC |
| | | | AAGGCTCTGTTGTGGTACCCGGGGGCGCGCCAGTGTCT |
| | | | GTCTGCACATTTCGTAGAGCGAGTGTTCCGATACTCTA |
| | | | ATCTCCCTAGGCAAGGTTCATATTTGTGTAGGTTACTT |
| | | | ATTCTCCTTTTGTTGACTAAGTCAATAATCAGAATCAG |
| | | | CAGGTTTGGAGTCAGCTTGGCAGGGATCAGCAGCCTG |
| | | | GGTTGGAAGGAGGGGGTATAAAAGCCCCTTCACCAGG |
| | | | AGAAGCCGTCACACAGATCCACAAGCTCCTGACAGGA |
| | | | AGAGGTAAGGGTTTAAGGGATGGTTGGTTGGTGGGGT |
| | | | ATTAATGTTTAATTACCTGGAGCACCTGCCTGAAATCA |
| | | | CTTTTTTTCAGGTTGGGCGGCCGCCACC |
| 180 | | EH38E1389831 | GGAATAATGATAGAATGAGAGTACTAAAACCCCCACA |
| | | | ACTGGCCCTACATGAATGGCCAGCTATCTCAAAAGAG |
| | | | GGACTGTGCTTGTCAGAGGGAATCCCTTCAGGGGACTC |
| | | | TTGGACAGGTTAAAGTGCCATGGATATGTTGTGTAATG |
| | | | GGAAGTGTAAACTTACAGGGACTTGATTTCAAAGGTC |
| | | | ATTAGAGAAGTTAGTCACAACTTCTAAAGCAACTATCA |
| | | | GAAAACAGCTTGGACTCACTCAAGTAAACAGGGGCTT |
| | | | TATTGGCTTATAGACCTGGGCAGTCCAGGTGTAGATCT |
| | | | GGTTCCAGACAAGGCTGATTCAGAGACTCCACGTGGTC |
| | | | AAGGCTCTGTTGT |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 33 | L25 | CYP2E1- | GGAGTGGGCTGCCTGGGGTTCATACGTCACCACACTTG |
| | | EH38E151321 | ATAATTTACAGAAGCCTTTCACGCGTTTTTTCCTGTGTC |
| | | 7-mTTRp- | TCAAACGGGGTACCAGGAGGCTCAGGGAAACAGCGCA |
| | | MVMi | GGCGCCTCCCCGGTGGCTTCTCTCTGGCGCCCTCTGGT |
| | | | GGGCGGGGCCCGGGTGAGCGGGGTCCCCTCGGGGCCT |
| | | | CAGGGAGCCGCAGCCCCAGCGGAACCAGGAGGGCGCC |
| | | | CTCCGCCCAGCCGCGCTGCCACTTCCGGGTTGTTTGCT |
| | | | CCCGGAGCCATAACCGCAGCTCAGCGTGCGGGCAGAG |
| | | | CCGGCTGACAGGAGACAGGACACAGCAGGTACCCGGG |
| | | | GGCGCGCCAGTGTCTGTCTGCACATTTCGTAGAGCGAG |
| | | | TGTTCCGATACTCTAATCTCCCTAGGCAAGGTTCATAT |
| | | | TTGTGTAGGTTACTTATTCTCCTTTTGTTGACTAAGTCA |
| | | | ATAATCAGAATCAGCAGGTTTGGAGTCAGCTTGGCAG |
| | | | GGATCAGCAGCCTGGGTTGGAAGGAGGGGGTATAAAA |
| | | | GCCCCTTCACCAGGAGAAGCCGTCACACAGATCCACA |
| | | | AGCTCCTGACAGGAAGAGGTAAGGGTTTAAGGGATGG |
| | | | TTGGTTGGTGGGGTATTAATGTTTAATTACCTGGAGCA |
| | | | CCTGCCTGAAATCACTTTTTTTCAGGTTGGGCGGCCGC |
| | | | CACC |
| 181 | | EH38E151321 | GGAGTGGGCTGCCTGGGGTTCATACGTCACCACACTTG |
| | | 7 | ATAATTTACAGAAGCCTTTCACGCGTTTTTTCCTGTGTC |
| | | | TCAAACGGGGTACCAGGAGGCTCAGGGAAACAGCGCA |
| | | | GGCGCCTCCCCGGTGGCTTCTCTCTGGCGCCCTCTGGT |
| | | | GGGCGGGGCCCGGGTGAGCGGGGTCCCCTCGGGGCCT |
| | | | CAGGGAGCCGCAGCCCCAGCGGAACCAGGAGGGCGCC |
| | | | CTCCGCCCAGCCGCGCTGCCACTTCCGGGTTGTTTGCT |
| | | | CCCGGAGCCATAACCGCAGCTCAGCGTGCGGGCAGAG |
| | | | CCGGCTGACAGGAGACAGGACACAGCA |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 34 | L26 | FGA- | ATCCTCATGGAAAACATCTTTTCTAAGGGTGGGGCTGG |
| | | EH38E233784 | CTCCTGAGGAGCACTCCAGCTGAAAGAAAGGATTGCT |
| | | 3-mTTRp- | GCCACTCCTAGTTCCCATCCTATTTAAATCTGCAAGAG |
| | | MVMi | GTTTGGTTAATCATTGGCTTTGTCCTGTGTAGACAGTC |
| | | | AACCCTCCCTCTACGTCCTTTGTTCCTCCTATGCTGTTG |
| | | | GTATCCCAGAAATTCTTGCTCAATTCCCCTCAGCTTGTT |
| | | | TGCTTCCGATAAGCTGTTGCAAAGGTAGCATTTGATTC |
| | | | TCCAGTCAACAATTAGGAGATCTCACATTCCAGGAACA |
| | | | GGGTGGCTTTGAGAATGAAAAGAGGAGCATAGGAGGG |
| | | | TATATCCGGTACCCGGGGGCGCGCCAGTGTCTGTCTGC |
| | | | ACATTTCGTAGAGCGAGTGTTCCGATACTCTAATCTCC |
| | | | CTAGGCAAGGTTCATATTTGTGTAGGTTACTTATTCTCC |
| | | | TTTTGTTGACTAAGTCAATAATCAGAATCAGCAGGTTT |
| | | | GGAGTCAGCTTGGCAGGGATCAGCAGCCTGGGTTGGA |
| | | | AGGAGGGGGTATAAAAGCCCCTTCACCAGGAGAAGCC |
| | | | GTCACACAGATCCACAAGCTCCTGACAGGAAGAGGTA |
| | | | AGGGTTTAAGGGATGGTTGGTTGGTGGGGTATTAATGT |
| | | | TTAATTACCTGGAGCACCTGCCTGAAATCACTTTTTTTC |
| | | | AGGTTGGGCGGCCGCCACC |
| 182 | | EH38E233784 | ATCCTCATGGAAAACATCTTTTCTAAGGGTGGGGCTGG |
| | | 3 | CTCCTGAGGAGCACTCCAGCTGAAAGAAAGGATTGCT |
| | | | GCCACTCCTAGTTCCCATCCTATTTAAATCTGCAAGAG |
| | | | GTTTGGTTAATCATTGGCTTTGTCCTGTGTAGACAGTC |
| | | | AACCCTCCCTCTACGTCCTTTGTTCCTCCTATGCTGTTG |
| | | | GTATCCCAGAAATTCTTGCTCAATTCCCCTCAGCTTGTT |
| | | | TGCTTCCGATAAGCTGTTGCAAAGGTAGCATTTGATTC |
| | | | TCCAGTCAACAATTAGGAGATCTCACATTCCAGGAACA |
| | | | GGGTGGCTTTGAGAATGAAAAGAGGAGCATAGGAGGG |
| | | | TATATCC |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 35 | L27 | FGB- | AAACAAGATACACATCTCTCTTTGAGGAGTGCCCTAAC |
| | | EH38E233783 | TTCCCATCATTTTGTCCAATTAAATGAATTGAAGAAAT |
| | | 1-mTTRp- | TTAATGTTTCTAAACTAGACCAACAAAGAATAATAGTT |
| | | MVMi | GTATGACAAGTAAATAAGCTTTGCTGGGAAGATGTTGC |
| | | | TTAAATGATAAAATGGTTCAGCCAACAAGTGAACCAA |
| | | | AAATTAAATATTAACTAAGGAAAGGTAACCATTTCTGA |
| | | | AGTCATTCCTAGCAGAGGACTCAGATATATATAGGATT |
| | | | GAAGATCTCTCAGTTGGTACCCGGGGGCGCGCCAGTGT |
| | | | CTGTCTGCACATTTCGTAGAGCGAGTGTTCCGATACTC |
| | | | TAATCTCCCTAGGCAAGGTTCATATTTGTGTAGGTTAC |
| | | | TTATTCTCCTTTTGTTGACTAAGTCAATAATCAGAATCA |
| | | | GCAGGTTTGGAGTCAGCTTGGCAGGGATCAGCAGCCT |
| | | | GGGTTGGAAGGAGGGGGTATAAAAGCCCCTTCACCAG |
| | | | GAGAAGCCGTCACACAGATCCACAAGCTCCTGACAGG |
| | | | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGGGCGGCCGCCACC |
| 183 | | EH38E233783 | AAACAAGATACACATCTCTCTTTGAGGAGTGCCCTAAC |
| | | 1 | TTCCCATCATTTTGTCCAATTAAATGAATTGAAGAAAT |
| | | | TTAATGTTTCTAAACTAGACCAACAAAGAATAATAGTT |
| | | | GTATGACAAGTAAATAAGCTTTGCTGGGAAGATGTTGC |
| | | | TTAAATGATAAAATGGTTCAGCCAACAAGTGAACCAA |
| | | | AAATTAAATATTAACTAAGGAAAGGTAACCATTTCTGA |
| | | | AGTCATTCCTAGCAGAGGACTCAGATATATATAGGATT |
| | | | GAAGATCTCTCAGTT |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 36 | L28 | FGG- | GCCCGGAGCTCCGAGCCTTGTAGTGTCAGCACTGTCAC |
| | | EH38E233785 | CTCTCAGGCTCCAATTGTCCCTTTATGTAAGCTCCTGG |
| | | 3-m TTRp- | GATAGCCAGGACTGCAAATGGCTGGAGCTGATCACGG |
| | | MVMi | GGCCTCCTTACCAGAAGGGTGGGCAGGTCTTTCCCGTT |
| | | | CCTTTTTCCTCATCCTGGCCCCCAACCTATACTGTCAGG |
| | | | TTCCCAGATTTTGCACACAGGTTGAAGCTCCTCCTTTTT |
| | | | GGCCCAGCTCAAGGTTTTTTCTCTGATCCAAAATTATC |
| | | | TGCAAAGGCTAGTGAAGCATTATCCATTGCTGTGAGGA |
| | | | GGAGGGCAGTCCTGGGGTACCCGGGGGCGCGCCAGTG |
| | | | TCTGTCTGCACATTTCGTAGAGCGAGTGTTCCGATACT |
| | | | CTAATCTCCCTAGGCAAGGTTCATATTTGTGTAGGTTA |
| | | | CTTATTCTCCTTTTGTTGACTAAGTCAATAATCAGAATC |
| | | | AGCAGGTTTGGAGTCAGCTTGGCAGGGATCAGCAGCC |
| | | | TGGGTTGGAAGGAGGGGGTATAAAAGCCCCTTCACCA |
| | | | GGAGAAGCCGTCACACAGATCCACAAGCTCCTGACAG |
| | | | GAAGAGGTAAGGGTTTAAGGGATGGTTGGTTGGTGGG |
| | | | GTATTAATGTTTAATTACCTGGAGCACCTGCCTGAAAT |
| | | | CACTTTTTTTCAGGTTGGGCGGCCGCCACC |
| 184 | | EH38E233785 | GCCCGGAGCTCCGAGCCTTGTAGTGTCAGCACTGTCAC |
| | | 3 | CTCTCAGGCTCCAATTGTCCCTTTATGTAAGCTCCTGG |
| | | | GATAGCCAGGACTGCAAATGGCTGGAGCTGATCACGG |
| | | | GGCCTCCTTACCAGAAGGGTGGGCAGGTCTTTCCCGTT |
| | | | CCTTTTTCCTCATCCTGGCCCCCAACCTATACTGTCAGG |
| | | | TTCCCAGATTTTGCACACAGGTTGAAGCTCCTCCTTTTT |
| | | | GGCCCAGCTCAAGGTTTTTTCTCTGATCCAAAATTATC |
| | | | TGCAAAGGCTAGTGAAGCATTATCCATTGCTGTGAGGA |
| | | | GGAGGGCAGTCCTGG |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 37 | L29 | SERPINA1- | TCCAAATGGAACAGACCACACATTACATAATACATGTT |
| | | EH38E173832 | TGTATTTTTCTTCAGCTAGCTAGTGTTGTTAGAACTAAA |
| | | 1-mTTRp- | GACGGTGCTAAAATTAGTGCTGTATATTTCCATCTCCC |
| | | MVMi | TTTAATTTCTCTTTCACTTCCCAACTGAACTTTGTGCAG |
| | | | TCCTTCATGGCTTATGGAAATTCCCCCATCATTACAGG |
| | | | GCTCAGTGGTCAGGAGGGAGTTGGGCCTCTGCAGGGT |
| | | | GGGGACACTAAATATGCTGAGTGGCCTCAGGGCCAGA |
| | | | GGTTGTGGCTTCTAGCCCTGGCAGGGCCATCACTATCC |
| | | | ACTTGGCCTTTAGCAAGAATCCTCTGGTCCTGGGGCCT |
| | | | CACTCTGGTACCCGGGGGCGCGCCAGTGTCTGTCTGCA |
| | | | CATTTCGTAGAGCGAGTGTTCCGATACTCTAATCTCCC |
| | | | TAGGCAAGGTTCATATTTGTGTAGGTTACTTATTCTCCT |
| | | | TTTGTTGACTAAGTCAATAATCAGAATCAGCAGGTTTG |
| | | | GAGTCAGCTTGGCAGGGATCAGCAGCCTGGGTTGGAA |
| | | | GGAGGGGGTATAAAAGCCCCTTCACCAGGAGAAGCCG |
| | | | TCACACAGATCCACAAGCTCCTGACAGGAAGAGGTAA |
| | | | GGGTTTAAGGGATGGTTGGTTGGTGGGGTATTAATGTT |
| | | | TAATTACCTGGAGCACCTGCCTGAAATCACTTTTTTTC |
| | | | AGGTTGGGCGGCCGCCACC |
| 185 | | EH38E1738321 | TCCAAATGGAACAGACCACACATTACATAATACATGTT |
| | | | TGTATTTTTCTTCAGCTAGCTAGTGTTGTTAGAACTAAA |
| | | | GACGGTGCTAAAATTAGTGCTGTATATTTCCATCTCCC |
| | | | TTTAATTTCTCTTTCACTTCCCAACTGAACTTTGTGCAG |
| | | | TCCTTCATGGCTTATGGAAATTCCCCCATCATTACAGG |
| | | | GCTCAGTGGTCAGGAGGGAGTTGGGCCTCTGCAGGGT |
| | | | GGGGACACTAAATATGCTGAGTGGCCTCAGGGCCAGA |
| | | | GGTTGTGGCTTCTAGCCCTGGCAGGGCCATCACTATCC |
| | | | ACTTGGCCTTTAGCAAGAATCCTCTGGTCCTGGGGCCT |
| | | | CACTCT |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 38 | L30 | SERPINA1- | CCCCGACAGACAGAGGAGCTGTGCAAACAGAAAGAAA |
| | | EH38E173832 | TGGCTGCGCTTTGTTGCTGTTGCTGTATCTTGGCTGGTG |
| | | 7-m TTRp- | TCCCCCATCCTGGGGTGCCAGGACTGCAGACCTAGACA |
| | | MVMi | CCCTCATCCACTTCTGCTTACAGGAACCAGTGTATCCA |
| | | | CCAGGAGGTACCGAGGGGGGACCGGGCAGGACTGGG |
| | | | AAACAGGACACAACCCTCATGGCTGCTGTTATTATGAA |
| | | | AATAGGAGCTCAGCTGCAGCCTCTCCATCTGCGGTACC |
| | | | CGGGGGCGCGCCAGTGTCTGTCTGCACATTTCGTAGAG |
| | | | CGAGTGTTCCGATACTCTAATCTCCCTAGGCAAGGTTC |
| | | | ATATTTGTGTAGGTTACTTATTCTCCTTTTGTTGACTAA |
| | | | GTCAATAATCAGAATCAGCAGGTTTGGAGTCAGCTTGG |
| | | | CAGGGATCAGCAGCCTGGGTTGGAAGGAGGGGGTATA |
| | | | AAAGCCCCTTCACCAGGAGAAGCCGTCACACAGATCC |
| | | | ACAAGCTCCTGACAGGAAGAGGTAAGGGTTTAAGGGA |
| | | | TGGTTGGTTGGTGGGGTATTAATGTTTAATTACCTGGA |
| | | | GCACCTGCCTGAAATCACTTTTTTTCAGGTTGGGCGGC |
| | | | CGCCACC |
| 186 | | EH38E173832 | CCCCGACAGACAGAGGAGCTGTGCAAACAGAAAGAAA |
| | | 7 | TGGCTGCGCTTTGTTGCTGTTGCTGTATCTTGGCTGGTG |
| | | | TCCCCCATCCTGGGGTGCCAGGACTGCAGACCTAGACA |
| | | | CCCTCATCCACTTCTGCTTACAGGAACCAGTGTATCCA |
| | | | CCAGGAGGTACCGAGGGGGGACCGGGCAGGACTGGG |
| | | | AAACAGGACACAACCCTCATGGCTGCTGTTATTATGAA |
| | | | AATAGGAGCTCAGCTGCAGCCTCTCCATCTGC |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 39 | L31 | SERPINA1- | ACTGTCCCAGGTCAGTGGTGGTGCCTGAAGCTGAGGA |
| | | EH38E173832 | GACAGGGCCCTGTCCTCGTCCGTATTTAAGCAGTGGAT |
| | | 8-mTTRp- | CCAGAGGGGCAACGGGGGAGGCTGCTGGTGAATATTA |
| | | MVMi | ACCAAGGTCACCCCAGTTATCGGAGGAGCAAACAGGG |
| | | | GCTAAGTCCACTGGCTGGGATCTGAGTCGCCCGCCTAC |
| | | | GCTGCCCGGACGCTTTGCCTGGGCAGTGTACAGCTTCC |
| | | | ACTGCACTTACCGAAAGGAGTCATTGTACCTGGCTCAG |
| | | | AAACCACAGCGTCCTGTGTCCAAGGTGGAGGGGGTGG |
| | | | CGTGGTACCCGGGGGCGCGCCAGTGTCTGTCTGCACAT |
| | | | TTCGTAGAGCGAGTGTTCCGATACTCTAATCTCCCTAG |
| | | | GCAAGGTTCATATTTGTGTAGGTTACTTATTCTCCTTTT |
| | | | GTTGACTAAGTCAATAATCAGAATCAGCAGGTTTGGA |
| | | | GTCAGCTTGGCAGGGATCAGCAGCCTGGGTTGGAAGG |
| | | | AGGGGGTATAAAAGCCCCTTCACCAGGAGAAGCCGTC |
| | | | ACACAGATCCACAAGCTCCTGACAGGAAGAGGTAAGG |
| | | | GTTTAAGGGATGGTTGGTTGGTGGGGTATTAATGTTTA |
| | | | ATTACCTGGAGCACCTGCCTGAAATCACTTTTTTTCAG |
| | | | GTTGGGCGGCCGCCACC |
| 187 | | EH38E1738328 | ACTGTCCCAGGTCAGTGGTGGTGCCTGAAGCTGAGGA |
| | | | GACAGGGCCCTGTCCTCGTCCGTATTTAAGCAGTGGAT |
| | | | CCAGAGGGGCAACGGGGGAGGCTGCTGGTGAATATTA |
| | | | ACCAAGGTCACCCCAGTTATCGGAGGAGCAAACAGGG |
| | | | GCTAAGTCCACTGGCTGGGATCTGAGTCGCCCGCCTAC |
| | | | GCTGCCCGGACGCTTTGCCTGGGCAGTGTACAGCTTCC |
| | | | ACTGCACTTACCGAAAGGAGTCATTGTACCTGGCTCAG |
| | | | AAACCACAGCGTCCTGTGTCCAAGGTGGAGGGGGTGG |
| | | | CGT |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 40 | L32 | synEnh_v2- | GAGTTAATAATTACCAGCGCGGGCCAAATAAATAATC |
| | | AATp | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACCACCCCCTCCACCTTG |
| | | | GACACAGGACGCTGTGGTTTCTGAGCCAGGTACAATG |
| | | | ACTCCTTTCGGTAAGTGCAGTGGAAGCTGTACACTGCC |
| | | | CAGGCAAAGCGTCCGGGCAGCGTAGGCGGGCGACTCA |
| | | | GATCCCAGCCAGTGGACTTAGCCCCTGTTTGCTCCTCC |
| | | | GATAACTGGGGTGACCTTGGTTAATATTCACCAGCAGC |
| | | | CTCCCCCGTTGCCCCTCTGGATCCACTGCTTAAATACG |
| | | | GACGAGGACAGGGCCCTGTCTCCTCAGCTTCAGGCACC |
| | | | ACCACTGACCTGGGACAGTGAATCGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 188 | | AAT_v2_p | CGCGTTCGCCCCGCCCCGGACCACCCCCTCCACCTTGG |
| | | | ACACAGGACGCTGTGGTTTCTGAGCCAGGTACAATGA |
| | | | CTCCTTTCGGTAAGTGCAGTGGAAGCTGTACACTGCCC |
| | | | AGGCAAAGCGTCCGGGCAGCGTAGGCGGGCGACTCAG |
| | | | ATCCCAGCCAGTGGACTTAGCCCCTGTTTGCTCCTCCG |
| | | | ATAACTGGGGTGACCTTGGTTAATATTCACCAGCAGCC |
| | | | TCCCCCGTTGCCCCTCTGGATCCACTGCTTAAATACGG |
| | | | ACGAGGACAGGGCCCTGTCTCCTCAGCTTCAGGCACCA |
| | | | CCACTGACCTGGGACAGTGAATCG |
| 41 | L33 | synEnh v2- | GAGTTAATAATTACCAGCGCGGGCCAAATAAATAATC |
| | | AAT v2 | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GGGTACCCGGGGGCGCGCCAGTCACCCCCTCCACCTTG |
| | | | GACACAGGACGCTGTGGTTTCTGAGCCAGGTACAATG |
| | | | ACTCCTTTCGGTAAGTGCAGTGGAAGCTGTACACTGCC |
| | | | CAGGCAAAGCGTCCGGGCAGCGTAGGCGGGCGACTCA |
| | | | GATCCCAGCCAGTGGACTTAGCCCCTGTTTGCTCCTCC |
| | | | GATAACTGGGGTGACCTTGGTTAATATTCACCAGCAGC |
| | | | CTCCCCCGTTGCCCCTCTGGATCCACTGCTTAAATACG |
| | | | GACGAGGACAGGGCCCTGTCTCCTCAGCTTCAGGCACC |
| | | | ACCACTGACCTGGGACAGTGAATCGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 250 | | AAT_v3_p | GGTACCCGGGGGCGCGCCAGTCACCCCCTCCACCTTGG |
| | | | ACACAGGACGCTGTGGTTTCTGAGCCAGGTACAATGA |
| | | | CTCCTTTCGGTAAGTGCAGTGGAAGCTGTACACTGCCC |
| | | | AGGCAAAGCGTCCGGGCAGCGTAGGCGGGCGACTCAG |
| | | | ATCCCAGCCAGTGGACTTAGCCCCTGTTTGCTCCTCCG |
| | | | ATAACTGGGGTGACCTTGGTTAATATTCACCAGCAGCC |
| | | | TCCCCCGTTGCCCCTCTGGATCCACTGCTTAAATACGG |
| | | | ACGAGGACAGGGCCCTGTCTCCTCAGCTTCAGGCACCA |
| | | | CCACTGACCTGGGACAGTGAATCG |
| 42 | L34 | SynEnh_v2- | GAGTTAATAATTACCAGCGCGGGCCAAATAAATAATC |
| | | mTTRcore | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GGGTACCCGGGGGCGCGCCAGTGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 43 | L35 | SynEnh_v2- | GAGTTAATAATTACCAGCGCGGGCCAAATAAATAATC |
| | | mTTRcore | CGCGAGGGGCAGCTGGACTTTGGACTCCGCGCGCTGG |
| | | MVMi (AAT) | TAATTATTAACCTCGCGAATATTGATTCGAGGCCGCGA |
| | | | TTGCCGCAATCGCGAGGGGCAGGTGACCTTTGCCCAGC |
| | | | GGGTACCCGGGGGCGCGCCAGTGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGGAAGAGGTAAGGGTT |
| | | | TAAGGGATGGTTGGTTGGTGGGGTATTAATGTTTAATT |
| | | | ACCTGGAGCACACCCCCTCCACCTTGGACACAGGACG |
| | | | CTGTGGTTTCTGAGCCAGGTACAATGACTCCTTTCGGT |
| | | | AAGTGCAGTGGAAGCTGTACACTGCCCAGGCAAAGCG |
| | | | TCCGGGCAGCGTAGGCGGGCGACTCAGATCCCAGCCA |
| | | | GTGGACTTAGCCCCTGTTTGCTCCTCCGATAACTGGGG |
| | | | TGACCTTGGTTAATATTCACCAGCAGCCTCCCCCGTTG |
| | | | CCCCTCTGGATCCACTGCTTAAATACGGACGAGGACAG |
| | | | GGCCCTGTCTCCTCAGCTTCAGGCACCACCACTGACCT |
| | | | GGGACAGTGAATCGCCTGCCTGAAATCACTTTTTTTCA |
| | | | GGTTGGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 171 | | MVMi-AATp | AAGAGGTAAGGGTTTAAGGGATGGTTGGTTGGTGGGG |
| | | intron | TATTAATGTTTAATTACCTGGAGCACACCCCCTCCACC |
| | | | TTGGACACAGGACGCTGTGGTTTCTGAGCCAGGTACAA |
| | | | TGACTCCTTTCGGTAAGTGCAGTGGAAGCTGTACACTG |
| | | | CCCAGGCAAAGCGTCCGGGCAGCGTAGGCGGGCGACT |
| | | | CAGATCCCAGCCAGTGGACTTAGCCCCTGTTTGCTCCT |
| | | | CCGATAACTGGGGTGACCTTGGTTAATATTCACCAGCA |
| | | | GCCTCCCCCGTTGCCCCTCTGGATCCACTGCTTAAATA |
| | | | CGGACGAGGACAGGGCCCTGTCTCCTCAGCTTCAGGC |
| | | | ACCACCACTGACCTGGGACAGTGAATCGCCTGCCTGA |
| | | | AATCACTTTTTTTCAGGTTGG |
| 150 | | AATp | CACCCCCTCCACCTTGGACACAGGACGCTGTGGTTTCT |
| | | | GAGCCAGGTACAATGACTCCTTTCGGTAAGTGCAGTGG |
| | | | AAGCTGTACACTGCCCAGGCAAAGCGTCCGGGCAGCG |
| | | | TAGGCGGGCGACTCAGATCCCAGCCAGTGGACTTAGC |
| | | | CCCTGTTTGCTCCTCCGATAACTGGGGTGACCTTGGTT |
| | | | AATATTCACCAGCAGCCTCCCCCGTTGCCCCTCTGGAT |
| | | | CCACTGCTTAAATACGGACGAGGACAGGGCCCTGTCTC |
| | | | CTCAGCTTCAGGCACCACCACTGACCTGGGACAGTGA |
| | | | ATCG |
| 44 | L36 | SynEnh_v2- | GAGTTAATAATTACCAGCGCGGGCCAAATAAATAATC |
| | | mTTRcore | CGCGAGGGGCAGCTGGACTTTGGACTCCGCGCGCTGG |
| | | MVMi | TAATTATTAACCTCGCGAATATTGATTCGAGGCCGCGA |
| | | | TTGCCGCAATCGCGAGGGGCAGGTGACCTTTGCCCAGC |
| | | | GGGTACCCGGGGGCGCGCCAGTGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGGAAGAGGTAAGGGTT |
| | | | TAAGGGATGGTTGGTTGGTGGGGTATTAATGTTTAATT |
| | | | ACCTGGAGCACCTGCCTGAAATCACTTTTTTTCAGGTT |
| | | | GGGCGGCCGCCACC |
| 153 | | SynEnh | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | G |
| 176 | | mTTR3p | GGTACCCGGGGGCGCGCCAGTGTCTGTCTGCACATTTC |
| | | | GTAGAGCGAGTGTTCCGATACTCTAATCTCCCTAGGCA |
| | | | AGGTTCATATTTGTGTAGGTTACTTATTCTCCTTTTGTT |
| | | | GACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCCTGACAGG |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 45 | L37 | ORM1 | TCCCATGTGGGGCTGCAGGAAAGGGTGAGGAAGCCTG |
| | | | CGGTATGAGTTGGCAAGTTGGGTTTGACTGCCATGGGG |
| | | | CCCTGGAGCAGAGCCATCCAGGAGGCAGCTGCCTGCG |
| | | | CTGCGAGGAAAGGAGAGAGGTCTGCACTGCACTCAGA |
| | | | GCTCTGGAGGCATCAGCAGTTTCTGCTAGGCCTGGAAG |
| | | | ACATGGGTAGAGATTGAAAAAGCCAGCCAGAAGGGCC |
| | | | AGAGGGGGAAAGCAAGAAGAGCCATGTTAGAACTGTG |
| | | | ACACTCACGACTCTCGGCCTGGTCTCCAATCCCAGGTG |
| | | | GGCCAGTGAGGATGCTGGAGCTGGGCCAGCTCATTCC |
| | | | CAGCAGGGGCAGATAGGAGGTTGTTGGGGACAGAAGG |
| | | | AAACTTTTCCTAAACTGGCAGTACCCACAGTTCCCAAT |
| | | | CTCCTGTTGCACAACTGAGTGTCAGGAAACAGTCAGGT |
| | | | GGACTTCCCAGGCTGGGCCAACTCCAGGGGAAAGCCC |
| | | | AGGCCAGCCTGTTTGCCCTGTTCACAACGAATCAAACC |
| | | | CTAGCTAAGGTCACAGGATCCGCCACC |
| 46 | L38 | APOC3-MPG | TGGTGGGGGCTGGGGAGGGCCCCAGACATGAGACCAG |
| | | | CTCCTCCCCCAGGGGATGTTATCAGTGGGTCCAGAGGG |
| | | | CAAAATAGGGAGCCTGGTGGAGGGAGGGGCAAAGGC |
| | | | CTCGGGCTCTGAGCGGCCTTGGCCCTTCTCCACCAACC |
| | | | CCTGCCCTACACTAAGGGGGAGGCAGCGGGGGGCACA |
| | | | CAGGGTGGGGGCGGGTGGGGGGCTGCTGGGTGAGCAG |
| | | | CACTCGCCTGCCTGGATTGAAACCCAGAGATGGAGGT |
| | | | GCTGGGAGGGGCTGTGAGAGCTCAGCCCTGTAACCAG |
| | | | GCCTTGCCGGAGCCACTGATGCCTGGTCTTCTGTGCCT |
| | | | TTACTCCAAACACCCCCCAGCCCAAGCCACCCACTTGT |
| | | | TCTCAAGTCTGAAGAAGCCCCTCACCCCTCTACTCCAG |
| | | | GCTGTGTTCAGGGCTTGGGGCTGGTGGAGGGAGGGGC |
| | | | CTGAAATTCCAGTGTGAAAGGCTGAGATGGGCCCGAG |
| | | | GCCCCTGGCCTATGTCCAAGCCATTTCCCCTCTCACCA |
| | | | GCCTCTCCCTGGGGAGCCAGTCAGCTAGGAAGGAATG |
| | | | AGGGCTCCCCAGGCCCACCCCCAGTTCCTGAGCTCATC |
| | | | TGGGCTGCAGGGCTGGCGGGACAGCAGCGTGGACTCA |
| | | | GTCTCCTAGGGATTTCCCAACTCTCCCGCCCGCTTGCT |
| | | | GCATCTGGACACCCTGCCTCAGGCCCTCATCTCCACTG |
| | | | GTCAGCAGGTGACCTTTGCCCAGCGCCCTGGGTCCTCA |
| | | | GTGCCTGCTGCCCTGGAGATGATATAAAACAGGTCAG |
| | | | AACCCTCCTGCCTGTCTGCTCAGTTCATCCCTAGAGGC |
| | | | AGCTGCTCCAGGTAATGCCCTCTGGGGAGGGGAAAGA |
| | | | GGAGGGGAGGAGGGGATCCGCCACC |
| 47 | L39 | Saal | TGACAGAGCCATTGAAATAAATCCTGATTCAGCTCAGC |
| | | | CTTACAAGTGGCGGGGGAAAGCACACAGACTTCTAGG |
| | | | CCACTGGGAAGAAGCAGCCCATGATCTTGCCTTTGCCT |
| | | | GTAAATTGGATTATGATGAAGATGCTAGTGCAATGCTG |
| | | | AAAGAAGTTCAACCTAGGGCACAGAAAATTGCAGAAC |
| | | | ATTGGAGAAAGTATGAGCGAAAACATGAAGAGCGAGA |
| | | | GATCAAAGAAAGAATAGAACGAGTTAAGAAGGCTCAA |
| | | | GAAGAGCAGGAGAGAGCCCAGAGGGAGGAAGAAGCC |
| | | | AGACGACAGTCAGGAGCTCACTATGGCCCTTTTCCAGG |
| | | | TGGCTTTCCTGGTGGAATGCCTGGTAATTTTCCCGGAG |
| | | | GAATGCCTGGAATGGGAGGGGACATGCCTGGAATGGC |
| | | | CGGAATGCCTGGACTCAATGAAATTCTTAGTGATCCAG |
| | | | AGGCTCTTGCAGCCATGCAGGATCCAGAGAGGAGCAG |
| | | | AGCTGGTCTCCTGCCCTGACAGCTGCCAGGCACATCTT |
| | | | GTTCCCTCAGGTTGCACAACTGGGATAAATGACCCGGG |
| | | | ATGAAGAAACCACTGGCATCCAGGAACTTGTCTTAGA |
| | | | CCGTTTTGTAGGGGAAATGACCTGCAGGGACTTTCCCC |
| | | | AGGGACCACATCCAGCTTTTCTTCGCTCCCAAGAAACC |
| | | | AGCAGGGAAGGCTCAGTATAAATAGCAGCCACCGCTC |
| | | | CCTGGCAGGCAGGGACCCGCAGCTCAGCTACAGCACA |
| | | | GATGGATCCGCCACC |
| 48 | L40 | APOA2 | CGTCTCATTACACATTAACTCAAAAACGGACAAGATCA |
| | | | TTTACACTTGCCCTCTTACCCGACCCTCATTCCCCTAAC |
| | | | CCCCATAGCCCTCAACCCTGTCCCTGATTTCAATTCCTT |
| | | | TCTCCTTTCTTCTGCTCCCCAATATCTCTCTGCCAAGTT |
| | | | GCAGTAAAGTGGGATAAGGTTGAGAGATGAGATCTAC |
| | | | CCATAATGGAATAAAGACACCATGAGCTTTCCATGGTA |
| | | | TGATGGGTTGATGGTATTCCATGGGTTGATATGTCAGA |
| | | | GCTTTCCAGAGAAATAACTTGGAATCCTGCTTCCTGTT |
| | | | GCACTCAAGTCCAAGGACCTCAGATCTCAAAAGAATG |
| | | | AACCTCAAATATACCTGAAGTGTACCCCCTTAGCCTCC |
| | | | ACTAAGAGCTGTACCCCCTGCCTCTCACCCCATCACCA |
| | | | TGAGTCTTCCATGTGCTTGTCCTCTCCTCCCCCATTTCT |
| | | | CCAACTTGTTTATCCTCACATAATCCCTGCCCCACTGG |
| | | | GCCCATCCATAGTCCCTGTCACCTGACAGGGGGTGGGT |
| | | | AAACAGACAGGTATATAGCCCCTTCCTCTCCAGCCAGG |
| | | | GCAGGCACAGACACCAAGGACAGAGACGCTGGCTAGG |
| | | | TAAGATAAGGAGGATCCGCCACC |
| 49 | L41 | SERPINA1 | CCCGCCCCTCCCCAGCCTACTGCCTCCACCCGAAGTCT |
| | | | ACTTCCTGGGTGGGCAGGAACTGGGCACTGTGCCCAG |
| | | | GGCATGCACTGCCTCCACGCAGCAACCCTCAGAGTCCT |
| | | | GAGCTGAACCAAGAAGGAGGAGGGGGTCGGGCCTCCG |
| | | | AGGAAGGCCTAGCCGCTGCTGCTGCCAGGAATTCCAG |
| | | | GTTGGAGGGGCGGCAACCTCCTGCCAGCCTTCAGGCC |
| | | | ACTCTCCTGTGCCTGCTCCGCTCTGAGCCTGTTTCCTCA |
| | | | TCTGTCAAATGGGCTCTAACCCACTCTGATCTCCCAGG |
| | | | GCGGCAGTAAGTCTTCAGCATCAGGCATTTTGGGGTGA |
| | | | CTCAGTAAATGGTAGATCTTGCTACCAGTGGAACAGCC |
| | | | ACTAAGGATTCTGCAGTGAGAGCAGAGGGCCAGCTAA |
| | | | GTGGTACTCTCCCAGAGACTGTCTGACTCACGCCACCC |
| | | | CCTCCACCTTGGACACAGGACGCTGTGGTTTCTGAGCC |
| | | | AGGTACAATGACTCCTTTCGGTAAGTGCAGTGGAAGCT |
| | | | GTACACTGCCCAGGCAAAGCGTCCGGGCAGCGTAGGC |
| | | | GGGCGACTCAGATCCCAGCCAGTGGACTTAGCCCCTGT |
| | | | TTGCTCCTCCGATAACTGGGGTGACCTTGGTTAATATT |
| | | | CACCAGCAGCCTCCCCCGTTGCCCCTCTGGATCCACTG |
| | | | CTTAAATACGGACGAGGACAGGGCCCTGTCTCCTCAGC |
| | | | TTCAGGCACCACCACTGACCTGGGACAGTGAATCGGA |
| | | | TCCGCCACC |
| 50 | L42 | ALB | TCCTTAAAATAATTATGAAACACTGTCTTATGGGTTGT |
| | | | GCCCTGTCAACAAGAATGCTATTGTTAAATAATTAGCA |
| | | | TGTTTGTTGATGCTGGCCAATATTTACTTTTAGGTATCA |
| | | | TCACTTCTGATGTGGATTTTTTGTCAATTTTCCACATAA |
| | | | CTGCAGATATAAGTTTGGACTCATCAACACAGCCATTT |
| | | | GCTTTCAAGACATTAAATGCTGTTTGGTTAGTAATTAC |
| | | | TAAACACTGAGAACTAAATTGCAAACACCAAGAACTA |
| | | | AAATGTTCAAGTGGGAAATTACAGTTAAATACCATGGT |
| | | | AATGAATAAAAGGTACAAATCGTTTAAACTCTTATGTA |
| | | | AAATTTGATAAGATGTTTTACACAACTTTAATACATTG |
| | | | ACAAGGTCTTGTGGAGAAAACAGTTCCAGATGGTAAA |
| | | | TATACACAAGGGATTTAGTCAAACAATTTTTTGGCAAG |
| | | | AATATTATGAATTTTGTAATCGGTTGGCAGCCAATGAA |
| | | | ATACAAAGATGAGTCTAGTTAATAATCTACAATTATTG |
| | | | GTTAAAGAAGTATATTAGTGCTAATTTCCCTCCGTTTG |
| | | | TCCTAGCTTTTCTCTTCTGTCAACCCCACACGCCTTTGG |
| | | | CACAGGATCCGCCACC |
| 51 | L43 | APOA1 | GGGAGGACAGGGCGGGGGAAGGGGGAGGGGAGTGAA |
| | | | GTAGTCTCCCTGGAATGCTGGTGGTGGGGGAGGCAGT |
| | | | CTCCTTGGTGGAGGAGTCCCAGCGTCCCTCCCCTCCCC |
| | | | TCCTCTGCCAACACAATGGACAATGGCAACTGCCCACA |
| | | | CACTCCCATGGAGGGGAAGGGGATGAGTGCAGGGAAC |
| | | | CCCGACCCCACCCGGGAGACCTGCAAGCCTGCAGACA |
| | | | CTCCCCTCCCGCCCCCACTGAACCCTTGACCCCTGCCC |
| | | | TGCAGCCCCCGCAGCTTGCTGTTTGCCCACTCTATTTGC |
| | | | CCAGCCCCAGGGACAGAGCTGATCCTTGAACTCTTAAG |
| | | | TTCCACATTGCCAGGACCAGTGAGCAGCAACAGGGCC |
| | | | GGGGCTGGGCTTATCAGCCTCCCAGCCCAGACCCTGGC |
| | | | TGCAGACATAAATAGGCCCTGCAAGAGCTGGCTGCTT |
| | | | AGAGACTGCGAGAAGGAGGTGCGTCCTGCTGCCTGCC |
| | | | CCGGTCACTCTGGGGATCCGCCACC |
| 52 | L44 | SAA2 | AGGCCTGTCAGCTCCTCACCCGCCCACCCACCCGCCCG |
| | | | CCTGCATTCTTCGGTCTAAACTTACCAGGTGGAAGCTA |
| | | | CAACTCTAGACAGGAAGTAACTAACTGGGTTGGATCC |
| | | | GGTCGAGTTCCGCTGCGCTTCTTAGAGCCGCTGCTGGG |
| | | | AAGAGGCCGAAACCCAGTGGCCGCAGCAGTAGCCACC |
| | | | GCCTCTGTTACTAGGGGGTCTCGCCTCCATCGCTGGCT |
| | | | GCGAGGAGTCTGAGGGCACGTGACCGGAAGTAGCAAG |
| | | | GCGGGCCCTGGGCACGTGACTCAAGAGCGCGGCCGCC |
| | | | ATCACACCTTCCGCTAGTTCCCTCCCTCCCCCACGGGTT |
| | | | CCCGCCTGTTTCGTTTCCTCTCTCCGGTCTCGAGCGGCG |
| | | | AAGGGTCCTGGGATCCTGGCTGGCCCAACCATTTGGCC |
| | | | TAGAGCTCCTCCGGTTCCAGTCGGCGCCGTGTCTCCAG |
| | | | CCCGCAACGGCCCTGCGGTCGCAGGCCGTAGAGAGCG |
| | | | TAATCGCGACCGCGGCCTGAGGGGGAGGGACCACGCG |
| | | | GAAGGAGGGGCGTGACTCGTCACGTGTTGAGGAAGTG |
| | | | CCGTTAGTAGCGTTGTTTCCAGTCTGCGGCTCTCCTGGT |
| | | | TGACGTCAGCAAGGAAGGGGAGGAACGCGAGGGGAG |
| | | | ACTGAAGTTCACGTTCCGCTCTTAGTGCGCGGTGAGTT |
| | | | CCCCGGCGCTAAGCCGGGAGACGGATCCGCCACC |
| 53 | L45 | FTL | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACCATAAAAGAAGCCGCCCTAGCCAC |
| | | | GTCCCCTCGCAGTTCGGCGGTCCCGCGGGTCTGTCTCT |
| | | | TGCTTCAACAGTGTTTGGACGGAACAGATCCGGGGACT |
| | | | CTCTTCCAGCCTCCGACCGCCCTCCGATTTCCTCTCCGC |
| | | | TTGCAACCTCCGGGACCATCTTCTCGGCCATCTCCTGC |
| | | | TTCTGGGACCTGCCAGCACCGTTTTTGTGGTTAGCTCCT |
| | | | TCTTGCCAACCAACCGGATCCGCCACC |
| 54 | L46 | FGA | TCTTTTCATTCTCAAAGCCACCCTGTTCCTGGAATGTGA |
| | | | GATCTCCTAATTGTTGACTGGAGAATCAAATGCTACCT |
| | | | TTGCAACAGCTTATCGGAAGCAAACAAGCTGAGGGGA |
| | | | ATTGAGCAAGAATTTCTGGGATACCAACAGCATAGGA |
| | | | GGAACAAAGGACGTAGAGGGAGGGTTGACTGTCTACA |
| | | | CAGGACAAAGCCAATGATTAACCAAACCTCTTGCAGA |
| | | | TTTAAATAGGATGGGAACTAGGAGTGGCAGCAATCCT |
| | | | TTCTTTCAGCTGGAGTGCTCCTCAGGAGCCAGCCCCAC |
| | | | CCTTAGAAAAGATGTTTTCCGGATCCGCCACC |
| 55 | L47 | CRP | TGTTCTCACTCATAGATGGGAATTGAACAATGAGAATA |
| | | | CTTGGACACAGGAAGGGGAACATCACACTCTGGGGAC |
| | | | TGTTGTGGGGTGGGGGGAGGGGGGAGGGAATGGCACA |
| | | | TAAAAAAATGTGTCCATGGCTCTGGGAGGAGCATGTTT |
| | | | GTTTTCCTCATTTCCCAGTCTGTAAATAAGCAAATTGA |
| | | | AAGGGGTTAGTGATAATGTCCATCTCCAGAAGCTGTCA |
| | | | GATTTCCTTTGTCAAACTCTATGATTTGGGCTGAAGTA |
| | | | GGTGTTGGAGAGGCAGCTACCACGTGCACCCAGATGG |
| | | | CCACTCGTTTAATATGTTACCATTTCCCATTATTTTCGC |
| | | | AGGATAGATAGCCAAAGTGGAGCCCTGAGAGATTTCT |
| | | | TCATTTTTCCTGTCATAAAGAATTGGTAATTCAGTAGT |
| | | | CATAGGAGTTTGTAATAAATAACTCACATTGATTTCTC |
| | | | TGTTCTGAAATAATTTTGCTTCCCCTCTTCCCGAAGCTC |
| | | | TGACACCTGCCCCAACAAGCAATGTTGGAAAATTATTT |
| | | | ACATAGTGGCGCAAACTCCCTTACTGCTTTGGATATAA |
| | | | ATCCAGGCAGGAGGAGGTAGCTCTAAGGCAAGAGATC |
| | | | TAGGACTTCTAGCCCCTGAACTTTCAGCCGAATACATC |
| | | | TTTTCCAGGATCCGCCACC |
| 56 | L48 | ORM2 | AGCCTGGGGACCCTCAAAGGTGCTTCATCATTAGGTTT |
| | | | GTGGCTGGGTCCTACTGAAGTAAGTCTTGGCACTCAGA |
| | | | GGGATAGGAATTGAATGAAGACATGAGATTCCTCTGC |
| | | | GGGAGGCCTCTCTAGGAAATCTGTGGACTCACACGTTT |
| | | | ACTAATGTTGCTGCAGCCCCGCACCCACCTTGGCCTTG |
| | | | GGCAGCCATACTCTAGGGCTTTTGTAACCTCTCCATGT |
| | | | GAGGAACTCAAATTAGACCTGGGTTTGGAGGCGGTGC |
| | | | TCCGAGCTGGCCTTTGGGGGAGGTTTTGTGCGAGGCAT |
| | | | TTCCCAAGTGCTGGCAGGATTGTGTCACAGACACAGA |
| | | | GTAAACTTTTGCTGGGCTCCAAGTGACCGCCCATAGTT |
| | | | TATTATAAAGGTGACTGCACCCTGCAGCCACCAGCACT |
| | | | GCCTGGCTCCACGTGCCTCCTGGTCTCAGTGGATCCGC |
| | | | CACC |
| 57 | L49 | HP | AGACTAGCTTTCCACTCCTCCTTGTCTTCTCTCTGCAGT |
| | | | GCCCTGGGAGCTGTCATTGCCCTCCTGCTCTGGGGACA |
| | | | GCTTTTTAAGAACAACTAACCCTTTTTAATGAATAAAT |
| | | | CATTCAGTTGACTTTCTCATCTGCCTATTTAGGCTAATC |
| | | | ACCAGACTATAAAACCAAGTTGTGTGATTTCTTGCAAA |
| | | | GAGCAATCAGAGAAGACACAATAAACACATTTACTGA |
| | | | TTTCAGGCAAAAGGAAGTACTTTCTTTAGAGCCCCACC |
| | | | TAAGCTAGGCTGCAGAAATGTCTACAATGGGTTTGAA |
| | | | AAAACTCAAAATGAGCCTTTCTGCAGTGTGAAAATCCT |
| | | | CCAAGATAAAGAGACAGATTGATGGTTCCTGCCGCCG |
| | | | CCCTGTCCTGCCCAGTTGCTGATTTCAGGAAATACTTT |
| | | | GGCAGGTTTGTGGGTCATAGAGTTGCCAGGTTTCTTGG |
| | | | GATTTGTAATAGAACATCACAAGAAAATCAAGTGTGA |
| | | | AGCAAGAGCTCAACTCTTAACAGGGGTATTGTTTGTGG |
| | | | TTTTGTTACTGGAAAAGATAGTGACCTTACCAGGGCCA |
| | | | AAGTTTGTAGACACAGGAATTACGAAATTGAGAAGGG |
| | | | GGAGAAGTGAGCTAGTGGCAGCATAAAAAGACCAGCA |
| | | | GGGATCCGCCACC |
| 256 | L50 | FGB | ACATTAATTTACCAGAAACAAGATACACATCTCTCTTT |
| | | | GAGGAGTGCCCTAACTTCCCATCATTTTGTCCAATTAA |
| | | | ATGAATTGAAGAAATTTAATGTTTCTAAACTAGACCAA |
| | | | CAAAGAATAATAGTTGTATGACAAGTAAATAAGCTTT |
| | | | GCTGGGAAGATGTTGCTTAAATGATAAAATGGTTCAGC |
| | | | CAACAAGTGAACCAAAAATTAAATATTAACTAAGGAA |
| | | | AGGTAACCATTTCTGAAGTCATTCCTAGCAGAGGACTC |
| | | | AGATATATATAGGATTGAAGATCTCTCAGTTAAGTCTA |
| | | | CATGAAAAGGGGATCCGCCACC |
| 58 | L51 | FGG | CCCTAGGCTAAAATAATAGTGTTAACTGATCCCTAAGC |
| | | | TAAGAAAGTTCTTTTGGTAATTCAGGTGATGGCAGCAG |
| | | | GACCCATCTTAAGGATAGACTAGGTTTGCTTAGTTCGA |
| | | | GGTCATATCTGTTTGCTCTCAGCCATGTACTGGAAGAA |
| | | | GTTGCATCACACAGCCTCCAGGACTGCCCTCCTCCTCA |
| | | | CAGCAATGGATAATGCTTCACTAGCCTTTGCAGATAAT |
| | | | TTTGGATCAGAGAAAAAACCTTGAGCTGGGCCAAAAA |
| | | | GGAGGAGCTTCAACCTGTGTGCAAAATCTGGGAACCT |
| | | | GACAGTATAGGTTGGGGGCCAGGATGAGGAAAAAGGA |
| | | | ACGGGAAAGACCTGCCCACCCTTCTGGTAAGGAGGCC |
| | | | CCGTGATCAGCTCCAGCCATTTGCAGTCCTGGCTATCC |
| | | | CAGGAGCTTACATAAAGGGACAATTGGAGCCTGAGAG |
| | | | GTGACAGTGCTGACACTACAAGGCTCGGAGCTCCGGG |
| | | | CACTCAGACATCGGATCCGCCACC |
| | | | ATTTTGCCCACAGGAATCCTCGTAGCCCACTACCTCCA |
| 59 | L52 | APOE | GAGCATCACGCCTTGCCTGGCCCTGGGTGGAGAGCTG |
| | | | GTCTACCACCGGCGGCCTGCTGAGCACCAGGGGCAGA |
| | | | GGCGGCCAGACAACCTCAGGGAGGAGTGTCCTGGCGT |
| | | | CCCCATCCTCCAAAGGGCCTGGGCCCGCCCCGAGGGG |
| | | | GCAGCGAGAGGAGCTTCCCCATCCCCGTCCTCTCCAGA |
| | | | TTACATTCATCCAGGCACAGGAAAGGACAGGGTCAGG |
| | | | AAAGGAGGACTCTGGGCGGCAGCCTCCACATTCCCCTT |
| | | | CCACGCTTGGCCCCCAGATTGGAGGAGGGTGTCTGTAT |
| | | | TACTGGGCGAGGTGTCCTCCCTTCCTGGGGACTGTGGG |
| | | | GGGTGGTCAAAAGACCTCTTTGCCCCACCTCCTTCCTC |
| | | | CCTCTGCCCTGCTGTGCCTGGGGCAGGGGGAGAACAG |
| | | | CCCACCTCGTGACTGGGGGCTGGCCCAGCCCGCCCTAT |
| | | | CCCTGGGGGAGGGGGCGGGACAGGGGGAGCCCTATAA |
| | | | TTGGACAAGTCTGGGATCCTTGAGTCCTACTCAGCCCC |
| | | | AGCGGAGGTGAAGGACGTCCTTCCCCAGGATCCGCCA |
| | | | CC |
| 60 | L53 | FABP1 | GTTCAAAGGAATTCTCTGGTATTTTTCTGTGTGTGTACA |
| | | | CATGCACCCACACACTTGTGTGTATATGTGTACAGACA |
| | | | TATATAAACACATGCATATAATGTGTATATATGCATTA |
| | | | CATATATGCACATTCATACATCTTTATGTACAAAATAC |
| | | | ATATATGTATATATAAACACCGATGTACAAACACATAC |
| | | | GCACACATCTATATACATACACATGTGTGTGCACATAT |
| | | | ACACATACCTGCATATACACACATTTCGTGGGGTGCGG |
| | | | AGAGTCACTTAAAGGCTGCAGGGCCATAAGGCTTCCT |
| | | | GCTTGACTGATATTCATTAATGTTTGCTGAATTACAGC |
| | | | AAACCTTTGCTGTGCCCATCCTGTTCTTTATCATTGACC |
| | | | ATTGCTCTCAGGAGTTAATGTTTGAACCTGGCCATAAA |
| | | | GGAATCAACAGCTGCTGACCTCTGGCCGCTATTCGAAG |
| | | | GGAAGGGAGCCCCCTATAAAACAGCCTACAGTGGACA |
| | | | GTCTGGTCGGCAGAGCCGCAGGTCAGTCGTGAAGAGG |
| | | | GAGCTCTATTGGATCCGCCACC |
| 61 | L54 | APOCI | TTTTGTGCCATTAACAAAAACGTTATGAAGACAGAAAC |
| | | | CATCTCCCAAAGATTTCATAACAGAGCCACATAAGTGG |
| | | | AAAGTAAATGATTAAAGAATGTGGGTCTCAGAGTTCC |
| | | | ATTCAAATCATGATACTTTATCTTCTATTTACAAAGAT |
| | | | AAAAGTACACCAGAAAATGGTTAATGTTTAAGCGCAA |
| | | | AACTAAAAGGCAGAAATGAAATCCACAAGCAGACAGC |
| | | | CCGCGCCACACCCTGGGCCTGGTGGTTAAAGATTGACC |
| | | | CCTGACCTAATCCGTTAGGTTATCTATAGATTACAGAC |
| | | | ATTGTATAGAAAAGCACTGTGAAAATCCCTATTCTGTT |
| | | | TTGTTCCGATCTAATTACCGGTGCATGCAGCCCCCAGT |
| | | | CACGCATCCCCTGCTTGTTCAATCGATCACGACCCTCT |
| | | | CACGTGCACCCACTTAGAGTTGTGAGCCCTTAAAAGGA |
| | | | ACAGGGATTGCTCACTCGGGGAGCTCGGCTCTTGAGAC |
| | | | AGGAATCTTGCCCATTCCCCGAACGAATAAACCCCTTC |
| | | | CTTAACTCAGCGTCTGAGGAATTTTGTCTGCGGCTCCT |
| | | | CCTGCTACATTCTGAGTGGGGAAAGGGACTAAGGTGG |
| | | | TCTGAGGACCCCACAGAGTCAGGAAGATTGAGAGGTG |
| | | | AGAGTGCTGAACGGGGAGGGGCTTTGGGGCTAAGGGA |
| | | | AGTGCCCGGGACCCCACCTGACCCCAACGCTCACGGG |
| | | | ACAGGGGCAGAGGAGAAAAACGTGGGTGGACAGAGG |
| | | | GAGGCAGGCGGTCAGGGGAAGGCTCAGGAGGAGGGA |
| | | | GATCAACATCAACCTGCCCCGCCCCCTCCCCAGCCTGA |
| | | | TAAAGGTCCTGCGGGCAGGACAGGACCTCCCAACCAA |
| | | | GCCCTCCAGCAAGGATTCAGGTTGGCCAGTGTGACTCA |
| | | | TCCACAATGATTTCTCCAGTGCTCATCTTGTTCTCGAGT |
| | | | TTTCTCTGCCGGATCCGCCACC |
| 62 | L55 | APOH | TTTTGTGCCATTAACAAAAACGTTATGAAGACAGAAAC |
| | | | CATCTCCCAAAGATTTCATAACAGAGCCACATAAGTGG |
| | | | AAAGTAAATGATTAAAGAATGTGGGTCTCAGAGTTCC |
| | | | ATTCAAATCATGATACTTTATCTTCTATTTACAAAGAT |
| | | | AAAAGTACACCAGAAAATGGTTAATGTTTAAGCGCTTT |
| | | | CATATTTGGCTCTGTCTTTTTAGCAGACGAAAACCACT |
| | | | TTGGTAGTGCCAGTGTGACTCATCCACAATGATTTCTC |
| | | | CAGTGCTCATCTTGTTCTCGAGTTTTCTCTGCCGGATCC |
| | | | GCCACC |
| 63 | L56 | C3 | CAGGTGGCCCTGACCCTGGGAGAGTCCAGGGCAGGGT |
| | | | GCAGCTGCATTCATGCTGCTGGGGAACATGCCCTCAGG |
| | | | TTACTCACCCCATGGACATGTTGGCCCCAGGGACTGAA |
| | | | AAGCTTAGGAAATGGTATTGAGAAATCTGGGGCAGCC |
| | | | CCAAAAGGGGAGAGGCCATGGGGAGAAGGGGGGGCT |
| | | | GAGTGGGGGAAAGGCAGGAGCCAGATAAAAAGCCAG |
| | | | CTCCAGCAGGCGCTGCTCACTCCTCCCCATCCTCTCCCT |
| | | | CTGTCCCTCGGATCCGCCACC |
| 64 | L57 | FTL2 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACCGGATCCG |
| | | | CCACC |
| 190 | | FTL-TImd | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAG |
| 192 | | TImd | TATAAAAGCCCCTTCACCAGGAGAAGCCGTCACACAG |
| | | | ATCCACAAGCTCGAGCCGAGTGGTCGT |
| 65 | L58 | FTL3 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGGGGCAGGTTGGTAT |
| | | | CAAGGTTACAAGACAGGTTTAAGGAGACCAATAGAAA |
| | | | CTGGGCATGTGGAGACAGAGAAGACTCTTGGGTTTCTG |
| | | | ATAGGCACTGACTCTCTCTGCCTATTGGTCTATTTTCCC |
| | | | ACCCTTAGGCTGCTTCTTGCCAACCAACC |
| 190 | | FTL-Timd | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAG |
| 192 | | TImd | TATAAAAGCCCCTTCACCAGGAGAAGCCGTCACACAG |
| | | | ATCCACAAGCTCGAGCCGAGTGGTCGT |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 66 | L59 | FTL4 | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTCGAGTTGCTGGTGCT |
| | | | TCCCCAGGCTGGAGATTGAGTTAATATTAACAGGCCCA |
| | | | AGGCGATGTGGGCTTGTGCAATCATAGGCCCGGCCTCG |
| | | | CCAGGTCACCTGAGGAGTTAATGAATACATATCTCCTT |
| | | | CCCATAAAACTCTAGCCTCCCAGAGGGACCCCAGCCCC |
| | | | CACCCTCCCGCCCACGAACCCCTGCATTTCCAGAATCA |
| | | | GCCCCAGGGCCCCAACCCCCCCAAGCCCCCATTTCACA |
| | | | ACACGCTGGCGCTACAGGCGCGTGACTTCCCCTTGCTT |
| | | | TGGGGCGGGGGGCTGAGACTCCTATGTGCTCCGGATTG |
| | | | GTCAGGCACGGCCTTCGGCCCCGCCTCCTGCCACCGCA |
| | | | GATTGGCCGCTAGCCCTCCCCGAGCGCCCTGCCTCCGA |
| | | | GGGCCGGCGCACTATAAAAGCCCCTTCACCAGGAGAA |
| | | | GCCGTCACACAGATCCACAAGCTCGAGCCGAGTGGTC |
| | | | GTGGGTCTGTCTCTTGCTTCAACAGTGTTTGGACGGAA |
| | | | CAGATCCGGGGACTCTCTTCCAGGGGCAGGTTGGTATC |
| | | | AAGGTTACAAGACAGGTTTAAGGAGACCAATAGAAAC |
| | | | TGGGCATGTGGAGACAGAGAAGACTCTTGGGTTTCTG |
| | | | ATAGGCACTGACTCTCTCTGCCTATTGGTCTATTTTCCC |
| | | | ACCCTTAGGCTGCTTCTTGCCAACCAACC |
| 162 | | ALBcre | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTC |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 190 | | FTL-Timd | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAG |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 67 | L60 | FTL5 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGGGGCAGGTTGGTAT |
| | | | CAAGGTTACAAGACAGGTTTAAGGAGACCAATAGAAA |
| | | | CTGGGCATGTGGAGACAGGCACTGGGAGGATGTTGAG |
| | | | TAAGATGGAAAACTACTGATGACCCTTGCAGAGACAG |
| | | | AGTATTAGGACATGTTTGAACAGGGGCCGGGCGATCA |
| | | | GCAGGTAGCTCTAGAGGATCCCCGTCTGTCTGCACATT |
| | | | TCGTAGAGCGAGTGTTCCGATACTCTAATCTCCCTAGG |
| | | | CAAGGTTCATATTTGTGTAGGTTACTTATTCTCCTTTTG |
| | | | TTGACTAAGTCAATAATCAGAATCAGCAGGTTTGGAGT |
| | | | CAGCTTGGCAGGGATCAGCAGCCTGGGTTGGAAGGAG |
| | | | GGGGTATAAAAGCCCCTTCACCAGGAGAAGCCGTCAC |
| | | | ACAGATCCACAAGCTCGAGCCGAGTGGTCGTAGAAGA |
| | | | CTCTTGGGTTTCTGATAGGCACTGACTCTCTCTGCCTAT |
| | | | TGGTCTATTTTCCCACCCTTAGGCTGCTTCTTGCCAACC |
| | | | AACC |
| 190 | | FTL-Timd | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAG |
| 195 | | HBBi-mTTRp | GGGCAGGTTGGTATCAAGGTTACAAGACAGGTTTAAG |
| | | intron | GAGACCAATAGAAACTGGGCATGTGGAGACAGGCACT |
| | | | GGGAGGATGTTGAGTAAGATGGAAAACTACTGATGAC |
| | | | CCTTGCAGAGACAGAGTATTAGGACATGTTTGAACAG |
| | | | GGGCCGGGCGATCAGCAGGTAGCTCTAGAGGATCCCC |
| | | | GTCTGTCTGCACATTTCGTAGAGCGAGTGTTCCGATAC |
| | | | TCTAATCTCCCTAGGCAAGGTTCATATTTGTGTAGGTT |
| | | | ACTTATTCTCCTTTTGTTGACTAAGTCAATAATCAGAAT |
| | | | CAGCAGGTTTGGAGTCAGCTTGGCAGGGATCAGCAGC |
| | | | CTGGGTTGGAAGGAGGGGGTATAAAAGCCCCTTCACC |
| | | | AGGAGAAGCCGTCACACAGATCCACAAGCTCGAGCCG |
| | | | AGTGGTCGTAGAAGACTCTTGGGTTTCTGATAGGCACT |
| | | | GACTCTCTCTGCCTATTGGTCTATTTTCCCACCCTTAGG |
| | | | CTGCT |
| 196 | | mTTRmd | GCACTGGGAGGATGTTGAGTAAGATGGAAAACTACTG |
| | | promoter5 | ATGACCCTTGCAGAGACAGAGTATTAGGACATGTTTGA |
| | | | ACAGGGGCCGGGCGATCAGCAGGTAGCTCTAGAGGAT |
| | | | CCCCGTCTGTCTGCACATTTCGTAGAGCGAGTGTTCCG |
| | | | ATACTCTAATCTCCCTAGGCAAGGTTCATATTTGTGTA |
| | | | GGTTACTTATTCTCCTTTTGTTGACTAAGTCAATAATCA |
| | | | GAATCAGCAGGTTTGGAGTCAGCTTGGCAGGGATCAG |
| | | | CAGCCTGGGTTGGAAGGAGGGGGTATAAAAGCCCCTT |
| | | | CACCAGGAGAAGCCGTCACACAGATCCACAAGCTCGA |
| | | | GCCGAGTGGTCGT |
| 68 | L61 | FTL6 | GAGTTGCTGGTGCTTCCCCAGGCTGGAGATTGAGTTAA |
| | | | TATTAACAGGCCCAAGGCGATGTGGGCTTGTGCAATCA |
| | | | TAGGCCCGGCCTCGCCAGGTCACCTGAGGAGTTAATG |
| | | | AATACATATCTCCTGAGTTAATAATTACCAGCGCGGGC |
| | | | CAAATAAATAATCCGCGAGGGGCAGCTGGACTTTGGA |
| | | | CTCCGCGCGCTGGTAATTATTAACCTCGCGAATATTGA |
| | | | TTCGAGGCCGCGATTGCCGCAATCGCGAGGGGCAGGT |
| | | | GACCTTTGCCCAGCGCGCGTTCGCCCCGCCCCGGACGG |
| | | | TATCGATAAGCTTAGGAGCTTGGGCTGCAGGTCGAGGT |
| | | | CCCATAAAACTCTAGCCTCCCAGAGGGACCCCAGCCCC |
| | | | CACCCTCCCGCCCACGAACCCCTGCATTTCCAGAATCA |
| | | | GCCCCAGGGCCCCAACCCCCCCAAGCCCCCATTTCACA |
| | | | ACACGCTGGCGCTACAGGCGCGTGACTTCCCCTTGCTT |
| | | | TGGGGCGGGGGGCTGAGACTCCTATGTGCTCCGGATTG |
| | | | GTCAGGCACGGCCTTCGGCCCCGCCTCCTGCCACCGCA |
| | | | GATTGGCCGCTAGCCCTCCCCGAGCGCCCTGCCTCCGA |
| | | | GGGCCGGCGCACTATAAAAGCCCCTTCACCAGGAGAA |
| | | | GCCGTCACACAGATCCACAAGCTCGAGCCGAGTGGTC |
| | | | GTGGGTCTGTCTCTTGCTTCAACAGTGTTTGGACGGAA |
| | | | CAGATCCGGGGACTCTCTTCCACCAACCAACCGGATCC |
| | | | GCCACC |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 197 | | SynEnh2 | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAG |
| 191 | | FTL-Timd | TCCCATAAAACTCTAGCCTCCCAGAGGGACCCCAGCCC |
| | | promoter_alt | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCA |
| 69 | L62 | ALBcre- | AGCCAATGAAATACAAAGATGAGTCTAGTTAATAATC |
| | | FGBcre- | TACAATTATTGGTTAAAGAAGTATATTAGTGCTAATTT |
| | | SERPINA 1cre- | CCCTCCGTTTGTCCTAGCTTTTCTCGAATTTTAATAAAT |
| | | SynE-mTTR | ACCTCTCTGTCCTTCTCCTAGCTTCACCCTTTGTTTGTT |
| | | | GAAAGGAGATACTTTAGTGCAGTATTGCAACAGCTTAT |
| | | | GTAGACATGCAACCTTGAGGTCACAGACCTATGAGGG |
| | | | ATTGAGACAAAGGCAAATTTAGCAAATATTTTCCAGA |
| | | | GTACTCTGCTGCAATAACGAGTTGCTGGTGCTTCCCCA |
| | | | GGCTGGAGATTGAGTTAATATTAACAGGCCCAAGGCG |
| | | | ATGTGGGCTTGTGCAATCATAGGCCCGGCCTCGCCAGG |
| | | | TCACCTGAGGAGTTAATGAATACATATCTCCTGAGTTA |
| | | | ATAATTACCAGCGCGGGCCAAATAAATAATCCGCGAG |
| | | | GGGCAGCTGGACTTTGGACTCCGCGCGCTGGTAATTAT |
| | | | TAACCTCGCGAATATTGATTCGAGGCCGCGATTGCCGC |
| | | | AATCGCGAGGGGCAGGTGACCTTTGCCCAGCGCGCGT |
| | | | TCGCCCCGCCCCGGACGGTATCGATAAGCTTAGGAGCT |
| | | | TGGGCTGCAGGTCGAGGGCACTGGGAGGATGTTGAGT |
| | | | AAGATGGAAAACTACTGATGACCCTTGCAGAGACAGA |
| | | | GTATTAGGACATGTTTGAACAGGGGCCGGGCGATCAG |
| | | | CAGGTAGCTCTAGAGGATCCCCGTCTGTCTGCACATTT |
| | | | CGTAGAGCGAGTGTTCCGATACTCTAATCTCCCTAGGC |
| | | | AAGGTTCATATTTGTGTAGGTTACTTATTCTCCTTTTGT |
| | | | TGACTAAGTCAATAATCAGAATCAGCAGGTTTGGAGTC |
| | | | AGCTTGGCAGGGATCAGCAGCCTGGGTTGGAAGGAGG |
| | | | GGGTATAAAAGCCCCTTCACCAGGAGAAGCCGTCACA |
| | | | CAGATCCACAAGCTCGAGCCGAGTGGTCGTGCCTCCAT |
| | | | AGAAGCGGCCGCCACC |
| 162 | | ALBcre | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTC |
| 166 | | FGBcre 1 | GAATTTTAATAAATACCTCTCTGTCCTTCTCCTAGCTTC |
| | | | ACCCTTTGTTTGTTGAAAGGAGATACTTTAGTGCAGTA |
| | | | TTGCAACAGCTTATGTAGACATGCAACCTTGAGGTCAC |
| | | | AGACCTATGAGGGATTGAGACAAAGGCAAATTTAGCA |
| | | | AATATTTTCCAGAGTACTCTGCTGCAATAAC |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 197 | | SynEnh2 | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAG |
| 196 | | mTTRmd | GCACTGGGAGGATGTTGAGTAAGATGGAAAACTACTG |
| | | promoter5 | ATGACCCTTGCAGAGACAGAGTATTAGGACATGTTTGA |
| | | | ACAGGGGCCGGGCGATCAGCAGGTAGCTCTAGAGGAT |
| | | | CCCCGTCTGTCTGCACATTTCGTAGAGCGAGTGTTCCG |
| | | | ATACTCTAATCTCCCTAGGCAAGGTTCATATTTGTGTA |
| | | | GGTTACTTATTCTCCTTTTGTTGACTAAGTCAATAATCA |
| | | | GAATCAGCAGGTTTGGAGTCAGCTTGGCAGGGATCAG |
| | | | CAGCCTGGGTTGGAAGGAGGGGGTATAAAAGCCCCTT |
| | | | CACCAGGAGAAGCCGTCACACAGATCCACAAGCTCGA |
| | | | GCCGAGTGGTCGT |
| 70 | L63 | TBG.Ci | AGGGCTGGAAGCTACCTTTGACATCATTTCCTCTGCGA |
| | | | ATGCATGTATAATTTCTACAGAACCTATTAGAAAGGAT |
| | | | CACCCAGCCTCTGCTTTTGTACAACTTTCCCTTAAAAA |
| | | | ACTGCCAATTCCACTGCTGTTTGGCCCAATAGTGAGAA |
| | | | CTTTTTCCTGCTGCCTCTTGGTGCTTTTGCCTATGGCCC |
| | | | CTATTCTGCCTGCTGAAGACACTCTTGCCAGCATGGAC |
| | | | TTAAACCCCTCCAGCTCTGACAATCCTCTTTCTCTTTTG |
| | | | TTTTACATGAAGGGTCTGGCAGCCAAAGCAATCACTCA |
| | | | AAGTTCAAACCTTATCATTTTTTGCTTTGTTCCTCTTGG |
| | | | CCTTGGTTTTGTACATCAGCTTTGAAAATACCATCCCA |
| | | | GGGTTAATGCTGGGGTTAATTTATAACTAAGAGTGCTC |
| | | | TAGTTTTGCAATACAGGACATGCTATAAAAATGGAAA |
| | | | GATGTTGCTTTCTGAGAGACTGCAGAAGTTGGTCGTGA |
| | | | GGCACTGGGCAGGTAAGTATCAAGGTTACAAGACAGG |
| | | | TTTAAGGAGACCAATAGAAACTGGGCTTGTCGAGACA |
| | | | GAGAAGACTCTTGCGTTTCTGATAGGCACCTATTGGTC |
| | | | TTACTGACATCCACTTTGCCTTTCTCTCCACAGGTGTCC |
| | | | AGGCGGCCGCC |
| 71 | L64 | TBG.MD.Ci | AGGGCTGGAAGCTACCTTTGACATCATTTCCTCTGCGA |
| | | | ATGCATGTATAATTTCTACAGAACCTATTAGAAAGGAT |
| | | | CACCCAGCCTCTGCTTTTGTACAACTTTCCCTTAAAAA |
| | | | ACTGCCAATTCCACTGCTGTTTGGCCCAATAGTGAGAA |
| | | | CTTTTTCCTGCTGCCTCTTGGTGCTTTTGCCTATGGCCC |
| | | | CTATTCTGCCTGCTGAAGACACTCTTGCCAGCATGGAC |
| | | | TTAAACCCCTCCAGCTCTGACAATCCTCTTTCTCTTTTG |
| | | | TTTTACATGAAGGGTCTGGCAGCCAAAGCAATCACTCA |
| | | | AAGTTCAAACCTTATCATTTTTTGCTTTGTTCCTCTTGG |
| | | | CCTTGGTTTTGTACATCAGCTTTGAAAATACCATCCCA |
| | | | GGGTTAATGCTGGGGTTAATTTATAACTAAGAGTGCTC |
| | | | TAGTTTTGCAATACAGGACATGCTATAAAAATGGAAA |
| | | | GATGTTGCTTTCTGATCAGTCCGCCTGGAGACCTCGAG |
| | | | CCGAGTGGTCGTGGCAGGTAAGTATCAAGGTTACAAG |
| | | | ACAGGTTTAAGGAGACCAATAGAAACTGGGCTTGTCG |
| | | | AGACAGAGAAGACTCTTGCGTTTCTGATAGGCACCTAT |
| | | | TGGTCTTACTGACATCCACTTTGCCTTTCTCTCCACAGG |
| | | | TGTCCAGGCGGCCGCC |
| | | TBG.Ci | |
| | | promoter with | |
| | | Imd insertion | |
| 198 | | Imd | TCAGTCCGCCTGGAGACCTCGAGCCGAGTGGTCGT |
| 72 | L65 | TBG.MD.Ci2 | AGGGCTGGAAGCTACCTTTGACATCATTTCCTCTGCGA |
| | | | ATGCATGTATAATTTCTACAGAACCTATTAGAAAGGAT |
| | | | CACCCAGCCTCTGCTTTTGTACAACTTTCCCTTAAAAA |
| | | | ACTGCCAATTCCACTGCTGTTTGGCCCAATAGTGAGAA |
| | | | CTTTTTCCTGCTGCCTCTTGGTGCTTTTGCCTATGGCCC |
| | | | CTATTCTGCCTGCTGAAGACACTCTTGCCAGCATGGAC |
| | | | TTAAACCCCTCCAGCTCTGACAATCCTCTTTCTCTTTTG |
| | | | TTTTACATGAAGGGTCTGGCAGCCAAAGCAATCACTCA |
| | | | AAGTTCAAACCTTATCATTTTTTGCTTTGTTCCTCTTGG |
| | | | CCTTGGTTTTGTACATCAGCTTTGAAAATACCATCCCA |
| | | | GGGTTAATGCTGGGGTTAATTTATAACTAAGAGTGCTC |
| | | | TAGTTTTGCAATACAGGACATGCTATAAAAATGGAAA |
| | | | GATGTTGCTTTCTGAGAGATGCAGGTGGATTCCGAGCC |
| | | | GAGTGGTCGTTTGGGCAGGGCAGGTTGGTATCAAGGTT |
| | | | ACAAGACAGGTTTAAGGAGACCAATAGAAACTGGGCA |
| | | | TGTGGAGACAGAGAAGACTCTTGGGTTTCTGATAGGC |
| | | | ACTGACTCTCTCTGCCTATTGGTCTATTTTCCCACCCTT |
| | | | AGGCTGCTTTCCTTCCAAA |
| 199 | | TBGmd | AGGGCTGGAAGCTACCTTTGACATCATTTCCTCTGCGA |
| | | promoter | ATGCATGTATAATTTCTACAGAACCTATTAGAAAGGAT |
| | | | CACCCAGCCTCTGCTTTTGTACAACTTTCCCTTAAAAA |
| | | | ACTGCCAATTCCACTGCTGTTTGGCCCAATAGTGAGAA |
| | | | CTTTTTCCTGCTGCCTCTTGGTGCTTTTGCCTATGGCCC |
| | | | CTATTCTGCCTGCTGAAGACACTCTTGCCAGCATGGAC |
| | | | TTAAACCCCTCCAGCTCTGACAATCCTCTTTCTCTTTTG |
| | | | TTTTACATGAAGGGTCTGGCAGCCAAAGCAATCACTCA |
| | | | AAGTTCAAACCTTATCATTTTTTGCTTTGTTCCTCTTGG |
| | | | CCTTGGTTTTGTACATCAGCTTTGAAAATACCATCCCA |
| | | | GGGTTAATGCTGGGGTTAATTTATAACTAAGAGTGCTC |
| | | | TAGTTTTGCAATACAGGACATGCTATAAAAATGGAAA |
| | | | GATGTTGCTTTCTGAGAGATGCAGGTGGATTCCGAGCC |
| | | | GAGTGGTCGTTTGGGCA |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 73 | L66 | ApoE-HCR- | CTGCAGGCTCAGAGGCACACAGGAGTTTCTGGGCTCA |
| | | hAAT | CCCTGCCCCCTTCCAACCCCTCAGTTCCCATCCTCCAGC |
| | | | AGCTGTTTGTGTGCTGCCTCTGAAGTCCACACTGAACA |
| | | | AACTTCAGCCTACTCATGTCCCTAAAATGGGCAAACAT |
| | | | TGCAAGCAGCAAACAGCAAACACACAGCCCTCCCTGC |
| | | | CTGCTGACCTTGGAGCTGGGGCAGAGGTCAGAGACCT |
| | | | CTCTGGGCCCATGCCACCTCCAACATCCACTCGACCCC |
| | | | TTGGAATTTCGGTGGAGAGGAGCAGAGGTTGTCCTGG |
| | | | CGTGGTTTAGGTAGTGTGAGAGGGTCCGGGTTCAAAA |
| | | | CCACTTGCTGGGTGGGGAGTCGTCAGTAAGTGGCTATG |
| | | | CCCCGACCCCGAAGCCTGTTTCCCCATCTGTACAATGG |
| | | | AAATGATAAAGACGCCCATCTGATAGGGTTTTTGTGGC |
| | | | AAATAAACATTTGGTTTTTTTGTTTTGTTTTGTTTTGTTT |
| | | | TTTGAGATGGAGGTTTGCTCTGTCGCCCAGGCTGGAGT |
| | | | GCAGTGACACAATCTCATCTCACCACAACCTTCCCCTG |
| | | | CCTCAGCCTCCCAAGTAGCTGGGATTACAAGCATGTGC |
| | | | CACCACACCTGGCTAATTTTCTATTTTTAGTAGAGACG |
| | | | GGTTTCTCCATGTTGGTCAGCCTCAGCCTCCCAAGTAA |
| | | | CTGGGATTACAGGCCTGTGCCACCACACCCGGCTAATT |
| | | | TTTTCTATTTTTGACAGGGACGGGGTTTCACCATGTTG |
| | | | GTCAGGCTCCTCTAGAGGTACCCGGGGATCTTGCTACC |
| | | | AGTGGAACAGCCACTAAGGATTCTGCAGTGAGAGCAG |
| | | | AGGGCCAGCTAAGTGGTACTCTCCCAGAGACTGTCTGA |
| | | | CTCACGCCACCCCCTCCACCTTGGACACAGGACGCTGT |
| | | | GGTTTCTGAGCCAGGTACAATGACTCCTTTCGGTAAGT |
| | | | GCAGTGGAAGCTGTACACTGCCCAGGCAAAGCGTCCG |
| | | | GGCAGCGTAGGCGGGCGACTCAGATCCCAGCCAGTGG |
| | | | ACTTAGCCCCTGTTTGCTCCTCCGATAACTGGGGTGAC |
| | | | CTTGGTTAATATTCACCAGCAGCCTCCCCCGTTGCCCC |
| | | | TCTGGATCCACTGCTTAAATACGGACGAGGACAGGGC |
| | | | CCTGTCTCCTCAGCTTCAGGCACCACCACTGACCTGGG |
| | | | ACAGTGAATGATCCCCCTGATCTGCGGCCTCGACGGTA |
| | | | TCGATAAGCTTGATATCGAATTCTAGTCGTCGACCACT |
| | | | TTCACAATCTGCGGCACCGGTACC |
| 164 | | ApoE-HCR | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGG |
| 200 | | hAAT | GATCTTGCTACCAGTGGAACAGCCACTAAGGATTCTGC |
| | | promoter | AGTGAGAGCAGAGGGCCAGCTAAGTGGTACTCTCCCA |
| | | | GAGACTGTCTGACTCACGCCACCCCCTCCACCTTGGAC |
| | | | ACAGGACGCTGTGGTTTCTGAGCCAGGTACAATGACTC |
| | | | CTTTCGGTAAGTGCAGTGGAAGCTGTACACTGCCCAGG |
| | | | CAAAGCGTCCGGGCAGCGTAGGCGGGCGACTCAGATC |
| | | | CCAGCCAGTGGACTTAGCCCCTGTTTGCTCCTCCGATA |
| | | | ACTGGGGTGACCTTGGTTAATATTCACCAGCAGCCTCC |
| | | | CCCGTTGCCCCTCTGGATCCACTGCT |
| 74 | L67 | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 75 | L68 | HLP | TGTTTGCTGCTTGCAATGTTTGCCCATTTTAGGGTGGAC |
| | | | ACAGGACGCTGTGGTTTCTGAGCCAGGGGGCGACTCA |
| | | | GATCCCAGCCAGTGGACTTAGCCCCTGTTTGCTCCTCC |
| | | | GATAACTGGGGTGACCTTGGTTAATATTCACCAGCAGC |
| | | | CTCCCCCGTTGCCCCTCTGGATCCACTGCTTAAATACG |
| | | | GACGAGGACAGGGCCCTGTCTCCTCAGCTTCAGGCACC |
| | | | ACCACTGACCTGGGACAGTGAATCGCGGCCGCCACC |
| 76 | L69 | AAT_v3 | CACTGGGAGGATGTTGAGTAAGATGGAAAACTACTGA |
| | | | TGACCCTTGCAGAGACAGAGTATTAGGACATGTTTGAA |
| | | | CAGGGGCCGGGCGATCAGCAGGTAGGTCTTGTGTCTG |
| | | | CCGGGCAATGAGCAAGGCTCCTTCCTGTCCAAGCTCCC |
| | | | CGCCCCTCCCCAGCCTACTGCCTCCACCCGAAGTCTAC |
| | | | TTCCTGGGTGGGCAGGAACTGGGCACTGTGCCCAGGG |
| | | | CATGGATCTTGCTACCAGTGGAACAGCCACTAAGGATT |
| | | | CTGCAGTGAGAGCAGAGGGCCAGCTAAGTGGTACTCT |
| | | | CCCAGAGACTGTCTGACTCACGCCACCCCCTCCACCTT |
| | | | GGACACAGGACGCTGTGGTTTCTGAGCCAGGTACAAT |
| | | | GACTCCTTTCGGTAAGTGCAGTGGAAGCTGTACACTGC |
| | | | CCAGGCAAAGCGTCCGGGCAGCGTAGGCGGGCGACTC |
| | | | AGATCCCAGCCAGTGGACTTAGCCCCTGTTTGCTCCTC |
| | | | CGATAACTGGGGTGACCTTGGTTAATATTCACCAGCAG |
| | | | CCTCCCCCGTTGCCCCTCTGGATCCACTGCTTATATAAC |
| | | | CCAGGGGCACAGGGGCTGCCCTCAGTCCGCCTGGAGA |
| | | | CCTCGAGCCGAGTGGTCGTCTCAGGAGCCGGGCAGGT |
| | | | TGGTATCAAGGTTACAAGACAGGTTTAAGGAGACCAA |
| | | | TAGAAACTGGGCATGTGGAGACAGAGAAGACTCTTGG |
| | | | GTTTCTGATAGGCACTGACTCTCTCTGCCTATTGGTCTA |
| | | | TTTTCCCACCCTTAGGCTGCTGCCACC |
| 189 | | AATv3 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 77 | L70 | FTL7 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACCGGGCAGG |
| | | | TTGGTATCAAGGTTACAAGACAGGTTTAAGGAGACCA |
| | | | ATAGAAACTGGGCATGTGGAGACAGAGAAGACTCTTG |
| | | | GGTTTCTGATAGGCACTGACTCTCTCTGCCTATTGGTCT |
| | | | ATTTTCCCACCCTTAGGCTGCTGGATCCGCCACC |
| 201 | | FTL-TImd2 | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 192 | | TImd (part of | TATAAAAGCCCCTTCACCAGGAGAAGCCGTCACACAG |
| | | FTL-TImd2 | ATCCACAAGCTCGAGCCGAGTGGTCGT |
| | | promoter) | |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 78 | L71 | FTL8 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTGGGCAGGTTGGTATCAAGGTTA |
| | | | CAAGACAGGTTTAAGGAGACCAATAGAAACTGGGCAT |
| | | | GTGGAGACAGAGAAGACTCTTGGGTTTCTGATAGGCA |
| | | | CTGACTCTCTCTGCCTATTGGTCTATTTTCCCACCCTTA |
| | | | GGCTGCTTCTTGCCAACCAACC |
| 202 | | FTL-TImd3 | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCT |
| 192 | | TImd(part of | TATAAAAGCCCCTTCACCAGGAGAAGCCGTCACACAG |
| | | FTL-TImd3 | ATCCACAAGCTCGAGCCGAGTGGTCGT |
| | | promoter) | |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 79 | L72 | FTL9 | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTCGAGTTGCTGGTGCT |
| | | | TCCCCAGGCTGGAGATTGAGTTAATATTAACAGGCCCA |
| | | | AGGCGATGTGGGCTTGTGCAATCATAGGCCCGGCCTCG |
| | | | CCAGGTCACCTGAGGAGTTAATGAATACATATCTCCTT |
| | | | CCCATAAAACTCTAGCCTCCCAGAGGGACCCCAGCCCC |
| | | | CACCCTCCCGCCCACGAACCCCTGCATTTCCAGAATCA |
| | | | GCCCCAGGGCCCCAACCCCCCCAAGCCCCCATTTCACA |
| | | | ACACGCTGGCGCTACAGGCGCGTGACTTCCCCTTGCTT |
| | | | TGGGGCGGGGGGCTGAGACTCCTATGTGCTCCGGATTG |
| | | | GTCAGGCACGGCCTTCGGCCCCGCCTCCTGCCACCGCA |
| | | | GATTGGCCGCTAGCCCTCCCCGAGCGCCCTGCCTCCGA |
| | | | GGGCCGGCGCACTATAAAAGCCCCTTCACCAGGAGAA |
| | | | GCCGTCACACAGATCCACAAGCTCGAGCCGAGTGGTC |
| | | | GTGGGTCTGTCTCTTGCTTCAACAGTGTTTGGACGGAA |
| | | | CAGATCCGGGGACTCTCTTCCAGCCTCCGACCGCCCTC |
| | | | CGATTTCCTCTCCGCTTGCAACCTCCGGGACCATCTTCT |
| | | | CGGCCATCTCCTGCTTCTGGGACCTGCCAGCACCGTTT |
| | | | TTGTGGTTAGCTCCTGGGCAGGTTGGTATCAAGGTTAC |
| | | | AAGACAGGTTTAAGGAGACCAATAGAAACTGGGCATG |
| | | | TGGAGACAGAGAAGACTCTTGGGTTTCTGATAGGCACT |
| | | | GACTCTCTCTGCCTATTGGTCTATTTTCCCACCCTTAGG |
| | | | CTGCTTCTTGCCAACCAACC |
| 162 | | ALBcre | AGCCAATGAAATACAAAGATGAGTCTAGTTAATAATC |
| | | | TACAATTATTGGTTAAAGAAGTATATTAGTGCTAATTT |
| | | | CCCTCCGTTTGTCCTAGCTTTTCTC |
| 168 | | SERPINAlcre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA 1cre | TCGCCAGGTCACCTGAGGAGTTAATGAATACATATCTC |
| | | 2 | CT |
| 202 | | FTL-TImd3 | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCT |
| 192 | | TImd (part of | TATAAAAGCCCCTTCACCAGGAGAAGCCGTCACACAG |
| | | FTL-TImd3 | ATCCACAAGCTCGAGCCGAGTGGTCGT |
| | | promoter) | |
| 193 | L73 | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 80 | | FTL10 | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTGGGCAGGTTGGTATCAAGGTTA |
| | | | CAAGACAGGTTTAAGGAGACCAATAGAAACTGGGCAT |
| | | | GTGGAGACAGGCACTGGGAGGATGTTGAGTAAGATGG |
| | | | AAAACTACTGATGACCCTTGCAGAGACAGAGTATTAG |
| | | | GACATGTTTGAACAGGGGCCGGGCGATCAGCAGGTAG |
| | | | CTCTAGAGGATCCCCGTCTGTCTGCACATTTCGTAGAG |
| | | | CGAGTGTTCCGATACTCTAATCTCCCTAGGCAAGGTTC |
| | | | ATATTTGTGTAGGTTACTTATTCTCCTTTTGTTGACTAA |
| | | | GTCAATAATCAGAATCAGCAGGTTTGGAGTCAGCTTGG |
| | | | CAGGGATCAGCAGCCTGGGTTGGAAGGAGGGGGTATA |
| | | | AAAGCCCCTTCACCAGGAGAAGCCGTCACACAGATCC |
| | | | ACAAGCTCGAGCCGAGTGGTCGTAGAAGACTCTTGGG |
| | | | TTTCTGATAGGCACTGACTCTCTCTGCCTATTGGTCTAT |
| | | | TTTCCCACCCTTAGGCTGCTTCTTGCCAACCAACC |
| 202 | | FTL-TImd3 | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCT |
| 195 | | HBBi-mTTRp | GGGCAGGTTGGTATCAAGGTTACAAGACAGGTTTAAG |
| | | intron | GAGACCAATAGAAACTGGGCATGTGGAGACAGGCACT |
| | | | GGGAGGATGTTGAGTAAGATGGAAAACTACTGATGAC |
| | | | CCTTGCAGAGACAGAGTATTAGGACATGTTTGAACAG |
| | | | GGGCCGGGCGATCAGCAGGTAGCTCTAGAGGATCCCC |
| | | | GTCTGTCTGCACATTTCGTAGAGCGAGTGTTCCGATAC |
| | | | TCTAATCTCCCTAGGCAAGGTTCATATTTGTGTAGGTT |
| | | | ACTTATTCTCCTTTTGTTGACTAAGTCAATAATCAGAAT |
| | | | CAGCAGGTTTGGAGTCAGCTTGGCAGGGATCAGCAGC |
| | | | CTGGGTTGGAAGGAGGGGGTATAAAAGCCCCTTCACC |
| | | | AGGAGAAGCCGTCACACAGATCCACAAGCTCGAGCCG |
| | | | AGTGGTCGTAGAAGACTCTTGGGTTTCTGATAGGCACT |
| | | | GACTCTCTCTGCCTATTGGTCTATTTTCCCACCCTTAGG |
| | | | CTGCT |
| 196 | | mTTRmd | GCACTGGGAGGATGTTGAGTAAGATGGAAAACTACTG |
| | | promoter5 | ATGACCCTTGCAGAGACAGAGTATTAGGACATGTTTGA |
| | | | ACAGGGGCCGGGCGATCAGCAGGTAGCTCTAGAGGAT |
| | | | CCCCGTCTGTCTGCACATTTCGTAGAGCGAGTGTTCCG |
| | | | ATACTCTAATCTCCCTAGGCAAGGTTCATATTTGTGTA |
| | | | GGTTACTTATTCTCCTTTTGTTGACTAAGTCAATAATCA |
| | | | GAATCAGCAGGTTTGGAGTCAGCTTGGCAGGGATCAG |
| | | | CAGCCTGGGTTGGAAGGAGGGGGTATAAAAGCCCCTT |
| | | | CACCAGGAGAAGCCGTCACACAGATCCACAAGCTCGA |
| | | | GCCGAGTGGTCGT |
| 81 | L74 | FTL11 | GAGTTGCTGGTGCTTCCCCAGGCTGGAGATTGAGTTAA |
| | | | TATTAACAGGCCCAAGGCGATGTGGGCTTGTGCAATCA |
| | | | TAGGCCCGGCCTCGCCAGGTCACCTGAGGAGTTAATG |
| | | | AATACATATCTCCTGAGTTAATAATTACCAGCGCGGGC |
| | | | CAAATAAATAATCCGCGAGGGGCAGCTGGACTTTGGA |
| | | | CTCCGCGCGCTGGTAATTATTAACCTCGCGAATATTGA |
| | | | TTCGAGGCCGCGATTGCCGCAATCGCGAGGGGCAGGT |
| | | | GACCTTTGCCCAGCGCGCGTTCGCCCCGCCCCGGACGG |
| | | | TATCGATAAGCTTAGGAGCTTGGGCTGCAGGTCGAGGT |
| | | | CCCATAAAACTCTAGCCTCCCAGAGGGACCCCAGCCCC |
| | | | CACCCTCCCGCCCACGAACCCCTGCATTTCCAGAATCA |
| | | | GCCCCAGGGCCCCAACCCCCCCAAGCCCCCATTTCACA |
| | | | ACACGCTGGCGCTACAGGCGCGTGACTTCCCCTTGCTT |
| | | | TGGGGCGGGGGGCTGAGACTCCTATGTGCTCCGGATTG |
| | | | GTCAGGCACGGCCTTCGGCCCCGCCTCCTGCCACCGCA |
| | | | GATTGGCCGCTAGCCCTCCCCGAGCGCCCTGCCTCCGA |
| | | | GGGCCGGCGCACTATAAAAGCCCCTTCACCAGGAGAA |
| | | | GCCGTCACACAGATCCACAAGCTCGAGCCGAGTGGTC |
| | | | GTGGGTCTGTCTCTTGCTTCAACAGTGTTTGGACGGAA |
| | | | CAGATCCGGGGACTCTCTTCCACCTCCGACCGCCCTCC |
| | | | GATTTCCTCTCCGCTTGCAACCTCCGGGACCATCTTCTC |
| | | | GGCCATCTCCTGCTTCTGGGACCTGCCAGCACCGTTTT |
| | | | TGTGGTTAGCTCCTCCAACCAACCGGATCCGCCACC |
| 168 | | SERPINA1cre | GAGTTGCTGGTGCTTCCCCAGGCTGGAGATTGAGTTAA |
| | | 1 | TATTAACAGGCCCAAGGCGATGTGGGCTTGTGCAATCA |
| | | | TAGGCCCGGCC |
| 169 | | SERPINA1cre | TCGCCAGGTCACCTGAGGAGTTAATGAATACATATCTC |
| | | 2 | CT |
| 197 | | SynEnh2 | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAG |
| 203 | | FTL-Timd4 | TCCCATAAAACTCTAGCCTCCCAGAGGGACCCCAGCCC |
| | | promoter | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCACCTCCGACCGCCCTC |
| | | | CGATTTCCTCTCCGCTTGCAACCTCCGGGACCATCTTCT |
| | | | CGGCCATCTCCTGCTTCTGGGACCTGCCAGCACCGTTT |
| | | | TTGTGGTTAGCTCCT |
| 82 | L75 | HCB-MVMi | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGAGCCAA |
| | | | GAGGTAAGGGTTTAAGGGATGGTTGGTTGGTGGGGTA |
| | | | TTAATGTTTAATTACCTGGAGCACCTGCCTGAAATCAC |
| | | | TTTTTTTCAGGTTGGGGATCCGCCACC |
| 204 | | HCB promoter | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 83 | L76 | HCB2-MVMi | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGTGAGCCAAGAGGTAAGG |
| | | | GTTTAAGGGATGGTTGGTTGGTGGGGTATTAATGTTTA |
| | | | ATTACCTGGAGCACCTGCCTGAAATCACTTTTTTTCAG |
| | | | GTTGGGGATCCGCCACC |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 84 | L77 | HCB3-MVMi | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGTGGGTCTGTCTCTTGCTT |
| | | | CAACAGTGTTTGGACGGAACAGATCCGGGGACGAGCC |
| | | | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGGGGATCCGCCACC |
| 238 | | HCB3 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGTGGGTCTGTCTCTTGCTT |
| | | | CAACAGTGTTTGGACGGAACAGATCCGGGGAC |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 85 | L78 | HCB4-MVMi | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | CCCCTTCACCAGGAGAAGCCGTCACACAGATCCACAA |
| | | | GCTCGAGCCGAGTGGTCGTGGGTCTGTCTCTTGCTTCA |
| | | | ACAGTGTTTGGACGGAACAGATCCGGGGACTCTCTTCC |
| | | | ACCTCCGACCGCCCTCCGATTTCCTCTCCGCTTGCAAC |
| | | | CTCCGGGACCATCTTCTCGGCCATCTCCTGCTTCTGGG |
| | | | ACCTGCCAGCACCGTTTTTGTGGTTAGCTCCTCCAACC |
| | | | AACCGAGCCAAGAGGTAAGGGTTTAAGGGATGGTTGG |
| | | | TTGGTGGGGTATTAATGTTTAATTACCTGGAGCACCTG |
| | | | CCTGAAATCACTTTTTTTCAGGTTGGGGATCCGCCACC |
| 237 | | HCB4 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | CCCCTTCACCAGGAGAAGCCGTCACACAGATCCACAA |
| | | | GCTCGAGCCGAGTGGTCGTGGGTCTGTCTCTTGCTTCA |
| | | | ACAGTGTTTGGACGGAACAGATCCGGGGACTCTCTTCC |
| | | | ACCTCCGACCGCCCTCCGATTTCCTCTCCGCTTGCAAC |
| | | | CTCCGGGACCATCTTCTCGGCCATCTCCTGCTTCTGGG |
| | | | ACCTGCCAGCACCGTTTTTGTGGTTAGCTCCTCCAACC |
| | | | AACC |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 86 | L78b | HCB4-MVMi | GTTAATCATTAAGTCGTTAATTTTTGTGGCCCTTGCGAT |
| | | v2 | GTTTGCTCTGGTTAATAATCTCAGGACAAACAGAGGTT |
| | | | AATAATTTTCCAGATCTCTCTGAGCAATAGTATAAAAG |
| | | | CCCCTTCACCAGGAGAAGCCGTCACACAGATCCACAA |
| | | | GCTCGAGCCGAGTGGTCGTGGGTCTGTCTCTTGCTTCA |
| | | | ACAGTGTTTGGACGGAACAGATCCGGGGACTCTCTTCC |
| | | | ACCTCCGACCGCCCTCCGATTTCCTCTCCGCTTGCAAC |
| | | | CTCCGGGACCATCTTCTCGGCCATCTCCTGCTTCTGGG |
| | | | ACCTGCCAGCACCGTTTTTGTGGTTAGCTCCTCCAACC |
| | | | AACCGAGCCGAGCCAAGAGGTAAGGGTTTAAGGGATG |
| | | | GTTGGTTGGTGGGGTATTAATGTTTAATTACCTGGAGC |
| | | | ACCTGCCTGAAATCACTTTTTTTCAGGTTGGGGATCCG |
| | | | CCACC |
| 236 | | HCB4b | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | CCCCTTCACCAGGAGAAGCCGTCACACAGATCCACAA |
| | | | GCTCGAGCCGAGTGGTCGTGGGTCTGTCTCTTGCTTCA |
| | | | ACAGTGTTTGGACGGAACAGATCCGGGGACTCTCTTCC |
| | | | ACCTCCGACCGCCCTCCGATTTCCTCTCCGCTTGCAAC |
| | | | CTCCGGGACCATCTTCTCGGCCATCTCCTGCTTCTGGG |
| | | | ACCTGCCAGCACCGTTTTTGTGGTTAGCTCCTCCAACC |
| | | | AACCGAGCC |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 87 | L79 | SERPINAe- | GAGTTGCTGGTGCTTCCCCAGGCTGGAGATTGAGTTAA |
| | | SynE-HCB- | TATTAACAGGCCCAAGGCGATGTGGGCTTGTGCAATCA |
| | | MVMi | TAGGCCCGGCCTCGCCAGGTCACCTGAGGAGTTAATG |
| | | | AATACATATCTCCTGAGTTAATAATTACCAGCGCGGGC |
| | | | CAAATAAATAATCCGCGAGGGGCAGCTGGACTTTGGA |
| | | | CTCCGCGCGCTGGTAATTATTAACCTCGCGAATATTGA |
| | | | TTCGAGGCCGCGATTGCCGCAATCGCGAGGGGCAGGT |
| | | | GACCTTTGCCCAGCGCGCGTTCGCCCCGCCCCGGACGG |
| | | | TATCGATAAGCTTAGGAGCTTGGGCTGCAGGTCGAGG |
| | | | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGAGCCAA |
| | | | GAGGTAAGGGTTTAAGGGATGGTTGGTTGGTGGGGTA |
| | | | TTAATGTTTAATTACCTGGAGCACCTGCCTGAAATCAC |
| | | | TTTTTTTCAGGTTGGGGATCCGCCACC |
| 168 | | SERPINAIc | GAGTTGCTGGTGCTTCCCCAGGCTGGAGATTGAGTTAA |
| | | re 1 | TATTAACAGGCCCAAGGCGATGTGGGCTTGTGCAATCA |
| | | | TAGGCCCGGCC |
| 169 | | SERPINA1c | TCGCCAGGTCACCTGAGGAGTTAATGAATACATATCTC |
| | | re2 | CT |
| 197 | | SynEnh2 | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAG |
| 204 | | HCB | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 88 | L80 | FTL2_EH38E1 | CACACACACCATGGACATACACACACACCACACAAGC |
| | | 513438 | ACCGTGCACACAAATATACACAAACCACATACACACG |
| | | | TGACACACATACACACATGCAACATGCACACACACCA |
| | | | CAAACACATGCACCATGCACACACATACACACCACAA |
| | | | ACATACATACACACGCATGCACCATGCACACACATAT |
| | | | ACACACCACGCACATACATGCATCATGCACGGACACA |
| | | | CCCCACACATATACACATGCACCATGCACATACACACC |
| | | | ACACACACACGCACCATGCACACACATATACAGCCAC |
| | | | CACATACATACATATGTCACACACACATACACAGGAC |
| | | | GTCTCTCAAGGATAAGTAAGTAATATTAAGGTACGGG |
| | | | AGGTATTGGACAGGCCGCAATAAAATATCTTTATTTTC |
| | | | ATTACATCTGTGTGTTGGTTTTTTGTGTGAATCGATAGT |
| | | | ACTAACATACGCTCTCCATCAAAACAAAACGAAACAA |
| | | | AACAAACTAGCAAAATAGGCTGTCCCCAGTGCAAGTG |
| | | | CAGGTGCCAGAACATTTCTCTGGCCTAACTGGCCGGTA |
| | | | CCTGAGCTCACGTCTCTCAAGGATAAGTAAGTAATATT |
| | | | AAGGTACGGGAGGTATTGGACAGGCCGCAATAAAATA |
| | | | TCTTTATTTTCATTACATCTGTGTGTTGGTTTTTTGTGTG |
| | | | AATCGATAGTACTAACATACGCTCTCCATCAAAACAAA |
| | | | ACGAAACAAAACAAACTAGCAAAATAGGCTGTCCCCA |
| | | | GTGCAAGTGCAGGTGCCAGAACATTTCTCTGGCCTAAC |
| | | | TGGCCGGTACCTGAGCTCACGTCTCTCAAGGATAAGTA |
| | | | AGTAATCCAATGATGGCGCCTGCTGAATTCTGTACGGG |
| | | | CCAGATATACGCGTTTCCCATAAAACTCTAGCCTCCCA |
| | | | GAGGGACCCCAGCCCCCACCCTCCCGCCCACGAACCC |
| | | | CTGCATTTCCAGAATCAGCCCCAGGGCCCCAACCCCCC |
| | | | CAAGCCCCCATTTCACAACACGCTGGCGCTACAGGCGC |
| | | | GTGACTTCCCCTTGCTTTGGGGCGGGGGGCTGAGACTC |
| | | | CTATGTGCTCCGGATTGGTCAGGCACGGCCTTCGGCCC |
| | | | CGCCTCCTGCCACCGCAGATTGGCCGCTAGCCCTCCCC |
| | | | GAGCGCCCTGCCTCCGAGGGCCGGCGCACTATAAAAG |
| | | | CCCCTTCACCAGGAGAAGCCGTCACACAGATCCACAA |
| | | | GCTCGAGCCGAGTGGTCGTGGGTCTGTCTCTTGCTTCA |
| | | | ACAGTGTTTGGACGGAACAGATCCGGGGACTCTCTTCC |
| | | | AGCCTCCGACCGCCCTCCGATTTCCTCTCCGCTTGCAA |
| | | | CCTCCGGGACCATCTTCTCGGCCATCTCCTGCTTCTGG |
| | | | GACCTGCCAGCACCGTTTTTGTGGTTAGCTCCTTCTTGC |
| | | | CAACCAACCGGATCCGCCACC |
| 239 | | EH38E1513438 | CACACACACCATGGACATACACACACACCACACAAGC |
| | | | ACCGTGCACACAAATATACACAAACCACATACACACG |
| | | | TGACACACATACACACATGCAACATGCACACACACCA |
| | | | CAAACACATGCACCATGCACACACATACACACCACAA |
| | | | ACATACATACACACGCATGCACCATGCACACACATAT |
| | | | ACACACCACGCACATACATGCATCATGCACGGACACA |
| | | | CCCCACACATATACACATGCACCATGCACATACACACC |
| | | | ACACACACACGCACCATGCACACACATATACAGCCAC |
| | | | CACATACATACATATGTCACACACACATACACAGGAC |
| | | | GTCTCTCAAGGATAAGTAAGTAATATTAAGGTACGGG |
| | | | AGGTATTGGACAGGCCGCAATAAAATATCTTTATTTTC |
| | | | ATTACATCTGTGTGTTGGTTTTTTGTGTGAATCGATAGT |
| | | | ACTAACATACGCTCTCCATCAAAACAAAACGAAACAA |
| | | | AACAAACTAGCAAAATAGGCTGTCCCCAGTGCAAGTG |
| | | | CAGGTGCCAGAACATTTCTCTGGCCTAACTGGCCGGTA |
| | | | CCTGAGCTCACGTCTCTCAAGGATAAGTAAGTAATATT |
| | | | AAGGTACGGGAGGTATTGGACAGGCCGCAATAAAATA |
| | | | TCTTTATTTTCATTACATCTGTGTGTTGGTTTTTTGTGTG |
| | | | AATCGATAGTACTAACATACGCTCTCCATCAAAACAAA |
| | | | ACGAAACAAAACAAACTAGCAAAATAGGCTGTCCCCA |
| | | | GTGCAAGTGCAGGTGCCAGAACATTTCTCTGGCCTAAC |
| | | | TGGCCGGTACCTGAGCTCACGTCTCTCAAGGATAAGTA |
| | | | AGTAATCCAATGATGGCGCCTGCTGAATTCTGTACGGG |
| | | | CCAGATATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 89 | L81 | FTL2_EH38E1 | CACACACACTCACACAAAGCACATTTACACACATGCA |
| | | 514777 | CACAGTCATACATGCACACCCTTGTACACACAACACAC |
| | | | ACACATGCACACAGGCACACACACATGCACACATAAC |
| | | | GTGAATATCACGTGCAGTCACACACATGCAAACACAC |
| | | | ACACAGTTACATACATTCAAACACATGTGCACATAATA |
| | | | CATGCATGCATAGAGTCACACACACATGCAGTCACAC |
| | | | ACAACACATACATGCACACAGTCACACAGAAGCAGTC |
| | | | ACACACACATGCACCACATACACAGACAAAAGCACAC |
| | | | AGGCACACACACTACACACACACACGAACACATATGC |
| | | | ACACAGTCTCACACACGTCTCTCAAGGATAAGTAAGTA |
| | | | ATATTAAGGTACGGGAGGTATTGGACAGGCCGCAATA |
| | | | AAATATCTTTATTTTCATTACATCTGTGTGTTGGTTTTT |
| | | | TGTGTGAATCGATAGTACTAACATACGCTCTCCATCAA |
| | | | AACAAAACGAAACAAAACAAACTAGCAAAATAGGCTG |
| | | | TCCCCAGTGCAAGTGCAGGTGCCAGAACATTTCTCTGG |
| | | | CCTAACTGGCCGGTACCTGAGCTCACGTCTCTCAAGGA |
| | | | TAAGTAAGTAATATTAAGGTACGGGAGGTATTGGACA |
| | | | GGCCGCAATAAAATATCTTTATTTTCATTACATCTGTGT |
| | | | GTTGGTTTTTTGTGTGAATCGATAGTACTAACATACGC |
| | | | TCTCCATCAAAACAAAACGAAACAAAACAAACTAGCC |
| | | | AATGATGGCGCCTGCTGAATTCTGTACGGGCCAGATAT |
| | | | ACGCGTTTCCCATAAAACTCTAGCCTCCCAGAGGGACC |
| | | | CCAGCCCCCACCCTCCCGCCCACGAACCCCTGCATTTC |
| | | | CAGAATCAGCCCCAGGGCCCCAACCCCCCCAAGCCCC |
| | | | CATTTCACAACACGCTGGCGCTACAGGCGCGTGACTTC |
| | | | CCCTTGCTTTGGGGCGGGGGGCTGAGACTCCTATGTGC |
| | | | TCCGGATTGGTCAGGCACGGCCTTCGGCCCCGCCTCCT |
| | | | GCCACCGCAGATTGGCCGCTAGCCCTCCCCGAGCGCCC |
| | | | TGCCTCCGAGGGCCGGCGCACTATAAAAGCCCCTTCAC |
| | | | CAGGAGAAGCCGTCACACAGATCCACAAGCTCGAGCC |
| | | | GAGTGGTCGTGGGTCTGTCTCTTGCTTCAACAGTGTTT |
| | | | GGACGGAACAGATCCGGGGACTCTCTTCCAGCCTCCG |
| | | | ACCGCCCTCCGATTTCCTCTCCGCTTGCAACCTCCGGG |
| | | | ACCATCTTCTCGGCCATCTCCTGCTTCTGGGACCTGCC |
| | | | AGCACCGTTTTTGTGGTTAGCTCCTTCTTGCCAACCAA |
| | | | CCGGATCCGCCACC |
| 240 | | EH38E1514777 | CACACACACTCACACAAAGCACATTTACACACATGCA |
| | | | CACAGTCATACATGCACACCCTTGTACACACAACACAC |
| | | | ACACATGCACACAGGCACACACACATGCACACATAAC |
| | | | GTGAATATCACGTGCAGTCACACACATGCAAACACAC |
| | | | ACACAGTTACATACATTCAAACACATGTGCACATAATA |
| | | | CATGCATGCATAGAGTCACACACACATGCAGTCACAC |
| | | | ACAACACATACATGCACACAGTCACACAGAAGCAGTC |
| | | | ACACACACATGCACCACATACACAGACAAAAGCACAC |
| | | | AGGCACACACACTACACACACACACGAACACATATGC |
| | | | ACACAGTCTCACACACGTCTCTCAAGGATAAGTAAGTA |
| | | | ATATTAAGGTACGGGAGGTATTGGACAGGCCGCAATA |
| | | | AAATATCTTTATTTTCATTACATCTGTGTGTTGGTTTTT |
| | | | TGTGTGAATCGATAGTACTAACATACGCTCTCCATCAA |
| | | | AACAAAACGAAACAAAACAAACTAGCAAAATAGGCTG |
| | | | TCCCCAGTGCAAGTGCAGGTGCCAGAACATTTCTCTGG |
| | | | CCTAACTGGCCGGTACCTGAGCTCACGTCTCTCAAGGA |
| | | | TAAGTAAGTAATATTAAGGTACGGGAGGTATTGGACA |
| | | | GGCCGCAATAAAATATCTTTATTTTCATTACATCTGTGT |
| | | | GTTGGTTTTTTGTGTGAATCGATAGTACTAACATACGC |
| | | | TCTCCATCAAAACAAAACGAAACAAAACAAACTAGCC |
| | | | AATGATGGCGCCTGCTGAATTCTGTACGGGCCAGATAT |
| | | | ACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 90 | L82 | FTL2_ | TCGAAACCAAATTGGGCGACATAGTGAGACCTGGTCT |
| | | EH38E1521375 | CTACAGCAAATACACACACACACACACACACACACTT |
| | | | ATGCACACATACACCATACATACACATACAGTTAGTCA |
| | | | CACATATACACACATACACACACCACACACACATACA |
| | | | CACATAGATACACAAATACATATACACCACACACATA |
| | | | CACACACGCCACACACCATACACACATACATACACAC |
| | | | ATCACACATACATATATACAAATACATATATACACAAA |
| | | | TACACACATACACACACATACTACACACACCACAAAC |
| | | | CATACACACATTCATACATACCCCCCACACACGTCTCT |
| | | | CAAGGATAAGTAAGTAATATTAAGGTACGGGAGGTAT |
| | | | TGGACAGGCCGCAATAAAATATCTTTATTTTCATTACA |
| | | | TCTGTGTGTTGGTTTTTTGTGTGAATCGATAGTACTAAC |
| | | | ATACGCTCTCCATCAAAACAAAACGAAACAAAACAAA |
| | | | CTAGCAAAATAGGCTGTCCCCAGTGCAAGTGCAGGTG |
| | | | CCAGAACATTTCTCTGGCCTAACTGGCCGGTACCTGAG |
| | | | CTCACGTCTCTCAAGGATAAGTAAGTAATATTAAGGTA |
| | | | CGGGAGGTATTGGACAGGCCGCAATAAAATATCTTTAT |
| | | | TTTCATTACATCTGTGTGTTGGTTTTTTGTGTGAATCGA |
| | | | TAGTACTAACATACGCTCTCCATCAAAACAAAACGAA |
| | | | ACAAAACAAACTAGCAAAATAGGCTGTCCCCAGTGCA |
| | | | AGTGCAGGTGCCAGAACATTTCTCTGGCCTAACTGGCC |
| | | | GGTACCTGAGCTCACGTCTCTCAAGGATAAGTAAGTAA |
| | | | TCCAATGATGGCGCCTGCTGAATTCTGTACGGGCCAGA |
| | | | TATACGCGTTTCCCATAAAACTCTAGCCTCCCAGAGGG |
| | | | ACCCCAGCCCCCACCCTCCCGCCCACGAACCCCTGCAT |
| | | | TTCCAGAATCAGCCCCAGGGCCCCAACCCCCCCAAGCC |
| | | | CCCATTTCACAACACGCTGGCGCTACAGGCGCGTGACT |
| | | | TCCCCTTGCTTTGGGGCGGGGGGCTGAGACTCCTATGT |
| | | | GCTCCGGATTGGTCAGGCACGGCCTTCGGCCCCGCCTC |
| | | | CTGCCACCGCAGATTGGCCGCTAGCCCTCCCCGAGCGC |
| | | | CCTGCCTCCGAGGGCCGGCGCACTATAAAAGCCCCTTC |
| | | | ACCAGGAGAAGCCGTCACACAGATCCACAAGCTCGAG |
| | | | CCGAGTGGTCGTGGGTCTGTCTCTTGCTTCAACAGTGT |
| | | | TTGGACGGAACAGATCCGGGGACTCTCTTCCAGCCTCC |
| | | | GACCGCCCTCCGATTTCCTCTCCGCTTGCAACCTCCGG |
| | | | GACCATCTTCTCGGCCATCTCCTGCTTCTGGGACCTGC |
| | | | CAGCACCGTTTTTGTGGTTAGCTCCTTCTTGCCAACCA |
| | | | ACCGGATCCGCCACC |
| 241 | | EH38E1521375 | TCGAAACCAAATTGGGCGACATAGTGAGACCTGGTCT |
| | | | CTACAGCAAATACACACACACACACACACACACACTT |
| | | | ATGCACACATACACCATACATACACATACAGTTAGTCA |
| | | | CACATATACACACATACACACACCACACACACATACA |
| | | | CACATAGATACACAAATACATATACACCACACACATA |
| | | | CACACACGCCACACACCATACACACATACATACACAC |
| | | | ATCACACATACATATATACAAATACATATATACACAAA |
| | | | TACACACATACACACACATACTACACACACCACAAAC |
| | | | CATACACACATTCATACATACCCCCCACACACGTCTCT |
| | | | CAAGGATAAGTAAGTAATATTAAGGTACGGGAGGTAT |
| | | | TGGACAGGCCGCAATAAAATATCTTTATTTTCATTACA |
| | | | TCTGTGTGTTGGTTTTTTGTGTGAATCGATAGTACTAAC |
| | | | ATACGCTCTCCATCAAAACAAAACGAAACAAAACAAA |
| | | | CTAGCAAAATAGGCTGTCCCCAGTGCAAGTGCAGGTG |
| | | | CCAGAACATTTCTCTGGCCTAACTGGCCGGTACCTGAG |
| | | | CTCACGTCTCTCAAGGATAAGTAAGTAATATTAAGGTA |
| | | | CGGGAGGTATTGGACAGGCCGCAATAAAATATCTTTAT |
| | | | TTTCATTACATCTGTGTGTTGGTTTTTTGTGTGAATCGA |
| | | | TAGTACTAACATACGCTCTCCATCAAAACAAAACGAA |
| | | | ACAAAACAAACTAGCAAAATAGGCTGTCCCCAGTGCA |
| | | | AGTGCAGGTGCCAGAACATTTCTCTGGCCTAACTGGCC |
| | | | GGTACCTGAGCTCACGTCTCTCAAGGATAAGTAAGTAA |
| | | | TCCAATGATGGCGCCTGCTGAATTCTGTACGGGCCAGA |
| | | | TATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 91 | L83 | FTL2_EH38E1 | AAAAGAACAAAATTGTTCAAAAGGTGATGTTTTAAAA |
| | | 761982 | TGTGATATCTACAGCTACATTTACGAAATCACAATCGA |
| | | | ATGTTTAAGGAAACAGCTGTATACTGCCTAAACACAA |
| | | | GTGAAAAGCATCATAGCTTACGTGATACCACTAAAAC |
| | | | CACAGACATCAGCTGAGATTGTCCACTACAGACTGCA |
| | | | GTACCATGTCAATATTGCACAAATTGGGACATCACGAC |
| | | | ATCAAGTAGTCATTCACAGCCAACCAATCACGCCACCC |
| | | | ACCATGCACAGCAAAATATATAACACACATTTCTTTCA |
| | | | ATTATTACACTGTCATAAACTCAAAATCCTTTTAGAAC |
| | | | AATTCTGTTCTTACGTCTCTCAAGGATAAGTAAGTAAT |
| | | | ATTAAGGTACGGGAGGTATTGGACAGGCCGCAATAAA |
| | | | ATATCTTTATTTTCATTACATCTGTGTGTTGGTTTTTTGT |
| | | | GTGAATCGATAGTACTAACATACGCTCTCCATCAAAAC |
| | | | AAAACGAAACAAAACAAACTAGCAAAATAGGCTGTCC |
| | | | CCAGTGCAAGTGCAGGTGCCAGAACATTTCTCTGGCCT |
| | | | AACTGGCCGGTACCTGAGCTCACGTCTCTCAAGGATAA |
| | | | GTAAGTAATATTAAGGTACGGGAGGTATTGGACAGGC |
| | | | CGCAATAAAATATCTTTATTTTCATTACATCTGTGTGTT |
| | | | GGTTTTTTGTGTGAATCGATAGTACTAACATACGCTCT |
| | | | CCATCAAAACAAAACGAAACAAAACAAACTAGCCAAT |
| | | | GATGGCGCCTGCTGAATTCTGTACGGGCCAGATATACG |
| | | | CGTTTCCCATAAAACTCTAGCCTCCCAGAGGGACCCCA |
| | | | GCCCCCACCCTCCCGCCCACGAACCCCTGCATTTCCAG |
| | | | AATCAGCCCCAGGGCCCCAACCCCCCCAAGCCCCCATT |
| | | | TCACAACACGCTGGCGCTACAGGCGCGTGACTTCCCCT |
| | | | TGCTTTGGGGCGGGGGGCTGAGACTCCTATGTGCTCCG |
| | | | GATTGGTCAGGCACGGCCTTCGGCCCCGCCTCCTGCCA |
| | | | CCGCAGATTGGCCGCTAGCCCTCCCCGAGCGCCCTGCC |
| | | | TCCGAGGGCCGGCGCACTATAAAAGCCCCTTCACCAG |
| | | | GAGAAGCCGTCACACAGATCCACAAGCTCGAGCCGAG |
| | | | TGGTCGTGGGTCTGTCTCTTGCTTCAACAGTGTTTGGA |
| | | | CGGAACAGATCCGGGGACTCTCTTCCAGCCTCCGACCG |
| | | | CCCTCCGATTTCCTCTCCGCTTGCAACCTCCGGGACCA |
| | | | TCTTCTCGGCCATCTCCTGCTTCTGGGACCTGCCAGCA |
| | | | CCGTTTTTGTGGTTAGCTCCTTCTTGCCAACCAACCGG |
| | | | ATCCGCCACC |
| 242 | | EH38E1761982 | AAAAGAACAAAATTGTTCAAAAGGTGATGTTTTAAAA |
| | | | TGTGATATCTACAGCTACATTTACGAAATCACAATCGA |
| | | | ATGTTTAAGGAAACAGCTGTATACTGCCTAAACACAA |
| | | | GTGAAAAGCATCATAGCTTACGTGATACCACTAAAAC |
| | | | CACAGACATCAGCTGAGATTGTCCACTACAGACTGCA |
| | | | GTACCATGTCAATATTGCACAAATTGGGACATCACGAC |
| | | | ATCAAGTAGTCATTCACAGCCAACCAATCACGCCACCC |
| | | | ACCATGCACAGCAAAATATATAACACACATTTCTTTCA |
| | | | ATTATTACACTGTCATAAACTCAAAATCCTTTTAGAAC |
| | | | AATTCTGTTCTTACGTCTCTCAAGGATAAGTAAGTAAT |
| | | | ATTAAGGTACGGGAGGTATTGGACAGGCCGCAATAAA |
| | | | ATATCTTTATTTTCATTACATCTGTGTGTTGGTTTTTTGT |
| | | | GTGAATCGATAGTACTAACATACGCTCTCCATCAAAAC |
| | | | AAAACGAAACAAAACAAACTAGCAAAATAGGCTGTCC |
| | | | CCAGTGCAAGTGCAGGTGCCAGAACATTTCTCTGGCCT |
| | | | AACTGGCCGGTACCTGAGCTCACGTCTCTCAAGGATAA |
| | | | GTAAGTAATATTAAGGTACGGGAGGTATTGGACAGGC |
| | | | CGCAATAAAATATCTTTATTTTCATTACATCTGTGTGTT |
| | | | GGTTTTTTGTGTGAATCGATAGTACTAACATACGCTCT |
| | | | CCATCAAAACAAAACGAAACAAAACAAACTAGCCAAT |
| | | | GATGGCGCCTGCTGAATTCTGTACGGGCCAGATATACG |
| | | | CGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 92 | L84 | FTL2_EH38E1 | AAATATGGATCAACAAAATACAGTATATCCATACCCTA |
| | | 841027 | AATATGGATCAACAAAATACAGTATATCCATACACTA |
| | | | AATATGAAAATACGGTATATCCATATAATAAATATGG |
| | | | GTCAACAAAATACGGTATATCCATACCCTAAATATGGA |
| | | | TCAACAAAATACGGTATATCCATACACTAAATATGGGT |
| | | | CAACAAAATACAGTATATCCATACAATAAATATGGGT |
| | | | CAACAAAATACGGTATATCCATACACTAAATATGGGTC |
| | | | AACAAAATACAGTATATCCATAAAATAAATAACGTCT |
| | | | CTCAAGGATAAGTAAGTAATATTAAGGTACGGGAGGT |
| | | | ATTGGACAGGCCGCAATAAAATATCTTTATTTTCATTA |
| | | | CATCTGTGTGTTGGTTTTTTGTGTGAATCGATAGTACTA |
| | | | ACATACGCTCTCCATCAAAACAAAACGAAACAAAACA |
| | | | AACTAGCAAAATAGGCTGTCCCCAGTGCAAGTGCAGG |
| | | | TGCCAGAACATTTCTCTGGCCTAACTGGCCGGTACCTG |
| | | | AGCTCACGTCTCTCAAGGATAAGTAAGTAATATTAAGG |
| | | | TACGGGAGGTATTGGACAGGCCGCAATAAAATATCTTT |
| | | | ATTTTCATTACATCTGTGTGTTGGTTTTTTGTGTGAATC |
| | | | GATAGTACTAACATACGCTCTCCATCAAAACAAAACG |
| | | | AAACAAAACAAACTAGCAAAATAGGCTGTCCCCAGTG |
| | | | CAAGTGCAGGTGCCAGAACATTTCTCTGGCCTAACTGG |
| | | | CCGGTACCTGAGCTCACGTCTCTCAAGGATAAGTAAGT |
| | | | AATCCAATGATGGCGCCTGCTGAATTCTGTACGGGCCA |
| | | | GATATACGCGTTTCCCATAAAACTCTAGCCTCCCAGAG |
| | | | GGACCCCAGCCCCCACCCTCCCGCCCACGAACCCCTGC |
| | | | ATTTCCAGAATCAGCCCCAGGGCCCCAACCCCCCCAAG |
| | | | CCCCCATTTCACAACACGCTGGCGCTACAGGCGCGTGA |
| | | | CTTCCCCTTGCTTTGGGGCGGGGGGCTGAGACTCCTAT |
| | | | GTGCTCCGGATTGGTCAGGCACGGCCTTCGGCCCCGCC |
| | | | TCCTGCCACCGCAGATTGGCCGCTAGCCCTCCCCGAGC |
| | | | GCCCTGCCTCCGAGGGCCGGCGCACTATAAAAGCCCCT |
| | | | TCACCAGGAGAAGCCGTCACACAGATCCACAAGCTCG |
| | | | AGCCGAGTGGTCGTGGGTCTGTCTCTTGCTTCAACAGT |
| | | | GTTTGGACGGAACAGATCCGGGGACTCTCTTCCAGCCT |
| | | | CCGACCGCCCTCCGATTTCCTCTCCGCTTGCAACCTCC |
| | | | GGGACCATCTTCTCGGCCATCTCCTGCTTCTGGGACCT |
| | | | GCCAGCACCGTTTTTGTGGTTAGCTCCTTCTTGCCAACC |
| | | | AACCGGATCCGCCACC |
| 243 | | EH38E1841027 | AAATATGGATCAACAAAATACAGTATATCCATACCCTA |
| | | | AATATGGATCAACAAAATACAGTATATCCATACACTA |
| | | | AATATGAAAATACGGTATATCCATATAATAAATATGG |
| | | | GTCAACAAAATACGGTATATCCATACCCTAAATATGGA |
| | | | TCAACAAAATACGGTATATCCATACACTAAATATGGGT |
| | | | CAACAAAATACAGTATATCCATACAATAAATATGGGT |
| | | | CAACAAAATACGGTATATCCATACACTAAATATGGGTC |
| | | | AACAAAATACAGTATATCCATAAAATAAATAACGTCT |
| | | | CTCAAGGATAAGTAAGTAATATTAAGGTACGGGAGGT |
| | | | ATTGGACAGGCCGCAATAAAATATCTTTATTTTCATTA |
| | | | CATCTGTGTGTTGGTTTTTTGTGTGAATCGATAGTACTA |
| | | | ACATACGCTCTCCATCAAAACAAAACGAAACAAAACA |
| | | | AACTAGCAAAATAGGCTGTCCCCAGTGCAAGTGCAGG |
| | | | TGCCAGAACATTTCTCTGGCCTAACTGGCCGGTACCTG |
| | | | AGCTCACGTCTCTCAAGGATAAGTAAGTAATATTAAGG |
| | | | TACGGGAGGTATTGGACAGGCCGCAATAAAATATCTTT |
| | | | ATTTTCATTACATCTGTGTGTTGGTTTTTTGTGTGAATC |
| | | | GATAGTACTAACATACGCTCTCCATCAAAACAAAACG |
| | | | AAACAAAACAAACTAGCAAAATAGGCTGTCCCCAGTG |
| | | | CAAGTGCAGGTGCCAGAACATTTCTCTGGCCTAACTGG |
| | | | CCGGTACCTGAGCTCACGTCTCTCAAGGATAAGTAAGT |
| | | | AATCCAATGATGGCGCCTGCTGAATTCTGTACGGGCCA |
| | | | GATATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 93 | L85 | FTL2_EH38E2 | GTGGTTGCTGTAACCAATGACCACAAACCTGGGGGTC |
| | | 144693 | ATGGAAAGAACACAAACTTTACACACACACACACACA |
| | | | CACACACACACAGGCACATACACACATGCACATATGC |
| | | | ATACAAAGACAGGCACACACGAGCATGCACACACACA |
| | | | AACACGTGCACATACACATGCGCACACACATGCACAC |
| | | | GCACATGCACACACACGCAGGTACACAAGCATGCAAA |
| | | | CACACATACACATACAAACGCGTGCATGCATACATGC |
| | | | ACACATGAGCACACAAACACATGCACACACTTGCACA |
| | | | CATGCACACAAAGGCATACACATGCAATGCAACGTCT |
| | | | CTCAAGGATAAGTAAGTAATATTAAGGTACGGGAGGT |
| | | | ATTGGACAGGCCGCAATAAAATATCTTTATTTTCATTA |
| | | | CATCTGTGTGTTGGTTTTTTGTGTGAATCGATAGTACTA |
| | | | ACATACGCTCTCCATCAAAACAAAACGAAACAAAACA |
| | | | AACTAGCAAAATAGGCTGTCCCCAGTGCAAGTGCAGG |
| | | | TGCCAGAACATTTCTCTGGCCTAACTGGCCGGTACCTG |
| | | | AGCTCACGTCTCTCAAGGATAAGTAAGTAATATTAAGG |
| | | | TACGGGAGGTATTGGACAGGCCGCAATAAAATATCTTT |
| | | | ATTTTCATTACATCTGTGTGTTGGTTTTTTGTGTGAATC |
| | | | GATAGTACTAACATACGCTCTCCATCAAAACAAAACG |
| | | | AAACAAAACAAACTAGCAAAATAGGCTGTCCCCAGTG |
| | | | CAAGTGCAGGTGCCAGAACATTTCTCTGGCCTAACTGG |
| | | | CCGGTACCTGAGCTCACGTCTCTCAAGGATAAGTAAGT |
| | | | AATCCAATGATGGCGCCTGCTGAATTCTGTACGGGCCA |
| | | | GATATACGCGTTTCCCATAAAACTCTAGCCTCCCAGAG |
| | | | GGACCCCAGCCCCCACCCTCCCGCCCACGAACCCCTGC |
| | | | ATTTCCAGAATCAGCCCCAGGGCCCCAACCCCCCCAAG |
| | | | CCCCCATTTCACAACACGCTGGCGCTACAGGCGCGTGA |
| | | | CTTCCCCTTGCTTTGGGGCGGGGGGCTGAGACTCCTAT |
| | | | GTGCTCCGGATTGGTCAGGCACGGCCTTCGGCCCCGCC |
| | | | TCCTGCCACCGCAGATTGGCCGCTAGCCCTCCCCGAGC |
| | | | GCCCTGCCTCCGAGGGCCGGCGCACTATAAAAGCCCCT |
| | | | TCACCAGGAGAAGCCGTCACACAGATCCACAAGCTCG |
| | | | AGCCGAGTGGTCGTGGGTCTGTCTCTTGCTTCAACAGT |
| | | | GTTTGGACGGAACAGATCCGGGGACTCTCTTCCAGCCT |
| | | | CCGACCGCCCTCCGATTTCCTCTCCGCTTGCAACCTCC |
| | | | GGGACCATCTTCTCGGCCATCTCCTGCTTCTGGGACCT |
| | | | GCCAGCACCGTTTTTGTGGTTAGCTCCTTCTTGCCAACC |
| | | | AACCGGATCCGCCACC |
| 244 | | EH38E2144693 | GTGGTTGCTGTAACCAATGACCACAAACCTGGGGGTC |
| | | | ATGGAAAGAACACAAACTTTACACACACACACACACA |
| | | | CACACACACACAGGCACATACACACATGCACATATGC |
| | | | ATACAAAGACAGGCACACACGAGCATGCACACACACA |
| | | | AACACGTGCACATACACATGCGCACACACATGCACAC |
| | | | GCACATGCACACACACGCAGGTACACAAGCATGCAAA |
| | | | CACACATACACATACAAACGCGTGCATGCATACATGC |
| | | | ACACATGAGCACACAAACACATGCACACACTTGCACA |
| | | | CATGCACACAAAGGCATACACATGCAATGCAACGTCT |
| | | | CTCAAGGATAAGTAAGTAATATTAAGGTACGGGAGGT |
| | | | ATTGGACAGGCCGCAATAAAATATCTTTATTTTCATTA |
| | | | CATCTGTGTGTTGGTTTTTTGTGTGAATCGATAGTACTA |
| | | | ACATACGCTCTCCATCAAAACAAAACGAAACAAAACA |
| | | | AACTAGCAAAATAGGCTGTCCCCAGTGCAAGTGCAGG |
| | | | TGCCAGAACATTTCTCTGGCCTAACTGGCCGGTACCTG |
| | | | AGCTCACGTCTCTCAAGGATAAGTAAGTAATATTAAGG |
| | | | TACGGGAGGTATTGGACAGGCCGCAATAAAATATCTTT |
| | | | ATTTTCATTACATCTGTGTGTTGGTTTTTTGTGTGAATC |
| | | | GATAGTACTAACATACGCTCTCCATCAAAACAAAACG |
| | | | AAACAAAACAAACTAGCAAAATAGGCTGTCCCCAGTG |
| | | | CAAGTGCAGGTGCCAGAACATTTCTCTGGCCTAACTGG |
| | | | CCGGTACCTGAGCTCACGTCTCTCAAGGATAAGTAAGT |
| | | | AATCCAATGATGGCGCCTGCTGAATTCTGTACGGGCCA |
| | | | GATATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 94 | L86 | FTL2_EH38E2 | ACACACCATGCACAAACCACACACACATGCAACACTC |
| | | 147276 | CACAGACATACACACAGATACACAGATACACCACGCC |
| | | | ACATAGACACACCATCACACCACACACACAAAACACA |
| | | | TCACATAGAGACACACACCACACACATACAAGATGCC |
| | | | ACATAGATACACCACACACAAACCACACACACACACC |
| | | | TCATAGACACACAAACCATGCACACTATGCACAAACC |
| | | | ACACACACGCATACATACCCACAACACACCACATACA |
| | | | CACCATGCATACCACACCCACACTCCACATAGACACAC |
| | | | AAACCACGCACACATACATACACACAACACACCATAT |
| | | | ACGTCTCTCAAGGATAAGTAAGTAATATTAAGGTACG |
| | | | GGAGGTATTGGACAGGCCGCAATAAAATATCTTTATTT |
| | | | TCATTACATCTGTGTGTTGGTTTTTTGTGTGAATCGATA |
| | | | GTACTAACATACGCTCTCCATCAAAACAAAACGAAAC |
| | | | AAAACAAACTAGCAAAATAGGCTGTCCCCAGTGCAAG |
| | | | TGCAGGTGCCAGAACATTTCTCTGGCCTAACTGGCCGG |
| | | | TACCTGAGCTCACGTCTCTCAAGGATAAGTAAGTAATA |
| | | | TTAAGGTACGGGAGGTATTGGACAGGCCGCAATAAAA |
| | | | TATCTTTATTTTCATTACATCTGTGTGTTGGTTTTTTGTG |
| | | | TGAATCGATAGTACTAACATACGCTCTCCATCAAAACA |
| | | | AAACGAAACAAAACAAACTAGCCAATGATGGCGCCTG |
| | | | CTGAATTCTGTACGGGCCAGATATACGCGTTTCCCATA |
| | | | AAACTCTAGCCTCCCAGAGGGACCCCAGCCCCCACCCT |
| | | | CCCGCCCACGAACCCCTGCATTTCCAGAATCAGCCCCA |
| | | | GGGCCCCAACCCCCCCAAGCCCCCATTTCACAACACGC |
| | | | TGGCGCTACAGGCGCGTGACTTCCCCTTGCTTTGGGGC |
| | | | GGGGGGCTGAGACTCCTATGTGCTCCGGATTGGTCAGG |
| 245 | | EH38E2147276 | CACGGCCTTCGGCCCCGCCTCCTGCCACCGCAGATTGG |
| | | | CCGCTAGCCCTCCCCGAGCGCCCTGCCTCCGAGGGCCG |
| | | | GCGCACTATAAAAGCCCCTTCACCAGGAGAAGCCGTC |
| | | | ACACAGATCCACAAGCTCGAGCCGAGTGGTCGTGGGT |
| | | | CTGTCTCTTGCTTCAACAGTGTTTGGACGGAACAGATC |
| | | | CGGGGACTCTCTTCCAGCCTCCGACCGCCCTCCGATTT |
| | | | CCTCTCCGCTTGCAACCTCCGGGACCATCTTCTCGGCC |
| | | | ATCTCCTGCTTCTGGGACCTGCCAGCACCGTTTTTGTG |
| | | | GTTAGCTCCTTCTTGCCAACCAACCGGATCCGCCACC |
| | | | ACACACCATGCACAAACCACACACACATGCAACACTC |
| | | | CACAGACATACACACAGATACACAGATACACCACGCC |
| | | | ACATAGACACACCATCACACCACACACACAAAACACA |
| | | | TCACATAGAGACACACACCACACACATACAAGATGCC |
| | | | ACATAGATACACCACACACAAACCACACACACACACC |
| | | | TCATAGACACACAAACCATGCACACTATGCACAAACC |
| | | | ACACACACGCATACATACCCACAACACACCACATACA |
| | | | CACCATGCATACCACACCCACACTCCACATAGACACAC |
| | | | AAACCACGCACACATACATACACACAACACACCATAT |
| | | | ACGTCTCTCAAGGATAAGTAAGTAATATTAAGGTACG |
| | | | GGAGGTATTGGACAGGCCGCAATAAAATATCTTTATTT |
| | | | TCATTACATCTGTGTGTTGGTTTTTTGTGTGAATCGATA |
| | | | GTACTAACATACGCTCTCCATCAAAACAAAACGAAAC |
| | | | AAAACAAACTAGCAAAATAGGCTGTCCCCAGTGCAAG |
| | | | TGCAGGTGCCAGAACATTTCTCTGGCCTAACTGGCCGG |
| | | | TACCTGAGCTCACGTCTCTCAAGGATAAGTAAGTAATA |
| | | | TTAAGGTACGGGAGGTATTGGACAGGCCGCAATAAAA |
| | | | TATCTTTATTTTCATTACATCTGTGTGTTGGTTTTTTGTG |
| | | | TGAATCGATAGTACTAACATACGCTCTCCATCAAAACA |
| | | | AAACGAAACAAAACAAACTAGCCAATGATGGCGCCTG |
| | | | CTGAATTCTGTACGGGCCAGATATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 95 | L87 | FTL2_EH38E2 | ACAAATGCACACACACAGATGCAGGGTACACACGAAT |
| | | 274269 | GCACACACAGCACACACAGATGCACAGTATACACGAA |
| | | | TGCACAGACAGCACACACATAGATGCAGGGTACACAT |
| | | | GAATACACACAGCACACACAGATGCACAGTACACATG |
| | | | AATGCACACACAGATGCATGGTACACACGGATGCACA |
| | | | GACAGCACACACACAGATGCAGGGTACACATGAATGC |
| | | | ACACACAGCACACACACAGATGCACAGTACACATGAA |
| | | | TGCACACACAGCACACAGATGCACAGTACACACGAAT |
| 246 | | EH38E2274269 | GCACACACAGCACATACACACATGCAGGGTACACACG |
| | | | AATGCAAAGCACGTCTCTCAAGGATAAGTAAGTAATA |
| | | | TTAAGGTACGGGAGGTATTGGACAGGCCGCAATAAAA |
| | | | TATCTTTATTTTCATTACATCTGTGTGTTGGTTTTTTGTG |
| | | | TGAATCGATAGTACTAACATACGCTCTCCATCAAAACA |
| | | | AAACGAAACAAAACAAACTAGCAAAATAGGCTGTCCC |
| | | | CAGTGCAAGTGCAGGTGCCAGAACATTTCTCTGGCCTA |
| | | | ACTGGCCGGTACCTGAGCTCACGTCTCTCAAGGATAAG |
| | | | TAAGTAATATTAAGGTACGGGAGGTATTGGACAGGCC |
| | | | GCAATAAAATATCTTTATTTTCATTACATCTGTGTGTTG |
| | | | GTTTTTTGTGTGAATCGATAGTACTAACATACGCTCTC |
| | | | CATCAAAACAAAACGAAACAAAACAAACTAGCAAAAT |
| | | | AGGCTGTCCCCAGTGCAAGTGCAGGTGCCAGAACATTT |
| | | | CTCTGGCCTAACTGGCCGGTACCTGAGCTCACGTCTCT |
| | | | CAAGGATAAGTAAGTAATCCAATGATGGCGCCTGCTG |
| | | | AATTCTGTACGGGCCAGATATACGCGTTTCCCATAAAA |
| | | | CTCTAGCCTCCCAGAGGGACCCCAGCCCCCACCCTCCC |
| | | | GCCCACGAACCCCTGCATTTCCAGAATCAGCCCCAGGG |
| | | | CCCCAACCCCCCCAAGCCCCCATTTCACAACACGCTGG |
| | | | CGCTACAGGCGCGTGACTTCCCCTTGCTTTGGGGCGGG |
| | | | GGGCTGAGACTCCTATGTGCTCCGGATTGGTCAGGCAC |
| | | | GGCCTTCGGCCCCGCCTCCTGCCACCGCAGATTGGCCG |
| | | | CTAGCCCTCCCCGAGCGCCCTGCCTCCGAGGGCCGGCG |
| | | | CACTATAAAAGCCCCTTCACCAGGAGAAGCCGTCACA |
| | | | CAGATCCACAAGCTCGAGCCGAGTGGTCGTGGGTCTGT |
| | | | CTCTTGCTTCAACAGTGTTTGGACGGAACAGATCCGGG |
| | | | GACTCTCTTCCAGCCTCCGACCGCCCTCCGATTTCCTCT |
| | | | CCGCTTGCAACCTCCGGGACCATCTTCTCGGCCATCTC |
| | | | CTGCTTCTGGGACCTGCCAGCACCGTTTTTGTGGTTAG |
| | | | CTCCTTCTTGCCAACCAACCGGATCCGCCACC |
| | | | ACAAATGCACACACACAGATGCAGGGTACACACGAAT |
| | | | GCACACACAGCACACACAGATGCACAGTATACACGAA |
| | | | TGCACAGACAGCACACACATAGATGCAGGGTACACAT |
| | | | GAATACACACAGCACACACAGATGCACAGTACACATG |
| | | | AATGCACACACAGATGCATGGTACACACGGATGCACA |
| | | | GACAGCACACACACAGATGCAGGGTACACATGAATGC |
| | | | ACACACAGCACACACACAGATGCACAGTACACATGAA |
| | | | TGCACACACAGCACACAGATGCACAGTACACACGAAT |
| | | | GCACACACAGCACATACACACATGCAGGGTACACACG |
| | | | AATGCAAAGCACGTCTCTCAAGGATAAGTAAGTAATA |
| | | | TTAAGGTACGGGAGGTATTGGACAGGCCGCAATAAAA |
| | | | TATCTTTATTTTCATTACATCTGTGTGTTGGTTTTTTGTG |
| | | | TGAATCGATAGTACTAACATACGCTCTCCATCAAAACA |
| | | | AAACGAAACAAAACAAACTAGCAAAATAGGCTGTCCC |
| | | | CAGTGCAAGTGCAGGTGCCAGAACATTTCTCTGGCCTA |
| | | | ACTGGCCGGTACCTGAGCTCACGTCTCTCAAGGATAAG |
| | | | TAAGTAATATTAAGGTACGGGAGGTATTGGACAGGCC |
| | | | GCAATAAAATATCTTTATTTTCATTACATCTGTGTGTTG |
| | | | GTTTTTTGTGTGAATCGATAGTACTAACATACGCTCTC |
| | | | CATCAAAACAAAACGAAACAAAACAAACTAGCAAAAT |
| | | | AGGCTGTCCCCAGTGCAAGTGCAGGTGCCAGAACATTT |
| | | | CTCTGGCCTAACTGGCCGGTACCTGAGCTCACGTCTCT |
| | | | CAAGGATAAGTAAGTAATCCAATGATGGCGCCTGCTG |
| | | | AATTCTGTACGGGCCAGATATACGCGTT |
| 248 | L88 | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 96 | | FTL2_EH38E2 | CACACAAATACACACACACACAAATACGCACACACAA |
| | | 724199 | ATACAGAAACACACACACATACACAAATACACACACA |
| | | | CACAAATACGCACACACACAAATACAGAAACACACAC |
| | | | ATACACAAATACACAAATACAAAAATACGCACAAATA |
| | | | CACAATACACACACAAATACACATAAATACACAAATA |
| | | | CGCACACACAAATACATATAAATACACACAAATACAC |
| | | | ACAAATACACACAGAAATACACACGCACACGCACACA |
| | | | CACAAAACACATACACATACACACGAATACACACAAA |
| | | | TACACACACAAATACACATACACGCACACACATGCAC |
| | | | AACGTCTCTCAAGGATAAGTAAGTAATATTAAGGTAC |
| | | | GGGAGGTATTGGACAGGCCGCAATAAAATATCTTTATT |
| | | | TTCATTACATCTGTGTGTTGGTTTTTTGTGTGAATCGAT |
| | | | AGTACTAACATACGCTCTCCATCAAAACAAAACGAAA |
| | | | CAAAACAAACTAGCAAAATAGGCTGTCCCCAGTGCAA |
| | | | GTGCAGGTGCCAGAACATTTCTCTGGCCTAACTGGCCG |
| | | | GTACCTGAGCTCACGTCTCTCAAGGATAAGTAAGTAAT |
| | | | ATTAAGGTACGGGAGGTATTGGACAGGCCGCAATAAA |
| | | | ATATCTTTATTTTCATTACATCTGTGTGTTGGTTTTTTGT |
| | | | GTGAATCGATAGTACTAACATACGCTCTCCATCAAAAC |
| | | | AAAACGAAACAAAACAAACTAGCCAATGATGGCGCCT |
| | | | GCTGAATTCTGTACGGGCCAGATATACGCGTTTCCCAT |
| | | | AAAACTCTAGCCTCCCAGAGGGACCCCAGCCCCCACC |
| | | | CTCCCGCCCACGAACCCCTGCATTTCCAGAATCAGCCC |
| | | | CAGGGCCCCAACCCCCCCAAGCCCCCATTTCACAACAC |
| | | | GCTGGCGCTACAGGCGCGTGACTTCCCCTTGCTTTGGG |
| | | | GCGGGGGGCTGAGACTCCTATGTGCTCCGGATTGGTCA |
| | | | GGCACGGCCTTCGGCCCCGCCTCCTGCCACCGCAGATT |
| | | | GGCCGCTAGCCCTCCCCGAGCGCCCTGCCTCCGAGGGC |
| | | | CGGCGCACTATAAAAGCCCCTTCACCAGGAGAAGCCG |
| | | | TCACACAGATCCACAAGCTCGAGCCGAGTGGTCGTGG |
| | | | GTCTGTCTCTTGCTTCAACAGTGTTTGGACGGAACAGA |
| | | | TCCGGGGACTCTCTTCCAGCCTCCGACCGCCCTCCGAT |
| | | | TTCCTCTCCGCTTGCAACCTCCGGGACCATCTTCTCGGC |
| | | | CATCTCCTGCTTCTGGGACCTGCCAGCACCGTTTTTGTG |
| | | | GTTAGCTCCTTCTTGCCAACCAACCGGATCCGCCACC |
| 247 | | EH38E272419 | CACACAAATACACACACACACAAATACGCACACACAA |
| | | 9 | ATACAGAAACACACACACATACACAAATACACACACA |
| | | | CACAAATACGCACACACACAAATACAGAAACACACAC |
| | | | ATACACAAATACACAAATACAAAAATACGCACAAATA |
| | | | CACAATACACACACAAATACACATAAATACACAAATA |
| | | | CGCACACACAAATACATATAAATACACACAAATACAC |
| | | | ACAAATACACACAGAAATACACACGCACACGCACACA |
| | | | CACAAAACACATACACATACACACGAATACACACAAA |
| | | | TACACACACAAATACACATACACGCACACACATGCAC |
| | | | AACGTCTCTCAAGGATAAGTAAGTAATATTAAGGTAC |
| | | | GGGAGGTATTGGACAGGCCGCAATAAAATATCTTTATT |
| | | | TTCATTACATCTGTGTGTTGGTTTTTTGTGTGAATCGAT |
| | | | AGTACTAACATACGCTCTCCATCAAAACAAAACGAAA |
| | | | CAAAACAAACTAGCAAAATAGGCTGTCCCCAGTGCAA |
| | | | GTGCAGGTGCCAGAACATTTCTCTGGCCTAACTGGCCG |
| | | | GTACCTGAGCTCACGTCTCTCAAGGATAAGTAAGTAAT |
| | | | ATTAAGGTACGGGAGGTATTGGACAGGCCGCAATAAA |
| | | | ATATCTTTATTTTCATTACATCTGTGTGTTGGTTTTTTGT |
| | | | GTGAATCGATAGTACTAACATACGCTCTCCATCAAAAC |
| | | | AAAACGAAACAAAACAAACTAGCCAATGATGGCGCCT |
| | | | GCTGAATTCTGTACGGGCCAGATATACGCGTT |
| 248 | | FTLmd | TCCCATAAAACTCTAGCCTCCCAGAGGGACCCCAGCCC |
| | | | CCACCCTCCCGCCCACGAACCCCTGCATTTCCAGAATC |
| | | | AGCCCCAGGGCCCCAACCCCCCCAAGCCCCCATTTCAC |
| | | | AACACGCTGGCGCTACAGGCGCGTGACTTCCCCTTGCT |
| | | | TTGGGGCGGGGGGCTGAGACTCCTATGTGCTCCGGATT |
| | | | GGTCAGGCACGGCCTTCGGCCCCGCCTCCTGCCACCGC |
| | | | AGATTGGCCGCTAGCCCTCCCCGAGCGCCCTGCCTCCG |
| | | | AGGGCCGGCGCACTATAAAAGCCCCTTCACCAGGAGA |
| | | | AGCCGTCACACAGATCCACAAGCTCGAGCCGAGTGGT |
| | | | CGTGGGTCTGTCTCTTGCTTCAACAGTGTTTGGACGGA |
| | | | ACAGATCCGGGGACTCTCTTCCAGCCTCCGACCGCCCT |
| | | | CCGATTTCCTCTCCGCTTGCAACCTCCGGGACCATCTTC |
| | | | TCGGCCATCTCCTGCTTCTGGGACCTGCCAGCACCGTT |
| | | | TTTGTGGTTAGCTCCTTCTTGCCAACCAACC |
| 97 | L89 | SERPINAe- | GAGTTGCTGGTGCTTCCCCAGGCTGGAGATTGAGTTAA |
| | | SynE-HCBmd- | TATTAACAGGCCCAAGGCGATGTGGGCTTGTGCAATCA |
| | | MVMi | TAGGCCCGGCCTCGCCAGGTCACCTGAGGAGTTAATG |
| | | | AATACATATCTCCTGAGTTAATAATTACCAGCGCGGGC |
| | | | CAAATAAATAATCCGCGAGGGGCAGCTGGACTTTGGA |
| | | | CTCCGCGCGCTGGTAATTATTAACCTCGCGAATATTGA |
| | | | TTCGAGGCCGCGATTGCCGCAATCGCGAGGGGCAGGT |
| | | | GACCTTTGCCCAGCGCGCGTTCGCCCCGCCCCGGACGG |
| | | | TATCGATAAGCTTAGGAGCTTGGGCTGCAGGTCGAGG |
| | | | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTGATCCACA |
| | | | AGCTCGAGCCGAGTGGTCGTTGAGCCAAGAGGTAAGG |
| | | | GTTTAAGGGATGGTTGGTTGGTGGGGTATTAATGTTTA |
| | | | ATTACCTGGAGCACCTGCCTGAAATCACTTTTTTTCAG |
| | | | GTTGGGGATCCGCCACC |
| 168 | | SERPINA1 | GAGTTGCTGGTGCTTCCCCAGGCTGGAGATTGAGTTAA |
| | | cre 1 | TATTAACAGGCCCAAGGCGATGTGGGCTTGTGCAATCA |
| | | | TAGGCCCGGCC |
| 169 | | SERPINA1 | TCGCCAGGTCACCTGAGGAGTTAATGAATACATATCTC |
| | | cre2 | CT |
| 197 | | SynEnh2 | GAGTTAATAATTACCAGCGCGGGCCAAATAAATAATC |
| | | | CGCGAGGGGCAGCTGGACTTTGGACTCCGCGCGCTGG |
| | | | TAATTATTAACCTCGCGAATATTGATTCGAGGCCGCGA |
| | | | TTGCCGCAATCGCGAGGGGCAGGTGACCTTTGCCCAGC |
| | | | GCGCGTTCGCCCCGCCCCGGACGGTATCGATAAGCTTA |
| | | | GGAGCTTGGGCTGCAGGTCGAG |
| 206 | | HCBmd | GTTAATCATTAAGTCGTTAATTTTTGTGGCCCTTGCGAT |
| | | | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTGATCCACA |
| | | | AGCTCGAGCCGAGTGGTCGT |
| 155 | | MVM | AAGAGGTAAGGGTTTAAGGGATGGTTGGTTGGTGGGG |
| | | intron | TATTAATGTTTAATTACCTGGAGCACCTGCCTGAAATC |
| | | | ACTTTTTTTCAGGTTGG |
| 98 | L90 | L57_best_ | TCCGGTAAAACGCTAGCCGCCGAAAATTGCGCAAGCC |
| | | mutated | CCCACCCTACCGCCTACGAAAGGAGCCGTTTCGGGAAT |
| | | | CCGCGGGGCCGCGGGGACCCCCCCAAGCCGCCATTAC |
| | | | GCGAAACGACCACGGGAACCTCCCGCTATGTCGGGTTT |
| | | | CTTCCCGGCGGCGGGCCGAGAGGAGGTCTTGTGCCGA |
| | | | AATCGTACGTCCCGGCATTCGGCGCGGCGTTGGTCCAC |
| | | | GGACATATCGCCGAAAGCCCTTCCCGAACGGCCGTCTG |
| | | | CCGAGGGCCGGCGAAAGACAAAAGCCGCTTTGGGGCA |
| | | | AAAAGCCGTTACGCCATACGGAAAGCTCGGGCCGAAC |
| | | | CGTCGTCGGTCGGTTGTGCTCTTTGAAAGTGTTTTGCC |
| | | | GGAAGAGTTTCGGGGACTCTCTTCGGGCGTCCGGCCGG |
| | | | CTTCCGTTTTCGGTTCCGCTTGCGACTGTCGGAACGAT |
| | | | CTTTTCGGCCGTTTTCTGTTTCCGGGACGGGCGGGTGC |
| | | | CGTTTTCGCCGTTAGCGCCTTCTTGCCAACCAACCGGA |
| | | | TCCGCCACC |
| 99 | L91 | LTF7-JeTmd | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATTCCTGACGTCCCAA |
| | | | TGATGGCGCCTGCTGAATTCGGGCGGAGTTAGGGCGG |
| | | | AGCCAATCAGCGTGCGCCGTTCCGAAAGTTGCCTTTTA |
| | | | TGGCTGGGCGGAGAATGGGCGGTGAACGCCGATGATT |
| | | | ATATAAGGACGCGCCGGGTGTGGCACAGCTAGTTCCG |
| | | | TCGCAGCCGGCTCGAGCCGAGTGGTCGTCTTGTTTGTG |
| | | | GATCCCTGTGATCGTCACTTGACAGGATCCGCCACC |
| 207 | | LTF7 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATT |
| 208 | | JeTmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 100 | L92 | LTF11-JeTmd | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAATCCTGACGTCCCAATGATGGCGC |
| | | | CTGCTGAATTCGGGCGGAGTTAGGGCGGAGCCAATCA |
| | | | GCGTGCGCCGTTCCGAAAGTTGCCTTTTATGGCTGGGC |
| | | | GGAGAATGGGCGGTGAACGCCGATGATTATATAAGGA |
| | | | CGCGCCGGGTGTGGCACAGCTAGTTCCGTCGCAGCCG |
| | | | GCTCGAGCCGAGTGGTCGTCTTGTTTGTGGATCCCTGT |
| | | | GATCGTCACTTGACA |
| 209 | | LTF11 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAAT |
| 208 | | JeTmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 101 | L93 | LTF22-JeTmd | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAACCCTGACGTCCCAATGATGGCGCCTGCTGAA |
| | | | TTCGGGCGGAGTTAGGGCGGAGCCAATCAGCGTGCGC |
| | | | CGTTCCGAAAGTTGCCTTTTATGGCTGGGCGGAGAATG |
| | | | GGCGGTGAACGCCGATGATTATATAAGGACGCGCCGG |
| | | | GTGTGGCACAGCTAGTTCCGTCGCAGCCGGCTCGAGCC |
| | | | GAGTGGTCGTCTTGTTTGTGGATCCCTGTGATCGTCAC |
| | | | TTGACA |
| 210 | | LTF22 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAAC |
| 208 | | Je Tmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 102 | L94 | LTF29-JeTmd | CAGTTTCATTTCCAATTGATGAGTCACCAATTTGACGT |
| | | | CAAATTATGATTGGTCCCCTGACGTCCCAATGATGGCG |
| | | | CCTGCTGAATTCGGGCGGAGTTAGGGCGGAGCCAATC |
| | | | AGCGTGCGCCGTTCCGAAAGTTGCCTTTTATGGCTGGG |
| | | | CGGAGAATGGGCGGTGAACGCCGATGATTATATAAGG |
| | | | ACGCGCCGGGTGTGGCACAGCTAGTTCCGTCGCAGCC |
| | | | GGCTCGAGCCGAGTGGTCGTCTTGTTTGTGGATCCCTG |
| | | | TGATCGTCACTTGACA |
| 211 | | LTF29 | CAGTTTCATTTCCAATTGATGAGTCACCAATTTGACGT |
| | | | CAAATTATGATTGGTCC |
| 208 | | Je Tmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 103 | L95 | LTF31-JeTmd | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTGCCTGACGTCCCAATGATGGCGCCT |
| | | | GCTGAATTCGGGCGGAGTTAGGGCGGAGCCAATCAGC |
| | | | GTGCGCCGTTCCGAAAGTTGCCTTTTATGGCTGGGCGG |
| | | | AGAATGGGCGGTGAACGCCGATGATTATATAAGGACG |
| | | | CGCCGGGTGTGGCACAGCTAGTTCCGTCGCAGCCGGCT |
| | | | CGAGCCGAGTGGTCGTCTTGTTTGTGGATCCCTGTGAT |
| | | | CGTCACTTGACA |
| 212 | | LTF31 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTG |
| 208 | | Je Tmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 104 | L96 | LTF32-JeTmd | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAATCCTGACGTCCCAATGATGGCG |
| | | | CCTGCTGAATTCGGGCGGAGTTAGGGCGGAGCCAATC |
| | | | AGCGTGCGCCGTTCCGAAAGTTGCCTTTTATGGCTGGG |
| | | | CGGAGAATGGGCGGTGAACGCCGATGATTATATAAGG |
| | | | ACGCGCCGGGTGTGGCACAGCTAGTTCCGTCGCAGCC |
| | | | GGCTCGAGCCGAGTGGTCGTCTTGTTTGTGGATCCCTG |
| | | | TGATCGTCACTTGACA |
| 213 | | LTF32 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAAT |
| 208 | | Je Tmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 105 | L97 | LTF35-JeTmd | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATTCCTGACGTC |
| | | | CCAATGATGGCGCCTGCTGAATTCGGGCGGAGTTAGG |
| | | | GCGGAGCCAATCAGCGTGCGCCGTTCCGAAAGTTGCCT |
| | | | TTTATGGCTGGGCGGAGAATGGGCGGTGAACGCCGAT |
| | | | GATTATATAAGGACGCGCCGGGTGTGGCACAGCTAGT |
| | | | TCCGTCGCAGCCGGCTCGAGCCGAGTGGTCGTCTTGTT |
| | | | TGTGGATCCCTGTGATCGTCACTTGACA |
| 214 | | LTF35 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATT |
| 208 | | JeTmd | GAATTCGGGCGGAGTTAGGGCGGAGCCAATCAGCGTG |
| | | | CGCCGTTCCGAAAGTTGCCTTTTATGGCTGGGCGGAGA |
| | | | ATGGGCGGTGAACGCCGATGATTATATAAGGACGCGC |
| | | | CGGGTGTGGCACAGCTAGTTCCGTCGCAGCCGGCTCGA |
| | | | GCCGAGTGGTCGTCTTGTTTGTGGATCCCTGTGATCGT |
| | | | CACTTGACAGGATCCGCCACC |
| 160 | | MTE/DPE2 | CTCGAGCCGAGTGGTCGT |
| 106 | L98 | LTF7-LP1 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATTCCCTAAAATGGGC |
| | | | AAACATTGCAAGCAGCAAACAGCAAACACACAGCCCT |
| | | | CCCTGCCTGCTGACCTTGGAGCTGGGGCAGAGGTCAG |
| | | | AGACCTCTCTGGGCCCATGCCACCTCCAACATCCACTC |
| | | | GACCCCTTGGAATTTCGGTGGAGAGGAGCAGAGGTTG |
| | | | TCCTGGCGTGGTTTAGGTAGTGTGAGAGGGGAATGACT |
| | | | CCTTTCGGTAAGTGCAGTGGAAGCTGTACACTGCCCAG |
| | | | GCAAAGCGTCCGGGCAGCGTAGGCGGGCGACTCAGAT |
| | | | CCCAGCCAGTGGACTTAGCCCCTGTTTGCTCCTCCGAT |
| | | | AACTGGGGTGACCTTGGTTAATATTCACCAGCAGCCTC |
| | | | CCCCGTTGCCCCTCTGGATCCACTGCTTAAATACGGAC |
| | | | GAGGACAGGGCCCTGTCTCCTCAGCTTCAGGCACCACC |
| | | | ACTGACCTGGGACAGTGAATTCGCGGCCGCCACC |
| 207 | | LTF7 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATT |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 107 | L99 | LTF11-LP1 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAATCCCTAAAATGGGCAAACATTGC |
| | | | AAGCAGCAAACAGCAAACACACAGCCCTCCCTGCCTG |
| | | | CTGACCTTGGAGCTGGGGCAGAGGTCAGAGACCTCTCT |
| | | | GGGCCCATGCCACCTCCAACATCCACTCGACCCCTTGG |
| | | | AATTTCGGTGGAGAGGAGCAGAGGTTGTCCTGGCGTG |
| | | | GTTTAGGTAGTGTGAGAGGGGAATGACTCCTTTCGGTA |
| | | | AGTGCAGTGGAAGCTGTACACTGCCCAGGCAAAGCGT |
| | | | CCGGGCAGCGTAGGCGGGCGACTCAGATCCCAGCCAG |
| | | | TGGACTTAGCCCCTGTTTGCTCCTCCGATAACTGGGGT |
| | | | GACCTTGGTTAATATTCACCAGCAGCCTCCCCCGTTGC |
| | | | CCCTCTGGATCCACTGCTTAAATACGGACGAGGACAG |
| | | | GGCCCTGTCTCCTCAGCTTCAGGCACCACCACTGACCT |
| | | | GGGACAGTGAATTCGCGGCCGCCACC |
| 209 | | LTF11 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAAT |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 108 | L100 | LTF22-LP1 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAACCCCTAAAATGGGCAAACATTGCAAGCAGC |
| | | | AAACAGCAAACACACAGCCCTCCCTGCCTGCTGACCTT |
| | | | GGAGCTGGGGCAGAGGTCAGAGACCTCTCTGGGCCCA |
| | | | TGCCACCTCCAACATCCACTCGACCCCTTGGAATTTCG |
| | | | GTGGAGAGGAGCAGAGGTTGTCCTGGCGTGGTTTAGG |
| | | | TAGTGTGAGAGGGGAATGACTCCTTTCGGTAAGTGCA |
| | | | GTGGAAGCTGTACACTGCCCAGGCAAAGCGTCCGGGC |
| | | | AGCGTAGGCGGGCGACTCAGATCCCAGCCAGTGGACT |
| | | | TAGCCCCTGTTTGCTCCTCCGATAACTGGGGTGACCTT |
| | | | GGTTAATATTCACCAGCAGCCTCCCCCGTTGCCCCTCT |
| | | | GGATCCACTGCTTAAATACGGACGAGGACAGGGCCCT |
| | | | GTCTCCTCAGCTTCAGGCACCACCACTGACCTGGGACA |
| | | | GTGAATTCGCGGCCGCCACC |
| 210 | | LTF22 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAAC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 109 | L101 | LTF29-LP1 | CAGTTTCATTTCCAATTGATGAGTCACCAATTTGACGT |
| | | | CAAATTATGATTGGTCCCCCTAAAATGGGCAAACATTG |
| | | | CAAGCAGCAAACAGCAAACACACAGCCCTCCCTGCCT |
| | | | GCTGACCTTGGAGCTGGGGCAGAGGTCAGAGACCTCT |
| | | | CTGGGCCCATGCCACCTCCAACATCCACTCGACCCCTT |
| | | | GGAATTTCGGTGGAGAGGAGCAGAGGTTGTCCTGGCG |
| | | | TGGTTTAGGTAGTGTGAGAGGGGAATGACTCCTTTCGG |
| | | | TAAGTGCAGTGGAAGCTGTACACTGCCCAGGCAAAGC |
| | | | GTCCGGGCAGCGTAGGCGGGCGACTCAGATCCCAGCC |
| | | | AGTGGACTTAGCCCCTGTTTGCTCCTCCGATAACTGGG |
| | | | GTGACCTTGGTTAATATTCACCAGCAGCCTCCCCCGTT |
| | | | GCCCCTCTGGATCCACTGCTTAAATACGGACGAGGACA |
| | | | GGGCCCTGTCTCCTCAGCTTCAGGCACCACCACTGACC |
| | | | TGGGACAGTGAATTCGCGGCCGCCACC |
| 211 | | LTF29 | CAGTTTCATTTCCAATTGATGAGTCACCAATTTGACGT |
| | | | CAAATTATGATTGGTCC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 110 | L102 | LTF31-LP1 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTGCCCTAAAATGGGCAAACATTGCAA |
| | | | GCAGCAAACAGCAAACACACAGCCCTCCCTGCCTGCT |
| | | | GACCTTGGAGCTGGGGCAGAGGTCAGAGACCTCTCTG |
| | | | GGCCCATGCCACCTCCAACATCCACTCGACCCCTTGGA |
| | | | ATTTCGGTGGAGAGGAGCAGAGGTTGTCCTGGCGTGG |
| | | | TTTAGGTAGTGTGAGAGGGGAATGACTCCTTTCGGTAA |
| | | | GTGCAGTGGAAGCTGTACACTGCCCAGGCAAAGCGTC |
| | | | CGGGCAGCGTAGGCGGGCGACTCAGATCCCAGCCAGT |
| | | | GGACTTAGCCCCTGTTTGCTCCTCCGATAACTGGGGTG |
| | | | ACCTTGGTTAATATTCACCAGCAGCCTCCCCCGTTGCC |
| | | | CCTCTGGATCCACTGCTTAAATACGGACGAGGACAGG |
| | | | GCCCTGTCTCCTCAGCTTCAGGCACCACCACTGACCTG |
| | | | GGACAGTGAATTCGCGGCCGCCACC |
| 212 | | LTF31 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTG |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 111 | L103 | LTF32-LP1 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAATCCCTAAAATGGGCAAACATTG |
| | | | CAAGCAGCAAACAGCAAACACACAGCCCTCCCTGCCT |
| | | | GCTGACCTTGGAGCTGGGGCAGAGGTCAGAGACCTCT |
| | | | CTGGGCCCATGCCACCTCCAACATCCACTCGACCCCTT |
| | | | GGAATTTCGGTGGAGAGGAGCAGAGGTTGTCCTGGCG |
| | | | TGGTTTAGGTAGTGTGAGAGGGGAATGACTCCTTTCGG |
| | | | TAAGTGCAGTGGAAGCTGTACACTGCCCAGGCAAAGC |
| | | | GTCCGGGCAGCGTAGGCGGGCGACTCAGATCCCAGCC |
| | | | AGTGGACTTAGCCCCTGTTTGCTCCTCCGATAACTGGG |
| | | | GTGACCTTGGTTAATATTCACCAGCAGCCTCCCCCGTT |
| | | | GCCCCTCTGGATCCACTGCTTAAATACGGACGAGGACA |
| | | | GGGCCCTGTCTCCTCAGCTTCAGGCACCACCACTGACC |
| | | | TGGGACAGTGAATTCGCGGCCGCCACC |
| 213 | | LTF32 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAAT |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 112 | L104 | LTF35-LP1 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATTCCCTAAAAT |
| | | | GGGCAAACATTGCAAGCAGCAAACAGCAAACACACAG |
| | | | CCCTCCCTGCCTGCTGACCTTGGAGCTGGGGCAGAGGT |
| | | | CAGAGACCTCTCTGGGCCCATGCCACCTCCAACATCCA |
| | | | CTCGACCCCTTGGAATTTCGGTGGAGAGGAGCAGAGG |
| | | | TTGTCCTGGCGTGGTTTAGGTAGTGTGAGAGGGGAATG |
| | | | ACTCCTTTCGGTAAGTGCAGTGGAAGCTGTACACTGCC |
| | | | CAGGCAAAGCGTCCGGGCAGCGTAGGCGGGCGACTCA |
| | | | GATCCCAGCCAGTGGACTTAGCCCCTGTTTGCTCCTCC |
| | | | GATAACTGGGGTGACCTTGGTTAATATTCACCAGCAGC |
| | | | CTCCCCCGTTGCCCCTCTGGATCCACTGCTTAAATACG |
| | | | GACGAGGACAGGGCCCTGTCTCCTCAGCTTCAGGCACC |
| | | | ACCACTGACCTGGGACAGTGAATTCGCGGCCGCCACC |
| 214 | | LTF35 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATT |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 113 | L105 | LTF7-AATv3 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATTGATCTTGCTACCA |
| | | | GTGGAACAGCCACTAAGGATTCTGCAGTGAGAGCAGA |
| | | | GGGCCAGCTAAGTGGTACTCTCCCAGAGACTGTCTGAC |
| | | | TCACGCCACCCCCTCCACCTTGGACACAGGACGCTGTG |
| | | | GTTTCTGAGCCAGGTACAATGACTCCTTTCGGTAAGTG |
| | | | CAGTGGAAGCTGTACACTGCCCAGGCAAAGCGTCCGG |
| | | | GCAGCGTAGGCGGGCGACTCAGATCCCAGCCAGTGGA |
| | | | CTTAGCCCCTGTTTGCTCCTCCGATAACTGGGGTGACC |
| | | | TTGGTTAATATTCACCAGCAGCCTCCCCCGTTGCCCCT |
| | | | CTGGATCCACTGCTTATATAACCCAGGGGCACAGGGG |
| | | | CTGCCCTCAGTCCGCCTGGAGACCTCGAGCCGAGTGGT |
| | | | CGTCTCAGGAGCCGGGCAGGTTGGTATCAAGGTTACA |
| | | | AGACAGGTTTAAGGAGACCAATAGAAACTGGGCATGT |
| | | | GGAGACAGAGAAGACTCTTGGGTTTCTGATAGGCACT |
| | | | GACTCTCTCTGCCTATTGGTCTATTTTCCCACCCTTAGG |
| | | | CTGCTGCCACC |
| 207 | | LTF7 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATT |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 114 | L106 | LTF11-AATv3 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAATGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCCGGGCAGGTTGGTATCAAGGTTACAAGACAGGTTT |
| | | | AAGGAGACCAATAGAAACTGGGCATGTGGAGACAGAG |
| | | | AAGACTCTTGGGTTTCTGATAGGCACTGACTCTCTCTG |
| | | | CCTATTGGTCTATTTTCCCACCCTTAGGCTGCTGCCACC |
| 209 | | LTF11 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAAT |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 115 | L107 | LTF22-AATv3 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAACGATCTTGCTACCAGTGGAACAGCCACTAAG |
| | | | GATTCTGCAGTGAGAGCAGAGGGCCAGCTAAGTGGTA |
| | | | CTCTCCCAGAGACTGTCTGACTCACGCCACCCCCTCCA |
| | | | CCTTGGACACAGGACGCTGTGGTTTCTGAGCCAGGTAC |
| | | | AATGACTCCTTTCGGTAAGTGCAGTGGAAGCTGTACAC |
| | | | TGCCCAGGCAAAGCGTCCGGGCAGCGTAGGCGGGCGA |
| | | | CTCAGATCCCAGCCAGTGGACTTAGCCCCTGTTTGCTC |
| | | | CTCCGATAACTGGGGTGACCTTGGTTAATATTCACCAG |
| | | | CAGCCTCCCCCGTTGCCCCTCTGGATCCACTGCTTATAT |
| | | | AACCCAGGGGCACAGGGGCTGCCCTCAGTCCGCCTGG |
| | | | AGACCTCGAGCCGAGTGGTCGTCTCAGGAGCCGGGCA |
| | | | GGTTGGTATCAAGGTTACAAGACAGGTTTAAGGAGAC |
| | | | CAATAGAAACTGGGCATGTGGAGACAGAGAAGACTCT |
| | | | TGGGTTTCTGATAGGCACTGACTCTCTCTGCCTATTGGT |
| | | | CTATTTTCCCACCCTTAGGCTGCTGCCACC |
| 210 | | LTF22 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAAC |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 116 | L108 | LTF29-AATv3 | CAGTTTCATTTCCAATTGATGAGTCACCAATTTGACGT |
| | | | CAAATTATGATTGGTCCGATCTTGCTACCAGTGGAACA |
| | | | GCCACTAAGGATTCTGCAGTGAGAGCAGAGGGCCAGC |
| | | | TAAGTGGTACTCTCCCAGAGACTGTCTGACTCACGCCA |
| | | | CCCCCTCCACCTTGGACACAGGACGCTGTGGTTTCTGA |
| | | | GCCAGGTACAATGACTCCTTTCGGTAAGTGCAGTGGAA |
| | | | GCTGTACACTGCCCAGGCAAAGCGTCCGGGCAGCGTA |
| | | | GGCGGGCGACTCAGATCCCAGCCAGTGGACTTAGCCC |
| | | | CTGTTTGCTCCTCCGATAACTGGGGTGACCTTGGTTAA |
| | | | TATTCACCAGCAGCCTCCCCCGTTGCCCCTCTGGATCC |
| | | | ACTGCTTATATAACCCAGGGGCACAGGGGCTGCCCTCA |
| | | | GTCCGCCTGGAGACCTCGAGCCGAGTGGTCGTCTCAGG |
| | | | AGCCGGGCAGGTTGGTATCAAGGTTACAAGACAGGTT |
| | | | TAAGGAGACCAATAGAAACTGGGCATGTGGAGACAGA |
| | | | GAAGACTCTTGGGTTTCTGATAGGCACTGACTCTCTCT |
| | | | GCCTATTGGTCTATTTTCCCACCCTTAGGCTGCTGCCAC |
| | | | C |
| 211 | | LTF29 | CAGTTTCATTTCCAATTGATGAGTCACCAATTTGACGT |
| | | | CAAATTATGATTGGTCC |
| 215 | | AATv3 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 117 | L109 | LTF31-AATv3 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTGGATCTTGCTACCAGTGGAACAGCC |
| | | | ACTAAGGATTCTGCAGTGAGAGCAGAGGGCCAGCTAA |
| | | | GTGGTACTCTCCCAGAGACTGTCTGACTCACGCCACCC |
| | | | CCTCCACCTTGGACACAGGACGCTGTGGTTTCTGAGCC |
| | | | AGGTACAATGACTCCTTTCGGTAAGTGCAGTGGAAGCT |
| | | | GTACACTGCCCAGGCAAAGCGTCCGGGCAGCGTAGGC |
| | | | GGGCGACTCAGATCCCAGCCAGTGGACTTAGCCCCTGT |
| | | | TTGCTCCTCCGATAACTGGGGTGACCTTGGTTAATATT |
| | | | CACCAGCAGCCTCCCCCGTTGCCCCTCTGGATCCACTG |
| | | | CTTATATAACCCAGGGGCACAGGGGCTGCCCTCAGTCC |
| | | | GCCTGGAGACCTCGAGCCGAGTGGTCGTCTCAGGAGC |
| | | | CGGGCAGGTTGGTATCAAGGTTACAAGACAGGTTTAA |
| | | | GGAGACCAATAGAAACTGGGCATGTGGAGACAGAGAA |
| | | | GACTCTTGGGTTTCTGATAGGCACTGACTCTCTCTGCCT |
| | | | ATTGGTCTATTTTCCCACCCTTAGGCTGCTGCCACC |
| 212 | | LTF31 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTG |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 118 | L110 | LTF32-AATv3 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAATGATCTTGCTACCAGTGGAACA |
| | | | GCCACTAAGGATTCTGCAGTGAGAGCAGAGGGCCAGC |
| | | | TAAGTGGTACTCTCCCAGAGACTGTCTGACTCACGCCA |
| | | | CCCCCTCCACCTTGGACACAGGACGCTGTGGTTTCTGA |
| | | | GCCAGGTACAATGACTCCTTTCGGTAAGTGCAGTGGAA |
| | | | GCTGTACACTGCCCAGGCAAAGCGTCCGGGCAGCGTA |
| | | | GGCGGGCGACTCAGATCCCAGCCAGTGGACTTAGCCC |
| | | | CTGTTTGCTCCTCCGATAACTGGGGTGACCTTGGTTAA |
| | | | TATTCACCAGCAGCCTCCCCCGTTGCCCCTCTGGATCC |
| | | | ACTGCTTATATAACCCAGGGGCACAGGGGCTGCCCTCA |
| | | | GTCCGCCTGGAGACCTCGAGCCGAGTGGTCGTCTCAGG |
| | | | AGCCGGGCAGGTTGGTATCAAGGTTACAAGACAGGTT |
| | | | TAAGGAGACCAATAGAAACTGGGCATGTGGAGACAGA |
| | | | GAAGACTCTTGGGTTTCTGATAGGCACTGACTCTCTCT |
| | | | GCCTATTGGTCTATTTTCCCACCCTTAGGCTGCTGCCAC |
| | | | C |
| 213 | | LTF32 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAAT |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 119 | L111 | LTF35-AATv3 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATTGATCTTGCT |
| | | | ACCAGTGGAACAGCCACTAAGGATTCTGCAGTGAGAG |
| | | | CAGAGGGCCAGCTAAGTGGTACTCTCCCAGAGACTGT |
| | | | CTGACTCACGCCACCCCCTCCACCTTGGACACAGGACG |
| | | | CTGTGGTTTCTGAGCCAGGTACAATGACTCCTTTCGGT |
| | | | AAGTGCAGTGGAAGCTGTACACTGCCCAGGCAAAGCG |
| | | | TCCGGGCAGCGTAGGCGGGCGACTCAGATCCCAGCCA |
| | | | GTGGACTTAGCCCCTGTTTGCTCCTCCGATAACTGGGG |
| | | | TGACCTTGGTTAATATTCACCAGCAGCCTCCCCCGTTG |
| | | | CCCCTCTGGATCCACTGCTTATATAACCCAGGGGCACA |
| | | | GGGGCTGCCCTCAGTCCGCCTGGAGACCTCGAGCCGA |
| | | | GTGGTCGTCTCAGGAGCCGGGCAGGTTGGTATCAAGG |
| | | | TTACAAGACAGGTTTAAGGAGACCAATAGAAACTGGG |
| | | | CATGTGGAGACAGAGAAGACTCTTGGGTTTCTGATAG |
| | | | GCACTGACTCTCTCTGCCTATTGGTCTATTTTCCCACCC |
| | | | TTAGGCTGCTGCCACC |
| 214 | | LTF35 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATT |
| 215 | | AATv4 | GTCTTGTGTCTGCCGGGCAATGAGCAAGGCTCCTTCCT |
| | | promoter | GTCCAAGCTCCCCGCCCCTCCCCAGCCTACTGCCTCCA |
| | | | CCCGAAGTCTACTTCCTGGGTGGGCAGGAACTGGGCA |
| | | | CTGTGCCCAGGGCATGGATCTTGCTACCAGTGGAACAG |
| | | | CCACTAAGGATTCTGCAGTGAGAGCAGAGGGCCAGCT |
| | | | AAGTGGTACTCTCCCAGAGACTGTCTGACTCACGCCAC |
| | | | CCCCTCCACCTTGGACACAGGACGCTGTGGTTTCTGAG |
| | | | CCAGGTACAATGACTCCTTTCGGTAAGTGCAGTGGAAG |
| | | | CTGTACACTGCCCAGGCAAAGCGTCCGGGCAGCGTAG |
| | | | GCGGGCGACTCAGATCCCAGCCAGTGGACTTAGCCCCT |
| | | | GTTTGCTCCTCCGATAACTGGGGTGACCTTGGTTAATA |
| | | | TTCACCAGCAGCCTCCCCCGTTGCCCCTCTGGATCCAC |
| | | | TGCTTATATAACCCAGGGGCACAGGGGCTGCCCTCAGT |
| | | | CCGCCTGGAGACCTCGAGCCGAGTGGTCGTCTCAGGA |
| | | | GCC |
| 193 | | HBB intron | GGGCAGGTTGGTATCAAGGTTACAAGACAGGTTTAAG |
| | | | GAGACCAATAGAAACTGGGCATGTGGAGACAGAGAAG |
| | | | ACTCTTGGGTTTCTGATAGGCACTGACTCTCTCTGCCTA |
| | | | TTGGTCTATTTTCCCACCCTTAGGCTGCT |
| 120 | L112 | LTF7-HCB2 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATTGTTAATCATTAAG |
| | | | TCGTTAATTTTTGTGGCCCTTGCGATGTTTGCTCTGGTT |
| | | | AATAATCTCAGGACAAACAGAGGTTAATAATTTTCCAG |
| | | | ATCTCTCTGAGCAATAGTATAAAAGGCCAGCAGCAGC |
| | | | CTGACCACATCTCATCCTTGATCCACAAGCTCGAGCCG |
| | | | AGTGGTCGTGAGCCAAGAGGTAAGGGTTTAAGGGATG |
| | | | GTTGGTTGGTGGGGTATTAATGTTTAATTACCTGGAGC |
| | | | ACCTGCCTGAAATCACTTTTTTTCAGGTTGGGGATCCG |
| | | | CCACC |
| 207 | | LTF7 | AATTGAGTCCAAAGTCCAGAATTATTGTGCAATAAATT |
| | | | TAAGGAAGAGGAATTTGTTACGTAATAATTCGATGACC |
| | | | TTTGACCCCTGGAATTCTGCCCCGCCCCCACTAATTTGT |
| | | | TACGTAATAATTAATGCCACGTCATCAATTATTACGTA |
| | | | ACAAATTGTTAATGATTAACTAATT |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 121 | L113 | LTF11-HCB2 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAATGTTAATCATTAAGTCGTTAATTT |
| | | | TTGTGGCCCTTGCGATGTTTGCTCTGGTTAATAATCTCA |
| | | | GGACAAACAGAGGTTAATAATTTTCCAGATCTCTCTGA |
| | | | GCAATAGTATAAAAGGCCAGCAGCAGCCTGACCACAT |
| | | | CTCATCCTTGATCCACAAGCTCGAGCCGAGTGGTCGTG |
| | | | AGCCAAGAGGTAAGGGTTTAAGGGATGGTTGGTTGGT |
| | | | GGGGTATTAATGTTTAATTACCTGGAGCACCTGCCTGA |
| | | | AATCACTTTTTTTCAGGTTGGGGATCCGCCACC |
| 209 | | LTF11 | AATTAGTTAATCATTAACAATTATCACGTGACAATTTT |
| | | | TCCCAGAAGAATTCTTCTGGGAAAAATTGTTAATGATT |
| | | | AACAATTTGACGTCAAATTCTGATTGGTCCAATTGGTC |
| | | | ACATGACAATTAATGCCACGTCATCAATTATTACGTAA |
| | | | CAAATTATTACGTAACAAATTATCACGTGATAATTATG |
| | | | AGTCATCCAATTGGATGACTCATAATTGCCGGCGAATT |
| | | | CTGACGTCAAATTGTGACGTAAAATTATGAGTCATCCA |
| | | | ATTACTTCCGGTCAAT |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 123 | L114 | LTF22-HCB2 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAACGTTAATCATTAAGTCGTTAATTTTTGTGGCC |
| | | | CTTGCGATGTTTGCTCTGGTTAATAATCTCAGGACAAA |
| | | | CAGAGGTTAATAATTTTCCAGATCTCTCTGAGCAATAG |
| | | | TATAAAAGGCCAGCAGCAGCCTGACCACATCTCATCCT |
| | | | TGATCCACAAGCTCGAGCCGAGTGGTCGTGAGCCAAG |
| | | | AGGTAAGGGTTTAAGGGATGGTTGGTTGGTGGGGTATT |
| | | | AATGTTTAATTACCTGGAGCACCTGCCTGAAATCACTT |
| | | | TTTTTCAGGTTGGGGATCCGCCACC |
| 210 | | LTF22 | ATTTGAGTCATTTCAATTAATGAGTCACAAATTGAGTC |
| | | | CAAAGTCCAGAATTATGAGTCATCCAATTATTGCACAA |
| | | | TAATTCAAAGTAAACAAATTCTGATTGGTCCAATTGAC |
| | | | CGGAAGTAATTGATGACGTGGCATTAATTATCACGTGA |
| | | | CAATTACTTCCGGTCAATTGCATCCATAGAATTGTTAA |
| | | | TCATTAAC |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 124 | L115 | LTF29-HCB2 | CAGTTTCATTTCCAATTGATGAGTCACCAATTTGACGT |
| | | | CAAATTATGATTGGTCCGTTAATCATTAAGTCGTTAAT |
| | | | TTTTGTGGCCCTTGCGATGTTTGCTCTGGTTAATAATCT |
| | | | CAGGACAAACAGAGGTTAATAATTTTCCAGATCTCTCT |
| | | | GAGCAATAGTATAAAAGGCCAGCAGCAGCCTGACCAC |
| | | | ATCTCATCCTTGATCCACAAGCTCGAGCCGAGTGGTCG |
| | | | TGAGCCAAGAGGTAAGGGTTTAAGGGATGGTTGGTTG |
| | | | GTGGGGTATTAATGTTTAATTACCTGGAGCACCTGCCT |
| | | | GAAATCACTTTTTTTCAGGTTGGGGATCCGCCACC |
| 211 | | LTF29 | CAGTTTCATTTCCAATTGATGAGTCACCAATTTGACGT |
| | | | CAAATTATGATTGGTCC |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 125 | L116 | LTF31-HCB2 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTGGTTAATCATTAAGTCGTTAATTTTT |
| | | | GTGGCCCTTGCGATGTTTGCTCTGGTTAATAATCTCAG |
| | | | GACAAACAGAGGTTAATAATTTTCCAGATCTCTCTGAG |
| | | | CAATAGTATAAAAGGCCAGCAGCAGCCTGACCACATC |
| | | | TCATCCTTGATCCACAAGCTCGAGCCGAGTGGTCGTGA |
| | | | GCCAAGAGGTAAGGGTTTAAGGGATGGTTGGTTGGTG |
| | | | GGGTATTAATGTTTAATTACCTGGAGCACCTGCCTGAA |
| | | | ATCACTTTTTTTCAGGTTGGGGATCCGCCACC |
| 212 | | LTF31 | AGAGTCCAAAGTCCAAATTTGACGTCAGAATTCCAGG |
| | | | GGTCAAAGGTCATCGAATTAATGAGTCACAAATTGTTA |
| | | | ATGATTAAGGAAGAGGAATTAATGAGTCACAAATTGG |
| | | | AAATGAAACTGAACTTCCGGTCAATTGATGAGTCACCA |
| | | | ATTGATGAGTCACCAATTGGTCACATGACAATTGGTGA |
| | | | CTCATCAATTAGAGTCCAAAGTCCAAATTCTGATTGGT |
| | | | CCAATTATCACGTG |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 126 | L117 | LTF32-HCB2 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAATGTTAATCATTAAGTCGTTAAT |
| | | | TTTTGTGGCCCTTGCGATGTTTGCTCTGGTTAATAATCT |
| | | | CAGGACAAACAGAGGTTAATAATTTTCCAGATCTCTCT |
| | | | GAGCAATAGTATAAAAGGCCAGCAGCAGCCTGACCAC |
| | | | ATCTCATCCTTGATCCACAAGCTCGAGCCGAGTGGTCG |
| | | | TGAGCCAAGAGGTAAGGGTTTAAGGGATGGTTGGTTG |
| | | | GTGGGGTATTAATGTTTAATTACCTGGAGCACCTGCCT |
| | | | GAAATCACTTTTTTTCAGGTTGGGGATCCGCCACC |
| 213 | | LTF32 | AATTAGTTAATCATTAACAATTTGACGTCAAATTTTTC |
| | | | CCAGAAGAATTTGTGACTCATTAATTATTGTGCAATAA |
| | | | TTTATTGTGCAATAATTTGACGTCAGAATTAATGAGTC |
| | | | ACAAATTAATGCCACGTCATCAATTGTCACGTGATAAT |
| | | | TAATGAGTCACAAATTTGACGTCAGAATTGTCATGTGA |
| | | | CCAATTAGTTAATCATTAACAATTCCTTTTCCTTAAATT |
| | | | AGAGTCCAAAGTCCAAATTCCTCTTCCTTAAATTATTG |
| | | | CACAATAAATTGCATCCATAGAATTGTTAATCATTAAC |
| | | | AATTATTGCACAATAAT |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 127 | L118 | LTF35-HCB2 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATTGTTAATCAT |
| | | | TAAGTCGTTAATTTTTGTGGCCCTTGCGATGTTTGCTCT |
| | | | GGTTAATAATCTCAGGACAAACAGAGGTTAATAATTTT |
| | | | CCAGATCTCTCTGAGCAATAGTATAAAAGGCCAGCAG |
| | | | CAGCCTGACCACATCTCATCCTTGATCCACAAGCTCGA |
| | | | GCCGAGTGGTCGTGAGCCAAGAGGTAAGGGTTTAAGG |
| | | | GATGGTTGGTTGGTGGGGTATTAATGTTTAATTACCTG |
| | | | GAGCACCTGCCTGAAATCACTTTTTTTCAGGTTGGGGA |
| | | | TCCGCCACC |
| 214 | | LTF35 | CGTGATAATTCTGACGTCAAATTGTCACGTGATAATTG |
| | | | GTGACTCATCAATTGCCGGCGAATTATTGTGCAATAAA |
| | | | TTAATGCCACGTCATCAATTGTCACGTGATAATTTTAC |
| | | | GTCACAATTCAAAGTAAACAAATTATCACGTGATAATT |
| | | | CGATGACCTTTGACCCCTGGAATTCCTCTTCCTTAAATT |
| | | | CGCCGGCAATTGTTAATGATTAACTAATT |
| 205 | | HCB2 | GTTAATCATTAAGTCGTTAATTTTTGTGGCCCTTGCGAT |
| | | promoter | GTTTGCTCTGGTTAATAATCTCAGGACAAACAGAGGTT |
| | | | AATAATTTTCCAGATCTCTCTGAGCAATAGTATAAAAG |
| | | | GCCAGCAGCAGCCTGACCACATCTCATCCTTGATCCAC |
| | | | AAGCTCGAGCCGAGTGGTCGT |
| 155 | | MVM intron | AAGAGGTAAGGGTTTAAGGGATGGTTGGTTGGTGGGG |
| | | | TATTAATGTTTAATTACCTGGAGCACCTGCCTGAAATC |
| | | | ACTTTTTTTCAGGTTGG |
| 128 | L119 | LP1- | CCCTAAAATGGGCAAACATTGCAAGCAGCAAACAGCA |
| | | EEF1Ali | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGGGAGAGCGGGCGGGTGAGTCACCCACACAAAGG |
| | | | AAAAGGGCCTTTCCGTCCTCAGCCGTCGCTTCATGTGA |
| | | | CTCCACGGAGTACCGGGCGCCGTCCAGGCACCTCGATT |
| | | | AGTTCTCGAGCTTTTGGAGTACGTCGTCTTTAGGTTGG |
| | | | GGGGAGGGGTTTTATGCGATGGAGTTTCCCCACACTGA |
| | | | GTGGGTGGAGACTGAAGTTAGGCCAGCTTGGCACTTG |
| | | | ATGTAATTCTCCTTGGAATTTGCCCTTTTTGAGTTTGGA |
| | | | TCTTGGTTCATTCTCAAGCCTCAGACAGTGGTTCAAAG |
| | | | TTTTTTTCTTCCATTTCAGGTGTCGTGAAAACTAGCCAC |
| | | | C |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 216 | | EF1-alpha | CGGGAGAGCGGGCGGGTGAGTCACCCACACAAAGGAA |
| | | intron A | AAGGGCCTTTCCGTCCTCAGCCGTCGCTTCATGTGACT |
| | | inserted at | CCACGGAGTACCGGGCGCCGTCCAGGCACCTCGATTA |
| | | nucleotide 455 | GTTCTCGAGCTTTTGGAGTACGTCGTCTTTAGGTTGGG |
| | | of LP1 | GGGAGGGGTTTTATGCGATGGAGTTTCCCCACACTGAG |
| | | | TGGGTGGAGACTGAAGTTAGGCCAGCTTGGCACTTGAT |
| | | | GTAATTCTCCTTGGAATTTGCCCTTTTTGAGTTTGGATC |
| | | | TTGGTTCATTCTCAAGCCTCAGACAGTGGTTCAAAGTT |
| | | | TTTTTCTTCCATTTCAGGTGTCGTGAAAACTA |
| 129 | L120 | LP1- | CCCTAAAATGGGCAAACATTGCAAGCAGCAAACAGCA |
| | | EEF1B2i | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCTCGGGAGCCGTGGAGGTACGAACTTAAGACATGCC |
| | | | TATTTTATTAATTTACTTCCAAACGCAACGAAAGGTCC |
| | | | ATGGACAATTTGTGGGCCATTTAATTCAGGGCCCCCAA |
| | | | TTCGTACGTGGAGAAGTGGGAATGCAAAAGTACTTTG |
| | | | ACCTTTAACCTTCGGTCCGGCGCGGTGGAGGGAAACG |
| | | | CCTCCGTCTCTATATAAGGAATTTTCCGGTCTCTTCGGG |
| | | | TCCTTTTTCCTCTCTTCAGCGTGGGGCGCCCACAATTTG |
| | | | CGCGCTCTCTTTCTGCTGCTCCCCAGCTCTCGGATACA |
| | | | GCCGACACCGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 217 | | EEF1B2 intron | TCGGGAGCCGTGGAGGTACGAACTTAAGACATGCCTA |
| | | inserted at | TTTTATTAATTTACTTCCAAACGCAACGAAAGGTCCAT |
| | | nucleotide 455 | GGACAATTTGTGGGCCATTTAATTCAGGGCCCCCAATT |
| | | of LP1 | CGTACGTGGAGAAGTGGGAATGCAAAAGTACTTTGAC |
| | | | CTTTAACCTTCGGTCCGGCGCGGTGGAGGGAAACGCCT |
| | | | CCGTCTCTATATAAGGAATTTTCCGGTCTCTTCGGGTCC |
| | | | TTTTTCCTCTCTTCAGCGTGGGGCGCCCACAATTTGCGC |
| | | | GCTCTCTTTCTGCTGCTCCCCAGCTCTCGGATACAGCC |
| | | | GACACC |
| 130 | L121 | LP1- | CCCTAAAATGGGCAAACATTGCAAGCAGCAAACAGCA |
| | | TMSB10i | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGAACGAGACTGCACGGTGAGTGCGGCGCCGGGGCG |
| | | | GGGGGCCCACCCAGGGTGTGGTCGGATCCGGTGCACC |
| | | | GGGCGGGCGCGCCGCAACCGCGACAGGCGCCCTTCTC |
| | | | GGACCGGACGCAGGGGCCGGCGACCACGCCCTGGGAC |
| | | | CGAGAAGAGGGGTGCGGGACGCGCCCAGATCCTCGGC |
| | | | CTTGGGGCTGCTCGGCACGCCTTGGCGCGAGTGCCACG |
| | | | TCGAGAGGCGTCGGCGGGGAGCGCGGAAGGGGACGCT |
| | | | GGCCCCCAGGCCCAGGTCAAGCGCCTTGGTTTGCCCAC |
| | | | TAGGATTGTTTTAAGAAAAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 218 | | TMSB10 intron | GAACGAGACTGCACGGTGAGTGCGGCGCCGGGGGGGG |
| | | inserted at | GGGCCCACCCAGGGTGTGGTCGGATCCGGTGCACCGG |
| | | nucleotide 455 | GCGGGCGCGCCGCAACCGCGACAGGCGCCCTTCTCGG |
| | | of LP1 | ACCGGACGCAGGGGCCGGCGACCACGCCCTGGGACCG |
| | | | AGAAGAGGGGTGCGGGACGCGCCCAGATCCTCGGCCT |
| | | | TGGGGCTGCTCGGCACGCCTTGGCGCGAGTGCCACGTC |
| | | | GAGAGGCGTCGGCGGGGAGCGCGGAAGGGGACGCTG |
| | | | GCCCCCAGGCCCAGGTCAAGCGCCTTGGTTTGCCCACT |
| | | | AGGATTGTTTTAAGAAAA |
| 131 | L122 | LP1- | CCCTAAAATGGGCAAACATTGCAAGCAGCAAACAGCA |
| | | S100A6i | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCGATTGTGCGTTGTGTAAGCCACGGCGCAGGGTG |
| | | | GGGCGCGGGCGGGACTTGGGCGGGCGGGGTGGGCTTG |
| | | | GCCGAGCTGGCCTCCGGGGCACCGACCGCTATAAGGC |
| | | | CAGTCGGACTGCGACACAGCCCATCCCCTCGACCGCTC |
| | | | GCGTCGCATTTGGCCGCCTCCCTACCGGTGAGTTCTCT |
| | | | CCAGGAGCCCTGGGTACTTGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 219 | | S100A6 intron | GCGATTGTGCGTTGTGTAAGCCACGGCGCAGGGTGGG |
| | | inserted at | GCGCGGGCGGGACTTGGGCGGGCGGGGTGGGCTTGGC |
| | | nucleotide 455 | CGAGCTGGCCTCCGGGGCACCGACCGCTATAAGGCCA |
| | | of LP1 | GTCGGACTGCGACACAGCCCATCCCCTCGACCGCTCGC |
| | | | GTCGCATTTGGCCGCCTCCCTACCGGTGAGTTCTCTCC |
| | | | AGGAGCCCTGGGTACTT |
| 132 | L123 | LP1-RPL27i | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCTGAAGGGCGGGACAGGAAGTGAGGTCACTCCT |
| | | | CACCGGGGTGAAAGGTTAGCGGAAGTGTCCTTCTTTCC |
| | | | TTTTTGCTGGTAGGGCCGGGTGGTTGCTGCCGGTAAGT |
| | | | AGAAGCTTGGGTTGAATCTTTCAATCCGCTGCCATCCG |
| | | | CGGCTTGGGGGTCGAAGGTCCGGGCGGCGCCGGGTGC |
| | | | ATTCCTTCCCTAGGTCTCTGGCGGCTACGTATCCGATC |
| | | | CTGTCCTGCGAGCAGGGCCCTCGGGCGGCCTTGACCAA |
| | | | GGCGACAGTGAACACAGTCTGAAGTTCCGGCCCAAGA |
| | | | CCTGGGCTTGCTGGCAGGAGTCAGCTCTGGGCATCTTT |
| | | | GGCTTTACGGATTTTTAAGTGGCCCTTTCTCCTTGCTCT |
| | | | CTGCAGAAAGGGGCAAGTTCAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 220 | | RPL27 intron | GCCTGAAGGGCGGGACAGGAAGTGAGGTCACTCCTCA |
| | | inserted at | CCGGGGTGAAAGGTTAGCGGAAGTGTCCTTCTTTCCTT |
| | | nucleotide 455 | TTTGCTGGTAGGGCCGGGTGGTTGCTGCCGGTAAGTAG |
| | | of LP1 | AAGCTTGGGTTGAATCTTTCAATCCGCTGCCATCCGCG |
| | | | GCTTGGGGGTCGAAGGTCCGGGCGGCGCCGGGTGCAT |
| | | | TCCTTCCCTAGGTCTCTGGCGGCTACGTATCCGATCCT |
| | | | GTCCTGCGAGCAGGGCCCTCGGGCGGCCTTGACCAAG |
| | | | GCGACAGTGAACACAGTCTGAAGTTCCGGCCCAAGAC |
| | | | CTGGGCTTGCTGGCAGGAGTCAGCTCTGGGCATCTTTG |
| | | | GCTTTACGGATTTTTAAGTGGCCCTTTCTCCTTGCTCTC |
| | | | TGCAGAAAGGGGCAAGTTCA |
| 133 | L124 | LP1-GFAPi | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCTCTCTGCGCGGCACGGTGAGCGCGGCCAGGGCAGG |
| | | | ACGGGGACGGGCGGAGCAGGGACGGCCACCCCAGGA |
| | | | GATGCCAGGGGAGAAGGGCCAGGGACGAGGACCTGG |
| | | | GTCGAGGGCGGGGCCTGAACGTTACCCCCGGGCTAGA |
| | | | GGCGGGGCCTGGGGCCGGGGGCCGGGCCTCGAAACCT |
| | | | CCCCAAGTCGGGGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 221 | | GFAP intron | TCTCTGCGCGGCACGGTGAGCGCGGCCAGGGCAGGAC |
| | | inserted at | GGGGACGGGCGGAGCAGGGACGGCCACCCCAGGAGA |
| | | nucleotide 455 | TGCCAGGGGAGAAGGGCCAGGGACGAGGACCTGGGTC |
| | | of LP1 | GAGGGCGGGGCCTGAACGTTACCCCCGGGCTAGAGGC |
| | | | GGGGCCTGGGGCCGGGGGCCGGGCCTCGAAACCTCCC |
| | | | CAAGTCGGG |
| 134 | L125 | LP1-UCHLi | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGCTGCGCACGGGGGGTTCGTACCCATCTGGCCGCG |
| | | | ACCGTCCGTTTCCCCCTCGCTTGGTTCTGCCCCTGCTCC |
| | | | CCCTGCACAGGCCTCACAGTGCGTCTGGCCGGCGCTTT |
| | | | ATAGCTGCAGCCTGGGCGGCTCCGCTAGCTGTTTTTCG |
| | | | TCTTCCCTAGGCTATTTCTGCCGGGGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 222 | | UCHL intron | CGCTGCGCACGGGGGGTTCGTACCCATCTGGCCGCGAC |
| | | inserted at | CGTCCGTTTCCCCCTCGCTTGGTTCTGCCCCTGCTCCCC |
| | | nucleotide 455 | CTGCACAGGCCTCACAGTGCGTCTGGCCGGCGCTTTAT |
| | | of LP1 | AGCTGCAGCCTGGGCGGCTCCGCTAGCTGTTTTTCGTC |
| | | | TTCCCTAGGCTATTTCTGCCGGG |
| 135 | L126 | LP1- | CCCTAAAATGGGCAAACATTGCAAGCAGCAAACAGCA |
| | | ALDOAi | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGGGGCTAGTGCTCGGGCAGGTGCCCTGGCTTCCCTT |
| | | | CCCCTCCACACGTCAACGATTCTATTTGAAGTTGGGCA |
| | | | GGGGGGTGGCGCTGCTCACCACACACAAGTGTTATAG |
| | | | GAGGAGTCTGGCCCTTGAGTACCGGGTACGCAGGGGT |
| | | | GCCTCAACCACACTCCGTCCACGGACTCTCCGTTATTT |
| | | | TAGGAGGTGAGTGTAGTGCCAGTATCTACTCTCCTTCT |
| | | | TAAAAAAAACCAGGGCTCCAGAGAATCAGAACAGCCA |
| | | | CCATCACCGCAGGGAGTCAAGGGAGGAGGGAGATTAG |
| | | | AGAAGGAGCCAGGGAGGGTGGCAGGGAGGCCACGTG |
| | | | ATCCGAGTCCCCTCACCCCTTTCCTTCCCACAGGTCCCT |
| | | | GGCCAAAGAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 223 | | ALDOA intron | GGGGCTAGTGCTCGGGCAGGTGCCCTGGCTTCCCTTCC |
| | | inserted at | CCTCCACACGTCAACGATTCTATTTGAAGTTGGGCAGG |
| | | nucleotide 455 | GGGGTGGCGCTGCTCACCACACACAAGTGTTATAGGA |
| | | of LP1 | GGAGTCTGGCCCTTGAGTACCGGGTACGCAGGGGTGC |
| | | | CTCAACCACACTCCGTCCACGGACTCTCCGTTATTTTA |
| | | | GGAGGTGAGTGTAGTGCCAGTATCTACTCTCCTTCTTA |
| | | | AAAAAAACCAGGGCTCCAGAGAATCAGAACAGCCACC |
| | | | ATCACCGCAGGGAGTCAAGGGAGGAGGGAGATTAGAG |
| | | | AAGGAGCCAGGGAGGGTGGCAGGGAGGCCACGTGATC |
| | | | CGAGTCCCCTCACCCCTTTCCTTCCCACAGGTCCCTGG |
| | | | CCAAAGA |
| 136 | L127 | LP1- | CCCTAAAATGGGCAAACATTGCAAGCAGCAAACAGCA |
| | | CAMK2Ai | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGCAGTGGAAGGCAGGCAGGTGTTGGGGAGGCAGT |
| | | | TACCGGGGCAACGGGAACAGGGCGTTTTGGAGGTGGT |
| | | | TGCCATGGGGACCTGGATGCTGACGAAGGCTCGCGAG |
| | | | GCTGTGAGCAGCCACAGTGCCCTGCTCAGAAGCCCCG |
| | | | GGCTCGTCAGTCAAACCGGTTCTCTGTTTGCACTCGGC |
| | | | AGCACGGGCAGGCAAGTGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 224 | | CAMK2A | CGCAGTGGAAGGCAGGCAGGTGTTGGGGAGGCAGTTA |
| | | intron inserted | CCGGGGCAACGGGAACAGGGCGTTTTGGAGGTGGTTG |
| | | at nucleotide | CCATGGGGACCTGGATGCTGACGAAGGCTCGCGAGGC |
| | | 455 of LP1 | TGTGAGCAGCCACAGTGCCCTGCTCAGAAGCCCCGGG |
| | | | CTCGTCAGTCAAACCGGTTCTCTGTTTGCACTCGGCAG |
| | | | CACGGGCAGGCAAGT |
| 137 | L128 | LP1-FHL li | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCAGACATTTGGAGAAGTATGCAGAACATCTTGGCA |
| | | | GTTTTCCTTTAACTCTAAGGGTTGTAAATACAAAGTAG |
| | | | AATGTGTTACCATGAGCCATTTCATAGGAAGTTGACTT |
| | | | TATTTTTTTAGAATTCAGAACTTTGCTTCTAAAAAGGT |
| | | | AACTTTGCCTCTTGATTCTCATTTTTTTCCCTAACTTAG |
| | | | GCACCTTGGAAGTTGGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 225 | | FHL 1 intron | CAGACATTTGGAGAAGTATGCAGAACATCTTGGCAGTT |
| | | inserted at | TTCCTTTAACTCTAAGGGTTGTAAATACAAAGTAGAAT |
| | | nucleotide 455 | GTGTTACCATGAGCCATTTCATAGGAAGTTGACTTTAT |
| | | of LP1 | TTTTTTAGAATTCAGAACTTTGCTTCTAAAAAGGTAAC |
| | | | TTTGCCTCTTGATTCTCATTTTTTTCCCTAACTTAGGCA |
| | | | CCTTGGAAGTTG |
| 138 | L129 | LP1- | CCCTAAAATGGGCAAACATTGCAAGCAGCAAACAGCA |
| | | CRYABi | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGAGGCAGTTGGCTGGTGCTGACATGTTGACCATCA |
| | | | CTGCTCTCTTCCAAGGACTCACAAAGAGTTAATGTCCC |
| | | | TGGGGCTCAGCCTAGGAAGATTCCAGTCCCTGCCCAGG |
| | | | CCCAAGATAGTTGCTGGCCTGATTCCCCTGGCATTCAG |
| | | | GACTGGAAAGGAGGAGGAGGGGCACACTACGCCGGCT |
| | | | CCCATCCTCCCCCCACCCCGCGTGCCTGCTTGGGATTC |
| | | | CTGACTCTGTACCAGCTTCAGAGAACAGGGGTGGGGG |
| | | | TGGGTGCCATTGGGTGTGGACAGAAAGCTAGTGAAAC |
| | | | AAGACCTTGACAAGTCACTGGCCGGCTCAGACGTGTTT |
| | | | GTGTCTCTCTTTTCTTAGCTCAGTGAGTACTGGGTTTGT |
| | | | GTCACATTGCCAAATCCCGGATCACAAGTCTCCTTGAA |
| | | | CTGCTGGTGAGCTAGGATAATAAAACCCCTGACATCAC |
| | | | CATTCCAGAAGCTTCACAAGACTGCATATATAAGGGG |
| | | | CTGGCTGTAGCTGCAGCTGAAGGAGCTGACCAGCCAG |
| | | | CTGAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 226 | | CRYAB intron | CGAGGCAGTTGGCTGGTGCTGACATGTTGACCATCACT |
| | | inserted at | GCTCTCTTCCAAGGACTCACAAAGAGTTAATGTCCCTG |
| | | nucleotide 455 | GGGCTCAGCCTAGGAAGATTCCAGTCCCTGCCCAGGCC |
| | | of LP1 | CAAGATAGTTGCTGGCCTGATTCCCCTGGCATTCAGGA |
| | | | CTGGAAAGGAGGAGGAGGGGCACACTACGCCGGCTCC |
| | | | CATCCTCCCCCCACCCCGCGTGCCTGCTTGGGATTCCT |
| | | | GACTCTGTACCAGCTTCAGAGAACAGGGGTGGGGGTG |
| | | | GGTGCCATTGGGTGTGGACAGAAAGCTAGTGAAACAA |
| | | | GACCTTGACAAGTCACTGGCCGGCTCAGACGTGTTTGT |
| | | | GTCTCTCTTTTCTTAGCTCAGTGAGTACTGGGTTTGTGT |
| | | | CACATTGCCAAATCCCGGATCACAAGTCTCCTTGAACT |
| | | | GCTGGTGAGCTAGGATAATAAAACCCCTGACATCACC |
| | | | ATTCCAGAAGCTTCACAAGACTGCATATATAAGGGGCT |
| | | | GGCTGTAGCTGCAGCTGAAGGAGCTGACCAGCCAGCT |
| | | | GA |
| 139 | L130 | LP1- | CCCTAAAATGGGCAAACATTGCAAGCAGCAAACAGCA |
| | | FXYDli | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCTCTGCCCTGCCAGGGTAGGTCTGGGAATCGGGGGC |
| | | | CTGCTGCGGGAGGTGGAGGCCCAAGGGAGGCCCCCCG |
| | | | GGGACTGTGTGTCTCACCCCCGTCCCTGCTACGTTGTG |
| | | | TTGTTGTGTGATCCCATCGTGGAGGTTGTTTTGGTGAC |
| | | | ACTGTGTCCCCACGAAGCTGGGGATACCCGTTTCTCTA |
| | | | GCTTGGAGCCACCAAGATAGAGGACAAACACTTCTGT |
| | | | GATTCAGTCCCCAGACTGTCTCTGACTTAATCCCTTGG |
| | | | GTTCAAGCCCTTTGTGGGAGAGCAAGGGCACACACTG |
| | | | CCTAATCCGTGGTGTCCCCCCCAGGACAATTGCGTCTC |
| | | | TGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 227 | | FXYD1 intron | CTCTGCCCTGCCAGGGTAGGTCTGGGAATCGGGGGCCT |
| | | inserted at | GCTGCGGGAGGTGGAGGCCCAAGGGAGGCCCCCCGGG |
| | | nucleotide 455 | GACTGTGTGTCTCACCCCCGTCCCTGCTACGTTGTGTTG |
| | | of LP1 | TTGTGTGATCCCATCGTGGAGGTTGTTTTGGTGACACT |
| | | | GTGTCCCCACGAAGCTGGGGATACCCGTTTCTCTAGCT |
| | | | TGGAGCCACCAAGATAGAGGACAAACACTTCTGTGAT |
| | | | TCAGTCCCCAGACTGTCTCTGACTTAATCCCTTGGGTTC |
| | | | AAGCCCTTTGTGGGAGAGCAAGGGCACACACTGCCTA |
| | | | ATCCGTGGTGTCCCCCCCAGGACAATTGCGTCTCT |
| 140 | L131 | LP1-RBP4i | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGTGCCCGGGCTCCGGTGAGTCAGGGCGCGTTATGC |
| | | | AAGTGCCCCCGGCGCCTCCCCTTCGGTCTTTCACCCCG |
| | | | CGCGGTTACGAAAGCGCGACCCCCTCCCCCCGGCGCTA |
| | | | TAAAGCAGCGGGGCGGCCGCGGCGCGCTCGCCTCCCT |
| | | | CGCTCCACGCGCGCCCGGACTCGGCGGCCAGGCTTGC |
| | | | GCGCGGTTCCCCTCCCGGTGGTGAGTCGCGGCCGGGGC |
| | | | CTCGGGGCCGGCGGCGGGTTGGGCCGGGGTGGGGTGG |
| | | | GGTGGGGTGAGGTGGGATACGGCGGCGGGGGTCGGCT |
| | | | GCCGGGCACTGACGGAGGCGCGTCTCCCGCAGGGCGG |
| | | | ATTCCTGGGCGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 228 | | RBP4 intron | CGTGCCCGGGCTCCGGTGAGTCAGGGCGCGTTATGCA |
| | | inserted at | AGTGCCCCCGGCGCCTCCCCTTCGGTCTTTCACCCCGC |
| | | nucleotide 455 | GCGGTTACGAAAGCGCGACCCCCTCCCCCCGGCGCTAT |
| | | of LP1 | AAAGCAGCGGGGCGGCCGCGGCGCGCTCGCCTCCCTC |
| | | | GCTCCACGCGCGCCCGGACTCGGCGGCCAGGCTTGCG |
| | | | CGCGGTTCCCCTCCCGGTGGTGAGTCGCGGCCGGGGCC |
| | | | TCGGGGCCGGCGGCGGGTTGGGCCGGGGTGGGGTGGG |
| | | | GTGGGGTGAGGTGGGATACGGCGGCGGGGGTCGGCTG |
| | | | CCGGGCACTGACGGAGGCGCGTCTCCCGCAGGGCGGA |
| | | | TTCCTGGGC |
| 141 | L132 | LP1-APOCi | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCAGGAAGATTGAGAGGTGAGAGTGCTGAACGGGGA |
| | | | GGGGCTTTGGGGCTAAGGGAAGTGCCCGGGACCCCAC |
| | | | CTGACCCCAACGCTCACGGGACAGGGGCAGAGGAGAA |
| | | | AAACGTGGGTGGACAGAGGGAGGCAGGCGGTCAGGG |
| | | | GAAGGCTCAGGAGGAGGGAGATCAACATCAACCTGCC |
| | | | CCGCCCCCTCCCCAGCCTGATAAAGGTCCTGCGGGCAG |
| | | | GACAGGACCTCCCAACCAAGCCCTCCAGCAAGGATTC |
| | | | AGGTTGGTGCTGAGTGCCTGGGAGGGACACCCGCCTA |
| | | | CACTCTGCAAGAAACTCAAAAAGGGAGTTGAGGGGAT |
| | | | CGTGGGAGGGAGGTAGGGAGGGAGGAGGGTGCCACT |
| | | | GATCCCCTGAACCCCTGCCTCTGCCTCCAGAGTGCCCC |
| | | | TCCGGCCGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 229 | | APOC intron | CAGGAAGATTGAGAGGTGAGAGTGCTGAACGGGGAGG |
| | | inserted at | GGCTTTGGGGCTAAGGGAAGTGCCCGGGACCCCACCT |
| | | nucleotide 455 | GACCCCAACGCTCACGGGACAGGGGCAGAGGAGAAA |
| | | of LP1 | AACGTGGGTGGACAGAGGGAGGCAGGCGGTCAGGGG |
| | | | AAGGCTCAGGAGGAGGGAGATCAACATCAACCTGCCC |
| | | | CGCCCCCTCCCCAGCCTGATAAAGGTCCTGCGGGCAGG |
| | | | ACAGGACCTCCCAACCAAGCCCTCCAGCAAGGATTCA |
| | | | GGTTGGTGCTGAGTGCCTGGGAGGGACACCCGCCTAC |
| | | | ACTCTGCAAGAAACTCAAAAAGGGAGTTGAGGGGATC |
| | | | GTGGGAGGGAGGTAGGGAGGGAGGAGGGTGCCACTG |
| | | | ATCCCCTGAACCCCTGCCTCTGCCTCCAGAGTGCCCCT |
| | | | CCGGCC |
| 142 | L133 | LP1-ATF5i | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCTCAGAGGGAAGAGGTCGGATCCCCAGCTGAGAG |
| | | | GGAGGAGGGTCCCGGACCCTAGGAGTGGGAAGGAAA |
| | | | GGCTCGGATCCCCTGATCCCCAGGAGGAGGGGACCCG |
| | | | GCTGCCTCCCGGTTGGGGCCGCGCGAGGGCGGGGCGC |
| | | | GGAAGGATCCGGGAGGGCCGTGCTCCGCCACCCAGTA |
| | | | TATATCTGTCCCCAGTCCCCGGGGCCGCCTCATTCCCT |
| | | | GTCCTCGGATCACAGTCTCTTCTCACTACAGTGTCGCC |
| | | | GCCTCTGCGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 230 | | ATF5 intron | GCTCAGAGGGAAGAGGTCGGATCCCCAGCTGAGAGGG |
| | | inserted at | AGGAGGGTCCCGGACCCTAGGAGTGGGAAGGAAAGGC |
| | | nucleotide 455 | TCGGATCCCCTGATCCCCAGGAGGAGGGGACCCGGCT |
| | | of LP1 | GCCTCCCGGTTGGGGCCGCGCGAGGGCGGGGCGCGGA |
| | | | AGGATCCGGGAGGGCCGTGCTCCGCCACCCAGTATAT |
| | | | ATCTGTCCCCAGTCCCCGGGGCCGCCTCATTCCCTGTC |
| | | | CTCGGATCACAGTCTCTTCTCACTACAGTGTCGCCGCC |
| | | | TCTGC |
| 143 | L134 | LP1-HPDi | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCCATCCCCCAGGTAGGCCAGGAGGCCTGGGCGGGA |
| | | | CCTGCTTGGGGTTAAATACTCCGGCCCAGCACTCCCCA |
| | | | GGCCTCTAGTCCCAGTAGGAGGTTTGACTAAGATCAAT |
| | | | CTTGGTAAGTGCCTTGGCCATCTCCCCTTGGGTGGGAC |
| | | | ATCTTCAGTAACGCTTCCAGAAGCACCTTGTGTTGGAA |
| | | | CCTCCAAGTCTCACTGTCCTTCTTTTTCTCCTTAGACGA |
| | | | CTTACAGTGACGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 231 | | HPD intron | CCATCCCCCAGGTAGGCCAGGAGGCCTGGGCGGGACC |
| | | inserted at | TGCTTGGGGTTAAATACTCCGGCCCAGCACTCCCCAGG |
| | | nucleotide 455 | CCTCTAGTCCCAGTAGGAGGTTTGACTAAGATCAATCT |
| | | of LP1 | TGGTAAGTGCCTTGGCCATCTCCCCTTGGGTGGGACAT |
| | | | CTTCAGTAACGCTTCCAGAAGCACCTTGTGTTGGAACC |
| | | | TCCAAGTCTCACTGTCCTTCTTTTTCTCCTTAGACGACT |
| | | | TACAGTGAC |
| 144 | L135 | LP1-FLOTli | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCCCGCGTCCCGGAAGGTGAGTGGGGGAGGGGAGAT |
| | | | GGAGCCTGGGGCTGCCGAGAAGCAGGGAGAGAGGGG |
| | | | AAAGGTCTGCAGGCCTCTGGGCGCGGGGGACCTTTATC |
| | | | AAAGGGAGCGGCGAGGGGAAGGGAGGGACCATACGC |
| | | | AGGCGGAGAAGAGGTAGCTGGGGATCTTAAGGATTTT |
| | | | GTAGGGAAAAGCCTTAGCGGACCCTTGGGTGTAGAAG |
| | | | GGACGGGCCGTGGCGGTTGCAGACTGGGAGGGCCTGA |
| | | | CGGGAAAGGGAAAGGTTGGGCCCCTGTGGACGCCACA |
| | | | GCGCGCCTTCCCCATCCGCAAAGGCTTTCCCTCCCTCT |
| | | | CCTCCAGCTCCAGCCTGAACCGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 232 | | FLOT1 intron | CCCGCGTCCCGGAAGGTGAGTGGGGGAGGGGAGATGG |
| | | inserted at | AGCCTGGGGCTGCCGAGAAGCAGGGAGAGAGGGGAA |
| | | nucleotide 455 | AGGTCTGCAGGCCTCTGGGCGCGGGGGACCTTTATCAA |
| | | of LP1 | AGGGAGCGGCGAGGGGAAGGGAGGGACCATACGCAG |
| | | | GCGGAGAAGAGGTAGCTGGGGATCTTAAGGATTTTGT |
| | | | AGGGAAAAGCCTTAGCGGACCCTTGGGTGTAGAAGGG |
| | | | ACGGGCCGTGGCGGTTGCAGACTGGGAGGGCCTGACG |
| | | | GGAAAGGGAAAGGTTGGGCCCCTGTGGACGCCACAGC |
| | | | GCGCCTTCCCCATCCGCAAAGGCTTTCCCTCCCTCTCCT |
| | | | CCAGCTCCAGCCTGAACC |
| 145 | L136 | LP1-HBBil | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCTGAGGCCCTGGGCAGGTTGGTATCAAGGTTACAAG |
| | | | ACAGGTTTAAGGAGACCAATAGAAACTGGGCATGTGG |
| | | | AGACAGAGAAGACTCTTGGGTTTCTGATAGGCACTGA |
| | | | CTCTCTCTGCCTATTGGTCTATTTTCCCACCCTTAGGCT |
| | | | GCTGGTGGTCTAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 194 | | HBB intron 1 | TGAGGCCCTGGGCAGGTTGGTATCAAGGTTACAAGAC |
| | | inserted at | AGGTTTAAGGAGACCAATAGAAACTGGGCATGTGGAG |
| | | nucleotide 455 | ACAGAGAAGACTCTTGGGTTTCTGATAGGCACTGACTC |
| | | of LP1 | TCTCTGCCTATTGGTCTATTTTCCCACCCTTAGGCTGCT |
| | | | GGTGGTCTA |
| 146 | L137 | LP1-HBBi2 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCCTGAGAACTTCAGGGTGAGTCTATGGGACGCTTGA |
| | | | TGTTTTCTTTCCCCTTCTTTTCTATGGTTAAGTTCATGTC |
| | | | ATAGGAAGGGGATAAGTAACAGGGTACAGTTTAGAAT |
| | | | GGGAAACAGACGAATGATTGCATCAGTGTGGAAGTCT |
| | | | CAGGATCGTTTTAGTTTCTTTTATTTGCTGTTCATAACA |
| | | | ATTGTTTTCTTTTGTTTAATTCTTGCTTTCTTTTTTTTTCT |
| | | | TCTCCGCAATTTTTACTATTATACTTAATGCCTTAACAT |
| | | | TGTGTATAACAAAAGGAAATATCTCTGAGATACATTAA |
| | | | GTAACTTAAAAAAAAACTTTACACAGTCTGCCTAGTAC |
| | | | ATTACTATTTGGAATATATGTGTGCTTATTTGCATATTC |
| | | | ATAATCTCCCTACTTTATTTTCTTTTATTTTTAATTGATA |
| | | | CATAATCATTATACATATTTATGGGTTAAAGTGTAATG |
| | | | TTTTAATATGTGTACACATATTGACCAAATCAGGGTAA |
| | | | TTTTGCATTTGTAATTTTAAAAAATGCTTTCTTCTTTTA |
| | | | ATATACTTTTTTGTTTATCTTATTTCTAATACTTTCCCTA |
| | | | ATCTCTTTCTTTCAGGGCAATAATGATACAATGTATCA |
| | | | TGCCTCTTTGCACCATTCTAAAGAATAACAGTGATAAT |
| | | | TTCTGGGTTAAGGCAATAGCAATATCTCTGCATATAAA |
| | | | TATTTCTGCATATAAATTGTAACTGATGTAAGAGGTTT |
| | | | CATATTGCTAATAGCAGCTACAATCCAGCTACCATTCT |
| | | | GCTTTTATTTTATGGTTGGGATAAGGCTGGATTATTCTG |
| | | | AGTCCAAGCTAGGCCCTTTTGCTAATCATGTTCATACC |
| | | | TCTTATCTTCCTCCCACAGCTCCTGGGCAACGTGGCCA |
| | | | CC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 233 | | HBB intron 2 | CCTGAGAACTTCAGGGTGAGTCTATGGGACGCTTGATG |
| | | inserted at | TTTTCTTTCCCCTTCTTTTCTATGGTTAAGTTCATGTCAT |
| | | nucleotide 455 | AGGAAGGGGATAAGTAACAGGGTACAGTTTAGAATGG |
| | | of LP1 | GAAACAGACGAATGATTGCATCAGTGTGGAAGTCTCA |
| | | | GGATCGTTTTAGTTTCTTTTATTTGCTGTTCATAACAAT |
| | | | TGTTTTCTTTTGTTTAATTCTTGCTTTCTTTTTTTTTCTTC |
| | | | TCCGCAATTTTTACTATTATACTTAATGCCTTAACATTG |
| | | | TGTATAACAAAAGGAAATATCTCTGAGATACATTAAGT |
| | | | AACTTAAAAAAAAACTTTACACAGTCTGCCTAGTACAT |
| | | | TACTATTTGGAATATATGTGTGCTTATTTGCATATTCAT |
| | | | AATCTCCCTACTTTATTTTCTTTTATTTTTAATTGATAC |
| | | | ATAATCATTATACATATTTATGGGTTAAAGTGTAATGT |
| | | | TTTAATATGTGTACACATATTGACCAAATCAGGGTAAT |
| | | | TTTGCATTTGTAATTTTAAAAAATGCTTTCTTCTTTTAA |
| | | | TATACTTTTTTGTTTATCTTATTTCTAATACTTTCCCTAA |
| | | | TCTCTTTCTTTCAGGGCAATAATGATACAATGTATCAT |
| | | | GCCTCTTTGCACCATTCTAAAGAATAACAGTGATAATT |
| | | | TCTGGGTTAAGGCAATAGCAATATCTCTGCATATAAAT |
| | | | ATTTCTGCATATAAATTGTAACTGATGTAAGAGGTTTC |
| | | | ATATTGCTAATAGCAGCTACAATCCAGCTACCATTCTG |
| | | | CTTTTATTTTATGGTTGGGATAAGGCTGGATTATTCTGA |
| | | | GTCCAAGCTAGGCCCTTTTGCTAATCATGTTCATACCT |
| | | | CTTATCTTCCTCCCACAGCTCCTGGGCAACGTG |
| 147 | L138 | LP1- | CCCTAAAATGGGCAAACATTGCAAGCAGCAAACAGCA |
| | | HBBi2xc | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCCTGAGAACTTCAGGGTGAGTCTATGGGACCCTTGA |
| | | | TGTTTTCTTTCCCCTTCTTTTCTATGGTTAAGTTCATGTC |
| | | | ATAGGAAGGGGAGAAGTAACAGGGTACACATATTGAC |
| | | | CAAATCAGGGTAATTTTGCATTTGTAATTTTAAAAAAT |
| | | | GCTTTCTTCTTTTAATATACTTTTTTGTTTATCTTATTTC |
| | | | TAATACTTTCCCTAATCTCTTTCTTTCAGGGCAATAATG |
| | | | ATACAATGTATCATGCCTCTTTGCACCATTCTAAAGAA |
| | | | TAACAGTGATAATTTCTGGGTTAAGGCAATAGCAATAT |
| | | | TTCTGCATATAAATATTTCTGCATATAAATTGTAACTG |
| | | | ATGTAAGAGGTTTCATATTGCTAATAGCAGCTACAATC |
| | | | CAGCTACCATTCTGCTTTTATTTTATGGTTGGGATAAG |
| | | | GCTGGATTATTCTGAGTCCAAGCTAGGCCCTTTTGCTA |
| | | | ATCATGTTCATACCTCTTATCTTCCTCCCACAGCTCCTG |
| | | | GGCAACGTGGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 234 | | synthetic intron | CCTGAGAACTTCAGGGTGAGTCTATGGGACCCTTGATG |
| | | based on HBB | TTTTCTTTCCCCTTCTTTTCTATGGTTAAGTTCATGTCAT |
| | | and beta-globin | AGGAAGGGGAGAAGTAACAGGGTACACATATTGACCA |
| | | inserted at | AATCAGGGTAATTTTGCATTTGTAATTTTAAAAAATGC |
| | | nucleotide 455 | TTTCTTCTTTTAATATACTTTTTTGTTTATCTTATTTCTA |
| | | of LP1 | ATACTTTCCCTAATCTCTTTCTTTCAGGGCAATAATGAT |
| | | | ACAATGTATCATGCCTCTTTGCACCATTCTAAAGAATA |
| | | | ACAGTGATAATTTCTGGGTTAAGGCAATAGCAATATTT |
| | | | CTGCATATAAATATTTCTGCATATAAATTGTAACTGAT |
| | | | GTAAGAGGTTTCATATTGCTAATAGCAGCTACAATCCA |
| | | | GCTACCATTCTGCTTTTATTTTATGGTTGGGATAAGGCT |
| | | | GGATTATTCTGAGTCCAAGCTAGGCCCTTTTGCTAATC |
| | | | ATGTTCATACCTCTTATCTTCCTCCCACAGCTCCTGGGC |
| | | | AACGTG |
| 148 | L139 | LP1- | CCCTAAAATGGGCAAACATTGCAAGCAGCAAACAGCA |
| | | IFI27L2i | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCTGTTGAGTGAGTCTTGCTTCCCCAGGGGGCTGGCGG |
| | | | GCTGGGCTGGGGGCAGGCTTCCCCAACCCCAGCCCAG |
| | | | GAAGGGACAGGGAATAGCTTCATTCCCTGACTTCATCA |
| | | | ACCAGCTGACCTACTGACTCCCTCTCCCTCCCTTCCTCC |
| | | | CTCCCCTTCCCTTCCTCCCTCCGCTCCTTGCTTCCCTCC |
| | | | CCCTCCCTCCCTTTCTGCCCTTCTCTCTCCTCTTTCTCCC |
| | | | CTTTCCCATCTCCCTCTGCCTTCCCCTGGCACTCTCTCT |
| | | | CTAGAACGGGCAGCTGCTGGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 235 | | IFI27L2 intron | TGTTGAGTGAGTCTTGCTTCCCCAGGGGGCTGGCGGGC |
| | | inserted at | TGGGCTGGGGGCAGGCTTCCCCAACCCCAGCCCAGGA |
| | | nucleotide 455 | AGGGACAGGGAATAGCTTCATTCCCTGACTTCATCAAC |
| | | of LP1 | CAGCTGACCTACTGACTCCCTCTCCCTCCCTTCCTCCCT |
| | | | CCCCTTCCCTTCCTCCCTCCGCTCCTTGCTTCCCTCCCC |
| | | | CTCCCTCCCTTTCTGCCCTTCTCTCTCCTCTTTCTCCCCT |
| | | | TTCCCATCTCCCTCTGCCTTCCCCTGGCACTCTCTCTCT |
| | | | AGAACGGGCAGCTGCTG |
| 149 | L140 | LP1-RPL26i | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCCGAAGTAACTTCTCGGTAAAGTCTTCACGGAATTTC |
| | | | CAGACCACACTTGCCCACTGGGAGGCTTTTAGGACCCG |
| | | | AGACGTGTGCAGGCTTTTCCAGGTTCGGGTTGGTGGGC |
| | | | CTTATTCATTTTGGCGGGGCTATACGTATTCACTCTTAA |
| | | | GCCTGTAGAAGATGAACGTTGATTGTACCTTCTAAAAC |
| | | | GTGAGGTTATAGTTAAGAGAATTGCGTTGTCGGTCTGC |
| | | | TCAGCAGAGATGTAAGTCCAGTCTTGATTTCCTCAGGC |
| | | | ACTACTTGAGGAGTTGGGAGAGTGTAACTAAAAGGTC |
| | | | TTATAACTAGGACAAGTGTTTTTGATGCAAAAGTCTTT |
| | | | AGTCCGTAAACATATACAGAAAACACCCGAATAAAAC |
| | | | AAAAAATAAAAAGTTTCGAGCTTCGGTTGATAGGCCCT |
| | | | TCCGATCAAACAGTTTTTGTTGAAGTTGCACAACATGA |
| | | | CACTGCCTATGATCATTCTAGACTATCAAGATAAAATC |
| | | | ATTGCGGTTATTTGTTTGGAAGCTGGATGAGATTTTGG |
| | | | TCATTTAAGAGTCTTTTTAACTTTTTATTTAGCCAAATT |
| | | | GAAGTTTAGCCACC |
| 74 | | LP1 | CCCTAAAATGGGCAAACATTGCAAGCAGCAAACAGCA |
| | | | AACACACAGCCCTCCCTGCCTGCTGACCTTGGAGCTGG |
| | | | GGCAGAGGTCAGAGACCTCTCTGGGCCCATGCCACCTC |
| | | | CAACATCCACTCGACCCCTTGGAATTTCGGTGGAGAGG |
| | | | AGCAGAGGTTGTCCTGGCGTGGTTTAGGTAGTGTGAGA |
| | | | GGGGAATGACTCCTTTCGGTAAGTGCAGTGGAAGCTGT |
| | | | ACACTGCCCAGGCAAAGCGTCCGGGCAGCGTAGGCGG |
| | | | GCGACTCAGATCCCAGCCAGTGGACTTAGCCCCTGTTT |
| | | | GCTCCTCCGATAACTGGGGTGACCTTGGTTAATATTCA |
| | | | CCAGCAGCCTCCCCCGTTGCCCCTCTGGATCCACTGCT |
| | | | TAAATACGGACGAGGACAGGGCCCTGTCTCCTCAGCTT |
| | | | CAGGCACCACCACTGACCTGGGACAGTGAATTCGCGG |
| | | | CCGCCACC |
| 249 | | RPL26 intron | CGAAGTAACTTCTCGGTAAAGTCTTCACGGAATTTCCA |
| | | inserted at | GACCACACTTGCCCACTGGGAGGCTTTTAGGACCCGAG |
| | | nucleotide 455 | ACGTGTGCAGGCTTTTCCAGGTTCGGGTTGGTGGGCCT |
| | | of LP1 | TATTCATTTTGGCGGGGCTATACGTATTCACTCTTAAG |
| | | | CCTGTAGAAGATGAACGTTGATTGTACCTTCTAAAACG |
| | | | TGAGGTTATAGTTAAGAGAATTGCGTTGTCGGTCTGCT |
| | | | CAGCAGAGATGTAAGTCCAGTCTTGATTTCCTCAGGCA |
| | | | CTACTTGAGGAGTTGGGAGAGTGTAACTAAAAGGTCTT |
| | | | ATAACTAGGACAAGTGTTTTTGATGCAAAAGTCTTTAG |
| | | | TCCGTAAACATATACAGAAAACACCCGAATAAAACAA |
| | | | AAAATAAAAAGTTTCGAGCTTCGGTTGATAGGCCCTTC |
| | | | CGATCAAACAGTTTTTGTTGAAGTTGCACAACATGACA |
| | | | CTGCCTATGATCATTCTAGACTATCAAGATAAAATCAT |
| | | | TGCGGTTATTTGTTTGGAAGCTGGATGAGATTTTGGTC |
| | | | ATTTAAGAGTCTTTTTAACTTTTTATTTAGCCAAATTGA |
| | | | AGTTTA |

We claim:

1. A NARE comprising: (i) a sequence that is at least 90% identical to SEQ ID NO: 74; and (ii) a sequence that is at least 90% identical to any one of SEQ ID NOs: 193, 194, 207, 209-214, 216-232, 233-235, or 249.

2. The NARE of claim 1, comprising: (i) a sequence that is at least 95% identical to SEQ ID NO: 74; and (ii) a sequence that is at least 95% identical to any one of SEQ ID NOs: 193, 194, 207, 209-214, 216-232, 233-235, or 249.

3. The NARE of claim 1, comprising: (i) SEQ ID NO: 74; and (ii) any one of SEQ ID NOs: 193, 194, 207, 209-214, 216-232, 233-235, or 249.

4. A NARE comprising: (i) a sequence that is at least 90% identical to SEQ ID NO: 150; and (ii) a sequence that is at least 90% identical to SEQ ID NO: 151 or SEQ ID NO: 155.

5. The NARE of claim 4, comprising: (i) a sequence that is at least 95% identical to SEQ ID NO: 150; and (ii) a sequence that is at least 95% identical to SEQ ID NO: 151 or SEQ ID NO: 155.

6. The NARE of claim 4, comprising: (i) SEQ ID NO: 150; and (ii) SEQ ID NO: 151 or SEQ ID NO: 155.

7. A NARE comprising: (i) a sequence that is at least 90% identical to any one of SEQ ID NOs: 154, 157, or 158; (ii) a sequence that is at least 90% identical to any one of SEQ ID NOs: 153, 156, 161, 162, 163, 166, 167, 168, or 174; and (iii) optionally, a sequence that is at least 90% identical to any one of SEQ ID NOs: 169, 172 or 173.

8. The NARE of claim 7, comprising: (i) a sequence that is at least 95% identical to any one of SEQ ID NOs: 154, 157, or 158; (ii) a sequence that is at least 95% identical to any one of SEQ ID NOs: 153, 156, 161, 162, 163, 166, 167, 168, or 174; and (iii) optionally, a sequence that is at least 95% identical to any one of SEQ ID NOs: 169, 172 or 173.

9. The NARE of claim 7, comprising: (i) any one of SEQ ID NOs: 154, 157, or 158 (ii) any one of SEQ ID NOs: 153, 156, 161, 162, 163, 166, 167, 168, or 174; and (iii) optionally any one of SEQ ID NOs: 169, 172 or 173.

10. A NARE comprising: (i) a sequence that is at least 90% identical to SEQ ID NO: 153; (ii) a sequence that is at least 90% identical to SEQ ID NO: 171; and (iii) a sequence that is at least 90% identical to any one of SEQ ID NOs: 154, 157, 158 or 176.

11. The NARE of claim 10, comprising: (i) a sequence that is at least 95% identical to SEQ ID NO: 153; (ii) a sequence that is at least 95% identical to SEQ ID NO: 171; and (iii) a sequence that is at least 95% identical to any one of SEQ ID NOs: 154, 157, 158 or 176.

12. The NARE of claim 10, comprising: (i) SEQ ID NO: 153; (ii) SEQ ID NO: 171; and (iii) any one of SEQ ID NOs: 154, 157, 158 or 176.

13. A NARE comprising: (i) a sequence that is at least 90% identical to SEQ ID NO: 153; (ii) a sequence that is at least 90% identical to any one of SEQ ID NOs: 154, 157, 158, 176, 188, or 250; and (iii) optionally, SEQ ID NO: 155 or SEQ ID NO: 171.

14. The NARE of claim 13, comprising: (i) a sequence that is at least 95% identical to SEQ ID NO: 153; (ii) a sequence that is at least 95% identical to any one of SEQ ID NOs: 154, 157, 158, 176, 188, or 250; and (iii) optionally, SEQ ID NO: 155 or SEQ ID NO: 171.

15. The NARE of claim 13, comprising: (i) SEQ ID NO: 153; (ii) any one of SEQ ID NOs: 154, 157, 158, 176, 188, or 250; and (iii) optionally, SEQ ID NO: 155 or SEQ ID NO: 171.

16. A NARE comprising: (i) a sequence that is at least 90% identical to SEQ ID NO: 155; (ii) a sequence that is at least 90% identical to SEQ ID NO: 176 and (iii) a sequence that is at least 90% identical to any one of SEQ ID NOs: 175 or 177-187.

17. The NARE of claim 16, comprising: (i) a sequence that is at least 95% identical to SEQ ID NO: 153; (ii) a sequence that is at least 95% identical to SEQ ID NO: 176 and (iii) a sequence that is at least 95% identical to any one of SEQ ID NOs: 175 or 177-187.

18. The NARE of claim 16, comprising: (i) SEQ ID NO: 153; (ii) SEQ ID NO: 176 and (iii) any one of SEQ ID NOs: 175 or 177-187.

19. A NARE comprising: (i) a sequence that is at least 90% identical to SEQ ID NO: 190 or SEQ ID NO: 191; (ii) a sequence that is at least 90% identical to any one of SEQ ID NOs: 193, 194, or 195; and (iii) optionally, a sequence that is at least 90% identical to SEQ ID NO: 162; (iv) optionally, a sequence that is at least 90% identical to SEQ ID NO: 168; and (v) optionally, SEQ ID NO: 169.

20. The NARE of claim 19, comprising: (i) a sequence that is at least 95% identical to SEQ ID NO: 190 or SEQ ID NO: 191; (ii) a sequence that is at least 95% identical to any one of SEQ ID NOs: 193, 194, or 195; and (iii) optionally, a sequence that is at least 95% identical to SEQ ID NO: 162; (iv) optionally, a sequence that is at least 95% identical to SEQ ID NO: 168; and (v) optionally, SEQ ID NO: 169.

21. The NARE of claim 19, comprising: (i) SEQ ID NO: 190 or SEQ ID NO: 191; (ii) any one of SEQ ID NOs: 193, 194, or 195; (iii) optionally, SEQ ID NO: 162; (iv) optionally, SEQ ID NO: 168; and (v) optionally, SEQ ID NO: 169.

22. A NARE comprising: (i) a sequence that is at least 90% identical to SEQ ID NO: 168; (ii) a sequence that is at least 90% identical to any one of SEQ ID NOs: 197, 202 or 203; (iv) optionally, a sequence that is at least 90% identical to any one of SEQ ID NOs: 190, 191, 192, 196, 202, 203, 204, 205 or 206; (v) optionally, a sequence that is at least 90% identical to SEQ ID NO: 155 or SEQ ID NO: 162; and (vi) optionally, a sequence that is at least 90% identical to any one of SEQ ID NOs: 166, 193, or 194.

23. The NARE of claim 22, comprising: (i) a sequence that is at least 95% identical to SEQ ID NO: 168; (ii) a sequence that is at least 95% identical to any one of SEQ ID NOs: 197, 202 or 203; (iv) optionally, a sequence that is at least 95% identical to any one of SEQ ID NOs: 190, 191, 192, 196, 202, 203, 204, 205 or 206; (v) optionally, a sequence that is at least 95% identical to SEQ ID NO: 155 or SEQ ID NO: 162; and (vi) optionally, a sequence that is at least 95% identical to any one of SEQ ID NOs: 166, 193, or 194.

24. The NARE of claim 22, comprising: (i) SEQ ID NO: 168; (ii) any one of SEQ ID NOs: 197, 202 or 203; (iv) optionally, any one of SEQ ID NOs: 190, 191, 192, 196, 202, 203, 204, 205 or 206; (v) optionally, SEQ ID NO: 155 or SEQ ID NO: 162; and (vi) optionally any one of SEQ ID NOs: 166, 193, or 194.

25. A NARE comprising: (i) a sequence that is at least 90% identical to SEQ ID NO: 192; (ii) a sequence that is at least 90% identical to SEQ ID NO: 193 or SEQ ID NO: 194; and (iii) a sequence that is at least 90% identical to any one of SEQ ID NOs: 201-203.

26. The NARE of claim 25, comprising: (i) a sequence that is at least 95% identical to SEQ ID NO: 192; (ii) a sequence that is at least 95% identical to SEQ ID NO: 193 or SEQ ID NO: 194; and (iii) a sequence that is at least 95% identical to any one of SEQ ID NOs: 201-203.

27. The NARE of claim 25, comprising: (i) SEQ ID NO: 192; (ii) SEQ ID NO: 193 or SEQ ID NO: 194; and (iii) any one of SEQ ID NOs: 201-203.

28. A NARE comprising: (i) a sequence that is at least 90% identical to SEQ ID NO: 155; (ii) a sequence that is at least 90% to any one of SEQ ID NOs: 204, 205, 206, or 236-238; (iii) optionally, a sequence that is at 90% identical to SEQ ID NO: 168; (iv) optionally, a sequence that is at least 90% identical to SEQ ID NO: 169; and (v) optionally, a sequence that is at least 90% identical to SEQ ID NO: 197.

29. The NARE of claim 28, comprising: (i) a sequence that is at least 95% identical to SEQ ID NO: 155; (ii) a sequence that is at least 95% to any one of SEQ ID NOs: 204, 205, 206, or 236-238; (iii) optionally, a sequence that is at 90% identical to SEQ ID NO: 168; (iv) optionally, a sequence that is at least 95% identical to SEQ ID NO: 169; and (v) optionally, a sequence that is at least 95% identical to SEQ ID NO: 197.

30. The NARE of claim 28, comprising: (i) SEQ ID NO: 155; (ii) a sequence that is at least 95% to any one of SEQ ID NOs: 204, 205, 206, or 236-238; (iii) optionally, a sequence that is at 90% identical to SEQ ID NO: 168; (iv) optionally, a sequence that is at least 95% identical to SEQ ID NO: 169; and (v) optionally, a sequence that is at least 95% identical to SEQ ID NO: 197.

31. A NARE comprising: (i) a sequence that is at least 90% identical to SEQ ID NO: 248; and (ii) a sequence that is at least 90% identical to any one of SEQ ID NOs: 239-247.

32. The NARE of claim 31, comprising: (i) a sequence that is at least 95% identical to SEQ ID NO: 248; and (ii) a sequence that is at least 95% identical to any one of SEQ ID NOs: 239-247.

33. The NARE of claim 31, comprising: (i) SEQ ID NO: 248; and (ii) any one of SEQ ID NOs: 239-247.

34. A NARE comprising: (i) a sequence that is at least 90% identical to SEQ ID NO: 208; and (ii) a sequence that is at least 90% identical to any one of SEQ ID NOs: 209-214.

35. The NARE of claim 34, comprising: (i) a sequence that is at least 95% identical to SEQ ID NO: 208; and (ii) a sequence that is at least 95% identical to any one of SEQ ID NOs: 209-214.

36. The NARE of claim 34, comprising: (i) SEQ ID NO: 208; and (ii) any one of SEQ ID NOs: 209-214.

37. A NARE comprising: (i) a sequence that is at least 90% identical to SEQ ID NO: 193 or SEQ ID NO: 194; (ii) a sequence that is at least 90% identical to SEQ ID NO: 215; and (iii) a sequence that is at least 90% identical to any one of SEQ ID NOs: 207 or 209-215.

38. The NARE of claim 37, comprising: (i) a sequence that is at least 95% identical to SEQ ID NO: 193 or SEQ ID NO: 194; (ii) a sequence that is at least 95% identical to SEQ ID NO: 215; and (iii) a sequence that is at least 95% identical to any one of SEQ ID NOs: 207 or 209-215.

39. The NARE of claim 37, comprising: (i) SEQ ID NO: 193 or SEQ ID NO: 194; (ii) SEQ ID NO: 215; and (iii) any one of SEQ ID NOs: 207 or 209-215.

40. A NARE comprising: (i) a sequence that is at least 90% identical to SEQ ID NO: 155; (ii) a sequence that is at least 90% identical to SEQ ID NO: 205 or SEQ ID NO: 206; and (iii) a sequence that is at least 90% identical to any one of SEQ ID NOs: 207 or 209-215.

41. The NARE of claim 40, comprising: (i) a sequence that is at least 95% identical to SEQ ID NO: 155; (ii) a sequence that is at least 95% identical to SEQ ID NO: 205 or SEQ ID NO: 206; and (iii) a sequence that is at least 95% identical to any one of SEQ ID NOs: 207 or 209-215.

42. The NARE of claim 40, comprising: (i) SEQ ID NO: 155; (ii) SEQ ID NO: 205 or SEQ ID NO: 206; and (iii) any one of SEQ ID NOs: 207 or 209-215.

43. A NARE comprising:
a) (i) a sequence that is at least 90% identical to SEQ ID NO: 53;
b) (i) a sequence that is at least 90% identical to SEQ ID NO: 195; and (ii) a sequence that is at least 90% identical to SEQ ID NO: 202;
c) (i) a sequence that is at least 90% identical to a sequence that is at least 90% identical to SEQ ID NO: 168; (ii) a sequence that is at least 90% identical to a sequence that is at least 90% identical to SEQ ID NO: 169; (iii) a sequence that is at least 90% identical to a sequence that is at least 90% identical to SEQ ID NO: 197; and (iv) a sequence that is at least 90% identical to a sequence that is at least 90% identical to SEQ ID NO: 203;
d) (i) a sequence that is at least 90% identical to SEQ ID NO: 74; and (ii) a sequence that is at least 90% identical to a sequence that is at least 90% identical to SEQ ID NO: 220;
e) (i) a sequence that is at least 90% identical to SEQ ID NO: 74; and (ii) a sequence that is at least 90% identical to a sequence that is at least 90% identical to SEQ ID NO: 229; or
f) (i) a sequence that is at least 90% identical to SEQ ID NO: 74; and (ii) a sequence that is at least 90% identical to a sequence that is at least 90% identical to SEQ ID NO: 230.

44. The NARE of claim 43, comprising:
a) (i) a sequence that is at least 95% identical to SEQ ID NO: 53;
b) (i) a sequence that is at least 95% identical to SEQ ID NO: 195; and (ii) a sequence that is at least 95% identical to SEQ ID NO: 202;
c) (i) a sequence that is at least 95% identical to a sequence that is at least 95% identical to SEQ ID NO: 168; (ii) a sequence that is at least 95% identical to a sequence that is at least 95% identical to SEQ ID NO: 169; (iii) a sequence that is at least 95% identical to a sequence that is at least 95% identical to SEQ ID NO: 197; and (iv) a sequence that is at least 95% identical to a sequence that is at least 95% identical to SEQ ID NO: 203;
d) (i) a sequence that is at least 95% identical to SEQ ID NO: 74; and (ii) a sequence that is at least 95% identical to a sequence that is at least 95% identical to SEQ ID NO: 220;
e) (i) a sequence that is at least 95% identical to SEQ ID NO: 74; and (ii) a sequence that is at least 95% identical to a sequence that is at least 95% identical to SEQ ID NO: 229; or
f) (i) a sequence that is at least 95% identical to SEQ ID NO: 74; and (ii) a sequence that is at least 95% identical to a sequence that is at least 95% identical to SEQ ID NO: 230.

45. The NARE of claim 43, comprising:
a) (i) SEQ ID NO: 53;
b) (i) SEQ ID NO: 195; and (ii) SEQ ID NO: 202;
c) (i) SEQ ID NO: 168; (ii) SEQ ID NO: 169; (iii) SEQ ID NO: 197; and (iv) to SEQ ID NO: 203;
d) (i) SEQ ID NO: 74; and (ii) SEQ ID NO: 220;
e) (i) SEQ ID NO: 74; and (ii) SEQ ID NO: 229; or
f) (i) SEQ ID NO: 74; and (ii) SEQ ID NO: 230.

46. A NARE comprising: (i) a sequence that is at least 90% identical to SEQ ID NO: 166 and (ii) a sequence that is at least 90% identical to any one of SEQ ID NOs: 154, 157, or 158.

47. The NARE of claim 46, comprising: (i) a sequence that is at least 95% identical to SEQ ID NO: 166 and (ii) a sequence that is at least 95% identical to any one of SEQ ID NOs: 154, 157, or 158.

48. The NARE of claim 46, comprising: (i) SEQ ID NO: 166; and (ii) any one of SEQ ID NOs: 154, 157, or 158.

49. A NARE comprising: (i) a sequence that is at least 90% identical to SEQ ID NO: 165 and (ii) a sequence that at least 90% identical to any one of SEQ ID NOs: 154, 157, or 158.

50. The NARE of claim 49, comprising: (i) a sequence that is at least 95% identical to SEQ ID NO: 165 and (ii) a sequence that at least 95% identical to any one of SEQ ID NOs: 154, 157, or 158.

51. The NARE of claim 49, comprising: (i) SEQ ID NO: 165 and (ii) any one of SEQ ID NOs: 154, 157, or 158.

52. A NARE comprising: (i) a sequence that is at least 90% identical to SEQ ID NO: 199; and (ii) a sequence that is at least 90% identical to SEQ ID NO: 193 or SEQ ID NO: 194.

53. The NARE of claim 52, comprising: (i) a sequence that is at least 95% identical to SEQ ID NO: 199; and (ii) a sequence that is at least 95% identical to SEQ ID NO: 193 or SEQ ID NO: 194.

54. The NARE of claim 52, comprising: (i) SEQ ID NO: 199; and (ii) SEQ ID NO: 193 or SEQ ID NO: 194.

55. A NARE comprising: (i) a sequence that is at least 90% identical to SEQ ID NO: 164; and (ii) a sequence that is least 90% identical to SEQ ID NO: 200.

56. The NARE of claim 55, comprising: (i) a sequence that is at least 95% identical to SEQ ID NO: 164; and (ii) a sequence that is least 95% identical to SEQ ID NO: 200.

57. The NARE of claim 55, comprising: (i) SEQ ID NO: 164; and (ii) SEQ ID NO: 200.

58. A NARE comprising: (i) a sequence that is at least 90% identical to SEQ ID NO: 189 and (ii) a sequence that is at least 90% identical to SEQ ID NO: 193 or SEQ ID NO: 194.

59. The NARE of claim 58, comprising: (i) a sequence that is at least 95% identical to SEQ ID NO: 189 and (ii) a sequence that is at least 95% identical to SEQ ID NO: 193 or SEQ ID NO: 194.

60. The NARE of claim 58, comprising: (i) SEQ ID NO: 189 and (ii) SEQ ID NO: 193 or SEQ ID NO: 194.

61. A NARE comprising: (i) a sequence that is at least 90% identical to SEQ ID NO: 202 or SEQ ID NO: 203; and (ii) a sequence that is at least 90% identical to SEQ ID NO: 195.

62. The NARE of claim 61, comprising: (i) a sequence that is at least 95% identical to SEQ ID NO: 202 or SEQ ID NO: 203; and (ii) a sequence that is at least 95% identical to SEQ ID NO: 195.

63. The NARE of claim 61, comprising: (i) SEQ ID NO: 202 or SEQ ID NO: 203; and (ii) SEQ ID NO: 195.

64. A nucleic acid regulatory element (NARE) comprising a sequence that is at least 90% identical to any one of SEQ ID NOs: 3-8, 11-69, 71-73, or 76-149.

65. The NARE of claim 64, comprising a sequence that is at least 95% identical to any one of SEQ ID NOs: 3-8, 11-69, 71-73, or 76-149.

66. The NARE of claim 64, comprising any one of SEQ ID NOs: 3-8, 11-69, 71-73, or 76-149.

67. The NARE of claim 64, comprising a sequence that is at least 90% identical to any one of SEQ ID NOs: 53, 80, 81, 132, 141, or 142.

68. The NARE of claim 67, comprising a sequence that is at least 95% identical to any one of SEQ ID NOs: 53, 80, 81, 132, 141, or 142.

69. The NARE of claim 67, comprising any one of SEQ ID NOs: 53, 80, 81, 132, 141, or 142.

70. An expression construct comprising the NARE of any one of the preceding claims and an operatively linked transgene.

71. The expression construct of claim 70, further comprising a polyadenylation sequence.

72. A vector comprising the expression construct of claim 70.

73. The vector of claim 70, wherein the vector is a non-viral vector.

74. The vector of claim 70, wherein the vector is a viral vector.

75. The vector of claim 74, wherein the vector is an adeno-associated virus (AAV) vector.

76. A vector comprising a nucleic acid sequence comprising (i) the expression construct of 70, and (ii) one or more inverted terminal repeats (ITR).

77. The vector of claim 76, wherein the nucleic acid sequence comprises a 5′ ITR and a 3′ ITR.

78. The vector of claim 77, wherein the 5′ ITR and a 3′ ITR are derived from adeno-associated virus (AAV) serotype AAV2.

79. A cell comprising the expression construct of claim 70 or the vector of any one of claims 72-78.

80. The cell of claim 79, wherein the cell is a liver cell.

81. A pharmaceutical composition comprising: (i) the expression construct of claim 70 or the vector of any one of claims 72-78 and (ii) a pharmaceutically acceptable excipient.

82. A method for expressing a transgene in a cell comprising the expression construct of claim 70 or the vector of any one of claims 72-78.

83. A method for regulating transgene expression in a cell comprising the expression construct of claim 70 or the vector of any one of claims 72-78.

84. The method of claim 82 or 83, wherein the cell is a liver cell.

85. A method of treating Wilson's disease in a subject in need thereof, the method comprising administering to the subject the expression construct of claim 70, the vector of any one of claims 72-78, or the pharmaceutical composition of claim 81.

86. The method of claim 85, wherein the subject has a mutation in the ATP7B gene.

87. The method of claim 86, wherein the subject is a human.
